(19) United States
(12) Patent Application Publication
(10) Pub. No.: US 2019/0262327 A1
(43) Pub. Date: Aug. 29, 2019

(54) COMBINATION OF A BACE INHIBITOR AND AN ANTIBODY OR ANTIGEN-BINDING FRAGMENT FOR THE TREATMENT OF A DISORDER ASSOCIATED WITH THE ACCUMULATION OF AMYLOID BETA

(71) Applicant: ASTRAZENECA AB, SÖDERTÄLJE (SE)

(72) Inventor: Conor Johnston, Cambridge (GB)

(21) Appl. No.: 16/085,180

(22) PCT Filed: Mar. 15, 2017
(86) PCT No.: PCT/EP2017/056180

§ 371 Date: Sep. 14, 2018

Related U.S. Application Data
(60) Provisional application No. 62/308,698, filed on Mar. 15, 2016.

(51) Int. Cl. A61K 31/4439; C07K 16/18; A61P 25/28; A61P 27/02; A61K 9/00

(52) U.S. Cl. CPC A61K 31/4439; C07K 16/18; A61P 25/28; A61K 2039/505; A61K 9/0053; A61K 9/0019; A61P 27/02

(57) ABSTRACT

The present disclosure provides for methods for treating a subject having a disease or disorder associated with the accumulation of amyloid beta, comprising administering to the subject a BACE inhibitor and an antibody or antigen-binding fragment that binds to amyloid beta n-42. In some embodiments, the disease or disorder is Alzheimer's Disease.

### CROSS-REFERENCE TO RELATED APPLICATION

This application claims the benefit of priority from U.S. Provisional Application No. 62/308,698, filed Mar. 15, 2016. The specification of the foregoing application is incorporated herein by reference in its entirety.

### BACKGROUND

Alzheimer's disease (AD) is a neurodegenerative disease that is characterized by worsening cognitive impairment and memory and that debilitates the patient's social and occupational functioning. This disease causes loss of nerve cells within the brain, which brings about cognitive difficulties with language and higher functioning, such as judgement, planning, organisation and reasoning, which can lead eventually to personality changes. The end stages of the disease are characterized by a complete loss of independent functioning.

Histologically, AD (sporadic and familial) is defined by the presence of intracellular neurofibrillary tangles (NFT's) and extracellular plaques. Plaques are aggregations of amyloid β peptide (Aβ) derived from the aberrant cleavage of the amyloid precursor protein (APP), a transmembrane protein found in neurons and astrocytes in the brain. Aβ deposits are also found in the blood vessels of AD patients. Cholinergic neurons are particularly vulnerable in AD, and the consequent neurotransmitter decline affects other neurotransmitter systems. Other symptoms of the disease include oxidative stress, inflammation and neuronal apoptosis (programmed cell death). In the AD patient, extensive neuronal cell death leads to cognitive decline and the eventual death of the patient. (Younkin, 1995; Borchelt et al., 1996; Selkoe, 1999). AD occurs three to five times more often among people with Down Syndrome than the general population. People with Down Syndrome are also more likely to develop AD at a younger age than other adults.

Current treatments are symptomatic only and are minimally effective and result in minor improvements in symptoms for only a limited duration of time. Overproduction or changes in Aβ levels are believed to be key events in the pathogenesis of sporadic and early onset AD, and, for this reason, Aβ has become a major target for the development of drugs designed to a) reduce its formation (Vassar et al., 1999), or b) activate mechanisms that accelerate its clearance from the brain.

The amyloid cascade hypothesis proposes that production of the Aβ peptide adversely affects neuron function, thereby leading to neuronal death and dementia in AD. Aβ is produced from the amyloid precursor protein (APP) which is cleaved sequentially by secretases to generate species of different lengths. Aβ ending at residue 42 is a minor component of the Aβ species produced by processing of APP. Other forms include Aβ1-40 and N-terminal truncates Aβn-40. However, Aβ ending at residue 42 is the most prone to aggregate and drives the deposition into anmyloid plaques. In addition to being more prone to aggregate, the Aβ1-42 peptide forms soluble low-n polymers (or oligomers) that have been shown to be toxic to neurons in culture. Unlike the larger conspicuous fibril deposits, oligomers are not detected in typical pathology assays. Oligomers having similar properties have been isolated from AD brains and these are more closely associated to disease progression than the plaques (Younkin, 1998; Walsh et al., 2005a; Walsh et al., 2005b). A number of isoforms of Aβ, including Aβ1-42, pGluAβ3-42, Aβ3-42 and 4-42, predominate in the Aβ brain, of which Aβ1-42 and Aβ4-42 are the main forms in the hippocampus and cortex of familial and sporadic AD (Portelius et al., 2010).

Several passive vaccination strategies have been previously investigated. The peripheral administration of antibodies against Aβ was sufficient to reduce amyloid burden (Bard et al., 2000). Despite relatively modest antibody serum levels achieved in these experiments, the passively administered antibodies were able to cross the blood-brain barrier and enter the central nervous system, decorate plaques and induce clearance of pre-existing amyloid. In a comparison between an Aβ1-40-specific antibody, an Aβ1-42-specific antibody and an antibody directed against residues 1-16 of Aβ, all antibodies were shown to reduce AB accumulation in mouse brain (Levites et al., 2006). Examples of representative useful anti-Aβ antibodies include those described in WO 2014/060444.

An additional attractive therapeutic target for treating diseases such as Alzheimer's Disease or Down syndrome is BACE inhibition. AB peptide results from the cleavage of APP at the C-terminus by one or more γ-secretases, and at the N-terminus by β-secretase enzyme, also known as aspartyl protease or Asp2 or Beta site APP Cleaving Enzyme (BACE), as part of the β-amyloidogenic pathway. BACE activity is correlated directly to the generation of AB peptide from APP (Sinha, et al, Nature, 1999, 402, 537-540), and studies increasingly indicate that the inhibition of BACE inhibits the production of AB peptide (Roberds, S. L., et al, Human Molecular Genetics, 2001, 10, 1317-1324). BACE is a membrane bound type 1 protein that is synthesized as a partially active proenzyme, and is abundantly expressed in brain tissue. It is thought to represent the major β-secretase activity, and is considered to be the rate-limiting step in the production of Aβ. Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof.

There is a need for novel therapies that both reduce Aβ formation (e.g., by inhibiting the enzymes responsible for its formation) and that activate mechanisms that accelerate Aβ clearance from the brain (e.g, by binding existing Aβ and targeting it for clearance).

### SUMMARY OF THE DISCLOSURE

In some embodiments, the disclosure provides for a method of treating a subject having a disease or disorder associated with the accumulation of Aβ3, comprising administering to the subject: a) a pharmaceutically effective amount of a BACE inhibitor, wherein the BACE inhibitor is:

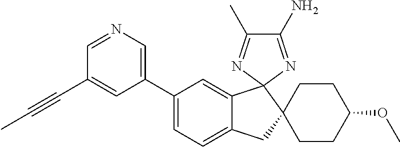

or a pharmaceutically acceptable salt thereof; and b) a pharmaceutically effective amount of an antibody or antigen-binding fragment comprising at least 1, 2, 3, 4, 5 or 6 CDRs from any one of Abet0380, Abet0342, Abet0369, Abet 0377 or Abet0382, or a germlined variant thereof. In some embodiments, the BACE inhibitor is a camsylate salt of:

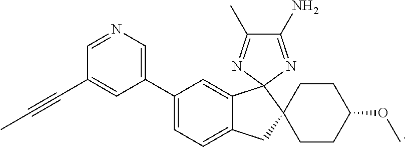

In some embodiments, the BACE inhibitor is:

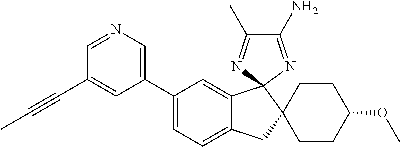 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof.
In some embodiments, the BACE inhibitor is a camsylate salt of

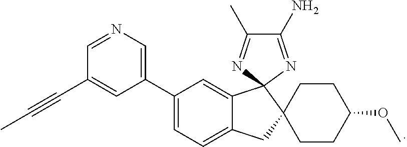 [SMILES: CC#Cc1cncc(-c2ccc3c(c2)[C@@]2(N=C(C)C(N)=N2)[C@]2(CC[C@H](OC)CC2)C3)c1]

In some embodiments, the BACE inhibitor is

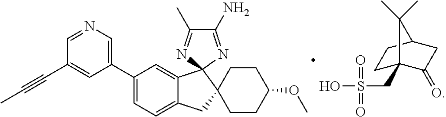 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

In some embodiments, the antibody or antigen-binding fragment for use in any of the methods disclosed herein comprises at least 1, 2, 3, 4, 5 or 6 CDRs of Abet0380, or a germlined variant thereof. In some embodiments, the antibody or antigen-binding fragment comprises the CDRs of the heavy chain of Abet0380, or a germlined variant thereof. In some embodiments, the antibody or antigen-binding fragment comprises the CDRs of the light chain of Abet0380, or a germlined variant thereof. In some embodiments, the antibody or antigen-binding fragment comprises a light chain variable (VL) domain and a heavy chain variable (VH) domain; wherein the VH domain comprises CDR1, CDR2 and CDR3 of the amino acid sequence set forth in SEQ ID NO: 524. In some embodiments, the antibody or antigen-binding fragment comprises a light chain variable (VL) domain and a heavy chain variable (VH) domain; wherein the VL domain comprises CDR1, CDR2 and CDR3 of the amino acid sequence set forth in SEQ ID NO: 533. In some embodiments, the VH domain comprises:

a VH CDR1 having the amino acid sequence of SEQ ID NO: 525;

a VH CDR2 having the amino acid sequence of SEQ ID NO: 526; and

a VH CDR3 having the amino acid sequence of SEQ ID NO: 527.

In some embodiments, the VL domain comprises:

a VL CDR1 having the amino acid sequence of SEQ ID NO: 534;

a VL CDR2 having the amino acid sequence of SEQ ID NO: 535; and

a VL CDR3 having the amino acid sequence of SEQ ID NO: 536.

In some embodiments, the VH domain comprises framework regions that are at least 90% identical to the amino acid sequences of SEQ ID NO: 528, SEQ ID NO: 529, SEQ ID NO: 530 and SEQ ID NO: 531. In some embodiments, the VH domain comprises framework regions having the amino acid sequences of SEQ ID NO: 528, SEQ ID NO: 529, SEQ ID NO: 530 and SEQ ID NO: 531. In some embodiments, the VL domain comprises framework regions that are at least 90% identical to the amino acid sequences of SEQ ID NO: 537, SEQ ID NO: 538, SEQ ID NO: 539 and SEQ ID NO: 540. In some embodiments, the VL domain comprises framework regions having the amino acid sequences of SEQ ID NO: 537, SEQ ID NO: 538, SEQ ID NO: 539 and SEQ ID NO: 540. In some embodiments, the VH domain comprises an amino acid sequence that is at least 90% identical to SEQ ID NO: 524. In some embodiments, the VL domain comprises an amino acid sequence that is at least 90% identical to SEQ ID NO: 533. In some embodiments, the VH domain comprises an amino acid sequence that is at least 95% identical to SEQ ID NO: 524. In some embodiments, the VL domain comprises an amino acid sequence that is at least 95% identical to SEQ ID NO: 533. In some embodiments, the VH domain comprises the amino acid sequence of SEQ ID NO: 524. In some embodiments, the VL domain comprises the amino acid sequence of SEQ ID NO: 533. In some embodiments, the antibody or antigen-binding fragment is an antigen-binding fragment. In some embodiments, the antigen-binding fragment is an scFv. In some embodiments, the antigen-binding fragment is a Fab′. In some embodiments, the antibody or antigen-binding fragment is an antibody. In some embodiments, the antibody is a monoclonal antibody. In some embodiments, the antibody is an IgG antibody. In some embodiments, the antibody is a human IgG1 or human IgG2. In some embodiments, the antibody is a human IgG1-TM. IgG1-YTE or IgG1-TM-YTE. In some embodiments, the antibody or antigen-binding fragment is humanized. In some embodiments, the antibody or antigen-binding fragment is human. In some embodiments, the antibody or antigen-binding fragment binds monomeric Aβ1-42 with a dissociation constant (KD) of 500 pM or less and either does not bind Aβ1-40 or binds Aβ1-40 with a KD greater than 1 mM. In some embodiments, the antibodies are useful because they bind more than one type of toxic or potentially toxic Aβ protein (e.g., Aβ1-42 and 3-pyro-42 amyloid beta). In some embodiments, the antibody or antigen-binding fragment binds amyloid beta 17-42 peptide (Aβ17-42) and anmyloid beta 29-42 peptide (Aβ29-42). In some embodiments, the antibody or antigen-binding fragment binds 3-pyro-42 amyloid beta peptide and 11-pyro-42 amyloid beta peptide. In some embodiments, the antibody or antigen-binding fragment binds amyloid beta 1-43 peptide (API-43).

In some embodiments, the disease or disorder to be treated using any of the methods disclosed herein is selected from the group consisting of: Alzheimer's disease, Down Syndrome, and/or macular degeneration. In some embodiments, the disease or disorder is Alzheimer's Disease. In some embodiments, the disease or disorder is Down Syndrome. In some embodiments, the disease or disorder is macular degeneration. In some embodiments, the BACE inhibitor and antibody or antigen-binding fragment are administered to the subject simultaneously. In some embodiments, the BACE inhibitor and antibody or antigen-binding fragment are administered separately. In some embodiments, the BACE inhibitor and antibody or antigen-binding fragment are in the same composition. In some embodiments, the BACE inhibitor is administered orally. In some embodiments, the antibody or antigen-binding fragment is administered intravenously. In some embodiments, the antibody or antigen-binding fragment is administered subcutaneously. In some embodiments, the subject is a human. In some embodiments, the method improves cognitive ability or prevents further cognitive impairment. In some embodiments, the method improves memory or prevents further dementia.

In some embodiments, the disclosure provides for a composition comprising a BACE inhibitor for use in combination with an antibody or antigen-binding fragment for treating a disease or disorder associated with Aβ accumulation, wherein the BACE inhibitor is:

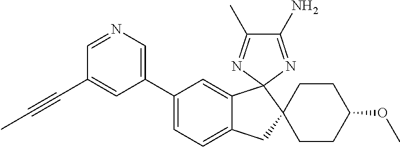

or a pharmaceutically acceptable salt thereof; and wherein the antibody or antigen-binding fragment comprises at least 1, 2, 3, 4, 5 or 6 CDRs from any one of Abet0380, Abet0342, Abet0369, Abet 0377 or Abet0382, or a germlined variant thereof. In some embodiments, the disclosure provides for a composition comprising an antibody or antigen-binding fragment for use in combination with a BACE inhibitor for treating a disease or disorder associated with Aβ accumulation, wherein the BACE inhibitor is:

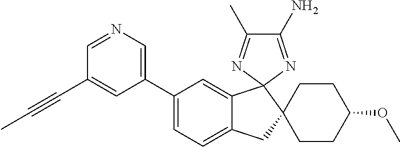

or a pharmaceutically acceptable salt thereof; and wherein the antibody or antigen-binding fragment comprises at least 1, 2, 3, 4, 5 or 6 CDRs from any one of Abet0380, Abet0342, Abet0369, Abet 0377 or Abet0382, or a germlined variant thereof. In some embodiments, the BACE inhibitor is

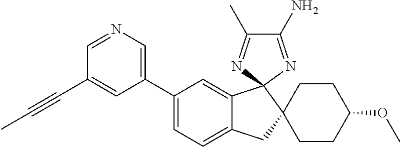 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof. In some embodiments, the BACE inhibitor is a camsylate salt of

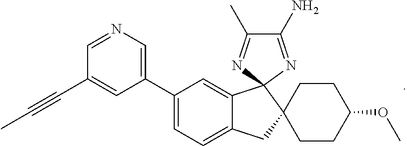 [SMILES: CC#Cc1cncc(-c2ccc3c(c2)[C@@]2(N=C(C)C(N)=N2)[C@]2(CC[C@H](OC)CC2)C3)c1]

In some embodiments, the BACE inhibitor is:

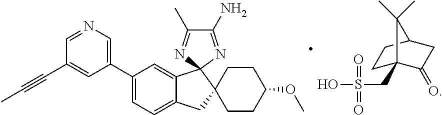 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

In some embodiments, the antibody or antigen-binding fragment comprises a light chain variable (VL) domain and a heavy chain variable (VH) domain; wherein the VH domain comprises CDR1, CDR2 and CDR3 of the amino acid sequence set forth in SEQ ID NO: 524. In some embodiments, the antibody or antigen-binding fragment comprises a light chain variable (VL) domain and a heavy chain variable (VH) domain; wherein the VL domain comprises CDR1, CDR2 and CDR3 of the amino acid sequence set forth in SEQ ID NO: 533. In some embodiments, wherein the VH domain comprises:

a VH CDR1 having the amino acid sequence of SEQ ID NO: 525;

a VH CDR2 having the amino acid sequence of SEQ ID NO: 526; and

a VH CDR3 having the amino acid sequence of SEQ ID NO: 527.

In some embodiments, the VL domain comprises:

a VL CDR1 having the amino acid sequence of SEQ ID NO: 534;

a VL CDR2 having the amino acid sequence of SEQ ID NO: 535; and

a VL CDR3 having the amino acid sequence of SEQ ID NO: 536.

In some embodiments, the disclosure provides for a kit comprising a BACE inhibitor and an antibody or antigen-binding fragment, wherein the BACE inhibitor is:

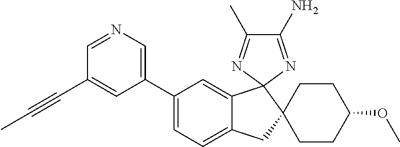

or a pharmaceutically acceptable salt thereof; and wherein the antibody or antigen-binding fragment comprises at least 1, 2, 3, 4, 5 or 6 CDRs from any one of Abet0380, Abet0342, Abet0369, Abet 0377 or Abet0382, or a germlined variant thereof. In some embodiments, the BACE inhibitor is

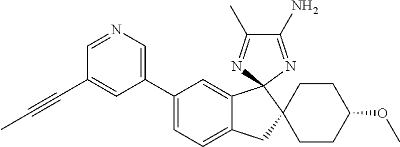 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof. In some embodiments, the BACE inhibitor is a camsylate salt of

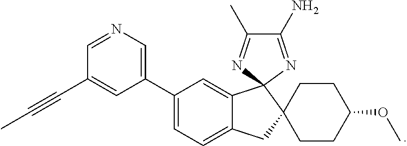 [SMILES: CC#Cc1cncc(-c2ccc3c(c2)[C@@]2(N=C(C)C(N)=N2)[C@]2(CC[C@H](OC)CC2)C3)c1]

In some embodiments, the BACE inhibitor is:

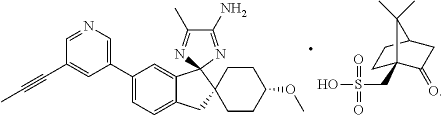 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

In some embodiments, the antibody or antigen-binding fragment comprises a light chain variable (VL) domain and a heavy chain variable (VH) domain; wherein the VH domain comprises CDR1, CDR2 and CDR3 of the amino acid sequence set forth in SEQ ID NO: 524. In some embodiments, the antibody or antigen-binding fragment comprises a light chain variable (VL) domain and a heavy chain variable (VH) domain; wherein the VL domain comprises CDR1, CDR2 and CDR3 of the amino acid sequence set forth in SEQ ID NO: 533. In some embodiments, the VH domain comprises:

a VH CDR1 having the amino acid sequence of SEQ ID NO: 525;

a VH CDR2 having the amino acid sequence of SEQ ID NO: 526; and

a VH CDR3 having the amino acid sequence of SEQ ID NO: 527. In some embodiments, the VL domain comprises:

a VL CDR1 having the amino acid sequence of SEQ ID NO: 534;

a VL CDR2 having the amino acid sequence of SEQ ID NO: 535; and

a VL CDR3 having the amino acid sequence of SEQ ID NO: 536.

### BRIEF DESCRIPTION OF THE DRAWINGS

FIG. 1 shows the inhibition of the formation of the human Amyloid beta 1-42 peptide and Abet0144-GL IgG1-TM complex by increasing concentrations of purified competitor scFv (∘). Four of the most potent scFv clones, Abet0369 (FIG. 1A), Abet0377 (FIG. 1B), Abet0380 (FIG. 1C) and Abet0382 (FIG. 1D) all show significant improvement in potency over the parent Abet0144-GL scFv sequence (●).

FIG. 2 shows the Surface Plasmon Resonance (BIAcore) traces for human Amyloid beta 1-42 peptide binding to immobilized Abet0380-GL IgG1-TM antibody at concentrations from 1024 nM (top trace) to 63 pM (bottom trace) peptide. Each trace is fitted to a 1:1 Langmuir model.

FIG. 3 shows the Surface Plasmon Resonance (BIAcore) traces for a series of Amyloid beta peptides binding to immobilized Abet0380-GL IgG1-TM antibody. There is clear binding to the biotinylated human Amyloid beta 1-42 peptide (top trace) and the unlabelled murine Amyloid beta 1-42 peptide (second trace). There is no discernable binding to biotinylated human Amyloid beta 1-40 peptide or unlabelled murine Amyloid beta 1-40 peptide (flat lines).

FIG. 4 shows sample images from the in vitro immunohistochemical staining of Abet0380-GL IgG1-TM. (A) A positive control antibody shows strong plaque recognition (score=4) on human A brain sections (ApoE genotype 3/3, Braak stage 6; 5 μg/ml antibody). (B) The Abet0380-GL IgG1-TM lead clone shows strong plaque recognition (score=3) on an adjacent brain section (10 μg/ml). (C) The same positive control antibody shows strong plaque recognition (score=4) on Tg2576 mouse brain sections (22 month old mice; 20 μg/ml antibody). (D) The Abet0380-GL IgG1-TM lead clone shows strong plaque recognition (score=4) on an adjacent mouse brain section (20 μg/ml).

FIG. 5 shows Western Blot analysis of Abeta 42 aggregate preparation and detection using the Abet0380-GL IgG1TM. (A) Abet0380-GL IgG1TM detection of non-photo cross-linked (non PICUP) Aβ42 aggregate. (B) Abet0380-GL IgG1TM detection of photo cross-linked Aβ342 aggregate (PICUP). Here we demonstrate that Abet0380-GL IgG1TM specifically recognises Aβ1-42 monomer and low n oligomer species up to and including pentamer.

FIG. 6 shows the dose-dependent reduction of the level of free Amyloid beta 1-42 peptide in the CSF (A), the increase of total Amyloid beta 1-42 peptide in brain tissue (B) and the unaffected levels of total Amyloid beta 1-40 peptide in brain tissue (C) by increasing doses of Abet0380-GL IgG1-TM antibody in Sprague-Dawley rats receiving repeated weekly doses over 14 days.

FIG. 7 shows sample images from the immunohistochemical analysis of binding of Abet0380-GL IgG1-TM to Amyloid beta plaques in vivo 168 hours after a peripheral dose to aged Tg2576 mice. A positive control antibody given at 30 mg/kg shows strong in vivo plaque recognition (A), whereas Abet0380-GL IgG1-TM given at 30(B) or 10(C) mg/kg does not show any in vivo plaque decoration.

FIG. 8 shows the specificity of Abet0380-GL IgG1-TM in competition binding experiments with a range of different concentrations (10 uM down to 0.17 nM) of a panel of full length, truncate and pyro human Abeta peptides (Abeta 1-42, Abeta 1-43, Abeta 1-16, Abeta 12-28, Abeta 17-42, Abeta pyro-3-42, or Abeta pyro-11-42). Key:

[symbol] Abeta 1-42

[symbol] Abeta 1-43

▾ Abeta 1-16

♦ Abeta 12-28

[symbol] Abeta 17-42

[symbol] Abeta Pyro-3-42

[symbol] Abeta Pyro 11-42

⋄ Vehicle 1 (DMSO)

● Vehicle 2 (NH₄OH)

The x-axis shows the concentration of Abeta peptide in log M, the y-axis shows % specific binding. Inhibition of Abet0380-GL IgG1-TM: N-terminal Biotin Abeta 1-42 binding was observed with Abeta 1-42, Abeta 1-43, Abeta 17-42. Abeta Pyro-3-42 & Abeta Pyro-11-42 with IC₅₀ values ranging from 10⁻⁸ to 10⁹ molar for this group. No inhibition of Abet0380-GL IgG1-TM: N-terminal Biotin Abeta 1-42 binding was observed with Abeta 1-16 or Abeta 12-28.

FIG. 9 shows the ability of antibody Abet0144-GL to sequester amyloid beta 1-42 in a normal rat PK-PD study. The x-axis shows vehicle or concentration of Abet0144-GL (10 mg/kg, or 40 mg/kg), the y-axis shows the concentration of total amyloid beta 1-42 in CSF in pg/ml. Free amyloid beta 1-42 in CSF was not significantly altered by either 10 or 40 mg/kg of Abet0144-GL (5 and 18% increase respectively when compared with vehicle). Total amyloid beta 1-42 in CSF was significantly increased by 38% at 10 mg/kg, and by 139% at 40 mg/kg. Total amyloid beta 1-42 in brain tissue was also significantly increased, by 16% and 50% at 10 and 40 mg/kg respectively. Data from this study in normal rats, demonstrate that Abet0144-GL had no significant effect on free amyloid beta 1-42 levels in CSF, whilst increasing total amyloid beta 1-42 levels in both CSF and brain.

### DETAILED DESCRIPTION

The present disclosure provides for methods of treating a subject in need thereof with any of the BACE inhibitors disclosed herein in combination with any of the antibodies or antigen-binding fragments disclosed herein. Kits and compositions are also provided.

### 1. Definitions

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs.

As used in this specification and the appended claims, the singular form “a”, “an” and “the” include plural referents unless the context clearly dictates otherwise.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

Throughout this specification, the word “comprise” or variations such as “comprises” or “comprising” will be understood to imply the inclusion of a stated integer or groups of integers but not the exclusion of any other integer or group of integers.

As used herein, the term “about,” when used in reference to a particular recited numerical value, means that the value may vary from the recited value by no more than 10%.

### 2. BACE Inhibitors

The present disclosure provides for the use of any of the BACE inhibitors disclosed herein in combination with any of the antibodies or antigen-binding fragments disclosed herein for treating a subject in need thereof.

In some embodiments, suitable BACE inhibitors for use in any of the methods described herein include those disclosed in U.S. Pat. Nos. 8,415,483, 8,865,911, and 9,248,129, and U.S. Patent application publication 2014/0031379, each of which is incorporated herein by reference.

In some embodiments, the BACE inhibitor suitable for use in the present disclosure is 4-methoxy-5′-methyl-6′-[5-(prop-1-yn-1-yl)pyridin-3-yl]-3′H-dispiro[cyclohexane-1,2′-indene-1′2″-imidazol]-4″-amine or a pharmaceutically acceptable salt thereof.

In some embodiments, the BACE inhibitor is (1r,4r)-4-methoxy-5″-methyl-6′-[5-(prop-1-yn-1-yl)pyridin-3-yl]-3′H-dispiro[cyclohexane-1,2-indene-1′2″-imidazol]-4″-amine:

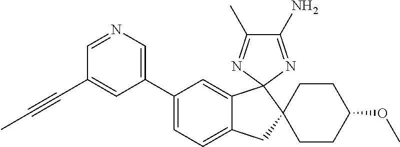

or a pharmaceutically acceptable salt thereof.

In some embodiments, the BACE inhibitor suitable for use in the present disclosure is (1r,1′R,4R)-4-methoxy-5″-methyl-6′-[5-(prop-1-yn-1-yl)pyridin-3-yl]-3′H-dispiro[cyclohexane-1,2′-indene-1′2″-imidazol]-4″-amine:

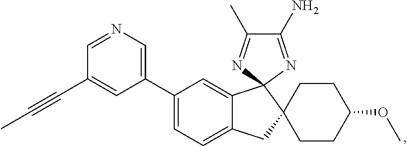 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the non-toxic salts or the quaternary ammonium salts of the parent compound formed, for example, from non-toxic inorganic or organic acids. For example, such non-toxic salts include those derived from inorganic acids such as hydrochloric acid. The pharmaceutically acceptable salts of the present disclosure can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two: generally, nonaqueous media like diethyl ether, ethyl acetate, ethanol, isopropanol, or acetonitrile are used.

In some embodiments, the BACE inhibitor suitable for use in the present disclosure is a camsylate salt of the compound: 4-methoxy-5″-methyl-6′-[5-(prop-1-yn-1-yl)pyridin-3-yl]-3′H-dispiro[cyclohexane-1,2′-indene-1′2″-imidazol]-4″-amine.

In some embodiments, the BACE inhibitor is a camsylate salt of (1r,4r)-4-methoxy-5″-methyl-6′-[5-(prop-1-yn-1-yl)pyridin-3-yl]-3′H-dispiro[cyclohexane-1,2′-indene-1′2″-imidazol]-4″-amine:

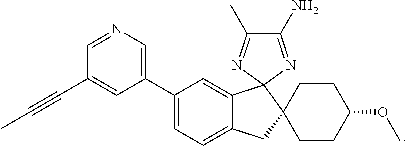

In some embodiments, the BACE inhibitor suitable for use in the present disclosure is a camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′2″-imidazol]-4″-amine:

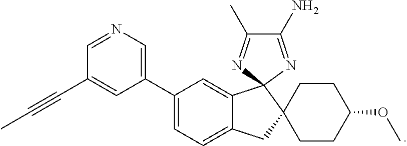 [SMILES: CC#Cc1cncc(-c2ccc3c(c2)[C@@]2(N=C(C)C(N)=N2)[C@]2(CC[C@H](OC)CC2)C3)c1]

In some embodiment, the BACE inhibitor is:

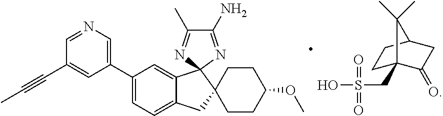 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

In some embodiments, the BACE inhibitor is:

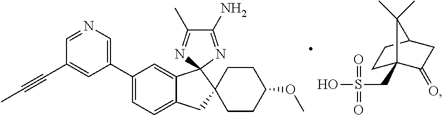 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

characterized in providing an X-ray powder diffraction (XRPD) pattern, exhibiting substantially the following peaks with d-spacing values as depicted in Table A:

TABLE A
Peaks identified on X-ray powder diffraction

| Corrected Angles | d-spacing (Å) | Relative intensity |
|---|---|---|
| 5.66 | 15.60 | vs |
| 7.72 | 11.44 | m |
| 8.11 | 10.89 | vw |
| 11.30 | 7.83 | m |
| 12.35 | 7.16 | s |
| 12.83 | 6.89 | m |
| 14.07 | 6.29 | w |
| 15.05 | 5.88 | w |
| 15.24 | 5.81 | m |
| 15.47 | 5.72 | m |
| 16.24 | 5.45 | w |
| 16.68 | 5.31 | w |
| 17.17 | 5.16 | m |
| 17.33 | 5.11 | w |
| 17.62 | 5.03 | vw |
| 17.84 | 4.97 | w |
| 18.13 | 4.89 | m |
| 19.71 | 4.50 | m |
| 20.18 | 4.40 | w |
| 20.77 | 4.27 | m |
| 21.12 | 4.20 | m |
| 21.67 | 4.10 | vw |
| 21.88 | 4.06 | vw |
| 22.09 | 4.02 | vw |
| 22.29 | 3.99 | w |
| 22.73 | 3.91 | w |
| 23.11 | 3.84 | vw |
| 23.63 | 3.76 | m |
| 24.50 | 3.63 | m |
| 26.18 | 3.40 | m |
| 26.54 | 3.36 | m |
| 27.72 | 3.22 | vw |
| 27.95 | 3.19 | vw |
| 28.80 | 3.10 | vw |
| 28.93 | 3.08 | vw |
| 29.71 | 3.00 | vw |
| 30.56 | 2.92 | vw |
| 31.14 | 2.87 | vw |
| 31.64 | 2.83 | vw |
| 31.74 | 2.82 | vw |
| 32.11 | 2.79 | vw |
| 32.84 | 2.72 | vw |
| 33.86 | 2.65 | vw |
| 34.30 | 2.61 | m |
| 36.78 | 2.44 | m |
| 37.49 | 2.40 | w |
| 40.23 | 2.24 | vw |
| 40.93 | 2.20 | vw |
| 41.32 | 2.18 | vw |
| 42.43 | 2.13 | w |
| 44.54 | 2.03 | vw |
| 46.29 | 1.96 | vw |
| 48.32 | 1.88 | vw |

In some embodiments, the BACE inhibitor is:

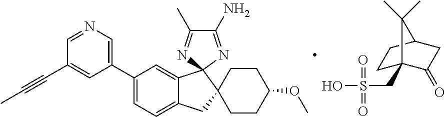 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

characterized in providing an X-ray powder diffraction pattern, exhibiting substantially the following very strong, strong and medium peaks with d-spacing values as depicted in Table B:

TABLE B
Peaks identified on X-ray powder diffraction

| Corrected Angles | d-spacing (Å) | Relative intensity |
|---|---|---|
| 5.66 | 15.60 | vs |
| 7.72 | 11.44 | m |
| 11.30 | 7.83 | m |
| 12.35 | 7.16 | s |
| 12.83 | 6.89 | m |
| 15.24 | 5.81 | m |
| 15.47 | 5.72 | m |
| 17.17 | 5.16 | m |
| 18.13 | 4.89 | m |
| 19.71 | 4.50 | m |
| 20.77 | 4.27 | m |
| 21.12 | 4.20 | m |
| 23.63 | 3.76 | m |
| 24.50 | 3.63 | m |
| 26.18 | 3.40 | m |
| 26.54 | 3.36 | m |
| 34.30 | 2.61 | m |
| 36.78 | 2.44 | m. |

As used herein the term camsylate salt also encompasses all solvates and co-crystals thereof.

Alternative salts of the BACE inhibitor suitable for use herein include the succinate the hydrochloric-, the phosphate-, the sulfate-, the fumarate- and the 1.5 naphthalenedisulfonate salt.

The present disclosure further includes all tautomeric forms of compounds of the disclosure. As used herein, “tautomer” means other structural isomers that exist in equilibrium resulting from the migration of a hydrogen atom. For example, keto-enol tautomerism where the resulting compound has the properties of both a ketone and an unsaturated alcohol. Other examples of tautomerism include 2H-imidazole-4-amine and its tautomer 1,2-dihydroimidazol-5-imine, and 2H-imidazol-4-thiol and its tautomer 1,2-dihydroimidazol-5-thione. It is understood that in compound representations throughout this description, only one of the possible tautomers of the compound is drawn or named.

Compounds of the disclosure further include hydrates and solvates.

Camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine:

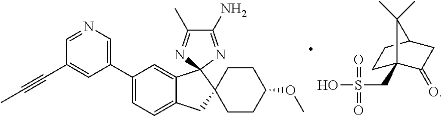 [SMILES: CC#Cc1cncc(-c2ccc3c(c2)[C@@]2(N=C(C)C(N)=N2)[C@]2(CC[C@H](OC)CC2)C3)c1 ; CC1(C)C2CC[C@@]1(CS(=O)(=O)O)C(=O)C2]

A camsylate salt of the compound (1r,1′R,4R)-4-methoxy-5″-methyl-6′-[5-(prop-1-yn-1-yl)pyridin-3-yl]-3′H-dispiro[cyclohexane-1,2′-indene-1′2″-imidazol]-4″-amine may be obtained by starting from a solution of (1 r,1′R,4R)-4-methoxy-5″-methyl-6′-[5-(prop-1-yn-1-yl)pyridin-3-yl]-3′H-dispiro[cyclohexane-1,2′-indene-1′2″-imidazol]-4″-amine in a suitable solvent, for example, 2-propanol, acetonitrile, or acetone or mixtures of these with water, followed by mixing the obtained solution with (1S)-(+)-10-camphorsulfonic acid directly or dissolved in a suitable solvent, for example, 2-propanol or water, at a temperature between room temperature and 80° C. Crystallization may be obtained by evaporation of solvent and/or by cooling the solution or directly as a salt reaction crystallization. Seed crystals may be used to start the crystallization. Seeds may be prepared from the batch itself by sampling a small volume of the solution and then rapidly cooling it to induce crystallization. Crystals are then added to the batch as seeds.

X-ray powder diffraction analysis (XRPD) may be performed on samples prepared according to standard methods, for example those described in Giacovazzo, C. et al (1995), Fundamentals of Crystallography, Oxford University Press; Jenkins, R. and Snyder, R. L. (1996), Introduction to X-Ray Powder Diffractometry, John Wiley & Sons, New York; Bunn, C. W. (1948). Chemical Crystallography, Clarendon Press, London; or Klug, H. P. & Alexander, L. E. (1974), X-ray Diffraction Procedures. John Wiley and Sons, New York. X-ray diffraction analyses were performed using a PANanlytical X'Pert PRO MPD diffractometer for 96 minutes from 1 to 60° 2θ. XRPD distance values may vary in the range ±2 on the last decimal place.

The relative intensities are derived from diffractograms measured with variable slits.

The measured relative intensities vs. the strongest peak are given as very strong (vs) above 50%, as strong (s) between 25 and 50%, as medium (m) between 10 and 25%, as weak (w) between 5 and 10% and as very weak (vw) under 5% relative peak height. It will be appreciated by a person skilled in the art that the XRPD intensities may vary between different samples and different sample preparations for a variety of reasons including preferred orientation. It will also be appreciated by a person skilled in the art that smaller shifts in the measured Angle and hence the d-spacing may occur for a variety of reasons including variation of sample surface level in the diffractometer.

### 3. Anti-Aβ Antibodies or Antigen-Binding Fragments

The present disclosure provides for the use of any of the antibodies or antigen-binding fragments disclosed herein in combination with any of the BACE inhibitors disclosed herein for treating a subject in need thereof.

In some embodiments, suitable antibodies or antigen-binding fragments for use in any of the methods described herein include those disclosed in WO 2014/060444 and US 2015/0299299, each of which is incorporated herein by reference.

As defined herein, an “antibody or antigen-binding fragment” comprises at least 1, 2, 3, 4, 5 or 6 CDRs of any one or more of the following antibody or antigen-binding fragments: Abet0380, Abet0319. Abet0321b. Abet0322b, Abet0323b, Abet0328, Abet0329, Abet0332, Abet0342, Abet0343, Abet0369, Abet0370, Abet0371, Abet0372, Abet0373, Abet0374, Abet0377, Abet0378, Abet0379, Abet0381, Abet0382 and Abet0383, or germlined variants thereof. In some embodiments, an “antibody or antigen-binding fragment” comprises at least 1, 2, 3, 4, 5 or 6 CDRs of any one or more of the following antibody or antigen-binding fragments: Abet0380, Abet0343, Abet0369, Abet0377 and Abet0382, or germlined variants thereof. In particular embodiments, an “antibody or antigen-binding fragment” comprises at least 1, 2, 3, 4, 5, or 6 CDRs of Abet0380, or a germlined variant thereof. Throughout the application, unless explicitly stated otherwise, CDRs are identified or defined using the Chothia. Kabat and/or IMGT system. When CDRs are indicated as being, as identified or as defined by the Chothia, Kabat or IMGT systems, what is meant is that the CDRs are in accordance with that system (e.g., the Chothia CDRs, Kabat CDRs or the IMGT CDRs). Any of these terms can be used to indicate whether the Chothia, Kabat or IMGT CDRs are being referred to.

By binding isoforms of Aβ peptide 1-42 and N-terminal truncates thereof (n-42) in plasma, brain and cerebrospinal fluid (CSF), an antibody or antigen-binding fragment according to the present disclosure may prevent accumulation or reverse the deposition of Aβn-42 (e.g., Aβ1-42, Aβ pyro 3-42, and/or Aβ4-42) isoforms within the brain and cerebrovasculature.

Antibodies or antigen-binding fragments according to the present disclosure may bind and precipitate soluble Aβ1-42 in blood plasma and/or in cerebrospinal fluid (CSF), thereby reducing the concentration of Aβ1-42 in the serum and/or CSF, respectively. These antibodies or antigen-binding fragments, when used in combination with any of the BACE inhibitors disclosed herein, represent a therapeutic approach for Alzheimer's disease and other conditions associated with amyloidosis.

In particular embodiments, antibodies or antigen-binding fragments of the disclosure are specific for the target epitope within Aβ 17-42 or within Aβ29-42, and bind this target epitope with high affinity relative to non-target epitopes, for example epitopes from Aβ1-40, thereby targeting the main toxic species linked with amyloid plaque formation. For example, an antibody or antigen-binding fragment may display a binding affinity for Aβ1-42 which is at least 10-fold, at least 100-fold, at least 1000-fold or at least 10,000-fold greater than for Aβ1-40. Thus, in some embodiments, the antibody or antigen-binding fragment is selective for binding Aβ1-42 over Aβ1-40. In some embodiments, the antibody or antigen-binding fragment may bind Aβ1-42 with a dissociation constant (KD) of 500 pM or less. In particular embodiments, the antibody or antigen-binding fragment shows no significant binding to Aβ1-40. In some embodiments, affinity and binding can be determined using surface plasmon resonance using monomeric Aβ peptide, as described in the Examples.

Binding to Aβ can also be measured in a homogenous time resolved fluorescence (HTRF™) assay, to determine whether the antibody is able to compete for binding to Aβ with a reference antibody molecule to the Aβ peptide, as described in the Examples.

An HTRF™ assay is a homogeneous assay technology that utilises fluorescence resonance energy transfer between a donor and acceptor fluorophore that are in close proximity.

Such assays can be used to measure macromolecular interactions by directly or indirectly coupling one of the molecules of interest to a donor fluorophore, europium (Eu3+) cryptate, and coupling the other molecule of interest to an acceptor fluorophore XL665. (a stable cross linked allophycocyanin). Excitation of the cryptate molecule (at 337 nm) results in fluorescence emission at 620 nm. The energy from this emission can be transferred to XL665 in close proximity to the cryptate, resulting in the emission of a specific long-lived fluorescence (at 665 nm) from the XL665. The specific signals of both the donor (at 620 nm) and the acceptor (at 665 nm) are measured, allowing the calculation of a 665/620 nm ratio that compensates for the presence of coloured compounds in the assay.

In some embodiments, an antibody or antigen-binding fragment according to the disclosure may compete for binding to Aβ1-42 and thus inhibit binding of the reference antibody in an HTFR™ competition assay with Aβ1-42, but not with Aβ1-40. In some embodiments, an antibody or antigen-binding fragment may show at least 70%, at least 75%, at least 80%, at least 85% or at least 90% inhibition of Abet0144GL for binding to Aβ1-42 in an HTRF™ assay.

Potency of inhibition of binding may be expressed as an IC₅₀ value, in nM unless otherwise stated. In functional assays. IC₅₀ is the concentration of an antibody molecule that reduces a biological response by 50% of its maximum. In ligand-binding studies, IC₅₀ is the concentration that reduces receptor binding by 50% of maximal specific binding level. IC₅₀ may be calculated by plotting % of maximal biological response as a function of the log of the antibody or antigen-binding fragment concentration, and using a software program, such as Prism (GraphPad) or Origin (Origin Labs) to fit a sigmoidal function to the data to generate IC₅₀ values. Suitable assays for measuring or determining potency are well known in the art.

In some embodiments, an antibody or antigen-binding fragment may have an IC₅₀ of 5 nM or less, e.g. 2 nM or less, e.g. 1 nM or less, in HTRF™ epitope competition assay with Abet0144-GL and Aβ1-42. Abet0144-GL is an antibody molecule having VH domain SEQ ID NO: 20 and VL domain SEQ ID NO: 29. It may be used in the assay in the same format as the antibody molecule to be tested, for example in scFv or IgG, e.g IgG1 format. Thus, IgG antibody molecules according to the disclosure may compete with Abet0144-GL IgG for binding to human Aβ1-42 in an HTRF epitope competition assay. Potency in such an assay may be less than 1 nM.

In particular embodiments, an antibody or antigen-binding fragment according to the disclosure may show specific binding for Aβ1-42 over Aβ1-40, as determined by an HTRF™ competition assay. In such an assay, Aβ1-40 may show no significant inhibition of the antibody or antigen-binding fragment binding to the Aβ1-42 peptide, e.g. it may show less than 20%, e.g less than 10% or less than 5%, inhibition in such an assay, and, in some embodiments, shows no significant inhibition in such an assay.

In some embodiments, antibodies or antigen-binding fragments according to the disclosure recognize an epitope within human Aβ17-42, more specifically within human Aβ29-42 and may also recognise their target epitope in Aβ from other species, e.g. mouse or rat. The potency of an antibody or antigen-binding fragment as calculated in an HTRF™ competition assay using Aβ1-42 from a first species (e.g human) may be compared with potency of the antibody or antigen-binding fragment in the same assay using Aβ1-42 from a second species (e.g. mouse Aβ1-42), in order to assess the extent of cross-reactivity of the antibody or antigen-binding fragment for API-42 of the two species. Potency, as determined by IC₅₀ measurements, may be within 10-fold or within 100-fold. As noted above, Abet0144GL may be used as a reference antibody in the HTRF™ competition assay. Antibodies or antigen-binding fragments described herein may have a greater potency in a human Aβ1-42 assay than in a non-human Aβ1-42 assay. In some embodiments, the antibodies are useful because they bind more than one type of toxic or potentially toxic Aβ protein species (e.g., Aβ1-42 and 3-pyro-42 amyloid beta).

In some embodiments, an antibody or antigen-binding fragment may comprise an antibody molecule or antigen-binding fragment thereof having one or more CDRs, e.g. a set of CDRs, within an antibody framework (i.e. an antibody antigen-binding domain). For example, an antibody molecule may comprise an antibody VH and/or VL domain. VH and VL domains of antibody molecules are also provided as part of the disclosure. As is well-known, VH and VL domains comprise complementarity determining regions, (“CDRs”), and framework regions, (“FWs”). A VH domain comprises a set of HCDRs and a VL domain comprises a set of LCDRs. An antibody molecule or antigen-binding fragment thereof may comprise an antibody VH domain comprising a VH CDR1, CDR2 and CDR3 and/or an antibody VL domain comprising a VL CDR1, CDR2 and CDR3. VH or VL domains may further comprise a framework. A VH or VL domain framework typically comprises four framework regions, FW1, FW2, FW3 and FW4, which are interspersed with CDRs in the following structure: FW1-CDR1-FW2-CDR2-FW3-CDR3-FW4.

Among the six short CDR sequences, the third CDR of the heavy chain (HCDR3) has greater size variability (greater diversity essentially due to the mechanisms of arrangement of the genes which give rise to it). It may be as short as 2 amino acids although the longest size known is 26. CDR length may also vary according to the length that can be accommodated by the particular underlying framework. Functionally, HCDR3 plays a role in part in the determination of the specificity of the antibody (Segal et al., PNAS, 71:4298-4302, 1974; Amit et al., Science, 233:747-753, 1986; Chothia et al., J. Mol. Biol., 196:901-917, 1987; Chothia et al., Nature, 342:877-883, 1989; Caton et al., J. Immunol., 144:1965-1968, 199; Sharon et al., PNAS, 87:4814-4817, 1990; Sharon et al., J. Immunol., 144:4863-4869, 1990; and Kabat et al., J. Immunol., 147:1709-1719, 1991).

Examples of antibody VH and VL domains, FWs and CDRs according to aspects of the disclosure are listed in Tables 3 and 4 and the appended sequence listing that forms part of the present disclosure. All VH and VL sequences, CDR sequences, sets of CDRs, sets of HCDRs and sets of LCDRs disclosed herein, as well as combinations of these elements, represent aspects of the disclosure. As described herein, a “set of CDRs” comprises CDR1, CDR2 and CDR3. Thus, a set of HCDRs refers to HCDR1, HCDR2 and HCDR3, and a set of LCDRs refers to LCDR1, LCDR2 and LCDR3.

In some embodiments, the antibody or antigen-binding fragment is an antibody. In some embodiments, the antibody is a monoclonal antibody.

In some embodiments, the antibody or antigen-binding fragment is an antigen-binding fragment. Antigen-binding fragments include, but are not limited to, molecules such as Fab, Fab′, Fab′-SH, scFv, Fv, dAb and Fd. Various other antibody molecules including one or more antibody antigen-binding sites have been engineered, including for example Fab2, Fab3, diabodies, triabodies, tetrabodies and minibodies. Antibody molecules and methods for their construction and use are described in Holliger & Hudson, Nature Biotechnology 23(9): 1126-1136 2005.

Through an extensive process of further optimisation and recombination of multiple libraries as described in the Examples, a panel of antibody clones was generated from Abet0144GL. These further optimized clones are designated Abet0380, Abet0319, Abet0321b, Abet0322b, Abet0323b, Abet0328, Abet0329, Abet0332, Abet0342, Abet0343, Abet0369, Abet0370, Abet0371, Abet0372, Abet0373, Abet0374, Abet0377. Abet0378, Abet0379, Abet0381, Abet0382 and Abet0383. Their CDR sequences and variable domain sequences are referenced in Tables 3 and 4 and set out in the sequence listing. Germlined VH and VL domain sequences Abet0380GL, Abet0377GL, Abet0343GL, Abet0369GL and Abet0382GL are shown in Table 6 and Table 7.

In some embodiments, the antibody or antigen-binding fragment comprises at least 1, 2, 3. 4, 5, or 6 of the CDRs of Abet0380. In some embodiments, the antibody or antigen-binding fragment comprises 1, 2, or 3 of the CDRs of the Abet0380 heavy chain. In some embodiments, the antibody or antigen-binding fragment comprises 1, 2 or 3 of the CDRs of the Abet0380 light chain. Tables 3 and 4 show that Abet0380 has a set of CDRs identified using the Kabat system, in which HCDR1 is SEQ ID NO: 525 (Kabat residues 31-35), HCDR2 is SEQ ID NO: 526 (Kabat residues 50-65), HCDR3 is SEQ ID NO: 527 (Kabat residues 95-102), LCDR1 is SEQ ID NO: 534 (Kabat residues 24-34), LCDR2 is SEQ ID NO: 535 (Kabat residues 50-56) and LCDR3 is SEQ ID NO: 536 (Kabat residues 89-97). The other optimized antibody clones are shown in Tables 3 and 4 in a similar was' and are also provided as aspects of the disclosure.

An antibody or antigen-binding fragment for human Aβn-42 in accordance with the disclosure may comprise one or more CDRs as described herein, e.g. a set of CDRs. The CDR or set of CDRs may be an Abet0380, Abet0319, Abet0321b, Abet0322b, Abet0323b, Abet0328. Abet0329, Abet0332. Abet0342, Abet0343, Abet0369, Abet0370, Abet0371, Abet0372, Abet0373, Abet0374, Abet0377, Abet0378, Abet0379, Abet0381, Abet0382 and Abet0383 set of CDRs, or a germlined version thereof, or may be a variant thereof as described herein.

In some embodiments:

HCDR1 may be 5 amino acids long, consisting of Kabat residues 31-35;
HCDR2 may be 17 amino acids long, consisting of Kabat residues 50-65:
HCDR3 may be 16 amino acids long, consisting of Kabat residues 95-102:
LCDR1 may be 11 amino acids long, consisting of Kabat residues 24-34;
LCDR2 may be 7 amino acids long, consisting of Kabat residues 50-56; and/or
LCDR3 may be 9 amino acids long, consisting of Kabat residues 89-97.

Antibodies or antigen-binding fragments may comprise a HCDR1, HCDR2 and/or HCDR3 and/or an LCDR1. LCDR2 and/or LCDR3 of any of the antibodies listed in Tables 3 and 4, e.g., a set of CDRs of any of the antibodies listed in Table 3 or 4. The antibody or antigen-binding fragment may comprise a set of VH CDRs of any one of these antibodies. Optionally, it may also comprise a set of VL CDRs of one of these antibodies. The VL CDRs may be from the same or a different antibody as the VH CDRs. A VH domain comprising a set of HCDRs of any of the antibodies listed in Tables 3, and/or a VL domain comprising a set of LCDRs of any of the antibodies listed in Tables 4, are also provided herein.

An antibody or antigen-binding fragment may comprise a set of H and/or L CDRs of any of the antibodies listed in Tables 3 and 4 with one or more amino acid mutations, e.g. up to 5, 10 or 15 mutations, within the disclosed set of H and/or L CDRs. A mutation may be an amino acid substitution, deletion or insertion. For example, an antibody molecule of the disclosure may comprise the set of H and/or L CDRs from any one of Abet0380, Abet0319, Abet0321b, Abet0322b, Abet0323b, Abet0328, Abet0329, Abet0332, Abet0342, Abet0343, Abet0369, Abet0370, Abet0371, Abet0372, Abet0373, Abet0374, Abet0377, Abet0378, Abet0379, Abet0381, Abet0382 and Abet0383, or a germlined version thereof, with one or two amino acid mutations, e.g. substitutions.

For example, the antibody or antigen-binding fragment may comprise

a VH domain comprising the Abet0380 or Abet0380GL set of HCDRs, wherein the amino acid sequences of the Abet0380 or Abet0380GL HCDRs are

### HCDR1 SEQ ID NO: 525,

### HCDR2 SEQ ID NO: 526, and

### HCDR3 SEQ ID NO: 527,

or comprising the Abet0380 set of HCDRs with one or two amino acid mutations, and

(ii) a VL domain comprising the Abet0380 or Abet0380GL set of LCDRs, wherein the amino acid sequences of the Abet0380 or Abet0380GL LCDRs are

### LCDR1 SEQ ID NO: 534

### LCDR2 SEQ ID NO: 535, and

### LCDR3 SEQ ID NO: 536,

or comprising the Abet0380 or Abet0380GL set of LCDRs with one or two amino acid mutations.

Mutations may potentially be made at any residue within the set of CDRs. In some embodiments, substitutions may be made at the positions substituted in any of Abet0380, Abet0319, Abet0321b, Abet0322b, Abet0323b, Abet0328, Abet0329, Abet0332, Abet0342, Abet0343, Abet0369, Abet0370, Abet0371, Abet0372, Abet0373, Abet0374, Abet0377, Abet0378, Abet0379, Abet0381, Abet0382 and Abet0383 compared with Abet0144GL, or at the positions substituted in any of Abet0319. Abet0321b, Abet0322b, Abet0323b, Abet0328, Abet0329, Abet0332, Abet0342, Abet0343, Abet0369, Abet0370, Abet0371, Abet0372, Abet0373, Abet0374, Abet0377, Abet0378, Abet0379, Abet0381. Abet0382 and Abet0383 compared with Abet0380, or germlined versions thereof, as shown in Tables 3 and 4.

For example, the one or more substitutions may be at one or more of the following Kabat residues:

26, 27, 28, 29 or 30 in VH FW1:

31, 32, 33, 34 or 35 in VH CDR1;

52a, 53, 54, 55, 56, 57, 58 or 62 in VH CDR2;

98, 99, 100 h or 102 in VH CDR3;

24, 25, 26, 27, 28, 29, 30, 31, 32, 33, 34 in VL CDR1:

89, 90, 92, 93, 94 or 97 in VL CDR3.

Examples of possible amino acid substitutions at particular Kabat residue positions are shown in Tables 10 and 12 for the VH domain and Tables 11 and 13 for the VL domain.

As described above, an antibody or antigen-binding fragment may comprise an antibody molecule having one or more CDRs, e.g. a set of CDRs, within an antibody framework. For example, one or more CDRs or a set of CDRs of an antibody may be grafted into a framework (e.g. human framework) to provide an antibody molecule. The framework regions may be of human germline gene segment sequences. Thus, the framework may be germlined, whereby one or more residues within the framework are changed to match the residues at the equivalent position in the most similar human germline framework. The skilled person can select a germline segment that is closest in sequence to the framework sequence of the antibody before germlining and test the affinity or activity of the antibodies to confirm that germlining does not significantly reduce antigen-binding or potency in assays described herein. Human germline gene segment sequences are known to those skilled in the art and can be accessed for example from the VBASE compilation (VBASE, MRC Centre of Protein Engineering, U K, 1997, http//mrc-cpe.cam.ac.uk).

An antibody or antigen-binding fragment as described herein may be an isolated human antibody molecule having a VH domain comprising a set of HCDRs in a human germline framework, e.g. Vh3-23 DP-47. Thus, the VH domain framework regions FW1, FW2 and/or FW3 may comprise framework regions of human germline gene segment Vh3-23 DP-47 and/or may be germlined by mutating framework residues to match the framework residues of this human germline gene segment. FW4 may comprise a framework region of a human germlinej segment.

The amino acid sequence of VH FW1 may be SEQ ID NO: 528. VH FW1 contains a series of residues at Kabat positions 26-30 that may contribute to antigen-binding and/or to be important for structural conformation of the CDR1 loop. Substitutions may be included in SEQ ID NO: 528, for example to synergize with the selected sequence of HCDR1. The one or more substitutions may optionally be selected from those shown in Table 10 or Table 12.

The amino acid sequence of VH FW2 may be SEQ ID NO: 529. The amino acid sequence of VH FW3 may be SEQ ID NO: 530. The amino acid sequence of VH FW4 may be SEQ ID NO: 531.

Normally the antibody or antigen-binding fragment also has a VL domain comprising a set of LCDRs, e.g. in a human germline framework, e.g. V lambda 23-3 DPL-23. Thus, the VL domain framework regions may comprise framework regions FW1, FW2 and/or FW3 of human germline gene segment V lambda 23-3 DPL-23 and/or may be germlined by mutating framework residues to match the framework residues of this human germline gene segment. FW4 may comprise a framework region of a human germlinej segment. The amino acid sequence of VL FW1 may be SEQ ID NO: 537. The amino acid sequence of VL FW2 may be SEQ ID NO: 538. The amino acid sequence of VL FW3 may be SEQ ID NO: 539. The amino acid sequence of VL FW4 may be SEQ ID NO: 540.

A germlined VH or VL domain may or may not be germlined at one or more Vernier residues, but is normally not.

For example, an antibody or antigen-binding fragment as described herein may comprise an amino acid sequence that is at least 85%, 90%, 91%, 92%, 93%, 94%, 95%, 96%, 97%, 98%, 99% or 100% identical to any one of the following set of heavy chain framework regions:

### FW1 SEQ ID NO: 528;

### FW2 SEQ ID NO: 529;

### FW3 SEQ ID NO: 530;

### FW4 SEQ ID NO: 531;

or may comprise the said set of heavy chain framework regions with 1, 2, 3, 4, 5, 6 or 7 amino acid mutations, e.g. substitutions.

An antibody or antigen-binding fragment as described herein may comprise an amino acid sequence that is at least 85%, 90%, 91%, 92%, 93%, 94%, 95%, 96%, 97%, 98%, 99% or 100% identical to any one of the following set of heavy chain framework regions:

### FW1 SEQ ID NO: 537;

### FW2 SEQ ID NO: 538;

### FW3 SEQ ID NO: 539;

### FW4 SEQ ID NO: 540:

or may comprise the said set of light chain framework regions with 1, 2, 3, 4, 5, or 6 amino acid mutations, e.g. substitutions.

A non-germlined antibody molecule has the same CDRs, but different frameworks, compared to a germlined antibody molecule. Of the antibody sequences shown herein in the appended sequence listing, sequences of Abet0144-GL. Abet0380-GL, Abet0377-GL, Abet0343-GL, Abet0369-GL, and Abet0382-GL are germlined. Germlined antibodies of other antibody molecules whose sequences are disclosed herein may be produced by germlining framework regions of their VH and VL domain sequences, optionally to Vh3-23 DP-47 in the VH domain and V lambda 23-3 DPL-23 in the VL domain.

Typically, a VH domain is paired with a VL domain to provide an antibody antigen-binding site, although as discussed above a VH or VL domain alone may be used to bind antigen. For example, the Abet0380-GL VH domain (SEQ ID NO: 524) may be paired with the Abet0380-GL VL domain (SEQ ID NO: 533), so that an antibody antigen-binding site is formed comprising both the Abet0380-GL VH and VL domains. Analogous embodiments are provided for the VH and VL domains of the other antibodies disclosed herein. In other embodiments, the Abet0380-GL VH is paired with a VL domain other than the Abet0380-GL VL. Light-chain promiscuity is well established in the art. Again, analogous embodiments are provided by the disclosure for the other VH and VL domains disclosed herein. Thus, a VH domain comprising the VH CDRs or the germlined VH domain sequence of any of Abet0319, Abet0321b, Abet0322b, Abet0323b, Abet0328, Abet0329, Abet0332, Abet0342, Abet0343, Abet0369, Abet0370, Abet0371, Abet0372, Abet0373, Abet0374, Abet0377, Abet0378, Abet0379, Abet0380, Abet0381, Abet0382 and Abet0383 may be paired with a VL domain comprising the VL CDRs or germlined VL domain from a different antibody e.g. the VH and VL domains may be from different antibodies selected from Abet0319, Abet0321 b, Abet0322b, Abet0323b, Abet0328, Abet0329. Abet0332, Abet0342, Abet0343, Abet0369, Abet0370, Abet0371, Abet0372, Abet0373, Abet0374, Abet0377, Abet0378, Abet0379, Abet0380, Abet0381, Abet0382 and Abet0383.

An antibody or antigen-binding fragment may comprise

(i) a VH domain amino acid sequence as shown in Table 14 or in the appended sequence listing for any of Abet0380, Abet0343, Abet0369, Abet0377 and Abet0382, or a germlined version thereof,

or comprising that amino acid sequence with one or two amino acid mutations; and

(ii) a VL domain amino acid sequence as shown in Table 14 or in the appended sequence listing for any of Abet0380, Abet0343, Abet0369, Abet0377 and Abet0382, or a germlined version thereof,

or comprising that amino acid sequence with one or two amino acid mutations.

An antibody molecule may comprise:

(i) a VH domain having an amino acid sequence at least 90%, 95% or 98% identical to a VH domain amino acid sequence shown in Table 14 for any of Abet0380, Abet0343, Abet0369, Abet0377 and Abet0382, or a germlined version thereof; and
(ii) a VL domain having an amino acid sequence at least 90%, 95% or 98% identical to a VL domain amino acid sequence shown in Table 14 for any of Abet0380, Abet0343. Abet0369, Abet0377 and Abet0382, or a germlined version thereof.

In some embodiments, an antibody or antigen-binding fragment may comprise a VH domain and a VL domain at least 90%, 95% or 98% identical with the VH domain and VL domain, respectively, of any of Abet0380, Abet0343, Abet0369, Abet0377 and Abet0382, or a germlined version thereof.

In some embodiments, an antibody or antigen-binding fragment comprises a VH domain, wherein the VH domain comprises:

a VH CDR1 having the amino acid sequence of SEQ ID NO: 525;

a VH CDR2 having the amino acid sequence of SEQ ID NO: 526; and

a VH CDR3 having the amino acid sequence of SEQ ID NO: 527.

In some embodiments, an antibody or antigen-binding fragment comprises a VH domain, wherein the VL domain comprises:

a VL CDR1 having the amino acid sequence of SEQ ID NO: 534;

a VL CDR2 having the amino acid sequence of SEQ ID NO: 535; and

a VL CDR3 having the amino acid sequence of SEQ ID NO: 536.

In some embodiments, an antibody or antigen-binding fragment comprises a VH domain and a VL domain, wherein the VH domain comprises:

a VH CDR1 having the amino acid sequence of SEQ ID NO: 525:

a VH CDR2 having the amino acid sequence of SEQ ID NO: 526; and

a VH CDR3 having the amino acid sequence of SEQ ID NO: 527; and wherein the VL domain comprises:

a VL CDR1 having the amino acid sequence of SEQ ID NO: 534;

a VL CDR2 having the amino acid sequence of SEQ ID NO: 535; and

a VL CDR3 having the amino acid sequence of SEQ ID NO: 536.

In some embodiments, the VH domain comprises framework regions that are at least 85%, 90%, 91%, 92%, 93%, 94%, 95%, 96%, 97%, 98%, 99% or 100% identical to the amino acid sequences of any one or more of SEQ ID NO: 528, SEQ ID NO: 529, SEQ ID NO: 530 and SEQ ID NO: 531. In some embodiments, the VL domain comprises framework regions that are at least 85%, 90%, 91%, 92%, 93%, 94%, 95%, 96%, 97%, 98%, 99% or 100% identical to the amino acid sequences of any one or more of SEQ ID NO: 537, SEQ ID NO: 538, SEQ ID NO: 539 and SEQ ID NO: 540. In some embodiments, the VH domain comprises an amino acid sequence that is at least 85%, 90%, 91%, 92%, 93%, 94%, 95%, 96%, 97%, 98%, 99% or 100% identical to SEQ ID NO: 524. In some embodiments, the VL domain comprises an amino acid sequence that is at least 85%, 90%, 91%, 92%, 93%, 94%, 95%, 96%, 97%, 98%, 99% or 100% identical to SEQ ID NO: 533.

In some embodiments, an antibody molecule or antigen-binding fragment comprise an antibody constant region. An antibody molecule may be a whole antibody such as an IgG, i.e. an IgG1, IgG2, or IgG4, or may be an antibody fragment or derivative as described below. Antibody molecules can also have other formats, e.g. IgG1 with YTE (Dall'Acqua et al. (2002) J. Immunology, 169: 5171-5180; Dall'Acqua et al. (2006) J Biol. Chem. 281(33):23514-24) and/or TM mutations (Oganesyan et al. (2008) Acta Cryst D64:700-4) in the Fc region.

The disclosure provides an antibody or antigen-binding fragment of the present disclosure with a variant Fc region, wherein the variant comprises a phenylalanine (F) residue at position 234, a phenylalanine (F) residue or a glutamic acid (E) residue at position 235 and a serine (S) residue at position 331, as numbered by the EU index as set forth in Kabat. Such mutation combinations are hereinafter referred to as the triple mutant (TM).

An antibody or antigen-binding fragment as described herein may comprise a CDR, VH domain, VL domain, antibody-antigen-binding site or antibody molecule which is encoded by the nucleic acid sequences and/or the vector of any of:

(i) deposit accession number NCIMB 41889 (Abet0007);

(ii) deposit accession number NCIMB 41890 (Abet0380-GL);

(iii) deposit accession number NCIMB 41891 (Abet0144-GL);

(iv) deposit accession number NCIMB 41892 (Abet0377-GL).

An antibody or antigen-binding fragment as described herein may be produced or producible from the nucleic acid, vector or cell line of deposit accession number NCIMB 41889, 41890, 41891 or 41892. For example, an antibody or antigen-binding fragment may be produced by expression of the nucleic acid or vector of the cell line of deposit accession number NCIMB 41890. The nucleic acid or vector may be expressed using any convenient expression system. Alternatively, the antibody or antigen-binding fragment may be expressed by the cell line of deposit accession number NCIMB 41889, 41890, 41891 or 41892.

Aspects of the disclosure also provide nucleic acids encoding the VH and/or VL domains, which is contained in the cell line of accession number 41889, 41890, 41891 or 41892; a vector comprising said nucleic acid, which is contained in the cell line of accession number 41889, 41890, 41891 or 41892; and the cells or cell line of accession number 41889, 41890, 41891 or 41892.

An antibody or antigen-binding fragment according to the present disclosure may comprise an antibody antigen-binding site or antibody molecule that competes for binding to human Aβ1-42 with any antibody molecule encoded by nucleic acid deposited under accession number 41889, 41890, 41891 or 41892, or with an antibody molecule that comprises the VH domain and VL domain amino acid sequences of Abet007. Abet0380-GL, Abet0144-GL or Abet0377-GL as set out in the appended sequence listing.

An antibody or antigen-binding fragment normally comprises a molecule having an antigen-binding site. For example, an antibody or antigen-binding fragment may be an antibody molecule or a non-antibody protein that comprises an antigen-binding site.

It is possible to take monoclonal and other antibodies and use techniques of recombinant DNA technology to produce other antibodies or chimeric molecules that bind the target antigen. Such techniques may involve introducing DNA encoding the immunoglobulin variable region, or the CDRs, of an antibody to the constant regions, or constant regions plus framework regions, of a different immunoglobulin. See, for instance, EP-A-184187, GB 2188638A or EP-A-239400, and a large body of subsequent literature. A hybridoma or other cell producing an antibody may be subject to genetic mutation or other changes, which may or may not alter the binding specificity of antibodies produced.

Further techniques available in the art of antibody engineering have made it possible to isolate human and humanized antibodies. For example, human hybridomas can be made as described by Kontermann & Dubel [Kontermann, R & Dubel, S, Antibody Engineering, Springer-Verlag New York, LLC; 2001, ISBN: 3540413545].

Transgenic mice in which the mouse antibody genes are inactivated and functionally replaced with human antibody genes while leaving intact other components of the mouse immune system, can be used for isolating human antibodies [Mendez, M. et al. (1997) Nature Genet, 15(2): 146-156]. Humanized antibodies can be produced using techniques known in the art such as those disclosed in for example WO91/09967, U.S. Pat. No. 5,585,089, EP592106, U.S. Pat. No. 565,332 and WO93/17105. Further, WO2004/006955 describes methods for humanising antibodies, based on selecting variable region framework sequences from human antibody genes by comparing canonical CDR structure types for CDR sequences of the variable region of a non-human antibody to canonical CDR structure types for corresponding CDRs from a library of human antibody sequences, e.g. germline antibody gene segments. Human antibody variable regions having similar canonical CDR structure types to the non-human CDRs form a subset of member human antibody sequences from which to select human framework sequences. The subset members may be further ranked by amino acid similarity between the human and the non-human CDR sequences. In the method of WO2004/006955, top ranking human sequences are selected to provide the framework sequences for constructing a chimeric antibody that functionally replaces human CDR sequences with the non-human CDR counterparts using the selected subset member human frameworks, thereby providing a humanized antibody of high affinity and low immunogenicity without need for comparing framework sequences between the non-human and human antibodies. Chimeric antibodies made according to the method are also disclosed.

Synthetic antibody molecules may be created by expression from genes generated by means of oligonucleotides synthesized and assembled within suitable expression vectors, for example as described by Knappik et al. [Knappik et al. J. Mol. Biol. (2000) 296, 57-86] or Krebs et al. [Krebs et al. Journal of Immunological Methods 254 2001 67-84].

It has been shown that fragments of a whole antibody (which may be referred to herein as antibody fragments or antigen-binding fragments) can perform the function of binding antigens. Examples of antigen-binding fragments are (i) the Fab fragment consisting of VL, VH, CL and CH1 domains: (ii) the Fd fragment consisting of the VH and CH1 domains; (iii) the Fv fragment consisting of the VL and VH domains of a single antibody; (iv) the dAb fragment [Ward, E. S. et al., Nature 341, 544-546 (1989); McCafferty et al. (1990) Nature, 348, 552-554; Holt et al. (2003) Trends in Biotechnology 21, 484-490], which consists of a VH or a VL domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen-binding site [Bird et al., Science, 242, 423-426, 1988; Huston et al., PNAS USA, 85, 5879-5883, 1988]; (viii) bispecific single chain Fv dimers (PCT/US92/09965) and (ix) “diabodies”, multivalent or multispecific fragments constructed by gene fusion (WO94/13804; Holliger, P. et al., Proc. Natl. Acad. Sci. USA 90 6444-6448, 1993). Fv, scFv or diabody molecules may be stabilized by the incorporation of disulphide bridges linking the VH and VL domains [Reiter, Y. et al., Nature Biotech, 14, 1239-1245, 1996]. Minibodies comprising a scFv joined to a CH3 domain may also be made [Hu, S. et al., Cancer Res., 56, 3055-3061, 1996]. Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

Antigen-binding fragments of the disclosure can be obtained starting from any of the antibodies listed herein, by methods such as digestion by enzymes e.g. pepsin or papain and/or by cleavage of the disulfide bridges by chemical reduction. In another manner, the antigen-binding fragments comprised in the present disclosure can be obtained by techniques of genetic recombination likewise well known to the person skilled in the art or else by peptide synthesis by means of, for example, automatic peptide synthesizers, such as those supplied by the company Applied Biosystems, etc., or by nucleic acid synthesis and expression.

Functional antibody fragments according to the present disclosure include any functional fragment whose half-life is increased by a chemical modification, especially by PEGylation, or by incorporation in a liposome.

In some embodiments, the antibody or antigen-binding fragment is a dAb. A dAb (domain antibody) is a small monomeric antigen-binding fragment of an antibody, namely the variable region of an antibody heavy or light chain. VH dAbs occur naturally in camelids (e.g., camel, llama) and may be produced by immunizing a camelid with a target antigen, isolating antigen-specific B cells and directly cloning dAb genes from individual B cells, dAbs are also producible in cell culture.

Various methods are available in the art for obtaining antibodies. The antibodies may be monoclonal antibodies, especially of human, murine, chimeric or humanized origin, which can be obtained according to the standard methods well known to the person skilled in the art.

In general, for the preparation of monoclonal antibodies or their functional fragments, especially of murine origin, it is possible to refer to techniques which are described in particular in the manual “Antibodies” [Harlow and Lane, Antibodies: A Laboratory Manual, Cold Spring Harbor Laboratory, Cold Spring Harbor N.Y., pp. 726, 1988] or to the technique of preparation from hybridomas described by Köhler and Milstein [Köhler and Milstein, Nature, 256:495-497, 1975].

In some embodiments, monoclonal antibodies can be obtained, for example, from an animal cell immunized with human Aβ1-42, or one of its fragments containing the epitope recognized by said monoclonal antibodies, e.g. Aβ17-42.

WO 2006/072620 describes engineering of antigen-binding sites in structural (non-CDR) loops extending between beta strands of immunoglobulin domains. An antigen-binding site may be engineered in a region of an antibody molecule separate from the natural location of the CDRs, e.g. in a framework region of a VH or VL domain, or in an antibody constant domain, e.g., CH1 and/or CH3. An antigen-binding site engineered in a structural region may be additional to, or instead of, an antigen-binding site formed by sets of CDRs of a VH and VL domain. Where multiple antigen-binding sites are present in an antibody molecule, they may bind the same antigen (target antigen), thereby increasing valency of the antibody or antigen-binding fragment. Alternatively, multiple antigen-binding sites may bind different antigens (the target antigen and one or more another antigen), and this may be used to add effector functions, prolong half-life or improve in vivo delivery of the antibody molecule.

Heterogeneous preparations comprising antibody molecules also form part of the disclosure. For example, such preparations may be mixtures of antibodies with full-length heavy chains and heavy chains lacking the C-terminal lysine, with various degrees of glycosylation and/or with derivatized amino acids, such as cyclization of an N-terminal glutamic acid to form a pyroglutamic acid residue.

As noted above, an antibody or antigen-binding fragment in accordance with the present disclosure binds human Aβ1-42. As described herein, antibodies or antigen-binding fragments of the present disclosure may be optimized for affinity and/or for potency of inhibition in an HTRF™ competition assay. Generally, potency optimization involves mutating the sequence of a selected antibody or antigen-binding fragment (normally the variable domain sequence of an antibody) to generate a library of antibodies or antigen-binding fragments, which are then assayed for potency and the more potent antibodies or antigen-binding fragments are selected. Thus selected “potency-optimized” antibodies or antigen-binding fragments tend to have a higher potency than the antibody or antigen-binding fragment from which the library was generated. Nevertheless, high potency antibodies or antigen-binding fragments may also be obtained without optimization, for example a high potency antibody or antigen-binding fragment may be obtained directly from an initial screen. Assays and potencies are described in more detail elsewhere herein. The skilled person can thus generate antibodies or antigen-binding fragments having high potency.

In some embodiments, an antibody or antigen-binding fragment may bind human Aβ1-42 with the affinity of any of the antibodies listed in Tables 3 and 4, e.g. scFv, IgG2, IgG1TM or IgG1, or with an affinity that is better. Representative antibody binding affinities are shown in Table 5. Binding affinity and neutralization potency of different antibodies or antigen-binding fragments can be compared under appropriate conditions.

Variants of the VH and VL domains and CDRs described herein, including those for which amino acid sequences are set out herein, and which can be employed in antibodies or antigen-binding fragments for Aβ1-42 can be obtained by means of methods of sequence alteration or mutation and screening for antigen antibodies or antigen-binding fragments with desired characteristics. Examples of desired characteristics include but are not limited to: increased binding affinity for antigen relative to known antibodies which are specific for the antigen, increased neutralization of an antigen activity relative to known antibodies which are specific for the antigen if the activity is known specified competitive ability with a known antibody or ligand to the antigen at a specific molar ratio, ability to immunoprecipitate complex, ability to bind to a specified epitope: a linear epitope, e.g., peptide sequence identified using peptide-binding scan as described herein, e.g., using peptides screened in linear and/or constrained conformation, or a conformational epitope, formed by non-continuous residues; and ability to modulate a new biological activity of human Aβ1-42. Such methods are also provided herein.

Variants of antibody molecules disclosed herein may be produced and used in the present disclosure. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of antibodies can be derived using well-known mathematical techniques, such as statistical regression, pattern recognition and classification [see for example Norman et al. Applied Regression Analysis. Wiley-Interscience; 3^{rd} edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H. et al Data Mining: Practical Machine Learning Tools and Techniques with Java Implementations. Morgan Kaufmann; (Oct. 11, 1999), ISBN: 1558605525: Denison David G. T. (Editor) et al Bayesian Methods for Nonlinear Classification and Regression (Wiley Series in Probability and Statistics). John Wiley & Sons: (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of antibodies can be derived from empirical and theoretical models (for example, analysis of likely contact residues or calculated physicochemical property) of antibody sequence, functional and three-dimensional structures and these properties can be considered individually and in combination.

In some embodiments, an antigen-binding site composed of a VH domain and a VL domain is typically formed by six loops of polypeptide: three from the light chain variable domain (VL) and three from the heavy chain variable domain (VH). Analysis of antibodies of known atomic structure has elucidated relationships between the sequence and three-dimensional structure of antibody combining sites [Chothia C. et al. Journal Molecular Biology (1992) 227, 799-817: Al-Lazikani, et al. Journal Molecular Biology (1997) 273(4), 927-948]. These relationships imply that, except for the third region (loop) in VH domains, binding site loops have one of a small number of main-chain conformations: canonical structures. The canonical structure formed in a particular loop has been shown to be determined by its size and the presence of certain residues at key sites in both the loop and in framework regions.

This study of sequence-structure relationship can be used for prediction of those residues in an antibody of known sequence, but of an unknown three-dimensional structure, which are important in maintaining the three-dimensional structure of its CDR loops and hence maintain binding specificity. These predictions can be backed up by comparison of the predictions to the output from lead optimization experiments. In a structural approach, a model can be created of the antibody molecule [Chothia, et al. Science, 223, 755-758 (1986)] using any freely available or commercial package, such as WAM [Whitelegg, N. R. u. and Rees, A. R (2000). Prot. Eng., 12, 815-824]. A protein visualisation and analysis software package, such as Insight II (Accelrys, Inc.) or Deep View [Guex, N. and Peitsch, M. C. Electrophoresis (1997) 18, 2714-2723] may then be used to evaluate possible substitutions at each position in the CDR. This information may then be used to make substitutions likely to have a minimal or beneficial effect on activity.

The techniques required to make substitutions within amino acid sequences of CDRs, antibody VH or VL domains and antibodies or antigen-binding fragments generally are available in the art. Variant sequences may be made, with substitutions that may or may not be predicted to have a minimal or beneficial effect on activity, and tested for ability to bind Aβ1-42 and/or for any other desired property.

Variable domain amino acid sequence variants of any of the VH and VL domains whose sequences are specifically disclosed herein may be employed in accordance with the present disclosure, as discussed.

As described above, aspects of the disclosure provide an antibody or antigen-binding fragment, such as an antibody molecule, comprising a VH domain that has at least 75%/0, at least 80%, at least 85%, at least 90%, at least 93%, at least 94%, at least 95%, at least 96%, at least 97%, at least 98% or at least 99% amino acid sequence identity with a VH domain of any of the antibodies listed in Table 8, for which VH domain sequences are shown in the appended sequence listing below, and/or comprising a VL domain that has at least 75%, at least 80%, at least 85%, at least 90%, at least 93%, at least 94%, at least 95%, at least 96%, at least 97%, at least 98% or at least 99% amino acid sequence identity with a VL domain of any of the antibodies listed in Table 9, for which VL domain sequences are shown in the appended sequence listing.

Aspects of the disclosure provide an antibody or antigen-binding fragment, such as an antibody molecule, comprising a VH domain having a set of VH CDRs that have at least 75%, at least 80%, at least 85%, at least 906, at least 93%, at least 94%, at least 95%, at least 96%, at least 97%, at least 98% or at least 99% amino acid sequence identity with the set of VH CDRs of any of the antibodies listed herein, for which VH CDR sequences are shown herein; and/or comprising a VL domain having a set of VL CDRs that have at that has at least 75%, at least 80%, at least 85%, at least 90%, at least 93%, at least 94%, at least 95%, at least 96%, at least 97%, at least 98% or at least 99% amino acid sequence identity with the set of VL CDRs of any of the antibodies listed herein, for which the VL CDR sequences are shown in herein.

Algorithms that can be used to calculate % identity of two amino acid sequences include e.g. BLAST [Altschul et al. (1990) J. Mol. Biol. 215: 405-410], FASTA [Pearson and Lipman (1988) PNAS USA 85: 2444-2448], or the Smith-Waterman algorithm [Smith and Waterman (1981) J. Mol Biol. 147: 195-197] e.g., employing default parameters.

Particular variable domains may include one or more amino acid sequence mutations (substitution, deletion, and/or insertion of an amino acid residue), and less than about 15 14, 13, 12, 11, 10, 9, 8, 7, 6, 5, 4, 3 or 2 mutations.

Mutations may be made in one or more framework regions and/or one or more CDRs. The mutations normally do not result in loss of function, so an antibody or antigen-binding fragment comprising a thus-altered amino acid sequence may retain an ability to bind human Aβ1-42. It may retain the same quantitative binding and/or neutralizing ability as an antibody or antigen-binding fragment in which the alteration is not made, e.g., as measured in an assay described herein. The antibody or antigen-binding fragment comprising a thus-altered amino acid sequence may have an improved ability to bind human Aβ1-42.

Mutation may comprise replacing one or more amino acid residues with a non-naturally occurring or non-standard amino acid, modifying one or more amino acid residue into a non-naturally occurring or non-standard form, or inserting one or more non-naturally occurring or non-standard amino acid into the sequence. Examples of numbers and locations of alterations in sequences of the disclosure are described elsewhere herein. Naturally occurring amino acids include the 20 “standard” L-amino acids identified as G, A, V, L, I, M, P, F, W, S, T, N, Q, Y, C, K, R. H, D, E by their standard single-letter codes. Non-standard amino acids include any other residue that may be incorporated into a polypeptide backbone or result from modification of an existing amino acid residue. Non-standard amino acids may be naturally occurring or non-naturally occurring. Several naturally occurring non-standard amino acids are known in the art, such as 4-hydroxyproline, 5-hydroxylysine, 3-methylhistidine, N-acetylserine, etc. [Voet & Voet, Biochemistry, 2nd Edition, (Wiley) 1995]. Those amino acid residues that are derivatized at their N-alpha position will only be located at the N-terminus of an amino-acid sequence. Normally in the present disclosure an amino acid is an L-amino acid, but it may be a D-amino acid. Alteration may therefore comprise modifying an L-amino acid into, or replacing it with, a D-amino acid. Methylated, acetylated and/or phosphorylated forms of amino acids are also known, and amino acids in the present disclosure may be subject to such modification.

Amino acid sequences in antibody domains and antibodies or antigen-binding fragments of the disclosure may comprise non-natural or non-standard amino acids described above. Non-standard amino acids (e.g. D-amino acids) may be incorporated into an amino acid sequence during synthesis, or by modification or replacement of the “original” standard amino acids after synthesis of the amino acid sequence.

Use of non-standard and/or non-naturally occurring amino acids increases structural and functional diversity, and can thus increase the potential for achieving desired binding and neutralising properties in an antibody or antigen-binding fragment of the disclosure. Additionally, D-amino acids and analogues have been shown to have different pharmacokinetic profiles compared with standard L-amino acids, owing to in vivo degradation of polypeptides having L-amino acids after administration to an animal, e.g., a human, meaning that D-amino acids are advantageous for some in vivo applications.

Novel VH or VL regions carrying CDR-derived sequences of the disclosure may be generated using random mutagenesis of one or more selected VH and/or VL genes to generate mutations within the entire variable domain. Such a technique is described by Gram et al. [Gram et al., 1992, Proc. Natl. Acad. Sci., USA, 89:3576-35801], who used error-prone PCR. In some embodiments one or two amino acid substitutions are made within an entire variable domain or set of CDRs.

Another method that may be used is to direct mutagenesis to CDR regions of VH or VL genes. Such techniques are disclosed by Barbas et al. [Barbas et al., 1994, Proc. Natl. Acad. Sci., USA, 91:3809-3813] and Schier et al. [Schier et al., 1996, J. Mol. Biol. 263:551-567].

All the above-described techniques are known as such in the art and the skilled person will be able to use such techniques to provide antibodies or antigen-binding fragments of the disclosure using routine methodology in the art.

A further aspect of the disclosure provides a method for obtaining an antibody antigen-binding site for human Aβ1-42, the method comprising providing by way of substitution, deletion, or insertion of one or more amino acids in the amino acid sequence of a VH domain set out herein a VH domain which is an amino acid sequence variant of the VH domain, optionally combining the VH domain thus provided with one or more VL domains, and testing the VH domain or VH/VL combination or combinations to identify an antibody or antigen-binding fragment or an antibody antigen-binding site for Aβ1-42 and optionally with one or more desired properties. Said VL domain may have an amino acid sequence which is substantially as set out herein. An analogous method may be employed in which one or more sequence variants of a VL domain disclosed herein are combined with one or more VH domains.

As noted above, a CDR amino acid sequence substantially as set out herein may be incorporated as a CDR in a human antibody variable domain or a substantial portion thereof. The HCDR3 sequences substantially as set out herein represent embodiments of the present disclosure and each of these may be incorporated as a HCDR3 in a human heavy chain variable domain or a substantial portion thereof.

Variable domains employed in the disclosure may be obtained or derived from any germline or rearranged human variable domain, or may be a synthetic variable domain based on consensus or actual sequences of known human variable domains. A variable domain can be derived from a non-human antibody. A CDR sequence of the disclosure (e.g. CDR3) may be introduced into a repertoire of variable domains lacking a CDR (e.g. CDR3), using recombinant DNA technology. For example, Marks et al. [Marks et al Bio/Technology, 1992, 10:779-783] describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al. further describe how this repertoire may be combined with a CDR3 of a particular antibody. Using analogous techniques, the CDR3-derived sequences of the present disclosure may be shuffled with repertoires of VH or VL domains lacking a CDR3, and the shuffled complete VH or VL domains combined with a cognate VL or VH domain to provide antibodies or antigen-binding fragments of the disclosure. The repertoire may then be displayed in a suitable host system, such as the phage display system of WO92/01047, which is herein incorporated by reference in its entirety, or any of a subsequent large body of literature, including Kay, Winter & McCafferty [Kay, B. K., Winter, J., and McCafferty, J. (1996) Phage Display of Peptides and Proteins: A Laboratory Manual, San Diego: Academic Press], so that suitable antibodies or antigen-binding fragments may be selected. A repertoire may consist of from anything from 10⁴ individual members upwards, for example at least 10⁵, at least 10⁶, at least 10⁷, at least 10⁸, at least 10⁹ or at least 10¹⁰ members or more. Other suitable host systems include, but are not limited to yeast display, bacterial display, T7 display, viral display, cell display, ribosome display and covalent display.

A method of preparing an antibody or antigen-binding fragment for human Aβ1-42 is provided, which method comprises:

(a) providing a starting repertoire of nucleic acids encoding a VH domain which either include a CDR3 to be replaced or lack a CDR3 encoding region;

(b) combining said repertoire with a donor nucleic acid encoding an amino acid sequence substantially as set out herein for a VH CDR3, for example a VH CDR3 shown in Table 9, such that said donor nucleic acid is inserted into the CDR3 region in the repertoire, so as to provide a product repertoire of nucleic acids encoding a VH domain;

(c) expressing the nucleic acids of said product repertoire;

(d) selecting an antibody or antigen-binding fragment for human Aβ1-42; and

(e) recovering said antibody or antigen-binding fragment or nucleic acid encoding it.

Again, an analogous method may be employed in which a VL CDR3 of the disclosure is combined with a repertoire of nucleic acids encoding a VL domain that either include a CDR3 to be replaced or lack a CDR3 encoding region.

Similarly, one or more, or all three CDRs may be grafted into a repertoire of VH or VL domains that are then screened for an antibody or antigen-binding fragment or antibodies or antigen-binding fragments for human Aβ1-42.

For example, an HCDR1, HCDR2 and/or HCDR3, e.g., a set of HCDRs, from one or more of the antibodies listed in Table 3 or Table 4 may be employed, and/or an LCDR1, LCDR2 and/or LCDR3, e.g., set of LCDRs, from one or more of the antibodies listed herein may be employed.

Similarly, other VH and VL domains, sets of CDRs and sets of HCDRs and/or sets of LCDRs disclosed herein may be employed.

A substantial portion of an immunoglobulin variable domain may comprise at least the three CDR regions, together with their intervening framework regions. The portion may also include at least about 50% of either or both of the first and fourth framework regions, the 50%/6 being the C-terminal 50% of the first framework region and the N-terminal 50% of the fourth framework region. Additional residues at the N-terminal or C-terminal end of the substantial part of the variable domain may be those not normally associated with naturally-occurring variable domain regions. For example, construction of antibodies or antigen-binding fragments of the present disclosure made by recombinant DNA techniques may result in the introduction of N- or C-terminal residues encoded by linkers introduced to facilitate cloning or other manipulation steps. Other manipulation steps include the introduction of linkers to join variable domains of the disclosure to further protein sequences including antibody constant regions, other variable domains (for example in the production of diabodies) or detectable/functional labels as discussed in more detail elsewhere herein.

Although in some aspects of the disclosure, antibodies or antigen-binding fragments comprise a pair of VH and VL domains, single binding domains based on either VH or VL domain sequences form further aspects of the disclosure. It is known that single immunoglobulin domains, especially VH domains, are capable of binding target antigens in a specific manner. For example, see the discussion of dAbs above.

In the case of either of the single binding domains, these domains may be used to screen for complementary domains capable of forming a two-domain antibody or antigen-binding fragment able to bind Aβ1-42. This may be achieved by phage display screening methods using the so-called hierarchical dual combinatorial approach as disclosed in WO92/01047, herein incorporated by reference in its entirety, in which an individual colony containing either an H or L chain clone is used to infect a complete library of clones encoding the other chain (L or H) and the resulting two-chain antibody or antigen-binding fragment is selected in accordance with phage display techniques, such as those described in that reference. This technique is also disclosed in Marks et al., Bio/Technology, 1992, 10:779-783.

Antibodies or antigen-binding fragments of the present disclosure may further comprise antibody constant regions or parts thereof, e.g., human antibody constant regions or parts thereof. For example, a VL domain may be attached at its C-terminal end to antibody light chain constant domains including human Cκ or Cλ chains. Similarly, an antibody or antigen-binding fragment based on a VH domain may be attached at its C-terminal end to all or part (e.g., a CH1 domain) of an immunoglobulin heavy chain derived from any antibody isotype, e.g. IgG, IgA, IgE and IgM and any of the isotype sub-classes, particularly IgG2, IgG1 and IgG4. IgG2 may be advantageous in some embodiments owing to its lack of effector functions. In other embodiments, IgG1 may be advantageous due to its effector function and ease of manufacture. Any synthetic or other constant region variant that has these properties and stabilizes variable regions may also be useful in the present disclosure.

An aspect of the disclosure provides a method comprising causing or allowing binding of an antibody or antigen-binding fragment as provided herein to human Aβ1-42. As noted, such binding may take place in vivo, e.g. following administration of an antibody or antigen-binding fragment, or nucleic acid encoding an antibody or antigen-binding fragment, or it may take place in vitro, for example in ELISA. Western blotting, immunocytochemistry, immunoprecipitation, affinity chromatography, and biochemical or cell-based assays.

The present disclosure also provides the use of an antibody or antigen-binding fragment as above for measuring antigen levels in a competition assay, that is to say a method of measuring the level of antigen in a sample by employing an antibody or antigen-binding fragment as provided by the present disclosure in a competition assay. This may be where the physical separation of bound from unbound antigen is not required. Linking a reporter molecule to the antibody or antigen-binding fragment so that a physical or optical change occurs on binding is one possibility. The reporter molecule may directly or indirectly generate detectable signals, which may be quantifiable. The linkage of reporter molecules may be directly or indirectly, covalently, e.g., via a peptide bond or non-covalently. Linkage via a peptide bond may be as a result of recombinant expression of a gene fusion encoding antibody and reporter molecule.

Competition between antibodies or antigen-binding fragments may be assayed easily in vitro, for example using ELISA and/or by a biochemical competition assay such as one tagging a specific reporter molecule to one antibody or antigen-binding fragment which can be detected in the presence of one or more other untagged antibodies or antigen-binding fragments, to enable identification of antibodies or antigen-binding fragments which bind the same epitope or an overlapping epitope. Such methods are readily known to one of ordinary skill in the art, and are described in more detail herein.

The present disclosure extends to an antibody or antigen-binding fragment that competes for binding to human Aβ1-42 with any antibody or antigen-binding fragment defined herein, e.g., any of the antibodies listed in Tables 3 and 4, e.g., in IgG2, IgG1 or IgG1 triple mutation (“TM”; Oganesyan et al. (2008) Acta Crystallogr D Biol Crystallogr, 64(Pt 6):700-4) format. Competition between antibodies or antigen-binding fragments may be assayed easily in vitro, for example by tagging a specific reporter molecule to one antibody or antigen-binding fragment which can be detected in the presence of other untagged antibody or antigen-binding fragment(s), to enable identification of antibodies or antigen-binding fragments which bind the same epitope or an overlapping epitope. Competition may be determined for example using ELISA in which Aβ1-42 is immobilized to a plate and a first tagged or labelled antibody or antigen-binding fragment along with one or more other untagged or unlabelled antibodies or antigen-binding fragments is added to the plate. Presence of an untagged antibody or antigen-binding fragment that competes with the tagged antibody or antigen-binding fragment is observed by a decrease in the signal emitted by the tagged antibody or antigen-binding fragment.

Competition assays can also be used in epitope mapping. In one instance epitope mapping may be used to identify the epitope bound by an antibody or antigen-binding fragment which optionally may have optimized neutralizing and/or modulating characteristics. Such an epitope can be linear or conformational. A conformational epitope can comprise at least two different fragments of Aβ, wherein said fragments are positioned in proximity to each other when the Aβ peptide is folded in its tertiary or quaternary structure to form a conformational epitope which is recognized by an inhibitor of Aβ, such as a Aβ-antibody or antigen-binding fragment. In testing for competition a peptide fragment of the antigen may be employed, especially a peptide including or consisting essentially of an epitope of interest. A peptide having the epitope sequence plus one or more amino acids at either end may be used. Antibodies or antigen-binding fragments according to the present disclosure may be such that their binding for antigen is inhibited by a peptide with or including the sequence given.

As used herein, the term “isolated” refers to the state in which antibodies or antigen-binding fragments of the disclosure, or nucleic acid encoding such antibodies or antigen-binding fragments, will generally be in accordance with the present disclosure. Thus, antibodies or antigen-binding fragments, VH and/or VL domains, and encoding nucleic acid molecules and vectors according to the present disclosure may be provided isolated and/or purified, e.g. from their natural environment, in substantially pure or homogeneous form, or, in the case of nucleic acid, free or substantially free of nucleic acid or genes of origin other than the sequence encoding a polypeptide with the required function. Isolated members and isolated nucleic acid will be free or substantially free of material with which they are naturally associated, such as other polypeptides or nucleic acids with which they are found in their natural environment, or the environment in which they are prepared (e.g. cell culture) when such preparation is by recombinant DNA technology practiced in vitro or in vivo. Members and nucleic acid may be formulated with diluents or adjuvants and still for practical purposes be isolated—for example the members will normally be mixed with gelatin or other carriers if used to coat microtitre plates for use in immunoassays, or will be mixed with pharmaceutically acceptable carriers or diluents when used in diagnosis or therapy. Antibodies or antigen-binding fragments may be glycosylated, either naturally or by systems of heterologous eukaryotic cells (e.g. CHO or NSO (ECACC 85110503) cells, or they may be (for example if produced by expression in a prokaryotic cell) unglycosylated.

### 4. Nucleic Acids, Cells and Methods of Production

In further aspects, the disclosure provides an isolated nucleic acid which comprises a sequence encoding an antibody or antigen-binding fragment, VH domain and/or VL domain according to the present disclosure, and methods of preparing an antibody or antigen-binding fragment, a VH domain and/or a VL domain of the disclosure, which comprise expressing said nucleic acid under conditions to bring about production of said antibody or antigen-binding fragment, VH domain and/or VL domain, and recovering it. Examples of encoding nucleic acid sequences are set out in the Tables and the appended sequence listing. Nucleic acid sequences according to the present disclosure may comprise DNA or RNA and may be wholly or partially synthetic. Reference to a nucleotide sequence as set out herein encompasses a DNA molecule with the specified sequence, and encompasses a RNA molecule with the specified sequence in which U is substituted for T, unless context requires otherwise

The present disclosure also provides constructs in the form of plasmids, vectors, such as a plasmid or phage vector, transcription or expression cassettes which comprise at least one polynucleotide as above, for example operably linked to a regulatory element.

A further aspect provides a host cell containing or transformed with the nucleic acids and/or vectors of the disclosure. The present disclosure also provides a recombinant host cell line that comprises one or more constructs as above. A nucleic acid sequence encoding any CDR or set of CDRs or VH domain or VL domain or antibody antigen-binding site or antibody molecule, e.g. scFv or IgG (e.g. IgG2, IgG1 or IgG1TM) as provided, forms an aspect of the present disclosure, along with a method of production of the encoded product, which method comprises expression from encoding nucleic acid sequences thereof. Expression may conveniently be achieved by culturing recombinant host cells containing the nucleic acid under appropriate conditions. Following production by expression a VH or VL domain, or antibody or antigen-binding fragment may be isolated and/or purified using any suitable technique, then used as appropriate.

Accordingly, another aspect of the disclosure is a method of production of an antibody VH variable domain, the method including causing expression from encoding nucleic acid sequences. Such a method may comprise culturing host cells under conditions for production of said antibody VH variable domain.

Analogous methods for production of VL variable domains and antibodies or antigen-binding fragments comprising a VH and/or VL domain are provided as further aspects of the present disclosure.

A method of production may comprise a step of isolation and/or purification of the product. A method of production may comprise formulating the product into a composition including at least one additional component, such as a pharmaceutically acceptable excipient.

Systems for cloning and expression of a polypeptide in a variety of different host cells are well known. Suitable host cells include bacteria, mammalian cells, plant cells, filamentous fungi, yeast and baculovirus systems and transgenic plants and animals. The expression of antibodies and antibody fragments in prokaryotic cells is well established in the art. For a review, see for example Plückthun [Plückthun, A. Bio/Technology 9: 545-551 (1991)]. A common bacterial host is E. coli.

Expression in eukaryotic cells in culture is also available to those skilled in the art as an option for production of an antibody or antigen-binding fragment [Chadd H E and Chamow S M (2001) Current Opinion in Biotechnology 12: 188-194; Andersen D C and Krummen L (2002) Current Opinion in Biotechnology 13: 117 Larrick J W and Thomas D W (2001) Current Opinion in Biotechnology 12:411-418].

Mammalian cell lines available in the art for expression of a heterologous polypeptide include Chinese hamster ovary (CHO) cells, HeLa cells, baby hamster kidney cells, NSO mouse melanoma cells, YB2/0 rat melanoma cells, human embryonic kidney cells, human embryonic retina cells and many others.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids e.g. phagemid, or viral, e.g. ‘phage, as appropriate [Sambrook and Russell, Molecular Cloning: a Laboratory Manual: 3rd edition, 2001, Cold Spring Harbor Laboratory Press]. Many known techniques and protocols for manipulation of nucleic acid, for example in preparation of nucleic acid constructs, mutagenesis, sequencing, introduction of DNA into cells and gene expression, and analysis of proteins, are described in detail in Ausubel et al. [Ausubel et al. eds., Short Protocols in Molecular Biology: A Compendium of Methods from Current Protocols in Molecular Biology. John Wiley & Sons, 4^{th} edition 1999].

A further aspect of the present disclosure provides a host cell containing nucleic acid as disclosed herein. Such a host cell may be in vitro and may be in culture. Such a host cell may be in vivo. In vivo presence of the host cell may allow intra-cellular expression of the antibodies or antigen-binding fragments of the present disclosure as “intrabodies” or intra-cellular antibodies. Intrabodies may be used for gene therapy.

Another aspect provides a method comprising introducing nucleic acid of the disclosure into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g., Vaccinia, or for insect cells, Baculovirus. Introducing nucleic acid in the host cell, in, particular a eukaryotic cell may use a viral or a plasmid based system. The plasmid system may be maintained episomally or may be incorporated into the host cell or into an artificial chromosome. Incorporation may be either by random or targeted integration of one or more copies at single or multiple loci. For bacterial cells, suitable techniques may include calcium chloride transformation, electroporation and transfection using bacteriophage.

The introduction may be followed by causing or allowing expression from the nucleic acid, e.g., by culturing host cells under conditions for expression of the gene. The purification of the expressed product may be achieved by methods known to one of skill in the art.

Nucleic acid of the disclosure may be integrated into the genome (e.g., chromosome) of the host cell. Integration may be promoted by inclusion of sequences that promote recombination with the genome, in accordance with standard techniques.

The present disclosure also provides a method that comprises using a construct as stated above in an expression system in order to express an antibody or antigen-binding fragment or polypeptide as above.

### 5. Methods of Treatment

The present disclosure provides for methods of treating a subject having a disease or disorder with any combination of any of the molecules disclosed herein. In some embodiments, the disclosure provides for a method of treating a subject having a disease or disorder with a) any of the antibodies or antigen-binding fragments disclosed herein, and b) any of the BACE inhibitors disclosed herein. In some embodiments, the antibody or antigen-binding fragment comprises:

a VH CDR1 having the amino acid sequence of SEQ ID NO: 525;

a VH CDR2 having the amino acid sequence of SEQ ID NO: 526;

a VH CDR3 having the amino acid sequence of SEQ ID NO: 527;

a VL CDR1 having the amino acid sequence of SEQ ID NO: 534;

a VL CDR2 having the amino acid sequence of SEQ ID NO: 535; and

a VL CDR3 having the amino acid sequence of SEQ ID NO: 536. In some embodiments, the BACE inhibitor is

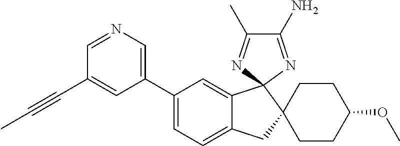 [SMILES: CC#Cc1cncc(-c2ccc3c(c2)[C@@]2(N=C(C)C(N)=N2)[C@]2(CC[C@H](OC)CC2)C3)c1]

or a pharmaceutically acceptable salt thereof. In some embodiments, the BACE inhibitor is a camsylate salt of

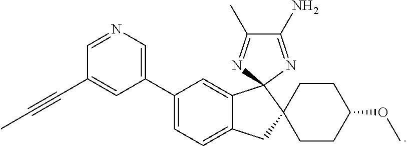 [SMILES: CC#Cc1cncc(-c2ccc3c(c2)[C@@]2(N=C(C)C(N)=N2)[C@]2(CC[C@H](OC)CC2)C3)c1]

In some embodiments, the BACE inhibitor is

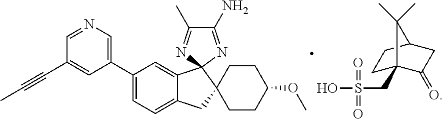

For any of the methods described herein, the disclosure contemplates the combination of any step or steps of one method with any step or steps from another method. These methods involve administering to an individual in need thereof an effective amount of any of the compounds of the disclosure appropriate for the particular disease or disorder. In specific embodiments, these methods involve delivering any of the antibodies or antigen-binding fragments disclosed herein in combination with any of the BACE inhibitors disclosed herein to a subject in need thereof.

In some embodiments, the disease or disorder is any a disease or disorder associated with the accumulation of Aβ. In some embodiments, the accumulation of Aβ is cerebral and/or hippocampal accumulation of Aβ. In some embodiments, the accumulation of Aβ is intraneuronal. In some embodiments, the accumulation of Aβ is extracellular. In some embodiments, the accumulation of Aβ is in endothelial cells. In some embodiments, the accumulation of Aβ is in the retina. In some embodiments, the accumulation of Aβ is in the cerebrovasculature. In some embodiments, any of the treatment methods disclosed herein is useful for preventing, reducing, or reversing (e.g., clearing) accumulation of Aβ.

In some embodiments, the disease or disorder is a neurodegenerative disease or disorder. In particular embodiments, the disease or disorder is Alzheimer's Disease, Down Syndrome, macular degeneration, or cognitive impairment. In some embodiments, the subject is a mammal. In particular embodiments, the subject is a human.

In some embodiments, the subject is administered a therapeutically effective dose of any of the BACE inhibitors disclosed herein in combination with a therapeutically effective dose of any of the antibodies or antigen-binding fragments disclosed herein. By the term “therapeutically effective dose” or “therapeutically effective amount” is meant a dose or amount that produces the desired effect for which it is administered. The exact dose will depend on the purpose of the treatment, and will be ascertainable by one skilled in the art using known techniques (see, e.g., Lloyd (1999) The Art, Science and Technology of Pharmaceutical Compounding).

The present disclosure is directed inter alia to treatment of Alzheimer's disease and other amyloidogenic diseases by administration of a therapeutic antibody of the disclosure to a patient under conditions that generate a beneficial therapeutic response in a patient (e.g., a reduction of Aβ1-42 in CSF, a reduction of plaque burden, inhibition of plaque formation, reduction of neuritic dystrophy, improvement in cognitive function, and/or reversal, reduction or prevention of cognitive decline) in the patient, for example, for the prevention or treatment of an amyloidogenic disease.

The terms “treatment”, “treating”. “alleviation” and the like are used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes any one or more of: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms). For example, “treatment” of Alzheimer's Disease encompasses a complete reversal or cure of the disease, or any range of improvement in conditions and/or adverse effects attributable to Alzheimer's Disease. Merely to illustrate, “treatment” of Alzheimer's Disease includes an improvement in any of the following effects associated with Alzheimer's Disease or combination thereof: mental decline, mental confusion, delusion, disorientation, forgetfulness, difficulty concentrating, inability to create new memories, aggression, agitation, irritability, personality changes, lack of restraint, anger, apathy, general discontent, loneliness, mood swings, depression, hallucination, paranoia, loss of appetite, restlessness, inability to combine muscle movements, jumbled speech, synaptic impairment, neuronal loss, amyloid beta accumulation, tau hyperphosphorylation, accumulation of tau protein, amyloid plaque formation, and neurofibrillary tangle formation. Improvements in any of these conditions can be readily assessed according to standard methods and techniques known in the art. Other symptoms not listed above may also be monitored in order to determine the effectiveness of treating neurodegenerative disease, such as Alzheimer's Disease. The population of subjects treated by the method of the disease includes subjects suffering from the undesirable condition or disease, as well as subjects at risk for development of the condition or disease.

In some embodiments, the treatments disclosed herein prevent the generation of and/or accumulation of Aβ n-42 species in the brain. In some embodiments, the Aβ n-42 species is one of more of Aβ 1-42, Aβ pyro 3-pyro-42, Aβ 4-42, or Aβ 11-pyro-42. In some embodiments, the treatments disclosed herein prevent the accumulation of Aβ 1-43. In some embodiments, the treatments disclosed herein prevent the generation of and/or accumulation of AB oligomers and/or plaques.

The disclosure provides methods of preventing or treating a disease associated with amyloid deposits of Aβ in the brain of a patient. Such diseases include Alzheimer's disease, Down syndrome, and cognitive impairment. Cognitive impairment can occur with or without other characteristics of an amyloidogenic disease. The disclosure provides methods of treatment of macular degeneration, a condition which is linked with Aβ. Methods of the disclosure may involve administering an effective dose to a patient of an antibody that specifically binds to 1-42 Aβ and N-terminal truncates thereof in combination with any of the BACE inhibitors disclosed herein.

Any of the antibodies or antigen-binding fragments disclosed herein may be used in combination with any of the BACE inhibitors disclosed herein in therapeutic regimes for preventing or ameliorating the neuropathology and, in some patients, the cognitive impairment associated with Alzheimer's disease.

Patients amenable to treatment include patients showing symptoms and also individuals at risk of disease but not showing symptoms. For Alzheimer's disease, potentially anyone is at risk if he or she lives for a sufficiently long time. Any of the antibodies or antigen-binding fragments disclosed herein may be used in combination with any of the BACE inhibitors disclosed herein and administered prophylactically to a subject without any assessment of the risk of the subject patient. Patients amenable to treatment include individuals who have a known genetic risk of Alzheimer's disease, for example individuals who have blood relatives with this disease and those whose risk is determined by analysis of genetic or biochemical markers. Genetic markers of predisposition towards Alzheimer's disease include mutations in the APP gene, particularly mutations at position 717 and positions 670 and 671 referred to as the Hardy and Swedish mutations respectively. Other markers of risk are mutations in the presenilin genes, PS1 and PS2, and ApoE4, a family history of AD, hypercholesterolemia or atherosclerosis. Individuals suffering from Alzheimer's disease can be diagnosed by the characteristic dementia associated with the disease, as well as by the presence of risk factors described above. A number of diagnostic tests are available to assist in identification Alzheimer's disease in an individual. These include measurement of CSF tau and Aβ1-42 levels. Elevated tau and decreased Aβ1-42 levels may signify the presence of AD. Individuals suffering from Alzheimer's disease can also be diagnosed by NINCDS-ADRDA or DSM-IV-TR criteria. In some embodiments, the Alzheimer's Disease to be treated is mild (early-stage), moderate (middle-stage), or severe (late-stage) Alzheimer's Disease.

In asymptomatic patients, treatment can begin at any age (e.g., at least 10, 20, 30 years of age). Generally, treatment is commenced in later life, for example when a patient reaches his or her 40's, 50's, 60's or 70's. Treatment may involve multiple doses over a period of time, which may be for the duration of the remaining life of the patient. The need for administration of repeat doses can be monitored by measuring antibody levels over time. As Alzheimer's Disease may have an early onset in Down Syndrome patients, administration of any of the antibodies or antigen-binding fragments disclosed herein in combination with any of the BACE inhibitors disclosed herein may be initiated at earlier stages of life (e.g., when the patient is at least 10, 20, 30 years of age) than in a non-Down Syndrome patient.

For prophylaxis, pharmaceutical compositions or medicaments are administered to a patient susceptible to, or otherwise at risk of. Alzheimer's disease in an amount sufficient to eliminate or reduce the risk, lessen the severity, or delay the outset of the disease, including biochemical, histologic, cognitive impairment and/or behavioural symptoms of the disease, its complications and intermediate pathological phenotypes presenting during development of the disease. For therapeutic applications, compositions or medicaments are administered to a patient suspected of, or already suffering from such a disease in an amount sufficient to cure, or at least partially arrest, the symptoms of the disease (biochemical, histologic, cognitive impairment and/or behavioural), including its complications and intermediate pathological phenotypes in development of the disease.

A method of treatment may comprise (i) identifying a patient having a condition associated with amyloidosis as mentioned herein, and (ii) administering a therapeutically effective dose of any of the antibodies or antigen-binding fragments disclosed herein in combination with a therapeutically effective dose of any of the BACE inhibitors disclosed herein, wherein levels of Aβ1-42 are decreased in blood plasma and/or CSF, and amyloidosis is reduced.

Accordingly, further aspects of the disclosure provide methods of treatment comprising administration of any of the antibodies or antigen-binding fragments disclosed herein in combination with any of the BACE inhibitors disclosed herein, pharmaceutical compositions comprising any of the antibodies or antigen-binding fragments disclosed herein alone or in combination with any of the BACE inhibitors disclosed herein, pharmaceutical compositions comprising any of the BACE inhibitors disclosed herein alone or in combination with any of the antibodies or antigen-binding fragments disclosed herein, and use of such an antibody or antigen-binding fragment and/or BACE inhibitor in the manufacture of a medicament for administration, for example in a method of making a medicament or pharmaceutical composition comprising formulating the antibody or antigen-binding fragment and/or BACE inhibitor with a pharmaceutically acceptable excipient. A pharmaceutically acceptable excipient may be a compound or a combination of compounds entering into a pharmaceutical composition not provoking secondary reactions and which allows, for example, facilitation of the administration of the antibody or antigen-binding fragment, an increase in its lifespan and/or in its efficacy in the body, an increase in its solubility in solution or else an improvement in its conservation. These pharmaceutically acceptable vehicles are well known and will be adapted by the person skilled in the art as a function of the nature and of the mode of administration of the active compound(s) chosen.

Antibodies or antigen-binding fragments as described herein will usually be administered in the form of a pharmaceutical composition, which may comprise at least one component in addition to the antibody or antigen-binding fragment. Thus pharmaceutical compositions according to the present disclosure, and for use in accordance with the present disclosure, may comprise, in addition to an antibody or antigen-binding fragment, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration.

BACE inhibitors as described herein will usually be administered in the form of a pharmaceutical composition, which may comprise at least one component in addition to the antibody or antigen-binding fragment. Thus pharmaceutical compositions according to the present disclosure, and for use in accordance with the present disclosure, may comprise, in addition to an antibody or antigen-binding fragment, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration.

In some embodiments, any of the BACE inhibitors disclosed herein and/or any of the antibodies or antigen-binding fragments thereof are administered to a subject by means of any one or more of the following routes of administration: parenteral, intradermal, intramuscular, intraperitoneal, intramyocardial, intravenous, subcutaneous, pulmonary, intranasal, intraocular, epidural, intrathecal, intracranial, intraventricular and oral routes.

In some embodiments, any of the antibodies or antigen-binding fragments disclosed herein is administered in the same composition with any of the BACE inhibitors disclosed herein. In some embodiments, any of the antibodies or antigen-binding fragments disclosed herein is administered in a separate composition as the composition comprising any of the BACE inhibitors disclosed herein. In some embodiments, if the composition comprising any of the antibodies or antigen-binding fragments disclosed herein is administered separately from the composition comprising any of the BACE inhibitors disclosed herein, the compositions are administered to the subject by the same route of administration. In some embodiments, the compositions are administered to the subject by a different route of administration. In some embodiments, the composition comprising any of the antibodies or antigen-binding fragments disclosed herein is administered to the subject via injection. In some embodiments, the injection is intravenous. In some embodiments, the injection is subcutaneous. In some embodiments, the composition comprising any of the BACE inhibitors disclosed herein is administered to the subject orally.

In some embodiments, the pharmaceutically effective dose of any of the BACE inhibitors disclosed herein is less when administered to a subject in combination with any of the antibodies or antigen-binding fragments disclosed herein as compared to the pharmaceutically effective dose of the BACE inhibitor when administered alone. In some embodiments, the pharmaceutically effective dose of any of the antibodies or antigen-binding fragments disclosed herein is less when administered to a subject in combination with any of the BACE inhibitors disclosed herein as compared to the pharmaceutically effective dose of the antibody or antigen-binding fragment when administered alone.

For injectable formulations, e.g., for intravenous or subcutaneous injection, the active ingredient will be in the form of a parenterally acceptable aqueous solution which is pyrogen-free and has suitable pH, isotonicity and stability. Antibodies or antigen-binding fragments as described herein may be formulated in liquid, semi-solid or solid forms depending on the physicochemical properties of the molecule and the route of delivery. Formulations may include excipients, or combinations of excipients, for example: sugars, amino acids and surfactants.

Liquid formulations may include a wide range of antibody concentrations and pH. Solid formulations may be produced by lyophilisation, spray drying, or drying by supercritical fluid technology, for example. Treatment may be given by injection (for example, subcutaneously, or intra-venously. The treatment may be administered by pulse infusion, particularly with declining doses of the antibody or antigen-binding fragment. The route of administration can be determined by the physicochemical characteristics of the treatment, by special considerations for the disease or by the requirement to optimize efficacy or to minimize side-effects. One particular route of administration is intravenous. Another route of administering pharmaceutical compositions of the present disclosure is subcutaneously. Subcutaneous injection using a needle-free device is also advantageous. In some embodiments, any of the antibodies or antigen-binding fragments disclosed herein is administered to the subject by means of injection.

Any of the antibodies or antigen-binding fragments disclosed herein and any of the BACE inhibitors disclosed herein may be administered to a subject either simultaneously or sequentially. In some embodiments, any of the antibody or antigen-binding fragment/BACE inhibitor combination therapies disclosed herein is further combined with additional treatments.

In some embodiments, any of the antibodies or antigen-binding fragments of the disclosure and any of the BACE inhibitors of the disclosure may be used in the manufacture of a medicament. The medicament may be for separate or combined administration to an individual, and accordingly may comprise the antibody or antigen-binding fragment and the BACE inhibitor as a combined preparation or as separate preparations. Separate preparations may be used to facilitate separate and sequential or simultaneous administration, and allow administration of the components by different routes, e.g. oral and injectable (e.g., intravenous and/or subcutaneous) administration.

In some embodiments, any of the combination therapies disclosed herein (e.g, any of the therapies involving the administration of any of the antibodies or antigen-binding fragments disclosed herein in combination with any of the BACE inhibitors disclosed herein) may be administered to a subject in combination with an additional therapy. In some embodiments, the additional therapy includes, but is not limited to, memory training exercises, memory aids, cognitive training, dietary therapy, occupational therapy, physical therapy, psychiatric therapy, massage, acupuncture, acupressure, mobility aids, assistance animals, and the like. In some embodiments, the additional therapy is the administration to the subject of an additional medicinal component. In some embodiments, the additional medicinal component may be used to provide significant synergistic effects, particularly the combination of an antibody or antigen-binding fragment with one or more other drugs. In some embodiments, the additional medicinal component is administered concurrently or sequentially or as a combined preparation with any of the BACE inhibitors disclosed herein and/or any of the antibodies or antigen-binding fragments disclosed herein, for the treatment of one or more of the conditions listed herein. In some embodiments, the additional medicinal component is a small molecule, a polypeptide, an antibody, an antisense oligonucleotide, and/or siRNA molecule. In some embodiments, the additional medicinal component is any one or more of: donepezil (Aricept), glantamine (Razadyne), memantine (Namenda), rivastigmine (Exelon), or tacrine (Cognex). In some embodiments, the additional medicinal component is an antidepressant, an anxiolytic, an antipsychotic, or a sleeping aid. In some embodiments, any of the antibodies or antigen-binding fragments of the disclosure and one or more of the above additional medicinal components may be used in the manufacture of a medicament. The medicament may be for separate or combined administration to an individual, and accordingly may comprise the antibody or antigen-binding fragment and the additional component as a combined preparation or as separate preparations. Separate preparations may be used to facilitate separate and sequential or simultaneous administration, and allow administration of the components by different routes e.g. oral, intravenous and parenteral administration.

In some embodiments, any of the BACE inhibitors of the disclosure and one or more of the above additional medicinal components may be used in the manufacture of a medicament. The medicament may be for separate or combined administration to an individual, and accordingly may comprise the BACE inhibitor and the additional component as a combined preparation or as separate preparations. Separate preparations may be used to facilitate separate and sequential or simultaneous administration, and allow administration of the components by different routes e.g. oral and parenteral administration.

In some embodiments, any of the antibodies or antigen-binding fragments of the disclosure and one or more of the above additional medicinal components may be used in the manufacture of a medicament. The medicament may be for separate or combined administration to an individual, and accordingly may comprise the antibody or antigen-binding fragment and the additional component as a combined preparation or as separate preparations. Separate preparations may be used to facilitate separate and sequential or simultaneous administration, and allow administration of the components by different routes e.g. oral and parenteral administration.

Compositions provided may be administered to mammals. Administration is normally in a therapeutically effective amount, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the type of antibody or antigen-binding fragment and/or BACE inhibitor, the method of administration, the scheduling of administration and other factors known to medical practitioners. Prescription of treatment, e.g. decisions on dosage etc, is within the responsibility of general practitioners and other medical doctors and may depend on the severity of the symptoms and/or progression of a disease being treated. A therapeutically effective amount or suitable dose of an antibody or antigen-binding fragment of the disclosure and/or a BACE inhibitor of the disclosure can be determined by comparing its in vitro activity and in vivo activity in an animal model. Methods for extrapolation of effective dosages in test animals to humans are known. An initial higher loading dose, followed by one or more lower doses, may be administered. Treatments may be repeated at daily, twice-weekly, weekly or monthly intervals, at the discretion of the physician. Treatments may be every two to four weeks for subcutaneous administration and every four to eight weeks for intra-venous administration. Treatment may be periodic, and the period between administrations is about two weeks or more, e.g., about three weeks or more, about four weeks or more, or about once a month.

Various further aspects and embodiments of the present disclosure will be apparent to those skilled in the art in view of the present disclosure.

All documents, including database references and accession numbers, patents, patent applications and publications, mentioned in this specification are incorporated herein by reference in their entirety for all purposes.

Unless context dictates otherwise, the descriptions and definitions of the features set out above are not limited to any particular aspect or embodiment of the disclosure and apply equally to all aspects and embodiments which are described.

Certain aspects and embodiments of the disclosure will now be illustrated by way of example and with reference to the accompanying figures and tables.

### 6. Kits

In some embodiments, the disclosure provides for a kit comprising any of the BACE inhibitors disclosed herein and any of the antibodies or antigen-binding fragments disclosed herein. In some embodiments, the BACE inhibitor is in a composition suitable for oral administration. In some embodiments, the antibody or antigen-binding fragment is in a composition suitable for intravenous or subcutaneous administration.

### EXAMPLES

The following sequences have been deposited with NCIMB, Ferguson Building, Craibstone Estate, Bucksburn, Aberdeen, AB21 9YA. Scotland, UK:

E. coli TOP10 cells Abet0007=NCIMB 41889

E. coli TOP10 cells Abet0380-GL=NCIMB 41890

E. coli TOP10 cells Abet0144-GL=NCIMB 41891

E. coli TOP10 cells Abet0377-GL=NCIMB 41892

Date of deposit=2 Nov. 2011

### Example 1. Antibody Optimisation of Abet0144-GL Through Mutation of all Six CDRs Including Flanking Vernier Residues

Provided below is a description of the optimization and characterization of new anti-Aβ antibodies from a particular anti-Aβ antibody parent clone, Abet0144-GL.

1.1 Conversion ofAbet0144-GL Parent Clone to scFv Format Compatible with Ribosome Display

The parent clone was converted from IgG1-TM format to single chain variable fragment (scFv) format in preparation for affinity optimisation. The codon-optimized variable heavy (VH) and variable light (VL) domains were amplified separately from their respective IgG vectors with the addition of specific cloning sites and a flexible linker region. Recombinatorial PCR was then performed to generate a complete scFv construct, which was cloned into a modified pUC vector (pUC-RD) containing the structural features necessary for ribosome display. These features include a 5′ and 3′ stem loop to prevent degradation of the mRNA transcript by exonucleases, a Shine-Dalgamo sequence to promote ribosome binding to the mRNA transcript, and a genellI spacer that allows the translated scFv molecule to fold while still remaining attached to the ribosome (Groves et al., 2005).

### 1.2 Optimisation of Abet0144-GL by Targeted Mutagenesis

The lead antibody (Abet0144-GL) was further optimized for improved affinity to human Amyloid beta 1-42 peptide using a targeted mutagenesis approach with affinity-based ribosome display selections. Large scFv-ribosome libraries derived from Abet0144-GL were created by oligonucleotide-directed mutagenesis of all six variable heavy (V_{H}) and variable light (V_{L}) chain complementarity determining regions (CDRs) using standard molecular biology techniques as described by Clackson and Lowman (Clackson et al., 2004). The mutated sequences from each CDR were affinity optimized as a separate library. The five Vernier residues preceding the V_{H}CDR1 (Kabat residues 26-30) were also randomized using targeted mutagenesis and these sequences were combined and matured with the remaining V_{H}CDR1 library. All libraries were subjected to affinity-based ribosome display selections in order to enrich for variants with higher affinity for human Amyloid beta 1-42 peptide. The selections were performed essentially as described previously (Hanes et al., 2000).

In brief, the six targeted mutagenesis libraries of the Abet0144-GL lead clone, one covering each CDR, were separately transcribed into mRNA. Using a process of stalled translation, mRNA-ribosome-scFv tertiary complexes were formed (Hanes et al., 1997). These complexes were then subjected to four rounds of selection incubated in the presence of decreasing concentrations of synthetic biotinylated human Amyloid beta 1-42 peptide (Bachem. Germany, cat: H-5642) (100 nM to 10 nM) to select for variants with higher affinity for human Amyloid beta 1-42 peptide. Those complexes that bound to the antigen were then captured on streptavidin-coated paramagnetic beads (Dynabeads™, Invitrogen, UK; cat: 112-05D) and non-specific ribosome complexes were washed away. mRNA was subsequently isolated from the bound ribosomal complexes, reverse transcribed to cDNA and them amplified by PCR. This DNA was used for the next round of selection.

After four rounds of affinity maturation, each selection output was cloned out for screening purposes. ScFv isolated by ribosome display were cloned into the phagemid vector pCANTAB6 by NotI/NcoI restriction endonuclease digestion of the ribosome display construct (New England BioLabs, USA; cat: R0189L, R0193L) followed by ligation into NotI/NcoI digested pCANTAB6 using T4 DNA ligase (New England BioLabs. USA: cat: M0202L) essentially as described by McCafferty et al. (McCafferty et al., 1994).

### 1.3 Identification of Improved Clones Using an Epitope Competition Assay

Two thousand and twenty four scFv chosen at random from selection rounds 3 and 4 of the targeted mutagenesis approach described in section 1.2 were expressed in bacteria to produce unpurified periplasmic scFv. Those scFv capable of binding synthetic human amyloid beta 1-42 peptide via the same epitope as Abet0144-GL IgG1-TM were elucidated in a competition format assay, using the HTRF™ platform. Specifically, fluorescence resonance energy transfer (FRET) was measured between streptavidin cryptate (associated with biotinylated amyloid beta 1-42 peptide) and anti-human Fc XL665 (associated with Abet0144-GL IgG1-TM) in the presence of a single concentration of each unpurified periplasmic test scFv. Successful occupation of the Abet0144-GL IgG1-TM epitope on the peptide by scFv resulted in a reduction in FRET, as measured on a fluorescence plate reader.

A ‘Total’ binding signal was determined by analysing the binding of Abet0144-GL IgG1-TM to synthetic human Amyloid beta 1-42 peptide in the absence of competitor peptide. The ‘Sample’ signals were derived from analysing the binding of Abet0144-GL IgG1-TM to synthetic human Amyloid beta 1-42 peptide in the presence of a test scFv sample. Finally, a ‘Cryptate Blank’ signal was determined by analysing the fluorescence mediated by the detection reagent cocktail alone.

Unpurified periplasmic scFv were supplied in sample buffer consisting of 50 mM MOPS, pH 7.4, 0.5 mM EDTA, and 0.5 M sucrose. For profiling, scFv samples were diluted in a 384-well V-bottom plate to 50% of the original stock concentration in assay buffer, consisting of 50 mM MOPS, pH 7.4, 0.4 M potassium fluoride, 0.1%6 fatty-acid-free bovine serum albumin and 0.1% Tween 20 (v/v), 5 μl of each newly-diluted scFv was transferred to the ‘Sample’ wells of a black, shallow, solid bottom, non-binding 384-well assay plate using a liquid handling robot. The remaining reagents (prepared in assay buffer) were added to the assay plate by multichannel pipette in the following order: 5 μl sample buffer (to ‘Total’ and ‘Cryptate Blank’ wells), 10 μl assay buffer (to ‘Cryptate Blank’ wells), 5 μl 2 nM Abet0144-GL IgG1-TM (to ‘Sample’ and ‘Total’ wells), 5 μl 5 nM biotinylated human Amyloid beta 1-42 peptide (to ‘Sample’ and ‘Total’ wells), and 5 μl detection cocktail, consisting of 6 nM streptavidin cryptate and 60 nM anti-His6-XL665 (to all wells). Assay plates were sealed and then incubated for 3 hours at room temperature in the dark, prior to measuring time-resolved fluorescence at 620 and 665 nm emission wavelengths on a fluorescence plate reader.

Data were analysed by calculating % Delta F values for each sample. % Delta F was determined according to equation 1.

$$\% Delta F = \frac{\begin{matrix}( Sample 665 nm / 620 nm ratio ) - \\ ( Cryptate Blank 665 nm / 620 nm ratio )\end{matrix}}{( Cryptate Blank 665 nm / 620 nm ratio )} \times 100 \qquad Equation 1$$

Delta F values were subsequently used to calculate normalized binding values as described in equation 2.

$$Normalized data ( \% Total ) = \frac{\% Delta F of sample}{\% Delta F of Total binding control} \times 100 \qquad Equation 2$$

Unpurified periplasmic scFv demonstrating significant inhibition of Abet0144-GL IgG1-TM binding to Amyloid beta 1-42 peptide were subjected to DNA sequencing (Osboum et al., 1996; Vaughan et al., 1996). The scFv found to have unique protein sequences were expressed in E. coli and purified by affinity chromatography followed by buffer exchange.

The potency of each purified scFv was determined by testing a dilution series of the scFv (typically 4 pM-1200 nM) in the epitope competition assay described above. Data were again analysed by calculating the % Delta F and % Total binding values for each sample. In addition, a % Inhibition value for each concentration of purified scFv was also calculated as described in Equation 3:

Inhibition=100−% Total Binding Equation 3:

ScFv sample concentration was plotted against % Inhibition using scientific graphing software, and any concentration-dependant responses were fitted with non-linear regression curves. IC₅₀ values were obtained from these analyses with Hill-slopes constrained to a value of −1. The most potent clone from this round of selections, Abet0286, had an IC₅₀ of 1.8 nM and came from the V_{L}CDR1 targeted mutagenesis library.

Reagent/Eqipment sources: MOPS (Sigma, UK; cat: M9381), potassium fluoride (BDH chemicals, USA; cat: A6003), fatty-acid-free bovine serum albumin (Sigma, UK; cat: A6003), Tween 20 (Sigma, UK; cat: P2287), Abet0144-GL IgG1-TM (produced in-house), biotinylated human Amyloid beta 1-42 peptide (rpeptide, USA; cat: A1117), Streptavidin cryptate (Cisbio, France; cat: 610SAKLB), anti-His6-XL665 (Cisbio, France; cat: 61HISXLB), 384-well assay plates (Corning, Costar Life Sciences: cat: 3676), 384-well dilution plates (Greiner BioOne, Germany; cat: 781280), liquid handling robot (MiniTrak™, Perkin Elmer, USA), fluorescence plate reader (Envision™, Perkin Elmer, USA), HTRF technology (Cisbio International. France), graphing/statistical software (Prism, Graphpad USA).

1.4 Recombination of Successful Selection Outputs to Produce “Binary” Libraries, and their Subsequent Affinity Optimisation

The epitope competition assay described in Section 1.3 was used to judge whether a particular scFv-ribosome library had been affinity matured over the first four rounds of selection. Two of the libraries, the V_{H}CDR3 and the V_{L}CDR2 targeted mutagenesis libraries, had shown no improvement over the parent Abet0144-GL clone and were not progressed further.

The remaining four targeted mutagenesis libraries, (covering the V_{H}CDR1, V_{H}CDR2, V_{L}CDR1 and V_{L}CDR3), had shown affinity improvements and were recombined in a pair-wise fashion to produce six “binary” recombination libraries in which two of the six CDRs were mutated. For example, the affinity matured library covering the V_{H}CDR1 was randomly recombined with the affinity matured V_{H}CDR2 library to generate a V_{H}1:V_{H}2 library. The remaining libraries were produced as: V_{H}1:V_{L}1, V_{H}1:V_{L}3, V_{H}2:V_{L}1, V_{H}2:V_{L}3 and V_{L}1:V_{L}3. A subset of each recombination library was cloned out as previously described (Section 1.2) and was sent for sequencing to verify the integrity of each library.

Selections were then continued as previously described (section 1.2) in the presence of decreasing concentrations of biotinylated synthetic human Amyloid beta 1-42 peptide (5 nM and 2 nM for rounds 5 and 6 respectively). As before, each selection output was cloned out for screening purposes (section 1.2).

One thousand nine hundred and thirty-six scFv, randomly selected from selection rounds 5 and 6, were screened in an epitope competition assay as described in section 1.3. Due to the increase in potency of these clones, the unpurified scFv were first diluted to 25% before addition to the assay plates. As previously, clones that showed significant inhibitory properties were sent for DNA sequencing, and unique clones were produced and analysed as purified scFv (section 1.3). The most potent clone from these selections, Abet0303, had a potency of 0.84 nM and came from the V_{H}1:V_{H}2 recombination library.

1.5 Recombination of Binary Selection Outputs to Produce “Ternary” Libraries, and their Subsequent Affinity Optimisation

The epitope competition assay described in Section 1.3 was used to judge whether each binary library had been affinity matured over the previous two rounds of selection (5 and 6). All libraries had shown affinity improvements, and were therefore considered for further affinity maturation.

The six binary libraries (section 1.4) were recombined with the successful round 4 outputs (section 1.2) in a pair-wise fashion to form four “ternary” recombination libraries in which three of the six CDRs were mutated. For example, the V_{H}2:V_{L}3 binary library (round 6 output) was recombined with the V_{H}CDR1 targeted mutagenesis library (round 4 output) to generate a V_{H}1:V_{H}2:V_{L}3 library. Similar constructs were also created by combining the V_{H}1:V_{H}2 binary library (round 6 output) with the V_{L}CDR3 targeted mutagenesis library (round 4 output). These two individual libraries were pooled to create the V_{H}1:V_{H}2:V_{L}3 ternary library.

Care was taken not to destroy the synergy between CDRs that had been co-optimized. For example, the V_{H}1:V_{L}3 binary library was not recombined with the V_{H}CDR2 targeted mutagenesis library since this manipulation would have destroyed the synergy between the co-optimized V_{H}CDR1 and V_{L}CDR3 sequences. A complete list of all ternary libraries and their derivations is given in Table 1. A subset of each recombination library was cloned out as previously described (Section 1.2) and was sent for sequencing to verify the integrity of each library.

TABLE 1
A description of the four ternary libraries that were matured
during rounds 7 and 8 of the second Lead Optimisation campaign.
Each library comprised two constituent libraries, generated from
a random pairwise recombination of a round 6 output binary library
and a round 4 output targeted mutagenesis library.

| Ternary | Constituent | Formed From | |
|---|---|---|---|
| Library | Libraries | Round 6 output | Round 4 output |
| V_{H}1:V_{H}2:V_{L}1 | V_{H}1:V_{H}2:V_{L}1 a | V_{H}1:V_{H}2 | V_{L}CDR1 |
| | V_{H}1:V_{H}2:V_{L}1 b | V_{H}2:V_{L}1 | V_{H}CDR1 |
| V_{H}1:V_{H}2:V_{L}3 | V_{H}1:V_{H}2:V_{L}3 a | V_{H}1:V_{H}2 | V_{L}CDR3 |
| | V_{H}1:V_{H}2:V_{L}3 b | V_{H}2:V_{L}3 | V_{H}CDR1 |
| V_{H}1:V_{L}1:V_{L}3 | V_{H}1:V_{L}1:V_{L}3 a | V_{H}1:V_{L}1 | V_{L}CDR3 |
| | V_{H}1:V_{L}1:V_{L}3 b | V_{L}1:V_{L}3 | V_{H}CDR1 |
| V_{H}2:V_{L}1:V_{L}3 | V_{H}2:V_{L}1:V_{L}3 a | V_{H}2:V_{L}1 | V_{L}CDR3 |
| | V_{H}2:V_{L}1:V_{L}3 b | V_{L}1:V_{L}3 | V_{H}CDR2 |

Selections were then continued as previously described (section 1.2) in the presence of decreasing concentrations of biotinylated synthetic human Amyloid beta 1-42 peptide (500 pM and 200 pM for rounds 7 and 8 respectively). As before, each selection output was cloned out for screening purposes (section 1.2).

One thousand four hundred and eight scFv, randomly selected from selection rounds 7 and 8, were screened in an epitope competition assay as described in section 1.3. As with the “binary” screen, the unpurified scFv were first diluted to 25% before addition to the assay plates.

As previously, clones that showed significant inhibitory properties were sent for DNA sequencing, and unique clones were produced and analysed as purified scFv (section 1.3). The most potent clone from these selections, Abet0343, had a potency of 0.48 nM and came from the V_{H}1:V_{H}2:V_{L}3 recombination library.

3.6 Recombination of Ternary Selection Outputs to Produce “Quaternary” Libraries, and their Subsequent Affinity Optimisation

The epitope competition assay described in Section 1.3 was used to judge whether each ternary library had been affinity matured over the previous two rounds of selection (7 and 8). All libraries had shown affinity improvements, and were therefore considered for further affinity maturation.

The V_{H}1:V_{H}2:V_{L}1 ternary library (round 8 output) was recombined with the V_{L}CDR3 targeted mutagenesis library (round 4 output) and the V_{H}2:V_{L}1:V_{L}3 ternary library (round 8 output) was recombined with the V_{H}CDR1 targeted mutagenesis library (round 4 output). Separately, the V_{H}1:V_{H}2 binary library (round 6 output) was recombined with the V_{L}1:V_{L}3 binary library (round 6 output). These three individual libraries were then pooled to create a single “quaternary” library, V_{H}1:V_{H}2:V_{L}1:V_{L}3, in which four of the six CDRs were mutated.

Care was taken not to destroy the synergy between CDRs that had been co-optimized. For example, the V_{H}1:V_{L}2:V_{L}3 ternary library was not recombined with the VLCDRI targeted mutagenesis library since this manipulation would have destroyed the synergy between the co-optimized V_{H}CDR1/V_{H}CDR2 and V_{L}CDR3 sequences. A subset of each recombination library was cloned out as previously described (Section 1.2) and was sent for sequencing to verify the integrity of each library.

Selections were then continued as previously described (section 1.2) in the presence of decreasing concentrations of biotinylated synthetic human Amyloid beta 1-42 peptide (50 pM to 10 pM for rounds 9 to 11). As before, each selection output was cloned out for screening purposes (section 1.2).

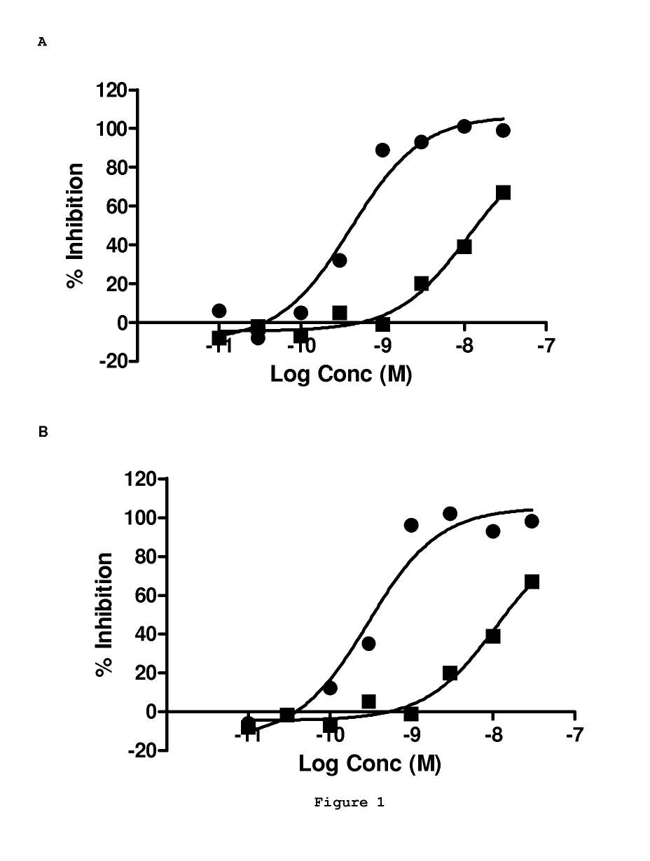

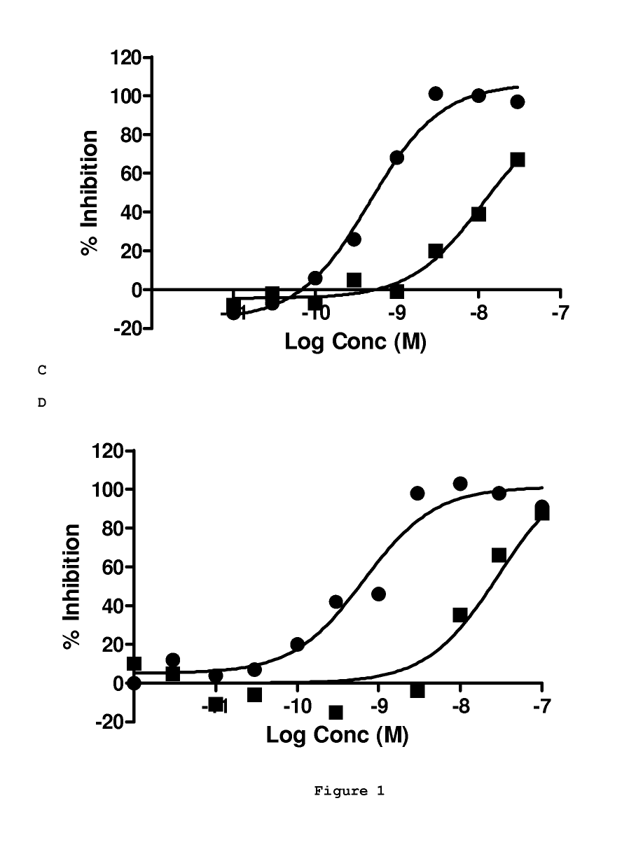

One thousand six hundred and seventy two scFv, randomly selected from selection rounds 9 to 11, were screened in an epitope competition assay as described in section 1.3. Due to the increase in potency of these clones, the unpurified scFv were first diluted to 3.13% before addition to the assay plates. As previously, clones that showed significant inhibitory properties were sent for DNA sequencing, and unique clones were produced and analysed as purified scFv (section 1.3). The most potent clone from these selections, Abet0377, had a potency of 0.32 nM (n=2 data). Sample inhibition curves are shown in FIG. 1, and data for 24 of the highest potency clones are shown in Table 2. The corresponding protein sequences are listed in Tables 3 and 4.

TABLE 2
Example potency data for optimized scFv clones when
evaluated in the Abet0144-GL HTRF ™ epitope
competition assay. Where the assay was performed more than
once, the absolute range of IC₅₀ values is provided.

| | Selection | | | Number of |
|---|---|---|---|---|
| Clone | round | IC₅₀ (nM) | Range | repeats |
| Abet0144-GL | — | 14 | 8.1-18 | 7 |
| Abet0319 | 7 | 0.68 | 0.52-0.76 | 3 |
| Abet0321b | 7 | 0.73 | 0.69-0.76 | 2 |
| Abet0322b | 7 | 0.71 | 0.43-0.98 | 2 |
| Abet0323b | 8 | 0.67 | 0.57-0.76 | 2 |
| Abet0328 | 8 | 0.55 | | 1 |
| Abet0329 | 8 | 0.63 | | 1 |
| Abet0332 | 8 | 0.91 | | 1 |
| Abet0342 | 8 | 0.59 | | 1 |
| Abet0343 | 8 | 0.48 | | 1 |
| Abet0344 | 7 | 0.77 | | 1 |
| Abet0368 | 11 | 0.55 | | 1 |
| Abet0369 | 10 | 0.36 | 0.30-0.41 | 3 |
| Abet0370 | 10 | 0.76 | | 1 |
| Abet0371 | 11 | 0.50 | 0.46-0.53 | 2 |
| Abet0372 | 10 | 0.38 | 0.26-0.49 | 2 |
| Abet0373 | 10 | 0.84 | | 1 |
| Abet0374 | 10 | 0.42 | 0.41-0.43 | 2 |
| Abet0377 | 10 | 0.32 | 0.29-0.35 | 2 |
| Abet0378 | 9 | 0.97 | | 1 |
| Abet0379 | 9 | 0.69 | | 1 |
| Abet0380 | 10 | 0.43 | 0.38-0.47 | 2 |
| Abet0381 | 10 | 0.47 | | 1 |
| Abet0382 | 10 | 0.66 | | 1 |
| Abet0383 | 11 | 0.75 | | 1 |

Table 3 (see below): Sequence alignment of the VH domains of the optimized non-germlined clones described herein. Changes from the parent sequence (Abet0144-GL) are italicized. Residues are designated according to the Kabat numbering system.
Table 4 (see below): Sequence alignment of the VL domains of the optimized non-germlined clones described herein. Changes from the parent sequence (Abet0144-GL) are italicized. Residues are designated according to the Kabat numbering system. Note that Abet0378 has an amber stop codon “B” present in the VL sequence at position 91, which was introduced as a change from glutamine during optimisation. The antibody was produced as an scFv fragment in the E. coli strain TG1 used for expression in which the amber stop codon is read as glutamine.

TABLE 3

| Kabat | FW 1 | | | | | | | | | | | | | | | | | | | | | | | |
|---|---|---|---|---|---|---|---|---|---|---|---|---|---|---|---|---|---|---|---|---|---|---|---|---|
| Numbering V_{H} | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
| Abet0144-GL | E | V | D | L | L | E | S | G | G | G | L | V | D | P | G | G | S | L | R | L | S | C | A | A |
| Abet0319 | | | | | | | | | | | | | | | | | | | | | | | | |
| Abet0321b | | | | | | | | | | | | | | | | | | | | | | | | |
| Abet0322b | | | | | | | | | | | | | | | | | | | | | | | | |
| Abet0323b | | | | | | | | | | | | | | | | | | | | | | | | |
| Abet0328 | | | | | | | | | | | | | | | | | | | | | | | | |
| Abet0329 | | | | | | | | | | | | | | | | | | | | | | | | |
| Abet0332 | | | | | | | | | | | | | | | | E | | | | | | | | |
| Abet0342 | | | | | | | | | | | | | | | | | | | | | | | | |
| Abet0343 | | | | | | | | | | | | | | | | | | | | | | | | |
| Abet0344 | | | | | | | | | | | | | | | | | | | S | | | | | |
| Abet0368 | | | | | | | | | | | | | | | | | | | | | | | | |
| Abet0369 | | | | | | | | | | | | | | | | | | | | | | | | |
| Abet0370 | | | | | | | | | | | | | | | | | | | | | | | | |
| Abet0371 | | | | | S | | | | | | | | | | | | | | | | | | | |
| Abet0372 | | | | | | | | | | | | | | | | | | | | | | | | |
| Abet0373 | | | | | | | | | | | | | | | | | | | | | | | Y | |
| Abet0374 | | | | | | | | | | | | | | | | | | | | | | | | |
| Abet0377 | | | | | | | | | | | | | | | | | | | | | | | | |
| Abet0378 | | | | | | | | | | | | | | | | | | | | | | | | |
| Abet0379 | | | | | | | | | | | | | | | | | | | | | | | | |
| Abet0380 | | | | | | | | | | | | | | | | | | | | | | | | |
| Abet0381 | | | | | | | | | | | | | | | | | | | | | | | | |
| Abet0382 | | | | | | | | | | | | | | | | | | | | | | | | |
| Abet0383 | | | | | | | | | | | | | | | | | | | K | | | | | |
| Kabat | FW 1 | | | | | | CDR 1 | | | | | FW 2 | | | | | | | | | | | | |
| Numbering V_{H} | 25 | 26 | 27 | 28 | 29 | 30 | 31 | 32 | 33 | 34 | 35 | 36 | 37 | 38 | 39 | 40 | 41 | 42 | 43 | 44 | 45 | 46 | 47 | 48 |
| Abet0144-GL | S | G | F | T | F | S | V | Y | T | M | W | W | V | R | O | A | P | G | K | G | L | E | W | V |
| Abet0319 | | Y | S | V | Y | N | K | V | | | | | | | | | | | | | | | | |
| Abet0321b | | A | Y | H | S | N | M | O | P | | | | | | | | | | | | | | | |
| Abet0322b | | N | E | E | | O | Y | N | P | | | | | | | | | | | | | | | |
| Abet0323b | | T | S | | | O | C | O | | | | | | | | | | | | | | | | |
| Abet0328 | | S | D | A | | K | T | O | | | | | | | | | | A | | | | | | |
| Abet0329 | | T | | N | L | K | R | E | | | | | | | | | | | | | | | | |
| Abet0332 | | S | D | S | H | M | T | O | I | | | | | | | | | | | A | | | | |
| Abet0342 | | | | D | | R | A | S | V | | | | | | | | | | | | | | | |
| Abet0343 | | | | N | | N | H | O | V | | | | | | | | | | | | | | | |
| Abet0344 | | | | | | | | | | | | | | | | | | | | | | | | |
| Abet0368 | | | | D | | G | P | S | P | | | | | | | | | | | | | | | |
| Abet0369 | | S | | O | I | | K | N | | | | | | | A | | | | | | | | | |
| Abet0370 | | | | M | | P | M | S | A | | | | | | | | | | | | | | | |
| Abet0371 | | M | D | A | | P | F | Q | | | | | | | | | | | | | | | | |
| Abet0372 | | S | D | M | | N | I | E | | | | | | | | | | | | | | | | |
| Abet0373 | | | | D | | E | R | S | V | | | | | | | | | | | A | | | | |
| Abet0374 | | | | O | | K | Q | T | P | | | | | | | | | | | | | | | |
| Abet0377 | | | | N | | N | E | O | | L | | | | | | | | | | | | | | |
| Abet0378 | | | | P | | E | T | O | I | | | | | | | | | | | | | | | |
| Abet0379 | | | | D | | A | E | T | P | L | | | | | | | | | E | A | | | | |
| Abet0380 | | M | G | N | | N | Y | O | | | | | | | | | | | H | | | | | |
| Abet0381 | | S | P | S | | P | A | E | | | | | | | | | | | | | | | | |
| Abet0382 | | | | H | | T | N | S | I | | | | | | | | | | | | | | | |
| Abet0383 | | | | | | D | W | | P | | | | | | | | | | | R | | | | T |

| Kabat | FW 2 | CDR 2 | | | | | | | | | | | | | | | | |
|---|---|---|---|---|---|---|---|---|---|---|---|---|---|---|---|---|---|---|
| Numbering V_{H} | 49 | 50 | 51 | 52 | 52a | 53 | 54 | 55 | 56 | 57 | 58 | 59 | 60 | 61 | 62 | 63 | 64 | 65 |
| Abet0144-GL | S | Y | I | G | S | S | G | G | T | T | V | Y | A | D | S | V | K | G |
| Abet0319 | | | | | | | | | | | | | | | | | | |
| Abet0321b | | | | | | | | | | | A | | | | | | | |
| Abet0322b | | | | | | | | | A | | | | | | A | | | |
| Abet0323b | | | | | P | N | P | K | H | N | A | | | | | | | |
| Abet0328 | | | | | A | H | T | N | M | S | A | | | | | | | |
| Abet0329 | | | | | | H | Q | E | R | | S | | | | | | | |
| Abet0332 | | | | | N | | N | K | K | T | A | | | | | | | |
| Abet0342 | | | | | A | O | T | O | N | K | A | | | | | | | |
| Abet0343 | | | | | K | T | N | E | N | I | A | | | | | | | |
| Abet0344 | | | | | G | N | E | T | A | K | A | | | | | | | |
| Abet0368 | | | | | K | D | T | O | N | S | T | | | | | | | |
| Abet0369 | | | | | K | D | E | T | A | E | N | | | | | | | |
| Abet0370 | | | | | E | T | P | E | R | Q | A | | | | | | | |
| Abet0371 | | | | | | | | | | | | | | | | | | |
| Abet0372 | | | | | K | G | M | N | N | V | S | | | | | | | |
| Abet0373 | | | | | | G | K | T | N | I | T | | | | | | | |
| Abet0374 | | | | | D | Q | N | M | K | K | A | | | | | | | |
| Abet0377 | | | | | Y | G | T | K | N | T | A | | | | T | | | |
| Abet0378 | | | | | T | N | T | O | N | V | A | | | | | | | |
| Abet0379 | | | | | | N | O | N | K | | A | | | | | | | |
| Abet0380 | | | | | K | T | N | E | N | I | A | | | | | | | |
| Abet0381 | | | | | T | O | P | N | H | L | T | | | | | | | |
| Abet0382 | | | | | | E | A | H | R | V | T | | | | | | | |
| Abet0383 | | | | | A | D | N | A | K | I | A | | | | | | | |

| Kabat | FW 3 | | | | | | | | | | | | | | | | | | | | | |
|---|---|---|---|---|---|---|---|---|---|---|---|---|---|---|---|---|---|---|---|---|---|---|
| Numbering V_{H} | 66 | 67 | 68 | 69 | 70 | 71 | 72 | 73 | 74 | 75 | 76 | 77 | 78 | 79 | 80 | 81 | 82 | 82a | 82b | 82c | 83 | 84 |
| Abet0144-GL | R | F | T | I | S | R | D | N | S | K | N | T | L | Y | L | Q | M | N | S | L | R | A |
| Abet0319 | | | | | | | | | | | | | | | | | | | | | | |
| Abet0321b | | | | | | | | | | | | | | | | | | | | | | |
| Abet0322b | | | | | | | | | | E | | | | | | | | | | | | |
| Abet0323b | | | | | | | | | | | | | | | | | | | | | | |
| Abet0328 | | | | | | | | | | | | | | | | | | | | | | |
| Abet0329 | | | | | | | | | | | | | | | | | | | | | | |
| Abet0332 | | | | | | | | | | | | | | | | | | | | | | |
| Abet0342 | | | | | | | | | | | | | | | | | | | | | | |
| Abet0343 | | | | | | | | | | | | R | | | | | | | | | | |
| Abet0344 | | | | | | | | | | | D | | | | | | | | | | K | |
| Abet0368 | | | | | | | | | | | | | | | | | | | | | | |
| Abet0369 | | | | | | | | | | | S | | | | | | | | | | | |
| Abet0370 | | | | V | | | | | | | | | | | | | | | | | | |
| Abet0371 | | | | | | | | | | | | | | | | | | | | | | |
| Abet0372 | | | | | | | | | | | | | | | | | | | | | | |
| Abet0373 | | | | | | | | | | | | | | | | | | | | | | |
| Abet0374 | | | | | | | | | | | | | | | | | | | | | | |
| Abet0377 | | | | | | | | | | | | | | | | | | | | | | |
| Abet0378 | | | | | | | | | | | D | | | | | | | | | | | |
| Abet0379 | | | | | | | | | | | | | | | | | | | | | | |
| Abet0380 | | | | | | | | | | | | | | | | | | | | | | |
| Abet0381 | | | | | | | | | | | | | | | | | | | | | | |
| Abet0382 | | | | | | | | | | | | | | | | | | | | | | |
| Abet0383 | | | | | | | | | | | | | | | | | | | | | | |

| Kabat | FW 3 | | | | | | | | | | CDR 3 | | | | | | | | | | |
|---|---|---|---|---|---|---|---|---|---|---|---|---|---|---|---|---|---|---|---|---|---|
| Numbering V_{H} | 85 | 86 | 87 | 88 | 89 | 90 | 91 | 92 | 93 | 94 | 95 | 96 | 97 | 98 | 99 | 100 | 100a | 100b | 100c | 100d | 100e |
| Abet0144-GL | E | D | T | A | Y | Y | Y | C | A | R | E | W | M | D | H | S | R | P | Y | Y | Y |
| Abet0319 | | | | | | | | | | | | | | | | | | | | | |
| Abet0321b | | | | | | | | | | | | | | | | | | | | | |
| Abet0322b | | | | | | | | | | | | | | | | | | | | | |
| Abet0323b | | | | | | | | | | | | | | | | | | | | | |
| Abet0328 | | | | | | | | | | | | | | | A | | | | | | |
| Abet0329 | | | | | | | | | | | | | | | | | | | | | |
| Abet0332 | | | | | | | | | | | | | | | | | | | | | |
| Abet0342 | | | | | | | | | | | | | | | | | | | | | |
| Abet0343 | | | | | | | | | | | | | | | | | | | | | |
| Abet0344 | | | | | | | | | | | | | | | | | | | | | |
| Abet0368 | | | | | | | | | | | | | | | | | | | | | |
| Abet0369 | | | | | | | | | | | | | | | | | | | | | |
| Abet0370 | | | | | | | | | | | | | | | | | | | | | |
| Abet0371 | | | | | | | | | | | | | | | | | | | | | |
| Abet0372 | | | | | | | | | | | | | | | | | | | | | |
| Abet0373 | | | | | | | | | | | | | | | | | | | | | |
| Abet0374 | | | | | | | | | | | | | | | | | | | | | |
| Abet0377 | | | | | | | | | | | | | | | | | | | | | |
| Abet0378 | | | | | | | | | | | | | | | | | | | | | |
| Abet0379 | | | | | | | | | | | | | | | | | | | | | |
| Abet0380 | | | | | | | | | | | | | | | | | | | | | |
| Abet0381 | | | | | | | | | | | | | | | | | | | | | |
| Abet0382 | | | | | | | | | | | | | | | | | | | | | |
| Abet0383 | | | | | | | | | | | | | | G | | | | | | | |

| Kabat | CDR 3 | | | | | FW 4 | | | | | | | | | | |
|---|---|---|---|---|---|---|---|---|---|---|---|---|---|---|---|---|
| Numbering V_{H} | 100f | 100g | 100h | 101 | 102 | 103 | 104 | 105 | 106 | 107 | 108 | 109 | 110 | 111 | 112 | 113 |
| Abet0144-GL | Y | G | M | D | V | W | G | Q | G | T | L | V | T | V | S | S |
| Abet0319 | | | | | | | | | | | | | | | | |
| Abet0321b | | | | | | | | | | | | | | | | |
| Abet0322b | | | | | | | | | | | | | | | | |
| Abet0323b | | | | | | | | | | | | | | | | |
| Abet0328 | | | | | | | | | | | | | | | | |
| Abet0329 | | | | | | | | | | | | | | | | |
| Abet0332 | | | | | | | | | | | | | | | | |
| Abet0342 | | | | | | | | | | | | | | | | |
| Abet0343 | | | | | | | | | | | | | | | | |
| Abet0344 | | | | | | | | | | | | | | | | |
| Abet0368 | | | | | | | | | | | | | | | | |
| Abet0369 | | | | | | | | | | | | | | | | |
| Abet0370 | | | | | | | | | | | | | | | | |
| Abet0371 | | | | | | | | | | | | | | | | |
| Abet0372 | | | | | | | | | | | | | | | | |
| Abet0373 | | | | | | | | | | | | | | | | |
| Abet0374 | | | | A | | | | | | | | | | | | |
| Abet0377 | | | | | | | | | | | | | | | | |
| Abet0378 | | | | | | | | | | | | | | | | |
| Abet0379 | | | | | | | | | | | | | | | | |
| Abet0380 | | | | | | | | | | | | | | | | |
| Abet0381 | | T | | | | | | | | | | | | | P | |
| Abet0382 | | | | | | | | | | | | | | | | |
| Abet0383 | | | | | | | | | | P | | | | | | |

TABLE 4

| Kabat | FW 1 | | | | | | | | | | | | | | | | | | | | | | | CDR 1 | | | | |
|---|---|---|---|---|---|---|---|---|---|---|---|---|---|---|---|---|---|---|---|---|---|---|---|---|---|---|---|---|
| Numbering V_{L} | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 |
| Abet0144-GL | S | Y | E | L | T | Q | P | P | S | — | V | S | V | S | P | G | Q | T | A | S | I | T | C | S | G | H | N | L |
| Abet0319 | | | | | | | | | | | | | | | | | | | | | | | | | | | | I |
| Abet0321b | | | | | | | | | | | | | | | | | | | | | | | | | | | | |
| Abet0322b | | | | | | | | | | | | | | | | | | | | | | | | | | | | |
| Abet0323b | | | | | | | | | | | | | | | | | | | | | | | | | | | | |
| Abet0328 | | | | | | | | | | | | | | | | | | | V | | | | | | | R | | |
| Abet0329 | | | | | | | | | | | | | | | | | | | | | | | | | | | | V |
| Abet0332 | | | | | | | | | | | | | | | | | | | | | | | | | | | | I |
| Abet0342 | | | | | | | | | | | | | | | | | | | | | | | | | | | | |
| Abet0343 | Q | S | V | | | | | | | | | | | | | | | | | | | | | | | | | |
| Abet0344 | Q | S | V | | | | | | | | | | | | | | | | | | | | | | | | | |
| Abet0368 | | | | | | | | | | | | | | | | | | | | | | | | | | | | |
| Abet0369 | | | G | | | | | | | | | | | | | | | | | | | | | | | R | | I |
| Abet0370 | | | | | | | | | | | | | | | | | | | | | | | | T | T | P | H | F |
| Abet0371 | | | | | | | | | | | | | | | | | | | | | | | | | | | | I |
| Abet0372 | | | | | | | | | | | | | | | | | | | | | | | | | | | | |
| Abet0373 | | | | | | | | | | | | | | | | | | | | | | | | | | | | |
| Abet0374 | | | | | | | | | | | | | | T | | | | | | | | | | | | | | |
| Abet0377 | | | | | | | | | | | | | | | | | | | | | | | | | | | | T |
| Abet0378 | | | | | | | | | | | | | | | | | | | | | | | | | | | | |
| Abet0379 | Q | S | V | | | | | | | | | | | | | | | | | | | | | | | | | |
| Abet0380 | | | | | | | | | | | | | | | | | | | | | | | | | | | | |
| Abet0381 | | | | | | | | | | | | | A | | | | | | | | | | | | | | | |
| Abet0382 | | | | | I | | | | | | | | | | | | | | | | | | | | | | | |
| Abet0383 | | | | | | | | | | | | | | | | | | | | | | | | | | | | |
| Kabat | CDR 1 | | | | | | FW 2 | | | | | | | | | | | | | | | CDR 2 | | | | | | |
| Numbering V_{L} | 29 | 30 | 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 |
| Abet0144-GL | E | D | K | F | A | S | W | Y | Q | Q | K | P | G | Q | S | P | V | L | V | I | Y | R | D | D | K | R | P | S |
| Abet0319 | M | | | W | V | | | | | | | | | R | | | A | | | | | | | | | | | |
| Abet0321b | | | | | | | | | | | | | | | | | | | I | | | | | | | | | |
| Abet0322b | G | | | | | | | | | | | | | | | | | | | | | | | | | | | |
| Abet0323b | | | | | | | | | | | | | | | | | | | | | | | | | | | | |
| Abet0328 | | | | | | | | | | | | | | | | | | | | | | | | | | | | |
| Abet0329 | S | | | W | M | T | | | | | | | | | | | | | | | | | | | | | | |
| Abet0332 | G | A | | W | V | | | | | | | | | | | | I | | | | | | | | | | | |
| Abet0342 | | | | | | | | | | | | | | | | | | | | | | | | | | | | |
| Abet0343 | | | | | | | | | | | | S | | | | | | | | | | | | | | | | |
| Abet0344 | | | | | | | | | | | | S | | | | | | | | | | | | | | | | |
| Abet0368 | | | | | T | | | | | | | S | | | | | | | | | | | | | | | | |
| Abet0369 | G | | S | W | V | A | | | | | | | | | | | | | | | | | | | | | | |
| Abet0370 | N | S | | | | | | | | | | | | | | | | | | | | | | | | | | |
| Abet0371 | S | S | S | W | V | | | | | | | | | | | | | | | | | | | | | | | |
| Abet0372 | | | | | | | | | | | | | | | | | | | | | | | | | | | | |
| Abet0373 | | | | | | | | | | | | | | | | | | | | | | | | | | | | |
| Abet0374 | G | G | | | | | | | | | | | | | | | | | | | | | | | | | | |
| Abet0377 | | H | | W | I | | | | | | | | | | | | | | | | | | | | | | | |
| Abet0378 | | | | | | | | | | | | | | | | | | | | | | | | | | | | |
| Abet0379 | | | | | | | | | | | | S | | | | | | | | | | | | | | | | |
| Abet0380 | | | | | | | | | | | | | | | | | | | | | | | | | | | | |
| Abet0381 | | | | V | | | | | | | | | | | | | | | | | | | | | | | | |
| Abet0382 | | | | | | | | | | | | | | | | | | | | | | | | | | | | |
| Abet0383 | G | | | | | | | | | | | | | | | | | | | | | | | | | | | |
| Kabat | FW 3 | | | | | | | | | | | | | | | | | | | | | | | | | | | |
| Numbering V_{L} | 57 | 58 | 59 | 60 | 61 | 62 | 63 | 64 | 65 | 66 | 67 | 68 | 69 | 70 | 71 | 72 | 73 | 74 | 75 | 76 | 77 | 78 | 79 | 80 | 81 | 82 | 83 | 84 |
| Abet0144-GL | G | I | P | E | R | F | S | A | S | N | S | G | H | T | A | T | L | T | I | S | G | T | Q | A | M | D | E | A |
| Abet0319 | | | | | | | | | | | | | | | | | | | | | | | | | T | | | |
| Abet0321b | | | | | | | | | | | | | | | | | | | | | | | | | T | | | |
| Abet0322b | E | | | | | | | | | | | | | | | | | | | | | | | | T | | | |
| Abet0323b | | V | | | | | | | | | | | | | | | | | | | | | | | T | | | |
| Abet0328 | | V | | | | | | | | | | | | | | | | | | | | | | | T | | | |
| Abet0329 | | | | | | | | | | | | | | | | | | | | | | | | | T | | | |
| Abet0332 | | | | | | | | | | | | | | | | | | | | | | | | | T | | | |
| Abet0342 | | | | | | | | | | | | | D | | | | | | | | | | | | | | | |
| Abet0343 | | | | | | | | | | | | | | | | | | | | | | | | | T | | | |
| Abet0344 | | | | | | | | | | | | | | | | | | | | | | | | | T | | | |
| Abet0368 | | | | | | | | | | | | | | | | | | | | | | A | | | T | | | |
| Abet0369 | | | | | | | | | | | | | | | | | | | | | | | | | T | | | |
| Abet0370 | | | | | | | | | | | | | | | | | | | | | | | | | | | | |
| Abet0371 | | | | | | | | | | | | | | | | | | | | | | | | | T | | | |
| Abet0372 | | | | | | | | | | | | | | | | | | | | | | | | | T | | | |
| Abet0373 | E | | | | | | | | | | | | | | | | | | | | | | | | T | | | |
| Abet0374 | | | | | | | | | | | F | | | | | | | | | | | | | | T | | | |
| Abet0377 | | | | | | | | | T | | | | | | | | | | | | | | | | T | | | |
| Abet0378 | | | | | | | | | | | | | | | | | | | | | | | | | T | | | |
| Abet0379 | | | | | | | | | | | | | | | | | | | | | | | | | T | | G | |
| Abet0380 | | | | | | | | | | | | | | | | | | | | | | | | | T | | | |
| Abet0381 | | | | | | | | | | | | | | | | | | | | | | | | | T | | | |
| Abet0382 | | | | | | | | | T | | | | | | | | | | | | | | | | T | | | |
| Abet0383 | | | | | | | | | | | | | | | | | | | | | | | | | T | | | |

| Kabat | FW 3 | | | | CDR 3 | | | | | | | | | FW 4 | | | | | | | | | |
|---|---|---|---|---|---|---|---|---|---|---|---|---|---|---|---|---|---|---|---|---|---|---|---|
| Numbering V_{L} | 85 | 86 | 87 | 88 | 89 | 90 | 91 | 92 | 93 | 94 | 95 | 96 | 97 | 98 | 99 | 100 | 101 | 102 | 103 | 104 | 105 | 106 | 107 |
| Abet0144-GL | D | Y | Y | C | Q | A | Q | D | S | T | T | R | V | F | G | G | G | T | K | L | T | V | L |
| Abet0319 | | | | | S | S | | | T | V | | | | | | | | | | | | | |
| Abet0321b | | | | | S | S | | | T | V | | | | | | | | | | | | | |
| Abet0322b | | | | | S | S | | | T | V | | | | | | | | | | | | | |
| Abet0323b | | | | | S | S | | | T | V | | | | | | | | | | | I | | |
| Abet0328 | | | | | S | S | | | T | V | | | | | | | | | | | | | |
| Abet0329 | | | | | S | S | | | T | V | | | | | | | | | | | | | |
| Abet0332 | | | | | | | | G | Q | V | | | S | | | | | | | | | | |
| Abet0342 | | | | | | | | | | | | | | | | | | | | | | | |
| Abet0343 | | | | | S | S | | | T | V | | | | | | | | | | | | | |
| Abet0344 | | | | | A | T | | | N | F | | | | | | | | | | | | | |
| Abet0368 | | | | | S | S | | | T | V | | | | | | | | | | | | | |
| Abet0369 | | | | | S | S | | | T | V | | | | | | | | | | | | | |
| Abet0370 | | | | | | | | | | | | | | | | | | | R | | | | |
| Abet0371 | | | | | S | S | | | T | V | | | | | | | | | | | | | |
| Abet0372 | | | | | S | S | | | T | V | | | | | | | | | | | | | |
| Abet0373 | | | | | S | S | | | T | V | | | | | | | | | | | | | |
| Abet0374 | | | | | S | S | | | T | V | | | | | | | | | | | | | |
| Abet0377 | | | | | S | S | | | T | V | | | | | | | | | | | | | |
| Abet0378 | | | | | S | S | B | | T | V | | | | | | | | | | | | | |
| Abet0379 | | | | | A | T | | | N | F | | | | | | | | | | | | | |
| Abet0380 | | | | | S | S | | | T | V | | | | | | | | | | | | | |
| Abet0381 | N | | | | S | S | | | T | V | | | A | | | | | | | | | | |
| Abet0382 | | | | | S | S | | | K | V | | | | | | | | | | | | | |
| Abet0383 | | | | | S | S | | | T | V | | | | | | | | | | | | | |

1.7 Kinetic Profiling of Affinity Improved Clones in Purified scFv Format by Surface Plasmon Resonance

Surface Plasmon Resonance was used to analyse the purified scFv clones that had shown significant improvement in binding affinity for human Amyloid beta 1-42 peptide over the parent sequence, Abet0144-GL, in the HTRF™ epitope competition assay (sections 1.3-1.6). Briefly, the ProteOn Protein Interaction Array System (BioRad, USA) was used to assess the kinetic parameters of the interaction between each purified scFv and synthetically produced human Amyloid beta 1-42 peptide. These experiments were performed essentially as described by Karlsson et al. (Karlsson et al., 1991).

The affinity of binding between each test scFv and human Amyloid beta 1-42 was estimated using assays in which biotinylated synthetic human Amyloid beta 1-42 peptide (rPeptide, USA; cat: Al 117) was non-covalently bound via a biotin/streptavidin interaction to a proprietary streptavidin chip (NTA 176-5021) at five different surface densities. The chip surface was regenerated between cycles by a single 60 second injection of 10 mM Glycine pH 2.0 to remove scFv bound to the peptide. The regeneration did not result in a significant loss of scFv binding capacity.

Each scFv at 100-200 nM was sequentially passed over the peptide surface for a sufficient amount of time to observe sensorgrams that could be fitted to an appropriate binding model with confidence. An irrelevant scFv blank was subtracted from the main dataset to reduce the impact of any buffer artefacts or non-specific binding effects. An appropriate binding model was then fitted to the data.

For Abet0380 scFv, the association rate constant (ka), dissociation rate constant (kd) and dissociation constant (KD) are 1.93×10⁵ M⁻¹ s⁻¹, 2.85×10⁻⁵ s⁻¹ and 148 pM respectively. These parameters were derived from a 1:1 Langmuir fit to the data.

TABLE 5
Example kinetic data for optimized scFv clones binding
to synthetic biotinylated human Amyloid beta 1-42 peptide,
as determined by Surface Plasmon Resonance.

| Clone | kₐ (M⁻¹ s⁻¹) | k_{d} (s⁻¹) | K_{D} (M) |
|---|---|---|---|
| Abet0144-GL | 1.16E+05 | 6.60E−03 | 5.87E−08 |
| Abet0319 | 3.29E+05 | 1.29E−04 | 3.91E−10 |
| Abet0321b | 1.50E+05 | 3.33E−05 | 2.22E−10 |
| Abet0322b | 2.03E+05 | 1.65E−04 | 8.12E−10 |
| Abet0323b | 2.10E+05 | 1.88E−04 | 8.94E−10 |
| Abet0328 | 1.41E+05 | 1.03E−04 | 7.29E−10 |
| Abet0329 | 1.97E+05 | 1.38E−04 | 7.01E−10 |
| Abet0332 | 3.29E+05 | 1.29E−04 | 3.91E−10 |
| Abet0342 | 1.36E+05 | 5.73E−05 | 4.21E−10 |
| Abet0343 | 1.20E+05 | 2.25E−05 | 1.88E−10 |
| Abet0344 | 7.75E+04 | 5.73E−05 | 7.39E−10 |
| Abet0368 | 1.87E+05 | 9.00E−05 | 4.82E−10 |
| Abet0369 | 3.27E+05 | 4.34E−05 | 1.33E−10 |
| Abet0370 | 1.19E+05 | 7.76E−05 | 6.51E−10 |
| Abet0371 | 3.57E+05 | 2.72E−04 | 7.62E−10 |
| Abet0372 | 2.43E+05 | 1.76E−04 | 7.24E−10 |
| Abet0373 | 1.85E+05 | 8.92E−05 | 4.83E−10 |
| Abet0374 | 2.56E+05 | 6.04E−05 | 2.36E−10 |
| Abet0377 | 1.96E+05 | 3.02E−05 | 1.54E−10 |
| Abet0378 | 1.36E+05 | 6.41E−05 | 4.72E−10 |
| Abet0379 | 1.34E+05 | 4.39E−05 | 3.27E−10 |
| Abet0380 | 1.93E+05 | 2.85E−05 | 1.48E−10 |
| Abet0381 | 2.13E+05 | 5.14E−05 | 2.41E−10 |
| Abet0382 | 2.25E+05 | 7.97E−05 | 3.54E−10 |
| Abet0383 | 1.81E+05 | 3.94E−05 | 2.17E−10 |

1.8 Reformatting of Affinity Improved scFv to Human IgG1-TM

ScFv were reformatted to IgG1-TM by subcloning the variable heavy chain (V_{H}) and variable light chain (V_{L}) domains into vectors expressing whole human antibody heavy and light chains respectively. The variable heavy chain was cloned into a mammalian expression vector (pEU 1.4) containing the human heavy chain constant domains and regulatory elements to express whole IgG1-TM heavy chain in mammalian cells. Similarly, the variable light chain domain was cloned into a mammalian expression vector (pEU 4.4) for the expression of the human lambda light chain constant domains and regulatory elements to express whole IgG light chain in mammalian cells.

To obtain antibodies as IgG, the heavy and light chain IgG expression vectors were transiently transfected into HEK293-EBNA mammalian cells (Invitrogen, UK; cat: R620-07) where the IgGs were expressed and secreted into the medium. Harvests were pooled and filtered prior to purification. The IgG was purified using Protein A chromatography. Culture supernatants were loaded onto an appropriate ceramic Protein A column (BioSepra—Pall, USA) and washed with 50 mM Tris-HCl pH 8.0, 250 mM NaCl. Bound IgG was eluted from the column using 0.1 M Sodium Citrate (pH 3.0) and neutralized by the addition of Tris-HCl (pH 9.0). The eluted material was buffer exchanged into PBS using NAP-10 buffer exchange columns (GE Healthcare, UK; cat: 17-0854-02) and the purified IgGs were passed through a 0.2 μm filter. The concentration of IgG was determined spectrophotometrically using an extinction coefficient based on the amino acid sequence of the IgG. The purified IgGs were analysed for aggregation or degradation using SEC-HPLC and by SDS-PAGE.

### 1.9 Germlining

Five of the most potent IgGs were selected for germlining, based on an experimental characterisation of their corresponding scFv. Purified scFv of clones Abet0343, Abet0369, Abet0377, Abet0380 and Abet0382 all exhibited IC₅₀ values of less than 750 pM, as determined by epitope competition assay (Table 2), and all had an experimental dissociation constant of less than 250 pM, as determined by Surface Plasmon Resonance, Table 5. The germlining process consisted of reverting framework residues in the Vii and V_{L} domains to the closest germline sequence to identically match human antibodies. For the V_{H} domains of the optimized antibody lineage this was Vh3-23 (DP-47) and for the V_{L} domains it was V λ3-3r (DPL-23). For Abet0380, 1 residue required changing in the V_{H} domain at Kabat position 43 (Table 6) and 1 residue required changing in the V_{L} domain at Kabat position 81 (Table 7). The remaining four sequences required between two and five changes (Tables 6 and 7). The Vernier residues (Foote et al., 1992), were not germlined, apart from residue 2 in the light chain sequence of Abet0343, which was germlined for at the same time as the flanking residues 1 and 3. Germlining of these amino acid residues was carried out using standard site-directed mutagenesis techniques with the appropriate mutagenic primers as described by Clackson and Lowman (Clackson et al., 2004).

TABLE 6
Sequence alignment of the V_{H} domains of the five clones selected for germlining. The two residues that were reverted to
germline are indicated by italics. The positions of the Vernier residues are indicated by circles (●).

| Kabat | FW 1 | | | | | | | | | | | | | | | | | | | | | | | |
|---|---|---|---|---|---|---|---|---|---|---|---|---|---|---|---|---|---|---|---|---|---|---|---|---|
| Numbering V_{H} | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 |
| Vernier | | ● | | | | | | | | | | | | | | | | | | | | | | |
| Abet0144-GL | E | V | Q | L | L | E | S | G | G | G | L | V | Q | P | G | G | S | L | R | L | S | C | A | A |
| Abet0343 | | | | | | | | | | | | | | | | | | | | | | | | |
| Abet0369 | | | | | | | | | | | | | | | | | | | | | | | | |
| Abet0377 | | | | | | | | | | | | | | | | | | | | | | | | |
| Abet0380 | | | | | | | | | | | | | | | | | | | | | | | | |
| Abet0382 | | | | | | | | | | | | | | | | | | | | | | | | |
| Kabat | FW 1 | | | | | | CDR 1 | | | | | FW 2 | | | | | | | | | | | | |
| Numbering V_{H} | 25 | 26 | 27 | 28 | 29 | 30 | 31 | 32 | 33 | 34 | 35 | 36 | 37 | 38 | 39 | 40 | 41 | 42 | 43 | 44 | 45 | 46 | 47 | 48 |
| Vernier | | | ● | ● | ● | ● | | | | | | | | | | | | | | | | | ● | ● |
| Abet0144-GL | S | G | F | T | F | S | V | Y | T | M | W | W | V | R | Q | A | P | G | K | G | L | E | W | V |
| Abet0343 | | | | N | | N | H | Q | V | | | | | | | | | | | | | | | |
| Abet0369 | | S | | Q | I | | K | N | | | | | | | R | | | | | | | | | |
| Abet0377 | | | | N | | N | E | Q | | L | | | | | | | | | | | | | | |
| Abet0380 | | M | G | N | | N | Y | Q | | | | | | | | | | | R | | | | | |
| Abet0382 | | | | H | | T | N | S | I | | | | | | | | | | | | | | | |

| Kabat | FW 2 | CDR 2 | | | | | | | | | | | | | | | | |
|---|---|---|---|---|---|---|---|---|---|---|---|---|---|---|---|---|---|---|
| Numbering V_{H} | 49 | 50 | 51 | 52 | 52a | 53 | 54 | 55 | 56 | 57 | 58 | 59 | 60 | 61 | 62 | 63 | 64 | 65 |
| Vernier | ● | | | | | | | | | | | | | | | | | |
| Abet0144-GL | S | V | I | G | S | S | G | G | T | T | V | Y | A | D | S | V | K | G |
| Abet0343 | | | | | K | T | N | E | N | I | A | | | | | | | |
| Abet0369 | | | | | K | D | E | T | R | F | N | | | | | | | |
| Abet0377 | | | | | V | G | T | K | N | I | A | | | | T | | | |
| Abet0380 | | | | | K | T | N | E | N | I | A | | | | | | | |
| Abet0382 | | | | | | E | A | H | R | V | T | | | | | | | |

| Kabat | FW 3 | | | | | | | | | | | | | | | | | | | | | |
|---|---|---|---|---|---|---|---|---|---|---|---|---|---|---|---|---|---|---|---|---|---|---|
| Numbering V_{H} | 66 | 67 | 68 | 69 | 70 | 71 | 72 | 73 | 74 | 75 | 76 | 77 | 78 | 79 | 80 | 81 | 82 | 82a | 82b | 82c | 83 | 84 |
| Vernier | | ● | | ● | | ● | | ● | | | | | ● | | | | | | | | | |
| Abet0144-GL | R | F | T | I | S | R | D | N | S | K | N | T | L | Y | I | Q | M | N | S | L | R | A |
| Abet0343 | | | | | | | | | | | | | | | | | | | | | | |
| Abet0369 | | | | | | | | | | | | | | | | | | | | | | |
| Abet0377 | | | | | | | | | | | | | | | | | | | | | | |
| Abet0380 | | | | | | | | | | | | | | | | | | | | | | |
| Abet0382 | | | | | | | | | | | | | | | | | | | | | | |

| Kabat | FW 3 | | | | | | | | | | CDR 3 | | | | | | | | | | |
|---|---|---|---|---|---|---|---|---|---|---|---|---|---|---|---|---|---|---|---|---|---|
| Numbering V_{H} | 85 | 86 | 87 | 88 | 89 | 90 | 91 | 92 | 93 | 94 | 95 | 96 | 97 | 98 | 99 | 100 | 100a | 100b | 100c | 100d | 100e |
| Vernier | | | | | | | | | ● | ● | | | | | | | | | | | |
| Abet0144-GL | E | D | T | A | V | Y | Y | C | A | R | E | W | M | D | H | S | R | P | Y | Y | Y |
| Abet0343 | | | | | | | | | | | | | | | | | | | | | |
| Abet0369 | | | | | | | | | | | | | | | | | | | | | |
| Abet0377 | | | | | | | | | | | | | | | | | | | | | |
| Abet0380 | | | | | | | | | | | | | | | | | | | | | |
| Abet0382 | | | | | | | | | | | | | | | | | | | | | |

| Kabat | CDR 3 | | | | | FW 4 | | | | | | | | | | |
|---|---|---|---|---|---|---|---|---|---|---|---|---|---|---|---|---|
| Numbering V_{H} | 100f | 100g | 100h | 101 | 102 | 103 | 104 | 105 | 106 | 107 | 108 | 109 | 110 | 111 | 112 | 113 |
| Vernier | | | | | | | | | | | | | | | ● | |
| Abet0144-GL | Y | G | M | D | V | W | G | Q | G | T | L | V | T | V | S | S |
| Abet0343 | | | | | | | | | | | | | | | | |
| Abet0369 | | | | | | | | | | | | | | | | |
| Abet0377 | | | | | | | | | | | | | | | | |
| Abet0380 | | | | | | | | | | | | | | | | |
| Abet0382 | | | | | | | | | | | | | | | | |

TABLE 7
Sequence alignment of the VL domains of the five clones selected for germlining. The thirteen residues that were reverted to germline are
indicated by italics. The positions of the Vernier residues are indicated by circles (●). The Vernier 2 residue in Abet0343 was reverted to germ-line
at the same time as residues 1 and 3. Reverting this residue did not impact on antibody potency.

| Kabat | FW 1 | | | | | | | | | | | | | | | | | | | | | | | CDR 1 | | | | |
|---|---|---|---|---|---|---|---|---|---|---|---|---|---|---|---|---|---|---|---|---|---|---|---|---|---|---|---|---|
| Numbering V_{L} | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | 28 |
| Vernier | | ● | | ● | | | | | | | | | | | | | | | | | | | | | | | | |
| Abet0144-GL | S | Y | E | L | T | Q | P | P | S | — | V | S | V | S | P | G | Q | T | A | S | I | T | C | S | G | H | N | L |
| Abet0343 | Q | S | V | | | | | | | | | | | | | | | | | | | | | | | | | |
| Abet0369 | | | G | | | | | | | | | | | | | | | | | | | | | | | R | | I |
| Abet0377 | | | | | | | | | | | | | | | | | | | | | | | | | | | | T |
| Abet0380 | | | | | | | | | | | | | | | | | | | | | | | | | | | | |
| Abet0382 | | | | | I | | | | | | | | | | | | | | | | | | | | | | | |
| Kabat | CDR 1 | | | | | | FW 2 | | | | | | | | | | | | | | | CDR 2 | | | | | | |
| Numbering V_{L} | 29 | 30 | 31 | 32 | 33 | 34 | 35 | 36 | 37 | 38 | 39 | 40 | 41 | 42 | 43 | 44 | 45 | 46 | 47 | 48 | 49 | 50 | 51 | 52 | 53 | 54 | 55 | 56 |
| Vernier | | | | | | | ● | ● | | | | | | | | | | ● | ● | ● | ● | | | | | | | |
| Abet0144-GL | E | D | K | F | A | S | W | Y | Q | Q | K | P | G | Q | S | P | V | L | V | I | Y | R | D | D | K | R | P | S |
| Abet0343 | | | | | | | | | | | | S | | | | | | | | | | | | | | | | |
| Abet0369 | G | | S | W | V | A | | | | | | | | | | | | | | | | | | | | | | |
| Abet0377 | | H | | W | I | | | | | | | | | | | | | | | | | | | | | | | |
| Abet0380 | | | | | | | | | | | | | | | | | | | | | | | | | | | | |
| Abet0382 | | | | | | | | | | | | | | | | | | | | | | | | | | | | |
| Kabat | FW 3 | | | | | | | | | | | | | | | | | | | | | | | | | | | |
| Numbering V_{L} | 57 | 58 | 59 | 60 | 61 | 62 | 63 | 64 | 65 | 66 | 67 | 68 | 69 | 70 | 71 | 72 | 73 | 74 | 75 | 76 | 77 | 78 | 79 | 80 | 81 | 82 | 83 | 84 |
| Vernier | | | | | | | | ● | | ● | | ● | ● | | ● | | | | | | | | | | | | | |
| Abet0144-GL | G | I | P | E | R | F | S | A | S | N | S | G | H | T | A | T | L | T | I | S | G | T | Q | A | M | D | E | A |
| Abet0343 | | | | | | | | | | | | | | | | | | | | | | | | | T | | | |
| Abet0369 | | | | | | | | | | | | | | | | | | | | | | | | | T | | | |
| Abet0377 | | | | | | | | | T | | | | | | | | | | | | | | | | T | | | |
| Abet0380 | | | | | | | | | | | | | | | | | | | | | | | | | T | | | |
| Abet0382 | | | | | | | | | T | | | | | | | | | | | | | | | | T | | | |

| Kabat | FW 3 | | | | CDR 3 | | | | | | | | | FW 4 | | | | | | | | | |
|---|---|---|---|---|---|---|---|---|---|---|---|---|---|---|---|---|---|---|---|---|---|---|---|
| Numbering V_{L} | 85 | 86 | 87 | 88 | 89 | 90 | 91 | 92 | 93 | 94 | 95 | 96 | 97 | 98 | 99 | 100 | 101 | 102 | 103 | 104 | 105 | 106 | 107 |
| Vernier | | | | | | | | | | | | | | | | | | | | | | | ● |
| Abet0144-GL | D | Y | Y | C | Q | A | Q | D | S | T | T | R | V | F | G | G | G | T | K | L | T | V | L |
| Abet0343 | | | | S | S | | | T | V | | | | | | | | | | | | | | |
| Abet0369 | | | | S | S | | | T | V | | | | | | | | | | | | | | |
| Abet0377 | | | | S | S | | | T | V | | | | | | | | | | | | | | |
| Abet0380 | | | | S | S | | | T | V | | | | | | | | | | | | | | |
| Abet0382 | | | | S | S | | | K | V | | | | | | | | | | | | | | |

### 1.10 Determination of the Binding Kinetics of Affinity-Optimized IgGs Using Surface Plasmon Resonance

Surface Plasmon Resonance was used to analyse the binding kinetics of the affinity-optimized IgGs (section 1.8) and their germlined counterparts (section 1.9). Briefly, the BIAcore T-100 (GE Healthcare, UK) biosensor instrument was used to assess the kinetic parameters of the interaction between each test IgG and synthetically-produced human Amyloid beta 1-42 peptide. These experiments were performed essentially as described by Karsson et al. (Karlsson et al., 1991).

The affinity of binding between each test IgG and human Amyloid beta 1-42 was estimated using assays in which each antibody was non-covalently captured by a protein G surface that was itself amine linked to a proprietary CM5 chip. The chip surface was regenerated between cycles by paired 40 second injections of 10 mM Glycine pH 2.0 to remove ligand and bound antibody. The test antibody was then reapplied for each peptide injection.

A series of dilutions of synthetic human Amyloid beta 1-42 peptide (0.063-1024 nM) were sequentially passed over the antibody surface for a sufficient amount of time to observe sensorgrams that could be fitted to an appropriate binding model with confidence. Blank reference flow-cell data were subtracted from each IgG dataset and a zero-concentration antibody-only buffer blank was double-reference subtracted from the main dataset. An appropriate binding model was then fitted simultaneously to the data from each analyte titration using the BIAevaluation software.

The validity of the data was assessed using the calculated Chi² value, with an acceptable value being under 2 RU². The overall success of the fit was estimated using the residuals, with a deviation of under 2 RUs being acceptable.

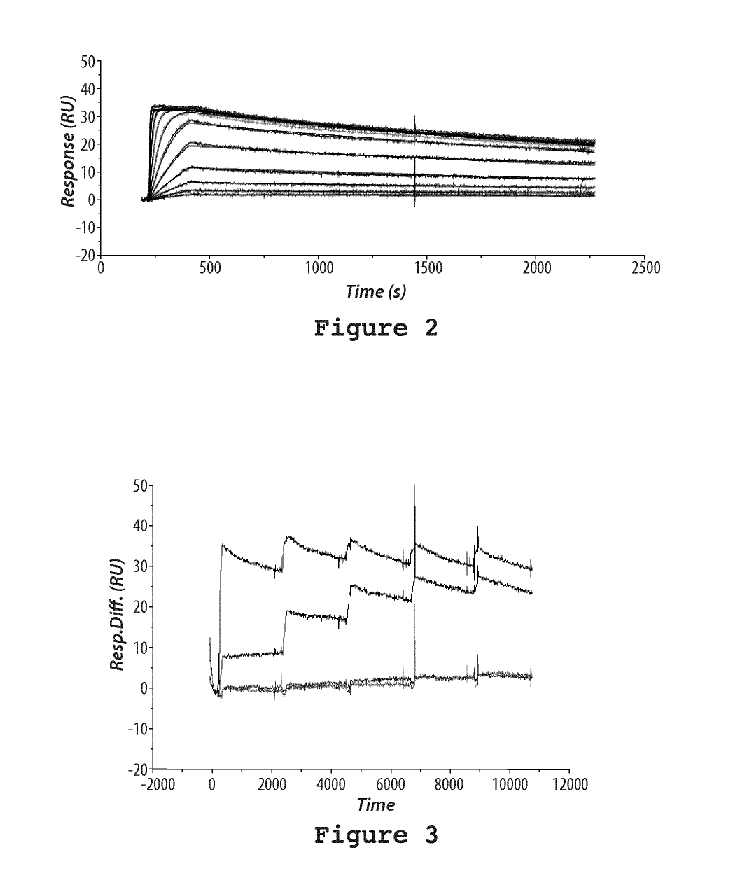

Example results for Abet0380-GL (germlined) IgG1-TM are shown in FIG. 2. The association rate constant (ka), dissociation rate constant (kd) and dissociation constant (KD) are 9.52×10⁵ M⁻¹ s⁻¹, 3.07×10⁻⁴ s⁻¹ and 322 pM respectively. These parameters were derived from a 1:1 Langmuir fit to the data

### 1.11 Specificity Profiling of Affinity-Optimized IgGs Using Surface Plasmon Resonance

Surface Plasmon Resonance was used to verify the specificity of the affinity-optimized IgGs for the human Amyloid beta 1-42 peptide. Briefly, the BIAcore2000 (GE Healthcare, UK) biosensor instrument was used to assess the kinetic parameters of the interaction between each test IgG and a range of small peptides including synthetically-produced human Amyloid beta 1-42 and human Amyloid beta 1-40. These experiments were performed essentially as described by Karlsson et al. (Karlsson et al., 1991).

The interaction between each test IgG and each peptide was estimated using assays in which the antibody was non-covalently captured by a protein G surface that was itself amine linked to a proprietary CM5 chip. The interaction between antibody and peptide was observed using a 5 application single cycle approach. The chip surface was regenerated between cycles by paired 40 second injections of 10 mM Glycine pH 2.0 to remove ligand and bound antibody. The test antibody was then reapplied for each peptide injection cycle.

Each test peptide (between 64 and 1024 nM) was sequentially passed over the antibody surface for a sufficient amount of time to observe sensorgrams that either showed no binding or that could be fitted to an appropriate binding model with confidence. Blank reference flow-cell data were subtracted from each IgG dataset and a zero-concentration antibody-only buffer blank was double-reference subtracted from the main dataset.

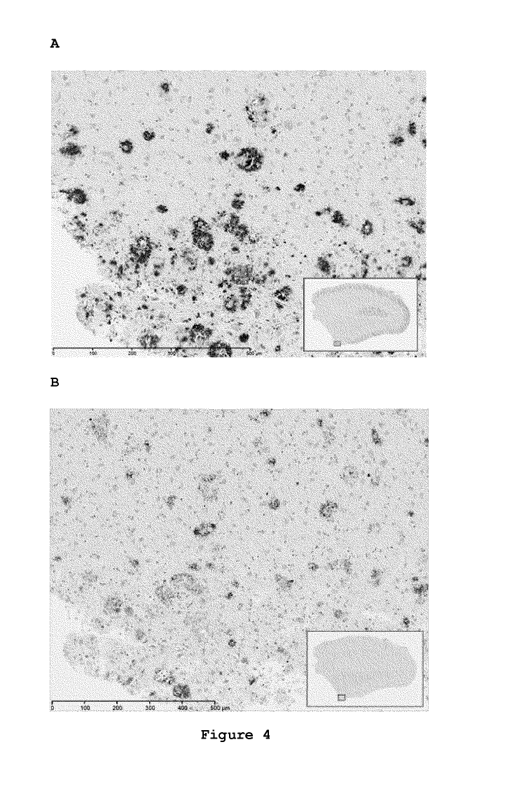

Example results for Abet0380-GL (germlined) IgG1-TM are shown in FIG. 3. Two peptides (biotinylated human Amyloid beta 1-42, (rPeptide, USA; cat: Al 117) and unlabelled murine Amyloid beta 1-42 (rPeptide, USA; cat: A1008) showed strong binding to the antibody, whilst two peptides biotinylated human Amyloid beta 1-40 (rPeptide, USA; cat: A1111) and unlabelled murine Amyloid beta 1-40 (rPeptide, USA; cat: A1007) showed no binding to the antibody.

### 1.12 Affinity of the Most Potent IgGs for Native Amyloid Beta Using In Vitro Immunohistochemistry

The most potent IgGs were tested for their ability to bind to Amyloid beta, with the aim of estimating the affinity of these clones for native forms of the Amyloid beta peptide. Briefly, the lead antibodies were screened on human Alzheimer's Disease brain sections and Tg2576 mouse brain sections to identify anti-Amyloid beta 1-42 antibodies that bound to Amyloid plaques in vitro.

In these experiments, human brain tissue was isolated from the frontal cortex of two individuals with severe Alzheimer's Disease (ApoE genotype 3/3, Braak stage 6 and ApoE genotype 4/3, Braak stage 5). As a control, equivalent tissue was isolated from one non-dementia individual (ApoE genotype 3/3, Braak stage 1). Mouse brain tissue was isolated from Tg2576 mice at an age of 15 months (2 mice) and 22 months (2 mice). Antibodies were tested at concentrations of 2, 5, 10 and 20 ug ml⁻¹.

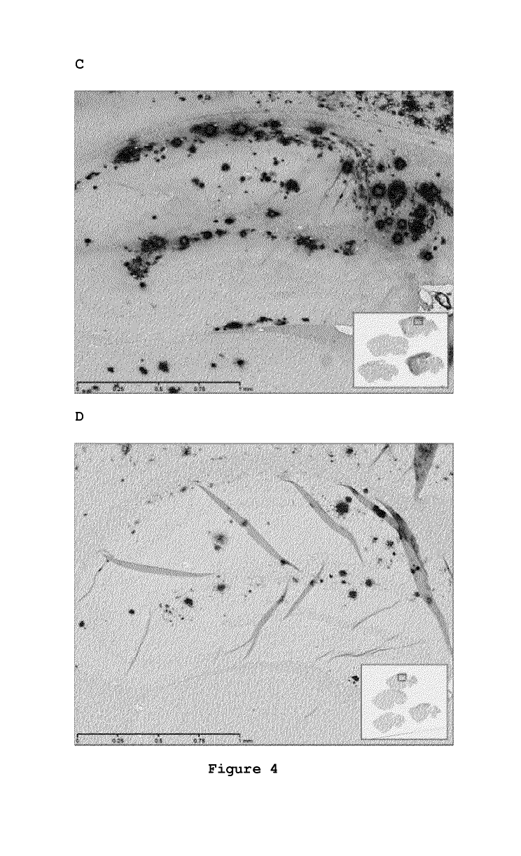

In one experiment, the Abet0380-GL IgG-TM antibody stained core plaques (CP) with a score of 4 on Tg2576 brain sections, and a score of 3 on human AD brain sections. It also stained diffuse plaques (DP) and cerebral amyloid angiopathy (CAA) plaques, but to a lesser extent. In contrast, a positive control antibody produced a score of 3-4 on all plaques (CP. DP, CAA) on adjacent sections under the same conditions. Representative images are shown in FIG. 4.

### 1.13 Demonstrating Abet00380-GL IgG1-TM Abeta42 Recognition Profile by Western Blot

To cross-link the A042 oligomers before SDS-PAGE, PICUP (photo-induced cross-linking of peptides) was carried out as follows. A 1 mM solution of Ru(Bpy) was created by adding 2 μl of stock (at 10 mM) to 18 μl of 1×PBS. In addition, a 20 mM solution of ammonium persulphate (APS) was created by adding 2 Cl of stock (at 200 mM) to 18 μl of 1×PBS. Unused stock was immediately snap-frozen on dry ice and returned to the −80° C. freezer. In the dark room, 5 μl of Ru(Bpy) was added to 80 μl of aggregate (neat 10 uM sample), followed by 5 μl of APS. Samples were irradiated with a lamp in the dark room for 10 secs. 30 uls of (4×) LDS Sample buffer was added immediately.

SDS-PAGE was then performed on cross-linked (PICUP) and non-cross-linked A□ 1-42 aggregate. The solutions were incubated in a hot block at 70° C. for 10 minutes. Meanwhile, a marker was created by combining 5 μl of Magic Mark XP Western Protein Standard, 5 μl of Novex Sharp Pre-stained Protein Standard. After the ten-minute incubation, the samples plus marker were loaded onto a NuPAGE Novex 4-12% Bis-Tris Gels (1.0 mm, 15 well, 15 μl per well) with MES running buffer. The gels were run at 200 V for 35 minutes.

The gel was then blotted onto a PVDF membrane using an iBlot machine from Invitrogen, for 7 minutes at 20V (program P3).

Once blotting was complete, the gel stack was disassembled and the PVDF membrane was then blocked in 50 ml of 4% MPBST (4% Marvel in PBST) for one hour at room temperature with gentle rotation. The blots were then cut with a scalpel for probing with individual antibodies. This was a 1 hour incubation with the primary antibody solution (2 ug/ml in 10 ml of 3% MPBST).

Next, the membrane was washed 5× with PBST, 5 minutes each, and was then incubated in secondary antibody solution (1 μl anti-human Fc specific—HRP conjugate in 10 ml of PBST) for 1 hour at room temperature. The membrane was washed 3× with PBST and 2× with PBS, 5 minutes each.

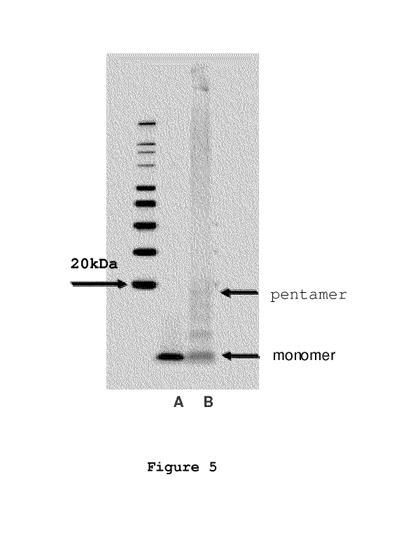

During the final washes, the chemi-luminescence SuperSignal West Dura substrate (Thermo Scientific; 34075) were allowed to warm to room temperature. 600 ul of each of the 2 solutions were combined. The PBS was decanted from the PVDF membrane, and then a pipette was used to cover the membrane with the mixed Dura reagents. The reaction was allowed to proceed for ˜5 minutes (during which time the VerscDoc Imaging System was set up) and then an image was taken with 30 sec exposure (with enhancement using the transform filter). A representative image is shown in FIG. 5.

### Example 2. Studies Demonstrating a Specific Functional Response of Abet0380-GL IgG1-TM Antibody In Vivo

### 2.1 Functional Characterisation of Abet0380-GL IgG1-TM by Reduction of Free Amyloid Beta 1-42 Peptide In Vivo

Eight-week old male albino Harlan Sprague-Dawley rats (n=8-12) received a single dose of Abet0380-GL IgG1-TM antibody by intravenous injection with a dosing vehicle of 25 mM Histidine, 7% Sucrose, 0.02% p80 surfactant, pH 6.0 at 5 ml/kg. Dosing solutions were made just before dosing. Animals were anaesthetized at the time indicated and cerebrospinal fluid (CSF) was aspirated from the cistema magna. CSF samples were centrifuged for 10 minutes at approximately 3000× g at 4° C. within 20 minutes of sampling to remove cells or debris. Samples were then frozen on dry ice and stored at −70° C. for subsequent analysis.

Animals were sacrificed by decapitation, brain tissue was dissected and Amyloid beta peptides were extracted from brain tissue in diethylamine (DEA; Fluka. Sigma. UK; cat: 31729). Briefly, frozen brain tissue was homogenized in 0.2% DEA and 50 mM NaCl (Merck, USA: cat: 1.06404.1000). Brain homogenates were ultracentrifuged at 133,000×g, for 1 hour. Recovered supernatants were neutralized to pH 8.0 with 2 M Tris-HCl (TRIZMA®-hydrochloride; Sigma, UK; cat: 93363) and stored at −70° C. until analysis. Animal experimentations were performed in accordance with relevant guidelines and regulations provided by the Swedish Board of Agriculture. The ethical permission was provided by an ethical board specialized in animal experimentations: the Stockholm Södra Animal Research Ethical Board.

Measurement of free Amyloid beta 1-42 peptide in rat CSF was conducted using immunoprecipitation to remove Abet0380-GL bound Amyloid beta 1-42 peptide, followed by analysis by a commercial ELISA kit obtained from Invitrogen. Briefly, a solution of protein A beads (Dynabeads® Protein A; Invitrogen, UK; cat: 100-02D) was added to a 96 well non-skirted plate (polypropylene 0.2 ml; VWR International, UK; cat: 10732-4828) and washed twice with TBST (50 mM TBS; Sigma, UK; cat: T6664 plus 0.1% Tween20) using a magnet (DynaMag™ 96 side; Invitrogen, UK; cat: 123.31 D) to separate the beads from the solution. Thawed rat CSF samples (40 μl) were added to each well and incubated at 40° C. with tilt rotation for 1 hour. The beads were then pelleted using the magnet and 30 μl of immunoprecipitated CSF samples were transferred to a 96 well plate from the ELISA kit (mouse Amyloid beta (1-42) colorimetric ELISA kit; Invitrogen, UK; cat: KMB3441) with 70 μl of the Standard Diluent Buffer already added (supplemented with protease inhibitor; Roche, UK; cat: 11836153001). Calibration standard samples were added to the plate in duplicate and the plate was incubated for 2 hours at room temperature with shaking. The plate was washed 4 times with 400 l of wash buffer, 100 μl of the detection antibody solution was added to each well and the plate was incubated for 1 hour at room temperature with shaking. Again, the plate was washed 4 times with 400 μl of wash buffer, 100 μl of the secondary antibody working solution was added to each well and the plate was incubated for 30 minutes at room temperature with shaking. Finally, the plate was washed 4 times with 400 μl of wash buffer, 100 μl of stabilized Chromogen was added to each well and the plate was incubated for 30 minutes at room temperature in the dark. To stop the reaction, 100 μl of Stop Solution was added to each well and the plate was read within 2 hours at an absorbance of 450 nm. Single CSF samples were analyzed and data analysis was performed using Prism 4 (GraphPad, USA) with one-way ANOVA on log transformed data without adjustment for multiple comparisons.

Measurement of total (free and Abet0380-GL bound) Amyloid beta 1-42 peptide in rat brain homogenates was performed using modifications of the mouse Amyloid beta (1-42) colorimetric ELISA kit (Invitrogen, UK: cat: KMB3441). The kit detection antibody was replaced by an excess of Abet0380-GL IgG1-TM antibody and the secondary antibody by an anti-human IgG HRP-conjugate antibody (Jackson ImmunoResearch, UK; cat: 109-035-098). Briefly, thawed brain homogenates of 50 μl diluted 1:2 in Sample Diluent (supplemented with protease inhibitor; Roche, UK; cat: 11836153001) and standard samples were added in duplicate to the 96 well ELISA plate. An excess of Abet0380-GL IgG1-TM antibody (50 μl, 4 μg/ml) was added to each well and the plate was then incubated for 3 hours at room temperature. The plate was washed 4 times with 400 μl of wash buffer, 100 μl of the secondary antibody working solution was added to each well and the plate was incubated for 30 minutes at room temperature. Finally, the plate was washed 4 times with 400 μl of wash buffer, 100 μl of stabilized Chromogen was added to each well and the plate was incubated for 15 minutes at room temperature in the dark. To stop the reaction, 100 μl of Stop Solution was added to each well and the plate was read within 2 hours at an absorbance of 450 nm. Data analysis was performed using Prism 4 (GraphPad, USA) with one-way ANOVA on log transformed data without adjustment for multiple comparisons.

Measurement of total Amyloid beta 1-40 peptide in rat brain homogenates was performed using the mouse Amyloid beta (1-40) colorimetric ELISA kit (Invitrogen, UK; cat: KMB3481). Briefly, thawed brain homogenates of 50 μl and standard samples, diluted in Sample Diluent (supplemented with protease inhibitor; Roche, UK: cat: 11836153001), were added in duplicate to the 96 well ELISA plate. 50 μl of the detection antibody solution were added to each well and the plate was incubated for 3 hours at room temperature. The plate was washed 4 times with 400 μl of wash buffer, 100 μl of the secondary antibody working solution was added to each well and the plate was incubated for 30 minutes at room temperature. Finally, the plate was washed 4 times with 400 μl of wash buffer, 100 μl of stabilized Chromogen was added to each well and the plate was incubated for 30 minutes at room temperature in the dark. To stop the reaction, 100 μl of Stop Solution was added to each well and the plate was read within 2 hours at an absorbance of 450 nm. Data analysis was performed using Prism 4 (GraphPad, USA) with one-way ANOVA on log transformed data without adjustment for multiple comparisons.

2.2 Functional Characterisation of Abet0380-GL IgG1-TM by Reduction of free Amyloid Beta 1-42 Peptide In Vivo

A single dose of the Abet0380-GL IgG1-TM antibody at 20 mg/kg reduced the CSF level of free Amyloid beta 1-42 peptide in rats to the limit of quantification at 72 or 168 hours after dose in the assay described in Section 2.1 (data not shown). To further investigate the effect of the Abet0380-GL IgG1-TM antibody in vivo, rats were administered weekly doses of 0.25, 0.5, 1, 5 or 10 mg/kg over 14 days. Animals were euthanized 168 hours after the second dose to measure levels of free Amyloid beta 1-42 peptide in CSF as well as total Amyloid beta 1-42 or 1-40 peptides in brain tissue.

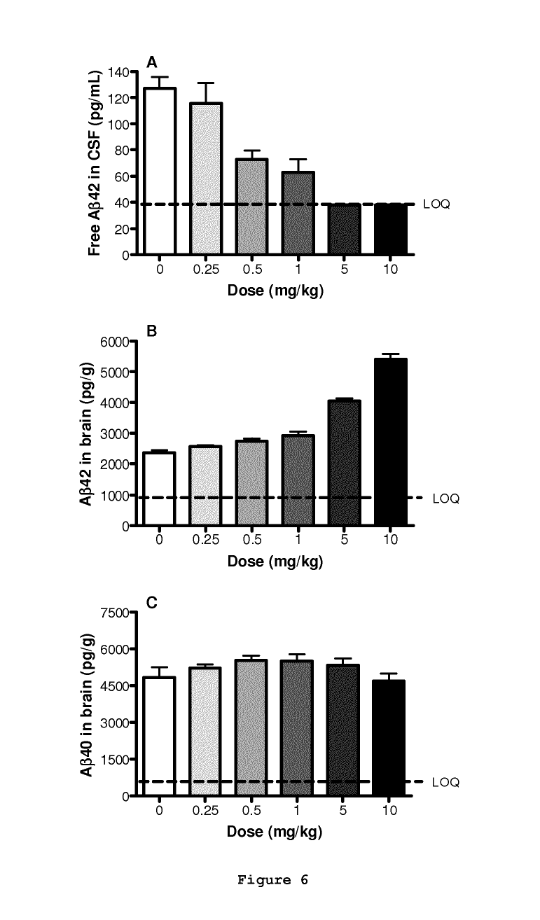

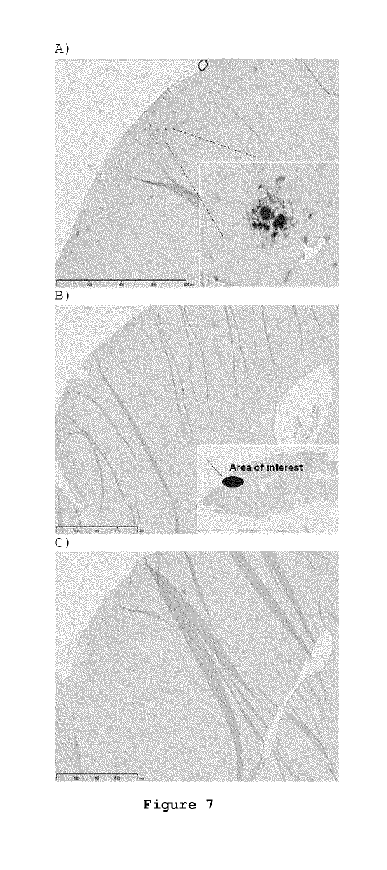

A dose-dependent decrease of free Amyloid beta 1-42 was demonstrated in CSF (FIG. 6A). The two highest doses of 5 and 10 mg/kg reduced Amyloid beta 1-42 peptide to the limit of quantification in the assay used, whereas doses of 0.5 and 1 mg/kg significantly reduced Amyloid beta 1-42 peptide by 47% and 61% respectively when compared to the vehicle control. The lowest dose, 0.25 mg/kg, gave a 14% reduction of free Amyloid beta 1-42 peptide in CSF, but failed to reach statistical significance. Due to sequestration of Amyloid beta 1-42 peptide by Abet0380-GL IgG1-TM antibody, a dose-dependent increase of total Amyloid beta 1-42 peptide was demonstrated in brain tissue (FIG. 6B). However, the level of total Amyloid beta 1-40 peptide in brain tissue was unaffected (FIG. 6C), thus demonstrating the specificity of Abet0380-GL IgG1-TM for Amyloid beta 1-42 peptide. In summary, the above results from rat studies showed that the Abet0380-GL IgG1-TM antibody reduced the level of free amyloid beta 1-42 peptide in CSF with an ED₅₀ between 0.5 and 1 mg/kg.

2.3 Functional Characterisation of Abet0380-GL IgG1TM—Demonstration of Non Plaque Binding In Vivo—No Binding of Abet0380-GL IgG-TM to Amyloid Beta Plaques In Vivo 168 Hours after a Peripheral Dose to Aged Tg2576 Mice

Abet0380-GL IgG1-TM was tested for its ability to bind to Amyloid beta plaques in aged Tg2576 mice after a single peripheral dose. Animal experimentations were performed in accordance with relevant guidelines and regulations provided by the Swedish Board of Agriculture. The ethical permission was provided by an ethical board specialized in animal experimentations: the Stockholm Sodra Animal Research Ethical Board. Seventeen-month old female Tg2576 mice (n=5) received a single dose of vehicle, a positive control antibody at 30 mg/kg or the Abet0380-GL IgG1-TM antibody at 10 or 30 mg/kg by intravenous injection with a dosing vehicle of 25 mM Histidine, 7% Sucrose, 0.02% p80 surfactant, pH 6.0 at 5 mL/kg. At 168 hours after dose, animals were deeply anaesthetized and perfused with room temperature PBS followed by cold (4° C.) phosphate buffered 4% paraformaldehyde (PFA). Animals were then sacrificed by decapitation and brains were dissected and immersions fix in PFA at 4° C. for 72 hours. The fixative was exchanged to PBS containing 0.1% sodium azide and tissues were stored at 4° C. until further processed.

Immunohistochemistry was performed on brain sections to evaluate the degree of binding of Abet0380-GL IgG1-TM to Amyloid beta plaques in vivo. Briefly, paraffin embedded brain sections were prepared for immunohistochemistry. Detection of Abet0380-GL IgG1-TM or the positive control antibody deposited within brain parenchyma was conducted using a rabbit-anti-mouse IgG1 and IgG2-specific secondary antibody from Epitomics. The staining was performed on the Ventana robot, using the OmniMap detection system (Ventana Medical Systems, USA). For spiking ex vivo, consecutive tissue sections were stained in vitro with the injected Abet0380-GL IgG1-TM or positive control antibody in excess. Secondary antibodies and chromogenes were the same as above.

Scoring of the staining was carried out in a blinded fashion under 10× optical magnification. The distribution of decorated plaques was noted. The intensity of plaque labelling was scored according to a relative intensity scale from 0 (no staining of plaques) up to 4 (intense decoration of plaques).

Abet0380-GL IgG1-TM did not decorate Amyloid beta plaques or cerebral amyloid angiopathy (CAA) in vivo at 168 hours after a peripheral dose of 10 or 30 mg/kg. The positive control antibody demonstrated intense to low in vivo plaque decoration. A partial and focal distribution pattern was apparent, with core plaques, diffuse plaques and CAA in all animals. Representative images are shown in FIG. 7. Spiking ex vivo of brain tissue from the same animals with Abet0380-GL IgG1-TM and the positive control antibody confirmed the previously demonstrated ex vivo plaque binding capacity of the injected antibodies (not shown).

### Example 3. Anti-Aβ1-42 Sequences

Examples of sequences of antibody molecules are listed in the appended sequence listing, including example antibody VH domains, VL domains, individual CDR sequences, sets of HCDRs, sets of LCDRs, and framework regions.

Sequences of the 24 optimized clones listed in Table 5 were compared. Tables 8 and 9 show % sequence identity between the VH and VL domains respectively.

TABLE 10
Examples of residues at each position within the
VH CDRs and Vernier Residues.

| | Kabat | Abet0380- | | | | | |
|---|---|---|---|---|---|---|---|
| | number | GL | Other example residues | | | | |
| VH FW1 | 26 | M | G | S | | | |
| | 27 | G | F | D | | | |
| | 28 | N | T | D | H | | |
| | 29 | F | | | | | |
| | 30 | N | S | K | P | | |
| VH CDR1 | 31 | Y | V | R | E | T | |
| | 32 | Q | Y | D | S | E | |
| | 33 | T | P | I | V | | |
| | 34 | M | | | | | |
| | 35 | W | | | | | |
| VH CDR2 | 50 | V | | | | | |
| | 51 | I | | | | | |
| | 52 | G | | | | | |
| | 52a | K | S | A | | | |
| | 53 | T | S | N | D | G | Q |
| | 54 | N | G | T | P | | |
| | 55 | E | G | N | K | T | |
| | 56 | N | T | R | K | | |
| | 57 | I | T | K | V | | |
| | 58 | A | V | T | | | |
| | 59 | Y | | | | | |
| | 60 | A | | | | | |
| | 61 | D | | | | | |
| | 62 | S | | | | | |
| | 63 | V | | | | | |
| | 64 | K | | | | | |
| | 65 | G | | | | | |
| VH CDR3 | 95 | E | | | | | |
| | 96 | W | | | | | |
| | 97 | M | | | | | |
| | 98 | D | | | | | |
| | 99 | H | | | | | |
| | 100 | S | | | | | |
| | 100a | R | | | | | |
| | 100b | P | | | | | |
| | 100c | Y | | | | | |
| | 100d | Y | | | | | |
| | 100e | Y | | | | | |
| | 100f | Y | | | | | |
| | 100g | G | | | | | |
| | 100h | M | | | | | |
| | 101 | D | | | | | |
| | 102 | V | | | | | |

TABLE 11
Examples of residues at each position within the V_{L} CDRs.

| | Kabat | Abet0380- | |
|---|---|---|---|
| | number | GL | Other example residues |
| VL CDR1 | 24 | S | |
| | 25 | G | |
| | 26 | H | |
| | 27 | N | |
| | 28 | L | I |
| | 29 | E | G |
| | 30 | D | |
| | 31 | K | |
| | 32 | F | W |
| | 33 | A | V |
| | 34 | S | |
| VL CDR2 | 50 | R | |
| | 51 | D | |
| | 52 | D | |
| | 53 | K | |
| | 54 | R | |
| | 55 | P | |
| | 56 | S | |
| VL CDR3 | 89 | S | Q |
| | 90 | S | A |
| | 91 | Q | |
| | 92 | D | |
| | 93 | T | S |
| | 94 | V | T |
| | 95 | T | |
| | 96 | R | |
| | 97 | V | |

TABLE 12
Substitutions observed in VH CDRs and FW1 in 24 optimized clones

| | Kabat | | |
|---|---|---|---|
| | number | 0380-GL | Substitutions in other optimized clones |
| VH FW1 | 26 | M | G, S, V, A, N, T, H |
| | 27 | G | F, S, Y, E, D, P |
| | 28 | N | Q, H, V, E, T, A, S, D, M, P |
| | 29 | F | I, Y, S, L, W |
| | 30 | N | S, T, Q, K, H, R, G, P, E, K, A, D |
| VH CDR1 | 31 | Y | H, K, E, N, T, R, V, P, M, F, I, D, W |
| | 32 | Q | Y, D, N, S, E, T |
| | 33 | T | P, I, V, A, I |
| | 34 | M | L |
| | 35 | W | |
| VH CDR2 | 50 | V | |
| | 51 | I | |
| | 52 | G | |
| | 52a | K | S, P, A, N, G, E, D, V, T |
| | 53 | T | S, N, H, Q, D, G, E |
| | 54 | N | G, P, T, Q, E, M, K, A |
| | 55 | E | G, K, N, Q, T, H, D, A |
| | 56 | N | T, A, R, K |
| | 57 | I | T, N, S, K, F, Q, V, L |
| | 58 | A | V, S, T, N |
| | 59 | Y | |
| | 60 | A | |
| | 61 | D | |
| | 62 | S | A, T |
| | 63 | V | |
| | 64 | K | |
| | 65 | G | |
| VH CDR3 | 95 | E | |
| | 96 | W | |
| | 97 | M | |
| | 98 | D | G |
| | 99 | H | R |
| | 100 | S | |
| | 100a | R | |
| | 100b | P | |
| | 100c | Y | |
| | 100d | Y | |
| | 100e | Y | |
| | 100f | Y | |
| | 100g | G | |
| | 100h | M | I |
| | 101 | D | |
| | 102 | V | A |

TABLE 13
Substitutions observed in VL CDRs in 24 optimized clones

| | Kabat | | Substitutions in other |
|---|---|---|---|
| | number | 0380-GL | optimized clones |
| VL CDR1 | 24 | S | T |
| | 25 | G | T |
| | 26 | H | R, P |
| | 27 | N | H |
| | 28 | L | I, V, F, T |
| | 29 | E | M, G, S, N |
| | 30 | D | A, S, G, H |
| | 31 | K | S |
| | 32 | F | W |
| | 33 | A | V, M, T, I |
| | 34 | S | T, A |
| VL CDR2 | 50 | R | |
| | 51 | D | |
| | 52 | D | |
| | 53 | K | |
| | 54 | R | |
| | 55 | P | |
| | 56 | S | |
| VL CDR3 | 89 | S | Q, A |
| | 90 | S | A, T |
| | 91 | Q | |
| | 92 | D | G |
| | 93 | T | Q, S, N, K |
| | 94 | V | T, F |
| | 95 | T | |
| | 96 | R | |
| | 97 | V | S, A |

TABLE 14
Correspondence between the antibody sequences
mentioned herein and the sequences in the Sequence
Listing at the end of this document.

| | | |
|---|---|---|
| 1 | Abet0007 | VH DNA |
| 2 | Abet0007 | VH PRT |
| 3 | Abet0007 | CDR1 PRT |
| 4 | Abet0007 | CDR2 PRT |
| 5 | Abet0007 | CDR3 PRT |
| 6 | Abet0007 | FW1 PRT |
| 7 | Abet0007 | FW2 PRT |
| 8 | Abet0007 | FW3 PRT |
| 9 | Abet0007 | FW4 PRT |
| 10 | Abet0007 | VL DNA |
| 11 | Abet0007 | VL PRT |
| 12 | Abet0007 | CDR1 PRT |
| 13 | Abet0007 | CDR2 PRT |
| 14 | Abet0007 | CDR3 PRT |
| 15 | Abet0007 | FW1 PRT |
| 16 | Abet0007 | FW2 PRT |
| 17 | Abet0007 | FW3 PRT |
| 18 | Abet0007 | FW4 PRT |
| 19 | Abet0144-GL | VH DNA |
| 20 | Abet0144-GL | VH PRT |
| 21 | Abet0144-GL | CDR1 PRT |
| 22 | Abet0144-GL | CDR2 PRT |
| 23 | Abet0144-GL | CDR3 PRT |
| 24 | Abet0144-GL | FW1 PRT |
| 25 | Abet0144-GL | FW2 PRT |
| 26 | Abet0144-GL | FW3 PRT |
| 27 | Abet0144-GL | FW4 PRT |
| 28 | Abet0144-GL | VL DNA |
| 29 | Abet0144-GL | VL PRT |
| 30 | Abet0144-GL | CDR1 PRT |
| 31 | Abet0144-GL | CDR2 PRT |
| 32 | Abet0144-GL | CDR3 PRT |
| 33 | Abet0144-GL | FW1 PRT |
| 34 | Abet0144-GL | FW2 PRT |
| 35 | Abet0144-GL | FW3 PRT |
| 36 | Abet0144-GL | FW4 PRT |
| 37 | Abet0319 | VH DNA |
| 38 | Abet0319 | VH PRT |
| 39 | Abet0319 | CDR1 PRT |
| 40 | Abet0319 | CDR2 PRT |
| 41 | Abet0319 | CDR3 PRT |
| 42 | Abet0319 | FW1 PRT |
| 43 | Abet0319 | FW2 PRT |
| 44 | Abet0319 | FW3 PRT |
| 45 | Abet0319 | FW4 PRT |
| 46 | Abet0319 | VL DNA |
| 47 | Abet0319 | VL PRT |
| 48 | Abet0319 | CDR1 PRT |
| 49 | Abet0319 | CDR2 PRT |
| 50 | Abet0319 | CDR3 PRT |
| 51 | Abet0319 | FW1 PRT |
| 52 | Abet0319 | FW2 PRT |
| 53 | Abet0319 | FW3 PRT |
| 54 | Abet0319 | FW4 PRT |
| 55 | Abet0321b | VH DNA |
| 56 | Abet0321b | VH PRT |
| 57 | Abet0321b | CDR1 PRT |
| 58 | Abet0321b | CDR2 PRT |
| 59 | Abet0321b | CDR3 PRT |
| 60 | Abet0321b | FW1 PRT |
| 61 | Abet0321b | FW2 PRT |
| 62 | Abet0321b | FW3 PRT |
| 63 | Abet0321b | FW4 PRT |
| 64 | Abet0321b | VL DNA |
| 65 | Abet0321b | VL PRT |
| 66 | Abet0321b | CDR1 PRT |
| 67 | Abet0321b | CDR2 PRT |
| 68 | Abet0321b | CDR3 PRT |
| 69 | Abet0321b | FW1 PRT |
| 70 | Abet0321b | FW2 PRT |
| 71 | Abet0321b | FW3 PRT |
| 72 | Abet0321b | FW4 PRT |
| 73 | Abet0322b | VH DNA |
| 74 | Abet0322b | VH PRT |
| 75 | Abet0322b | CDR1 PRT |
| 76 | Abet0322b | CDR2 PRT |
| 77 | Abet0322b | CDR3 PRT |
| 78 | Abet0322b | FW1 PRT |
| 79 | Abet0322b | FW2 PRT |
| 80 | Abet0322b | FW3 PRT |
| 81 | Abet0322b | FW4 PRT |
| 82 | Abet0322b | VL DNA |
| 83 | Abet0322b | VL PRT |
| 84 | Abet0322b | CDR1 PRT |
| 85 | Abet0322b | CDR2 PRT |
| 86 | Abet0322b | CDR3 PRT |
| 87 | Abet0322b | FW1 PRT |
| 88 | Abet0322b | FW2 PRT |
| 89 | Abet0322b | FW3 PRT |
| 90 | Abet0322b | FW4 PRT |
| 91 | Abet0323b | VH DNA |
| 92 | Abet0323b | VH PRT |
| 93 | Abet0323b | CDR1 PRT |
| 94 | Abet0323b | CDR2 PRT |
| 95 | Abet0323b | CDR3 PRT |
| 96 | Abet0323b | FW1 PRT |
| 97 | Abet0323b | FW2 PRT |
| 98 | Abet0323b | FW3 PRT |
| 99 | Abet0323b | FW4 PRT |
| 100 | Abet0323b | VL DNA |
| 101 | Abet0323b | VL PRT |
| 102 | Abet0323b | CDR1 PRT |
| 103 | Abet0323b | CDR2 PRT |
| 104 | Abet0323b | CDR3 PRT |
| 105 | Abet0323b | FW1 PRT |
| 106 | Abet0323b | FW2 PRT |
| 107 | Abet0323b | FW3 PRT |
| 108 | Abet0323b | FW4 PRT |
| 109 | Abet0328 | VH DNA |
| 110 | Abet0328 | VH PRT |
| 111 | Abet0328 | CDR1 PRT |
| 112 | Abet0328 | CDR2 PRT |
| 113 | Abet0328 | CDR3 PRT |
| 114 | Abet0328 | FW1 PRT |
| 115 | Abet0328 | FW2 PRT |
| 116 | Abet0328 | FW3 PRT |
| 117 | Abet0328 | FW4 PRT |
| 118 | Abet0328 | VL DNA |
| 119 | Abet0328 | VL PRT |
| 120 | Abet0328 | CDR1 PRT |
| 121 | Abet0328 | CDR2 PRT |
| 122 | Abet0328 | CDR3 PRT |
| 123 | Abet0328 | FW1 PRT |
| 124 | Abet0328 | FW2 PRT |
| 125 | Abet0328 | FW3 PRT |
| 126 | Abet0328 | FW4 PRT |
| 127 | Abet0329 | VH DNA |
| 128 | Abet0329 | VH PRT |
| 129 | Abet0329 | CDR1 PRT |
| 130 | Abet0329 | CDR2 PRT |
| 131 | Abet0329 | CDR3 PRT |
| 132 | Abet0329 | FW1 PRT |
| 133 | Abet0329 | FW2 PRT |
| 134 | Abet0329 | FW3 PRT |
| 135 | Abet0329 | FW4 PRT |
| 136 | Abet0329 | VL DNA |
| 137 | Abet0329 | VL PRT |
| 138 | Abet0329 | CDR1 PRT |
| 139 | Abet0329 | CDR2 PRT |
| 140 | Abet0329 | CDR3 PRT |
| 141 | Abet0329 | FW1 PRT |
| 142 | Abet0329 | FW2 PRT |
| 143 | Abet0329 | FW3 PRT |
| 144 | Abet0329 | FW4 PRT |
| 145 | Abet0332 | VH DNA |
| 146 | Abet0332 | VH PRT |
| 147 | Abet0332 | CDR1 PRT |
| 148 | Abet0332 | CDR2 PRT |
| 149 | Abet0332 | CDR3 PRT |
| 150 | Abet0332 | FW1 PRT |
| 151 | Abet0332 | FW2 PRT |
| 152 | Abet0332 | FW3 PRT |
| 153 | Abet0332 | FW4 PRT |
| 154 | Abet0332 | VL DNA |
| 155 | Abet0332 | VL PRT |
| 156 | Abet0332 | CDR1 PRT |
| 157 | Abet0332 | CDR2 PRT |
| 158 | Abet0332 | CDR3 PRT |
| 159 | Abet0332 | FW1 PRT |
| 160 | Abet0332 | FW2 PRT |
| 161 | Abet0332 | FW3 PRT |
| 162 | Abet0332 | FW4 PRT |
| 163 | Abet0342 | VH DNA |
| 164 | Abet0342 | VH PRT |
| 165 | Abet0342 | CDR1 PRT |
| 166 | Abet0342 | CDR2 PRT |
| 167 | Abet0342 | CDR3 PRT |
| 168 | Abet0342 | FW1 PRT |
| 169 | Abet0342 | FW2 PRT |
| 170 | Abet0342 | FW3 PRT |
| 171 | Abet0342 | FW4 PRT |
| 172 | Abet0342 | VL DNA |
| 173 | Abet0342 | VL PRT |
| 174 | Abet0342 | CDR1 PRT |
| 175 | Abet0342 | CDR2 PRT |
| 176 | Abet0342 | CDR3 PRT |
| 177 | Abet0342 | FW1 PRT |
| 178 | Abet0342 | FW2 PRT |
| 179 | Abet0342 | FW3 PRT |
| 180 | Abet0342 | FW4 PRT |
| 181 | Abet0343 | VH DNA |
| 182 | Abet0343 | VH PRT |
| 183 | Abet0343 | CDR1 PRT |
| 184 | Abet0343 | CDR2 PRT |
| 185 | Abet0343 | CDR3 PRT |
| 186 | Abet0343 | FW1 PRT |
| 187 | Abet0343 | FW2 PRT |
| 188 | Abet0343 | FW3 PRT |
| 189 | Abet0343 | FW4 PRT |
| 190 | Abet0343 | VL DNA |
| 191 | Abet0343 | VL PRT |
| 192 | Abet0343 | CDR1 PRT |
| 193 | Abet0343 | CDR2 PRT |
| 194 | Abet0343 | CDR3 PRT |
| 195 | Abet0343 | FW1 PRT |
| 196 | Abet0343 | FW2 PRT |
| 197 | Abet0343 | FW3 PRT |
| 198 | Abet0343 | FW4 PRT |
| 199 | Abet0344 | VH DNA |
| 200 | Abet0344 | VH PRT |
| 201 | Abet0344 | CDR1 PRT |
| 202 | Abet0344 | CDR2 PRT |
| 203 | Abet0344 | CDR3 PRT |
| 204 | Abet0344 | FW1 PRT |
| 205 | Abet0344 | FW2 PRT |
| 206 | Abet0344 | FW3 PRT |
| 207 | Abet0344 | FW4 PRT |
| 208 | Abet0344 | VL DNA |
| 209 | Abet0344 | VL PRT |
| 210 | Abet0344 | CDR1 PRT |
| 211 | Abet0344 | CDR2 PRT |
| 212 | Abet0344 | CDR3 PRT |
| 213 | Abet0344 | FW1 PRT |
| 214 | Abet0344 | FW2 PRT |
| 215 | Abet0344 | FW3 PRT |
| 216 | Abet0344 | FW4 PRT |
| 217 | Abet0368 | VH DNA |
| 218 | Abet0368 | VH PRT |
| 219 | Abet0368 | CDR1 PRT |
| 220 | Abet0368 | CDR2 PRT |
| 221 | Abet0368 | CDR3 PRT |
| 222 | Abet0368 | FW1 PRT |
| 223 | Abet0368 | FW2 PRT |
| 224 | Abet0368 | FW3 PRT |
| 225 | Abet0368 | FW4 PRT |
| 226 | Abet0368 | VL DNA |
| 227 | Abet0368 | VL PRT |
| 228 | Abet0368 | CDR1 PRT |
| 229 | Abet0368 | CDR2 PRT |
| 230 | Abet0368 | CDR3 PRT |
| 231 | Abet0368 | FW1 PRT |
| 232 | Abet0368 | FW2 PRT |
| 233 | Abet0368 | FW3 PRT |
| 234 | Abet0368 | FW4 PRT |
| 235 | Abet0369 | VH DNA |
| 236 | Abet0369 | VH PRT |
| 237 | Abet0369 | CDR1 PRT |
| 238 | Abet0369 | CDR2 PRT |
| 239 | Abet0369 | CDR3 PRT |
| 240 | Abet0369 | FW1 PRT |
| 241 | Abet0369 | FW2 PRT |
| 242 | Abet0369 | FW3 PRT |
| 243 | Abet0369 | FW4 PRT |
| 244 | Abet0369 | VL DNA |
| 245 | Abet0369 | VL PRT |
| 246 | Abet0369 | CDR1 PRT |
| 247 | Abet0369 | CDR2 PRT |
| 248 | Abet0369 | CDR3 PRT |
| 249 | Abet0369 | FW1 PRT |
| 250 | Abet0369 | FW2 PRT |
| 251 | Abet0369 | FW3 PRT |
| 252 | Abet0369 | FW4 PRT |
| 253 | Abet0370 | VH DNA |
| 254 | Abet0370 | VH PRT |
| 255 | Abet0370 | CDR1 PRT |
| 256 | Abet0370 | CDR2 PRT |
| 257 | Abet0370 | CDR3 PRT |
| 258 | Abet0370 | FW1 PRT |
| 259 | Abet0370 | FW2 PRT |
| 260 | Abet0370 | FW3 PRT |
| 261 | Abet0370 | FW4 PRT |
| 262 | Abet0370 | VL DNA |
| 263 | Abet0370 | VL PRT |
| 264 | Abet0370 | CDR1 PRT |
| 265 | Abet0370 | CDR2 PRT |
| 266 | Abet0370 | CDR3 PRT |
| 267 | Abet0370 | FW1 PRT |
| 268 | Abet0370 | FW2 PRT |
| 269 | Abet0370 | FW3 PRT |
| 270 | Abet0370 | FW4 PRT |
| 271 | Abet0371 | VH DNA |
| 272 | Abet0371 | VH PRT |
| 273 | Abet0371 | CDR1 PRT |
| 274 | Abet0371 | CDR2 PRT |
| 275 | Abet0371 | CDR3 PRT |
| 276 | Abet0371 | FW1 PRT |
| 277 | Abet0371 | FW2 PRT |
| 278 | Abet0371 | FW3 PRT |
| 279 | Abet0371 | FW4 PRT |
| 280 | Abet0371 | VL DNA |
| 281 | Abet0371 | VL PRT |
| 282 | Abet0371 | CDR1 PRT |
| 283 | Abet0371 | CDR2 PRT |
| 284 | Abet0371 | CDR3 PRT |
| 285 | Abet0371 | FW1 PRT |
| 286 | Abet0371 | FW2 PRT |
| 287 | Abet0371 | FW3 PRT |
| 288 | Abet0371 | FW4 PRT |
| 289 | Abet0372 | VH DNA |
| 290 | Abet0372 | VH PRT |
| 291 | Abet0372 | CDR1 PRT |
| 292 | Abet0372 | CDR2 PRT |
| 293 | Abet0372 | CDR3 PRT |
| 294 | Abet0372 | FW1 PRT |
| 295 | Abet0372 | FW2 PRT |
| 296 | Abet0372 | FW3 PRT |
| 297 | Abet0372 | FW4 PRT |
| 298 | Abet0372 | VL DNA |
| 299 | Abet0372 | VL PRT |
| 300 | Abet0372 | CDR1 PRT |
| 301 | Abet0372 | CDR2 PRT |
| 302 | Abet0372 | CDR3 PRT |
| 303 | Abet0372 | FW1 PRT |
| 304 | Abet0372 | FW2 PRT |
| 305 | Abet0372 | FW3 PRT |
| 306 | Abet0372 | FW4 PRT |
| 307 | Abet0373 | VH DNA |
| 308 | Abet0373 | VH PRT |
| 309 | Abet0373 | CDR1 PRT |
| 310 | Abet0373 | CDR2 PRT |
| 311 | Abet0373 | CDR3 PRT |
| 312 | Abet0373 | FW1 PRT |
| 313 | Abet0373 | FW2 PRT |
| 314 | Abet0373 | FW3 PRT |
| 315 | Abet0373 | FW4 PRT |
| 316 | Abet0373 | VL DNA |
| 317 | Abet0373 | VL PRT |
| 318 | Abet0373 | CDR1 PRT |
| 319 | Abet0373 | CDR2 PRT |
| 320 | Abet0373 | CDR3 PRT |
| 321 | Abet0373 | FW1 PRT |
| 322 | Abet0373 | FW2 PRT |
| 323 | Abet0373 | FW3 PRT |
| 324 | Abet0373 | FW4 PRT |
| 325 | Abet0374 | VH DNA |
| 326 | Abet0374 | VH PRT |
| 327 | Abet0374 | CDR1 PRT |
| 328 | Abet0374 | CDR2 PRT |
| 329 | Abet0374 | CDR3 PRT |
| 330 | Abet0374 | FW1 PRT |
| 331 | Abet0374 | FW2 PRT |
| 332 | Abet0374 | FW3 PRT |
| 333 | Abet0374 | FW4 PRT |
| 334 | Abet0374 | VL DNA |
| 335 | Abet0374 | VL PRT |
| 336 | Abet0374 | CDR1 PRT |
| 337 | Abet0374 | CDR2 PRT |
| 338 | Abet0374 | CDR3 PRT |
| 339 | Abet0374 | FW1 PRT |
| 340 | Abet0374 | FW2 PRT |
| 341 | Abet0374 | FW3 PRT |
| 342 | Abet0374 | FW4 PRT |
| 343 | Abet0377 | VH DNA |
| 344 | Abet0377 | VH PRT |
| 345 | Abet0377 | CDR1 PRT |
| 346 | Abet0377 | CDR2 PRT |
| 347 | Abet0377 | CDR3 PRT |
| 348 | Abet0377 | FW1 PRT |
| 349 | Abet0377 | FW2 PRT |
| 350 | Abet0377 | FW3 PRT |
| 351 | Abet0377 | FW4 PRT |
| 352 | Abet0377 | VL DNA |
| 353 | Abet0377 | VL PRT |
| 354 | Abet0377 | CDR1 PRT |
| 355 | Abet0377 | CDR2 PRT |
| 356 | Abet0377 | CDR3 PRT |
| 357 | Abet0377 | FW1 PRT |
| 358 | Abet0377 | FW2 PRT |
| 359 | Abet0377 | FW3 PRT |
| 360 | Abet0377 | FW4 PRT |
| 361 | Abet0378 | VH DNA |
| 362 | Abet0378 | VH PRT |
| 363 | Abet0378 | CDR1 PRT |
| 364 | Abet0378 | CDR2 PRT |
| 365 | Abet0378 | CDR3 PRT |
| 366 | Abet0378 | FW1 PRT |
| 367 | Abet0378 | FW2 PRT |
| 368 | Abet0378 | FW3 PRT |
| 369 | Abet0378 | FW4 PRT |
| 370 | Abet0378 | VL DNA |
| 371 | Abet0378 | VL PRT |
| 372 | Abet0378 | CDR1 PRT |
| 373 | Abet0378 | CDR2 PRT |
| 374 | Abet0378 | CDR3 PRT |
| 375 | Abet0378 | FW1 PRT |
| 376 | Abet0378 | FW2 PRT |
| 377 | Abet0378 | FW3 PRT |
| 378 | Abet0378 | FW4 PRT |
| 379 | Abet0379 | VH DNA |
| 380 | Abet0379 | VH PRT |
| 381 | Abet0379 | CDR1 PRT |
| 382 | Abet0379 | CDR2 PRT |
| 383 | Abet0379 | CDR3 PRT |
| 384 | Abet0379 | FW1 PRT |
| 385 | Abet0379 | FW2 PRT |
| 386 | Abet0379 | FW3 PRT |
| 387 | Abet0379 | FW4 PRT |
| 388 | Abet0379 | VL DNA |
| 389 | Abet0379 | VL PRT |
| 390 | Abet0379 | CDR1 PRT |
| 391 | Abet0379 | CDR2 PRT |
| 392 | Abet0379 | CDR3 PRT |
| 393 | Abet0379 | FW1 PRT |
| 394 | Abet0379 | FW2 PRT |
| 395 | Abet0379 | FW3 PRT |
| 396 | Abet0379 | FW4 PRT |
| 397 | Abet0380 | VH DNA |
| 398 | Abet0380 | VH PRT |
| 399 | Abet0380 | CDR1 PRT |
| 400 | Abet0380 | CDR2 PRT |
| 401 | Abet0380 | CDR3 PRT |
| 402 | Abet0380 | FW1 PRT |
| 403 | Abet0380 | FW2 PRT |
| 404 | Abet0380 | FW3 PRT |
| 405 | Abet0380 | FW4 PRT |
| 406 | Abet0380 | VL DNA |
| 407 | Abet0380 | VL PRT |
| 408 | Abet0380 | CDR1 PRT |
| 409 | Abet0380 | CDR2 PRT |
| 410 | Abet0380 | CDR3 PRT |
| 411 | Abet0380 | FW1 PRT |
| 412 | Abet0380 | FW2 PRT |
| 413 | Abet0380 | FW3 PRT |
| 414 | Abet0380 | FW4 PRT |
| 415 | Abet0381 | VH DNA |
| 416 | Abet0381 | VH PRT |
| 417 | Abet0381 | CDR1 PRT |
| 418 | Abet0381 | CDR2 PRT |
| 419 | Abet0381 | CDR3 PRT |
| 420 | Abet0381 | FW1 PRT |
| 421 | Abet0381 | FW2 PRT |
| 422 | Abet0381 | FW3 PRT |
| 423 | Abet0381 | FW4 PRT |
| 424 | Abet0381 | VL DNA |
| 425 | Abet0381 | VL PRT |
| 426 | Abet0381 | CDR1 PRT |
| 427 | Abet0381 | CDR2 PRT |
| 428 | Abet0381 | CDR3 PRT |
| 429 | Abet0381 | FW1 PRT |
| 430 | Abet0381 | FW2 PRT |
| 431 | Abet0381 | FW3 PRT |
| 432 | Abet0381 | FW4 PRT |
| 433 | Abet0382 | VH DNA |
| 434 | Abet0382 | VH PRT |
| 435 | Abet0382 | CDR1 PRT |
| 436 | Abet0382 | CDR2 PRT |
| 437 | Abet0382 | CDR3 PRT |
| 438 | Abet0382 | FW1 PRT |
| 439 | Abet0382 | FW2 PRT |
| 440 | Abet0382 | FW3 PRT |
| 441 | Abet0382 | FW4 PRT |
| 442 | Abet0382 | VL DNA |
| 443 | Abet0382 | VL PRT |
| 444 | Abet0382 | CDR1 PRT |
| 445 | Abet0382 | CDR2 PRT |
| 446 | Abet0382 | CDR3 PRT |
| 447 | Abet0382 | FW1 PRT |
| 448 | Abet0382 | FW2 PRT |
| 449 | Abet0382 | FW3 PRT |
| 450 | Abet0382 | FW4 PRT |
| 451 | Abet0383 | VH DNA |
| 452 | Abet0383 | VH PRT |
| 453 | Abet0383 | CDR1 PRT |
| 454 | Abet0383 | CDR2 PRT |
| 455 | Abet0383 | CDR3 PRT |
| 456 | Abet0383 | FW1 PRT |
| 457 | Abet0383 | FW2 PRT |
| 458 | Abet0383 | FW3 PRT |
| 459 | Abet0383 | FW4 PRT |
| 460 | Abet0383 | VL DNA |
| 461 | Abet0383 | VL PRT |
| 462 | Abet0383 | CDR1 PRT |
| 463 | Abet0383 | CDR2 PRT |
| 464 | Abet0383 | CDR3 PRT |
| 465 | Abet0383 | FW1 PRT |
| 466 | Abet0383 | FW2 PRT |
| 467 | Abet0383 | FW3 PRT |
| 468 | Abet0383 | FW4 PRT |
| 469 | Abet0343-GL | VH DNA |
| 470 | Abet0343-GL | VH PRT |
| 471 | Abet0343-GL | CDR1 PRT |
| 472 | Abet0343-GL | CDR2 PRT |
| 473 | Abet0343-GL | CDR3 PRT |
| 474 | Abet0343-GL | FW1 PRT |
| 475 | Abet0343-GL | FW2 PRT |
| 476 | Abet0343-GL | FW3 PRT |
| 477 | Abet0343-GL | FW4 PRT |
| 478 | Abet0343-GL | VL DNA |
| 479 | Abet0343-GL | VL PRT |
| 480 | Abet0343-GL | CDR1 PRT |
| 481 | Abet0343-GL | CDR2 PRT |
| 482 | Abet0343-GL | CDR3 PRT |
| 483 | Abet0343-GL | FW1 PRT |
| 484 | Abet0343-GL | FW2 PRT |
| 485 | Abet0343-GL | FW3 PRT |
| 486 | Abet0343-GL | FW4 PRT |
| 487 | Abet0369-GL | VH DNA |
| 488 | Abet0369-GL | VH PRT |
| 489 | Abet0369-GL | CDR1 PRT |
| 490 | Abet0369-GL | CDR2 PRT |
| 491 | Abet0369-GL | CDR3 PRT |
| 492 | Abet0369-GL | FW1 PRT |
| 493 | Abet0369-GL | FW2 PRT |
| 494 | Abet0369-GL | FW3 PRT |
| 495 | Abet0369-GL | FW4 PRT |
| 496 | Abet0369-GL | VL DNA |
| 497 | Abet0369-GL | VL PRT |
| 498 | Abet0369-GL | CDR1 PRT |
| 499 | Abet0369-GL | CDR2 PRT |
| 500 | Abet0369-GL | CDR3 PRT |
| 501 | Abet0369-GL | FW1 PRT |
| 502 | Abet0369-GL | FW2 PRT |
| 503 | Abet0369-GL | FW3 PRT |
| 504 | Abet0369-GL | FW4 PRT |
| 505 | Abet0377-GL | VH DNA |
| 506 | Abet0377-GL | VH PRT |
| 507 | Abet0377-GL | CDR1 PRT |
| 508 | Abet0377-GL | CDR2 PRT |
| 509 | Abet0377-GL | CDR3 PRT |
| 510 | Abet0377-GL | FW1 PRT |
| 511 | Abet0377-GL | FW2 PRT |
| 512 | Abet0377-GL | FW3 PRT |
| 513 | Abet0377-GL | FW4 PRT |
| 514 | Abet0377-GL | VL DNA |
| 515 | Abet0377-GL | VL PRT |
| 516 | Abet0377-GL | CDR1 PRT |
| 517 | Abet0377-GL | CDR2 PRT |
| 518 | Abet0377-GL | CDR3 PRT |
| 519 | Abet0377-GL | FW1 PRT |
| 520 | Abet0377-GL | FW2 PRT |
| 521 | Abet0377-GL | FW3 PRT |
| 522 | Abet0377-GL | FW4 PRT |
| 523 | Abet0380-GL | VH DNA |
| 524 | Abet0380-GL | VH PRT |
| 525 | Abet0380-GL | CDR1 PRT |
| 526 | Abet0380-GL | CDR2 PRT |
| 527 | Abet0380-GL | CDR3 PRT |
| 528 | Abet0380-GL | FW1 PRT |
| 529 | Abet0380-GL | FW2 PRT |
| 530 | Abet0380-GL | FW3 PRT |
| 531 | Abet0380-GL | FW4 PRT |
| 532 | Abet0380-GL | VL DNA |
| 533 | Abet0380-GL | VL PRT |
| 534 | Abet0380-GL | CDR1 PRT |
| 535 | Abet0380-GL | CDR2 PRT |
| 536 | Abet0380-GL | CDR3 PRT |
| 537 | Abet0380-GL | FW1 PRT |
| 538 | Abet0380-GL | FW2 PRT |
| 539 | Abet0380-GL | FW3 PRT |
| 540 | Abet0380-GL | FW4 PRT |
| 541 | Abet0382-GL | VH DNA |
| 542 | Abet0382-GL | VH PRT |
| 543 | Abet0382-GL | CDR1 PRT |
| 544 | Abet0382-GL | CDR2 PRT |
| 545 | Abet0382-GL | CDR3 PRT |
| 546 | Abet0382-GL | FW1 PRT |
| 547 | Abet0382-GL | FW2 PRT |
| 548 | Abet0382-GL | FW3 PRT |
| 549 | Abet0382-GL | FW4 PRT |
| 550 | Abet0382-GL | VL DNA |
| 551 | Abet0382-GL | VL PRT |
| 552 | Abet0382-GL | CDR1 PRT |
| 553 | Abet0382-GL | CDR2 PRT |
| 554 | Abet0382-GL | CDR3 PRT |
| 555 | Abet0382-GL | FW1 PRT |
| 556 | Abet0382-GL | FW2 PRT |
| 557 | Abet0382-GL | FW3 PRT |
| 558 | Abet0382-GL | FW4 PRT |

### Example 4: Specificity of Abet0380-GL IgG-TM in Competition Binding Experiments

The specificity of Abet0380-GL IgG1-TM was examined in competition binding experiments. In brief Abet0380-GL IgG1-TM (0.5 nM) was incubated (1 hr at room temperature) with a range of different concentrations (10 uM down to 0.17 nM) of a panel of full length, truncate and pyro human Abeta peptides (Abeta 1-42, Abeta 1-43, Abeta 1-16, Abeta 12-28, Abeta 17-42, Abeta pyro-3-42, or Abeta pyro-11-42).

Following the incubation between Abet0380-GL IgG1-TM and the Abeta peptides N-terminal biotin Abeta 1-42 (1.5 nM) was added followed by a europium cryptate labelled anti-human Fc antibody (0.8 nM) (CisBio Cat. No. 61HFCKLB) and streptavidin-XL^{entl} (5 nM) (CisBio Cat. No. 611SAXLB). The assay was then incubated for a further 2 hrs at room temperature before reading on an Envision plate reader (PerkinElmer) using a standard homogeneous time resolved fluorescence (HTRF) read protocol. In the absence of competition, the interaction of N-terminal biotin Abeta 1-42 with Abet0380-GL IgG1-TM (in complex with streptavidin-XL^{entl} and and europium cryptate labelled anti-human Fc antibody, respectively) could then be measured via time resolved fluorescence resonance energy transfer (TR-FRET) due to the proximity of the europium cryptate donor and XL⁶⁶⁵ acceptor fluorophores. Competition of the Abet0380-GL IgG1-TM: N-terminal biotin Abeta 1-42 interaction by test peptides therefore resulted in a reduction in assay signal. Results were expressed as % specific binding where 100% specific binding was derived from wells containing streptavidin-XL^{entl} (5 nM), N-terminal biotin Abeta 1-42 (1.5 nM), Abet0380-GL IgG1-TM (0.5 nM) & europium crptate labelled anti-human Fc antibody (0.8 nM), 0% specific binding was derived from wells in which Abet0380-GL IgG1-TM had been omitted.

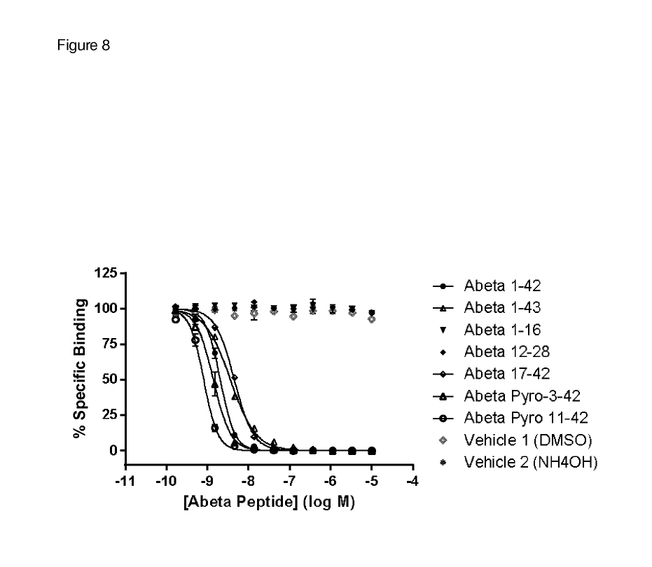

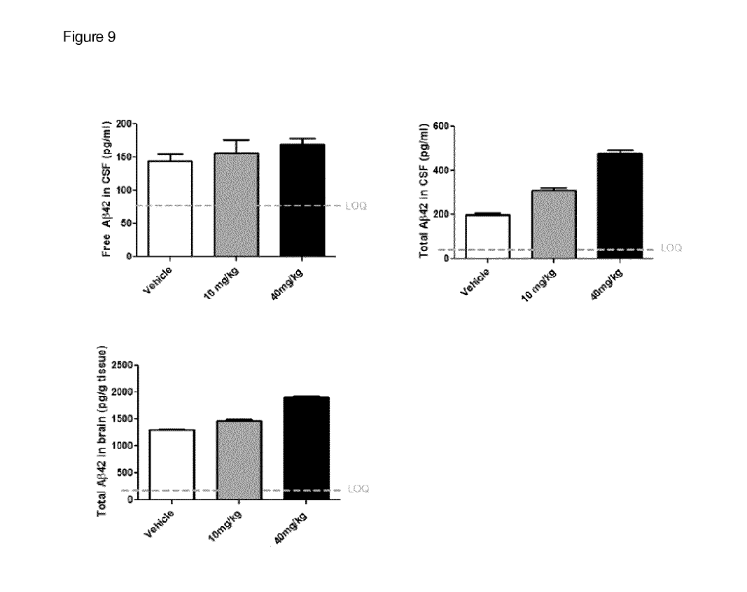

The final assay volume was 20 μl and all reagents were prepared in an assay buffer comprising MOPS pH7.4 (50 mM), potassium fluoride (0.4M), tween 20 (0.1%) & fatty acid free BSA (0.1%). The assay was performed in low volume 384 well black assay plates (Costar 3676). In summary, inhibition of Abet0380-GL IgG1-TM: N-terminal Biotin Abeta 1-42 binding was observed with Abeta 1-42, Abeta 1-43, Abeta 17-42, Abeta Pyro-3-42 & Abeta Pyro-1-42 with IC₅₀ values ranging from 10$ to 10^{A} molar for this group. No inhibition of Abet0380-GL IgG1-TM: N-terminal Biotin Abeta 1-42 binding was observed with Abeta 1-16 or Abeta 12-28 (FIG. 8).

### Example 5: Ability of Antibody Abet0144-GL to Sequester Amyloid Beta 1-42 in a Normal Rat PK-PD Study

The ability of antibody Abet0144-GL to sequester amyloid beta 1-42 was investigated in a PK-PD study in normal rats. Rats were intravenously administered Abet0144-GL (10 or 40 mg/kg) or vehicle weekly for 2 weeks (on days 0 and 7), and sacrificed a week after the 2nd dose. CSF was sampled for free and total amyloid beta 1-42, and brain was sampled for total amyloid beta 1-42 measurement. Free and total amyloid beta 1-42 levels were measured using assays described above.

As shown in FIG. 9, free amyloid beta 1-42 in CSF was not significantly altered by either 10 or 40 mg/kg of Abet0144-GL (5 and 18% increase, respectively when compared with vehicle; FIG. 9). Total amyloid beta 1-42 in CSF was significantly increased by 38% at 10 mg/kg, and by 139% at 40 mg/kg. Total amyloid beta 1-42 in brain tissue was also significantly increased, by 16% and 50% at 10 and 40 mg/kg, respectively. In summary, data from this study in normal rats, demonstrated that Abet0144-GL had no significant effect on free amyloid beta 1-42 levels in CSF, whilst increasing total amyloid beta 1-42 levels in both CSF and brain. This was the profile that would be expected from an antibody with an affinity for target in the tens of nM range.

### Example 6: 6′-Bromospiro[cyclohexane-1,2′-indene]-1′,4(3′H)-dione

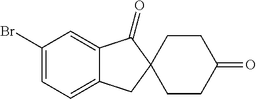 [SMILES: O=C1CCC2(CC1)Cc1ccc(Br)cc1C2=O]

Potassium tert-butoxide (223 g, 1.99 mol) was charged to a 100 L reactor containing a stirred mixture of 6-bromo-1-indanone (8.38 kg, 39.7 mol) in THF (16.75 L) at 20-30° C. Methyl acrylate (2.33 L, 25.8 mol) was then charged to the mixture during 15 minutes keeping the temperature between 20-30° C. A solution of potassium tert-butoxide (89.1 g, 0.79 mol) dissolved in THF (400 mL) was added were after methyl acrylate (2.33 L, 25.8 mol) was added during 20 minutes at 20-30° C. A third portion of potassium tert-butoxide (90 g, 0.80 mol) dissolved in THF (400 mL) was then added, followed by a third addition of methyl acrylate (2.33 L, 25.8 mol) during 20 minutes at 20-30° C. Potassium tert-butoxide (4.86 kg, 43.3 mol) dissolved in THF (21.9 L) was charged to the reactor during 1 hour at 20-30° C. The reaction was heated to approximately 65° C. and 23 L of solvent was distilled off. Reaction temperature was lowered to 60° C. and 50% aqueous potassium hydroxide (2.42 L, 31.7 mol) dissolved in water (51.1 L) was added to the mixture during 30 minutes at 55-60° C. were after the mixture was stirred for 6 hours at 60° C., cooled to 20° C. during 2 hours. After stirring for 12 hours at 20° C. the solid material was filtered off, washed twice with a mixture of water (8.4 L) and THF (4.2 L) and then dried at 50° C. under vacuum to yield 6′-bromospiro[cyclohexane-1,2′-indene]-1′,4(3′H)-dione (7.78 kg, 26.6 mol). ¹H NMR (500 MHz, DMSO-d₆) δ ppm 1.78-1.84 (m, 2H), 1.95 (td, 2H), 2.32-2.38 (m 2H), 2.51-2.59 (m, 2H), 3.27 (s, 2H), 7.60 (d, 1H), 7.81 (m, 1H), 7.89 (m, 1H).

### Example 7: (1r,4r)-6′-Bromo-4-methoxyspiro[cyclohexane-1,2′-indene]-1′(3′H)-one

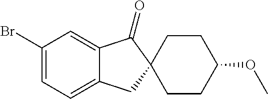 [SMILES: CO[C@H]1CC[C@]2(CC1)Cc1ccc(Br)cc1C2=O]

Borane tert-butylamine complex (845 g, 9.7 mol) dissolved in DCM (3.8 L) was charged to a slurry of 6′-Bromospiro[cyclohexane-1,2′-indene]-1′,4(3′H)-dione (7.7 kg, 26.3 mol) in DCM (42.4 L) at approximately 0-5° C. over approximately 25 minutes. The reaction was left with stirring at 0-5° C. for 1 hour were after analysis confirmed that the conversion was >98%. A solution prepared from sodium chloride (2.77 kg), water (13.3 L) and 37% hydrochloric acid (2.61 L, 32 mol) was charged. The mixture was warmed to approximately 15° C. and the phases separated after settling into layers. The organic phase was returned to the reactor, together with methyl methanesulfonate (2.68 L, 31.6 mol) and tetrabutylammonium chloride (131 g, 0.47 mol) and the mixture was vigorously agitated at 20° C. 50% Sodium hydroxide (12.5 L, 236 mol) was then charged to the vigorously agitated reaction mixture over approximately 1 hour and the reaction was left with vigorously agitation overnight at 20° C. Water (19 L) was added and the aqueous phase discarded after separation. The organic layer was heated to approximately 40° C. and 33 L of solvent were distilled off. Ethanol (21 L) was charged and the distillation resumed with increasing temperature (22 L distilled off at up to 79° C.). Ethanol (13.9 L) was charged at approximately 75° C. Water (14.6 L) was charged over 30 minutes keeping the temperature between 72-75° C. Approximately 400 mL of the solution is withdrawn to a 500 mL polythene bottle and the sample crystallized spontaneously. The batch was cooled to 50° C. were the crystallized slurry sample was added back to the solution. The mixture was cooled to 40° C. The mixture was cooled to 20° C. during 4 hours were after it was stirred overnight. The solid was filtered off, washed with a mixture of ethanol (6.6 L) and water (5 L) and dried at 50° C. under vacuum to yield (1r,4r)-6′-bromo-4-methoxyspiro[cyclohexane-1,2′-indene]-1′(3′H)-one (5.83 kg, 18.9 mol) ¹H NMR (500 MHz, DMSO-d₆) δ ppm 1.22-1.32 (m, 2H), 1.41-1.48 (m, 2H), 1.56 (td, 2H), 1.99-2.07 (m, 2H), 3.01 (s, 2H), 3.16-3.23 (m, 1H), 3.27 (s, 3H), 7.56 (d, 1H), 7.77 (d, 1H), 7.86 (dd, 1H).

### Example 8: (1r,4r)-6′-Bromo-4-methoxyspiro[cyclohexane-1,2′-indene]-1′(3′H)-imine hydrochloride

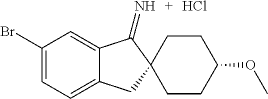 [SMILES: Cl ; CO[C@H]1CC[C@]2(CC1)Cc1ccc(Br)cc1C2=N]

(1r,4r)-6′-Bromo-4-methoxyspiro[cyclohexane-1,2′-indene]-1′(3′H)-one (5.82 kg: 17.7 mol) was charged to a 100 L reactor at ambient temperature followed by titanium (IV)ethoxide (7.4 L; 35.4 mol) and a solution of tert-butylsulfinamide (2.94 kg; 23.0 mol) in 2-methyltetrahydrofuran (13.7 L). The mixture was stirred and heated to 82° C. After 30 minutes at 82° C. the temperature was increased further (up to 97° C.) and 8 L of solvent was distilled off. The reaction was cooled to 87° C. and 2-methyltetrahydrofuran (8.2 L) was added giving a reaction temperature of 82° C. The reaction was left with stirring at 82° C. overnight. The reaction temperature was raised (to 97° C.) and 8.5 L of solvent was distilled off. The reaction was cooled down to 87° C. and 2-methyltetrahydrofuran (8.2 L) was added giving a reaction temperature of 82° C. After 3.5 hours the reaction temperature was increased further (to 97° C.) and 8 L of solvent was distilled off. The reaction was cooled to 87° C. and 2-methyltetrahydrofuran (8.2 L) was added giving a reaction temperature of 82° C. After 2 hours the reaction temperature was increased further (to 97° C.) and 8.2 L of solvent was distilled off. The reaction was cooled to 87° C. and 2-methyltetrahydrofuran (8.2 L) was added giving a reaction temperature of 82° C. The reaction was stirred overnight at 82° C. The reaction temperature was increased further (to 97° C.) and 8 L of solvent was distilled off. The reaction was cooled down to 25° C. Dichloromethane (16.4 L) was charged. To a separate reactor water (30 L) was added and agitated vigorously and sodium sulfate (7.54 kg) was added and the resulting solution was cooled to 10° C. Sulfuric acid (2.3 L, 42.4 mol) was added to the water solution and the temperature was adjusted to 20° C. 6 L of the acidic water solution was withdrawn and saved for later. The organic reaction mixture was charged to the acidic water solution over 5 minutes with good agitation. The organic reaction vessel was washed with dichloromethane (16.4 L), and the dichloromethane wash solution was also added to the acidic water. The mixture was stirred for 15 minutes and then allowed to settle for 20 minutes. The lower aqueous phase was run off, and the saved 6 L of acidic wash was added followed by water (5.5 L). The mixture was stirred for 15 minutes and then allowed to settle for 20 minutes. The lower organic layer was run off to carboys and the upper water layer was discarded. The organic layer was charged back to the vessel followed by sodium sulfate (2.74 kg), and the mixture was agitated for 30 minutes. The sodium sulfate was filtered off and washed with dichloromethane (5.5 L) and the combined organic phases were charged to a clean vessel. The batch was heated for distillation (collected 31 L max temperature 57° C.). The batch was cooled to 40° C. and dichloromethane (16.4 L) was added. The batch was heated for distillation (collected 17 L max temperature 54° C.). The batch was cooled to 20° C. and dichloromethane (5.5 L) and ethanol (2.7 L) were. 2 M hydrogen chloride in diethyl ether (10.6 L; 21.2 mol) was charged to the reaction over 45 minutes keeping the temperature between 16-23° C. The resulting slurry was stirred at 20° C. for 1 hour whereafter the solid was filtered off and washed 3 times with a 1:1 mixture of dichloromethane and diethyl ether (3×5.5 L). The solid was dried at 50° C. under vacuum to yield (1r,4r)-6′-bromo-4-methoxyspiro[cyclohexane-1,2′-indene]-1′(3′H)-imine hydrochloride (6.0 kg; 14.3 mol; assay 82% w/w by ¹H NMR) ¹H NMR (500 MHz, DMSO-d₆) L ppm 130 (m, 2H), 1.70 (d, 2H), 1.98 (m, 2H), 2.10 (m, 2H), 3.17 (s, 2H), 3.23 (m, 1H), 3.29 (s, 3H), 7.61 (d, 1H), 8.04 (dd, 1H), 8.75 (d, 1H), 12.90 (br s, 2H).

### Example 9: (1r,4r)-6′-Bromo-4-methoxy-5″-methyl-3′H-dispiro[cyclohexane-1,2′-indene-1,2″-imidazol]-4″(3″H)-thione

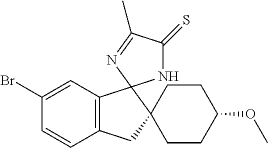

Trimethylorthoformate (4.95 L; 45.2 mol) and diisopropylethylamine (3.5 L; 20.0 mol) was charged to a reactor containing (1r,4r)-6′-bromo-4-methoxyspiro[cyclohexane-1,2′-indene]-1′(3′H)-imine hydrochloride (6.25 kg; 14.9 mol) in isopropanol (50.5 L). The reaction mixture was stirred and heated to 75° C. during 1 hour so that a clear solution was obtained. The temperature was set to 70° C. and a 2 M solution of 2-oxopropanethioamide in isopropanol (19.5 kg: 40.6 mol) was charged over 1 hour, were after the reaction was stirred overnight at 69° C. The batch was seeded with (1r,4r)-6′-bromo-4-methoxy-5″-methyl-3′H-dispiro[cyclohexane-1,2′-indene-1′2″-imidazol]-4′(3″H)-thione (3 g; 7.6 mmol) and the temperature was lowered to 60° C. and stirred for 1 hour. The mixture was concentrated by distillation (distillation temperature approximately 60° C.; 31 L distilled off). Water (31 L) was added during 1 hour and 60° C. before the temperature was lowered to 25° C. during 90 minutes were after the mixture was stirred for 3 hours. The solid was filtered off, washed with isopropanol twice (2×5.2 L) and dried under vacuum at 40° C. to yield (1r,4r)-6′-bromo-4-methoxy-5″-methyl-3′H-dispiro[cyclohexane-1,2′-indene-1′2″-imidazol]-4″(3″H)-thione (4.87 kg; 10.8 mol; assay of 87% w/w by ¹H NMR).

### Example 10: (1r,1′R,4R)-6′-Bromo-4-methoxy-5″-methyl-3′H-dispiro[cyclohexane-1,2′-indene-1′2″-imidazol]-4″-amine D(+)-10-Camphorsulfonic acid salt

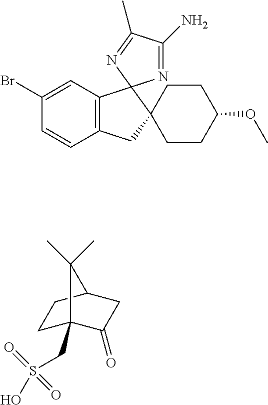

7 M Ammonia in methanol (32 L: 224 mol) was charged to a reactor containing (1r,4r)-6′-bromo-4-methoxy-5″-methyl-3′H-dispiro[cyclohexane-1,2′-indene-1′2″-imidazol]-4″(3H)-thione (5.10 kg; 11.4 mol) and zinc acetate dihydrate (3.02 kg: 13.8 mol). The reactor was sealed and the mixture was heated to 80° C. and stirred for 24 hours, were after it was cooled to 30° C. 1-Butanol (51 L) was charged and the reaction mixture was concentrated by vacuum distilling off approximately 50 L. 1-Butanol (25 L) was added and the mixture was concentrated by vacuum distilling of 27 L. The mixture was cooled to 30° C. and 1 M sodium hydroxide (30 L; 30 mol) was charged. The biphasic mixture was agitated for 15 minutes. The lower aqueous phase was separated off Water (20 L) was charged and the mixture was agitated for 30 minutes. The lower aqueous phase was separated off. The organic phase was heated to 70° C. were after (1S)-(+)-10-camphorsulfonic acid (2.4 kg; 10.3 mol) was charged. The mixture was stirred for 1 hour at 70° C. and then ramped down to 20° C. over 3 hours. The solid was filtered off, washed with ethanol (20 L) and dried in vacuum at 50° C. to yield (1r,4r)-6′-bromo-4-methoxy-5″-methyl-3′H-dispiro[cyclohexane-1,2′-indene-1′2″-imidazole]-4″-amine (+)-10-Camphor sulfonic acid salt (3.12 kg; 5.13 mol: assay 102% w/w by ¹H NMR).

### Example 11: (1r,1′R,4R)-4-methoxy-5″-methyl-6′-[5-(prop-1-yn-1-yl)pyridin-3-yl]-3′H-dispiro[cyclohexane-1,2′-indene-1′2″-imidazol]-4″-amine

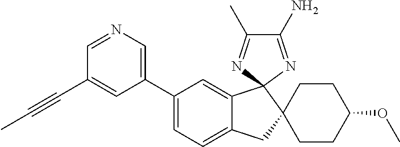 [SMILES: CC#Cc1cncc(-c2ccc3c(c2)[C@@]2(N=C(C)C(N)=N2)[C@]2(CC[C@H](OC)CC2)C3)c1]

Na₂PdCl₄ (1.4 g; 4.76 mmol) and 3-(di-tert-butylphosphonium)propane sulfonate (2.6 g; 9.69 mmol) dissolved in water (0.1 L) was charged to a vessel containing (1r,4r)-6′-bromo-4-methoxy-5″-methyl-3H-dispiro[cyclohexane-1,2-indene-1′2″-imidazol]-4″-amine (+)-10-camphorsulfonic acid salt (1 kg; 1.58 mol), potassium carbonate (0.763 kg; 5.52 mol) in a mixture of 1-butanol (7.7 L) and water (2.6 L). The mixture is carefully inerted with nitrogen whereafter 5-(prop-1-ynyl)pyridine-3-yl boronic acid (0.29 kg; 1.62 mol) is charged and the mixture is again carefully inerted with nitrogen. The reaction mixture is heated to 75° C. and stirred for 2 hours were after analysis showed full conversion. Temperature was adjusted to 45° C. Stirring was stopped and the lower aqueous phase was separated off. The organic layer was washed 3 times with water (3×4 L). The reaction temperature was adjusted to 22° C. and Phosphonics SPM32 scavenger (0.195 kg) was charged and the mixture was agitated overnight. The scavenger was filtered off and washed with 1-butanol (1 L). The reaction is concentrated by distillation under reduced pressure to 3 L. Butyl acetate (7.7 L) is charged and the mixture is again concentrated down to 3 L by distillation under reduced pressure. Butyl acetate (4.8 L) was charged and the mixture was heated to 60° C. The mixture was stirred for 1 hour were after it was concentrated down to approximately 4 L by distillation under reduced pressure. The temperature was set to 60° C. and heptanes (3.8 L) was added over 20 minutes. The mixture was cooled down to 20° C. over 3 hours and then left with stirring overnight. The solid was filtered off and washed twice with a 1:1 mixture of butyl acetate:heptane (2×2 L). The product was dried under vacuum at 50° C. to yield (1r,1′R,4R)-4-methoxy-5″-methyl-6′-[5-(prop-1-yn-1-yl)pyridin-3-yl]-3′H-dispiro[cyclohexane-1,2′-indene-1′2″-imidazol]-4″-amine (0.562 kg: 1.36 mol; assay 100% w/w by ¹H NMR). ¹H NMR (500 MHz, DMSO-d₆) □ ppm 0.97 (d, 1H), 1.12-1.30 (m, 2H), 1.37-1.51 (m, 3H), 1.83 (d, 2H), 2.09 (s, 3H), 2.17 (s, 2H), 2.89-3.12 (m, 3H), 3.20 (s, 3H), 6.54 (s, 2H), 6.83 (s, 1H), 7.40 (d, 1H), 7.54 (d, 1H), 7.90 (s, 1H), 8.51 (d, 1H), 8.67 (d, 1H)

### Example 12: Preparation of camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine

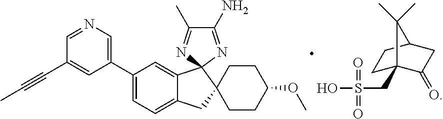

1.105 kg (1 r,1′R,4R)-4-methoxy-5″-methyl-6′-[5-(prop-1-yn-1-yl)pyridin-3-yl]-3′H-dispiro[cyclohexane-1,2′-indene-1′2″-imidazol]-4″-amine was dissolved in 8.10 L2-propanol and 475 mL water at 60° C. Then 1.0 mole equivalent (622 gram) (1S)-(+)-10 camphorsulfonic acid was charged at 60° C. The slurry was agitated until all (1S)-(+)-10 camphorsulfonic acid was dissolved. A second portion of 2-propanol was added (6.0 L) at 60° C. and then the contents were distilled until 4.3 L distillate was collected. Then 9.1 L Heptane was charged at 65° C. After a delay of one hour the batch became opaque. Then an additional distillation was performed at about 75° C. and 8.2 L distillate was collected. The batch was then cooled to 20° C. over 2 hrs and held at that temperature overnight. Then the batch was filtered and washed with a mixture of 1.8 L 2-propanol and 2.7 L heptane. Finally the substance was dried at reduced pressure and 50° C. The yield was 1.44 kg (83.6% w/w). ¹H NMR (400 MHz DMSO-d₆) δ ppm 12.12 (1H, s), 9.70 (2H, d, J 40.2), 8.81 (1H, d, J 2.1), 8.55 (1H, d, J 1.7), 8.05 (1H, dd, J 2.1, 1.7), 7.77 (1H, dd. J 7.8, 1.2), 7.50 (2H, m), 3.22 (3H, s), 3.19 (1H, d, J 16.1), 3.10 (1H, d, J 16.1), 3.02 (1H, m), 2.90 (1H, d, J 14.7), 2.60 (1H, m), 2.41 (1H, d, J 14.7), 2.40 (3H, s), 2.22 (1H, m), 2.10 (3H, s), 1.91 (3H, m), 1.81 (1H, m), 1.77 (1H, d, 1.81), 1.50 (2H, m), 1.25 (6H, m), 0.98 (3H, s), 0.69 (3H, s).

### Example 13: Testing Activity of (1r,1′R,4R)-4-methoxy-5-methyl-6′-[5-(prop-1-yn-1-yl)pyridine-3-yl]-3′H-dispiro[cyclohexane-1,2′-indene-1′2″-imidazol]-4″-amine

The level of activity of (1r,1′R,4R)-4-methoxy-5″-methyl-6′-[5-(prop-1-yn-1-yl)pyridin-3-yl]-3′H-dispiro[cyclohexane-1,2′-indene-1′2″-imidazol]-4″-amine was tested using the following methods:

TR-FRET Assay

The β-secretase enzyme used in the TR-FRET is prepared as follows: The cDNA for the soluble part of the human β-Secretase (AA 1-AA 460) was cloned using the ASP2-Fc 10-1-IRES-GFP-neoK mammalian expression vector. The gene was fused to the Fc domain of IgG1 (affinity tag) and stably cloned into HEK 293 cells. Purified sBACE-Fc was stored in −80° C. in Tris buffer, pH 9.2 and had a purity of 40%.

The enzyme (truncated form) was diluted to 6 pg/mL (stock 1.3 mg/mL) and the substrate (Europium) CEVNLDAEFK (Qsy7) to 200 nM (stock 120 μM) in reaction buffer (NaAcetate, chaps, triton x-100, EDTA pH4.5). The robotic systems Biomek FX and Velocity 11 were used for all liquid handling and the enzyme and substrate solutions were kept on ice until they were placed in the robotic system. Enzyme (9 μl) was added to the plate then 1 μl of compound in dimethylsulphoxide was added, mixed and pre-incubated for 10 minutes. Substrate (10 μl) was then added, mixed and the reaction proceeded for 15 minutes at r.t. The reaction was stopped with the addition of Stop solution (7 μl, aAcetate, pH 9). The fluorescence of the product was measured on a Victor II plate reader with an excitation wavelength of 340 nm and an emission wavelength of 615 nm. The assay was performed in a Costar 384 well round bottom, low volume, non-binding surface plate (Corning #3676). The final concentration of the enzyme was 2.7 pg/ml; the final concentration of substrate was 100 nM (Km of ˜250 nM). The dimethylsulphoxide control, instead of test compound, defined the 100% activity level and 0% activity was defined by wells lacking enzyme (replaced with reaction buffer). A control inhibitor was also used in dose response assays and had an IC₅₀ of ˜150 nM.

Diluted TR-FRET Assay The compound was further tested in a diluted TR-FRET assay, conditions as described above for the TR-FRET assay, but with 50 times less enzyme and a 6.5 h long reaction time at r.t. in the dark.

sAPPβ Release Assay

SH-SY5Y cells were cultured in DMEM/F-12 with Glutamax, 10% FCS and 1% non-essential amino acids and cryopreserved and stored at −140) ° C. at a concentration of 7.5-9.5×10⁶ cells per vial. Thaw cells and seed at a conc. of around 10000 cells/well in DMEM/F-12 with Glutamax, 10% FCS and 1% non-essential amino acids to a 384-well tissue culture treated plate, 100 μL cell susp/well. The cell plates were then incubated for 7-24 h at 37° C., 5% CO₂. The cell medium was removed, followed by addition of 30 μL compound diluted in DMEM/F-12 with Glutamax, 10% FCS, 1% non-essential amino acids and 1% PeSt to a final conc. of 1% DMSO. The compound was incubated with the cells for 17 h (overnight) at 37° C., 5% CO₂. Meso Scale Discovery (MSD) plates were used for the detection of sAPPβ release. MSD sAPPβ plates were blocked in 1% BSA in Tris wash buffer (40 μL/well) for 1 h on shake at r.t. and washed 1 time in Tris wash buffer (40 μL/well). 20 μL of medium was transferred to the pre-blocked and washed MSD sAPPβ microplates, and the cell plates were further used in an ATP assay to measure cytotoxicity. The MSD plates were incubated with shaking at r.t. for 2 h and the media discarded. 10 μL detection antibody was added (1 nM) per well followed by incubation with shaking at r.t. for 2 h and then discarded. 40 μL Read Buffer was added per well and the plates were read in a SECTOR Imager.

ATP Assay

As indicated in the sAPPβ release assay, after transferring 20 μL medium from the cell plates for sAPPβ detection, the plates were used to analyse cytotoxicity using the ViaLight™ Plus cell proliferation/cytotoxicity kit from Cambrex BioScience that measures total cellular ATP. The assay was performed according to the manufacture's protocol. Briefly, 10 μL cell lysis reagent was added per well. The plates were incubated at r.t. for 10 min. Two min after addition of 25 μL reconstituted ViaLight™ Plus ATP reagent, the luminescence was measured in a Wallac Victor2 1420 multilabel counter. Tox threshold is a signal below 75% of the control.

Results

IC₅₀ values for (1r,1′R,4R)-4-methoxy-5″-methyl-6′-[5-(prop-1-yn-1-yl)pyridin-3-yl]-3′H-dispiro[cyclohexane-1,2′-indene-1′2″ ‘ ’-imidazol]-4″-amine isomers are summarized below in Table 15.

| IC₅₀ in TR- | IC₅₀ in sAPPβ |
|---|---|
| FRET assay (nM) | release assay (nM) |
| 0.57^{a} | 0.10 |

^{a}IC₅₀ from the diluted FRET assay.

### Example 14: Activity of the camsylate salt of (1r,1′R,4R)-4-methoxy-5″-methyl-6′-[5-(prop-1-yn-1-yl)pyridin-3-yl]-3′H-dispiro[cyclohexane-1,2′-indene-1′2″-imidazol]-4″-amine

The level of activity of the camsylate salt of (1r,1′R4R)-4-methoxy-5″-methyl-6′-[5-(prop-1-yn-1-yl)pyridin-3-yl]-3′H-dispiro[cyclohexane-1,2′-indene-1′,2″-imidazol]-4″-amine can be tested using the following methods:

TR-FRET Assay

The β-secretase enzyme used in the TR-FRET is prepared as follows: The cDNA for the soluble part of the human β-Secretase (AA 1-AA 460) was cloned using the ASP2-Fc 10-1-IRES-GFP-neoK mammalian expression vector. The gene was fused to the Fc domain of IgG1 (affinity tag) and stably cloned into HEK 293 cells. Purified sBACE-Fc was stored in −80° C. in 50 mM Glycine pH 2.5, adjusted to pH 7.4 with 1 M Tris and had a purity of 40%.

The enzyme (truncated form) was diluted to 6 μg/mL (stock 1.3 mg/mL) and TruPoint BACE 1 Substrate to 200 nM (stock 120 μM) in reaction buffer (NaAcetate, chaps, triton x-100, EDTA pH4.5). Enzyme and compound in dimethylsulphoxide (final DMSO concentration 5%) was mixed and pre-incubated for 10 minutes at RT. Substrate was then added and the reaction was incubated for 15 minutes at RT. The reaction was stopped with the addition of 0.35 vol Stop solution (NaAcetate. pH 9). The fluorescence of the product was measured on a Victor II plate reader with excitation wavelengths of 340-485 nm and emission wavelengths of 590-615 nm. The final concentration of the enzyme was 2.7 μg/ml: the final concentration of substrate was 100 nM (Km of ˜250 nM). The dimethylsulphoxide control, instead of test compound, defined the 100% activity level and 0% activity was defined by wells lacking enzyme (replaced with reaction buffer) or by a saturating dose of a known inhibitor, 2-amino-6-[3-(3-methoxyphenyl)phenyl]-3,6-dimethyl-5H-pyrimidin-4-one. A control inhibitor was also used in dose response assays and had an IC50 of ˜150 nM.

The camsylate salt of (1r,1′R,4R)-4-methoxy-5″-methyl-6′-[5-(prop-1-yn-1-yl)pyridin-3-yl]-3′H-dispiro[cyclohexane-1,2′-indene-1,2″-imidazol]-4″-amine had an average IC₅₀ of 0.2 nM in this assay.

sAPPβ Release Assay

SH-SY5Y cells are cultured in DMEM/F-12 with Glutamax, 10% FCS and 1% non-essential amino acids and cryopreserved and stored at −140° C. at a concentration of 7.5-9.5×10⁶ cells per vial. Cells are thawed and seeded at a conc. of around 10000 cells/well in DMEM/F-12 with Glutamax, 10% FCS and 1% non-essential amino acids to a 384-well tissue culture treated plate, 100 μL cell susp/well. The cell plates are then incubated for 7-24 h at 37° C., 5% CO₂. The cell medium is removed, followed by addition of 30 μL compound diluted in DMEM/F-12 with Glutamax, 10°/o FCS, 1% non-essential amino acids and 1% PeSt to a final conc. of 1% DMSO. The compound was incubated with the cells for 17 h (overnight) at 37° C., 5% CO₂. Meso Scale Discovery (MSD) plates are used for the detection of sAPPβ release. MSD sAPPβ plates are blocked in 1% BSA in Tris wash buffer (40) μL/well) for 1 h on shake at r.t. and washed 1 time in Tris wash buffer (40 μL/well). 20 μL of medium is transferred to the pre-blocked and washed MSD sAPPβ microplates, and the cell plates are further used in an ATP assay to measure cytotoxicity. The MSD plates are incubated with shaking at r.t. for 2 h and the media discarded. 10 μL detection antibody is added (1 nM) per well followed by incubation with shaking at r.t. for 2 h and then discarded. 40 μL Read Buffer is added per well and the plates are read in a SECTOR Imager.

ATP Assay

As indicated in the sAPP β release assay, after transferring 20 μL medium from the cell plates for sAPPβ detection, the plates are used to analyse cytotoxicity using a ViaLight™ Plus cell proliferation/cytotoxicity kit from Cambrex BioScience that measures total cellular ATP. The assay is performed according to the manufacture's protocol. Briefly, 10 μL cell lysis reagent is added per well. The plates are incubated at r.t. for 10 min. Two min after addition of 25 μL reconstituted ViaLight™ Plus ATP reagent, luminescence is measured. Tox threshold is a signal below 75% of the control.

### Example 15: Administration of an Antibody or Antigen-Binding Fragment and BACE Inhibitor to an Animal Model of Alzheimer's Disease

A representative antibody or antigen-binding fragment (e.g., Abet0380-GL) and a representative BACE inhibitor (e.g., the camsylate salt of

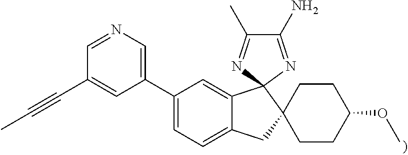 [SMILES: CC#Cc1cncc(-c2ccc3c(c2)[C@@]2(N=C(C)C(N)=N2)[C@]2(CC[C@H](OC)CC2)C3)c1]

are administered in combination to any one of the following representative animal models: the PDAPP mice described in Games et al., 1995. Nature, 373(6514):523-7; the C57BL/6 mice or Dunkin-Hartley guinea pigs described in Eketjall et al., 2016, Journal of Alzheimer's Disease, 50(4): 1109-1123; the Sprague-Dawley rats or Tg2576 mice described in Example 2 above. Control animal models will be administered corresponding dosages of the antibody or antigen-binding fragment alone, the BACE inhibitor alone, or of vehicle control. The antibody or antigen-binding fragment is administered intravenously in a manner consistent with that described in Example 2. The BACE inhibitor is administered orally in a manner similar to that described in Eketjall et al. Mice are monitored for any signs that the combination therapy is toxic to the mice (e.g., monitored for signs of weakness, lethargy, weight loss, death), and the dose of each drug is adjusted accordingly to achieve a maximum therapeutic effect while minimizing any cytotoxic effects. Bioanalysis of brain, plasma and CSF samples (e.g., bioanalysis of AB levels in those samples) is monitored from the animals in a manner similar to that described in Example 2 above and in Eketjall et al. The effects of the different treatment conditions will also be assessed in mice using behavioural and/or cognitive assays known in the art. An improvement in a tested parameter, such as Aβₙ₋₄₂ levels (e.g., a greater reduction in Aβ₁₋₄₂ levels), that is greater in the animal models administered the combination therapy than in the control animal models is suggestive that the combination therapy is more effective in addressing that parameter than treatment with either the BACE inhibitor or the antibody or antigen-binding fragment alone. The skilled worker is aware of other models, and other parameters, in which to test the effect of the combination therapy. See. e.g., Bogstedt et al., 2015, Journal of Alzheimer's Disease, 46:1091-1101.

### REFERENCES

Bannister, et al., (2006). Biotechnol. Bioeng. 94, 931-937.
Bard, F, et al. (2000). Nat. Med. 6, 916-919.
Borchelt, et al. (1996). Neuron 17, 1005-1013.
Citron, M., et al. (1998). Neurobiol. Dis. 5, 107-116.
Clackson, T. and Lowman, H. B. (2004). Phage display: a practical approach, Oxford University Press.
De Strooper, B. (2007). EMBO Rep. 8, 141-146.
DeMattos, R. B., et al. (2001). Proc. Natl. Acad. Sci. USA 98, 8850-8855.
Duff, K., et al. (1996). Nature 383, 710-713.
Foote. J. and Winter, G. (1992). J. Mol. Biol. 224, 487-499.
Gilman, S., et al. (2005). Neurology 64, 1553-1562.
Glabe, C. (2000). Nat. Med. 6, 133-134.
Golde, T. E., Das, P. and Levites, Y. (2009). CNS & Neuro. Dis.—Drug Targets 8, 31-49
Greeve, I., et al. (2004). J. Neurosci. 24, 3899-3906.
Groves, M. A. and Osbourn, J. K. (2005). Expert Opin. Biol. Ther. 5, 125-135.
Hanes, J., Jermutus, L. and Pluckthun, A. (2000). Methods Enzymol. 328, 404-430.
Hanes, J. and Pluckthun, A. (1997). Proc. Natl. Acad. Sci. USA 94, 4937-4942.
Hawkins, R E., Russell, S. J. and Winter, G. (1992). J. Mol. Biol. 226, 889-896.
Hoet, R. M., et al. (2005). Nat. Biotechnol. 23, 344-348.
Iijima, K., et al. (2004). Proc. Natl. Acad. Sci. USA 101, 6623-6628.
Karlsson, R., Michaelsson, A. and Mattsson, L. (1991). J. Immunol Methods 145, 229-240.
Kuperstein, I., et al. (2010). EMBO J. 29, 3408-3420.
Lambert, M. P., et al. (1998). Proc. Natl. Acad. Sci. USA 95, 6448-6453.
Levites. Y., et al. (2006). J. Clin. Invest. 116, 193-201.
Matsuoka. Y., et al. (2003). J. Neurosci. 23, 29-33.
McCafferty, J., et al. (1994). Appl. Biochem. Biotechnol. 47, 157-171: discussion 171-153.
McGowan. E., et al. (2005). Neuron 47, 191-199.
Mucke, L., et al. (2000). J. Neurosci. 20, 4050-4058.
Oganesyan, V., et al. (2008). Acta Crvstallogr. D Biol. Crystallogr. 64, 700-704.
Orgogozo, J. M., et al. (2003). Neurology 61, 46-54.
Osbourn, J. K., et al. (1996). Immunotechnology 2, 181-196.
Persic, L., et al. (1997). Gene 187, 9-18.
Portelius, E., et al. (2010). Acta Neuropathol. 120, 185-193.
Pride, M., et al. (2008). Neurodegener. Dis. 5, 194-196.
Schenk, D., et al. (1999). Nature 400, 173-177.
Schenk, D. B., et al. (2000). Arch. Neurol. 57, 934-936.
Scheuner, D., et al. (1996). Nat. Med. 2, 864-870.
Schier, R., et al. (1996). J. Mol. Biol. 255, 28-43.
Selkoe, D. J. (1999). Nature 399, A23-31.
Thompson, J., et al. (1996). J. Mol. Biol. 256, 77-88.
Tomlinson, I. M., et al. (1992). J. Mol. Biol. 227, 776-798.
Vassar, R., et al. (1999). Science 286, 735-741.
Vaughan, T. J., et al. (1996). Nat. Biotechnol. 14, 309-314.
Walsh, D. M., et al. (2002). Nature 416, 535-539.
Walsh, D. M., et al. (2005a). Biochem. Soc. Trans. 33, 1087-1090.
Walsh, D. M., et al. (2005b). J. Neurosci. 25, 2455-2462.
Wang, H. W., et al. (2002). Brain Res. 924, 133-140.
Weller, R. O. and Nicoll, J. A. (2003). Neurol. Res. 25, 611-616.
Wilcock, D. M., et al. (2006). J. Neurosci. 26, 5340-5346.
Wilcock, D. M. and Colton, C. A. (2009). CNS Neurol. Disord Drug. Targets. 8, 50-64.
Younkin, S. G. (1995). Ann. Neurol. 37, 287-288.
Younkin, S. G. (1998). J. Physiol. Paris 92, 289-292.

Other references are included in the text.

1. A method of treating a subject having a disease or disorder associated with the accumulation of Aβ, comprising administering to the subject:
a) a pharmaceutically effective amount of a BACE inhibitor, wherein the BACE inhibitor is: 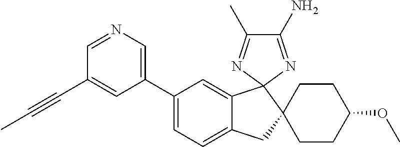
or a pharmaceutically acceptable salt thereof; and
b) a pharmaceutically effective amount of an antibody or antigen-binding fragment comprising at least 1, 2, 3, 4, 5 or 6 CDRs from any one of Abet0380, Abet0342, Abet0369, Abet 0377 or Abet0382, or a germlined variant thereof.

2. The method of claim 1, wherein the BACE inhibitor is: 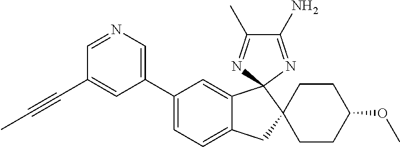 [SMILES: CC#Cc1cncc(-c2ccc3c(c2)[C@@]2(N=C(C)C(N)=N2)[C@]2(CC[C@H](OC)CC2)C3)c1]
or a pharmaceutically acceptable salt thereof.

3. The method of claim 1, wherein the BACE inhibitor is a camsylate salt

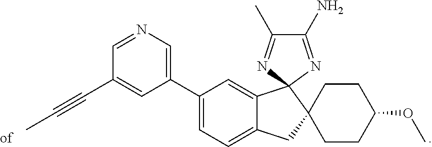 [SMILES: CC#Cc1cncc(-c2ccc3c(c2)[C@@]2(N=C(C)C(N)=N2)[C@]2(CC[C@H](OC)CC2)C3)c1]

4. The method of claim 1, wherein the BACE inhibitor is:

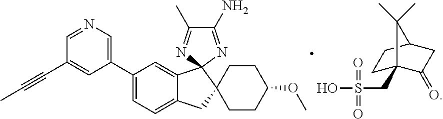

5-9. (canceled)

10. The method of claim 1, wherein the VH domain comprises:
a VH CDR1 having the amino acid sequence of SEQ ID NO: 525;
a VH CDR2 having the amino acid sequence of SEQ ID NO: 526; and
a VH CDR3 having the amino acid sequence of SEQ ID NO: 527.

11. The method of claim 10, wherein the VL domain comprises:
a VL CDR1 having the amino acid sequence of SEQ ID NO: 534;
a VL CDR2 having the amino acid sequence of SEQ ID NO: 535; and
a VL CDR3 having the amino acid sequence of SEQ ID NO: 536.

12-15. (canceled)

16. The method of claim 1, wherein the VH domain comprises an amino acid sequence that is at least 90% identical to SEQ ID NO: 524.

17. The method of claim 1, wherein the VL domain comprises an amino acid sequence that is at least 90% identical to SEQ ID NO: 533.

18-19. (canceled)

20. The method of claim 1, wherein the VH domain comprises the amino acid sequence of SEQ ID NO: 524.

21. The method of claim 1, wherein the VL domain comprises the amino acid sequence of SEQ ID NO: 533.

22-24. (canceled)

25. The method of claim 1, wherein the antibody or antigen-binding fragment is an antibody.

26-29. (canceled)

30. The method of claim 1, wherein the antibody or antigen-binding fragment is humanized or human.

31. (canceled)

32. The method of claim 1, wherein the antibody or antigen-binding fragment binds monomeric Aβ1-42 with a dissociation constant (KD) of 500 pM or less and either does not bind Aβ1-40 or binds Aβ1-40 with a KD greater than 1 mM.

33. The method of claim 1, wherein the antibody or antigen-binding fragment binds amyloid beta 17-42 peptide (Aβ17-42) and amyloid beta 29-42 peptide (Aβ29-42).

34. The method of claim 1, wherein the antibody or antigen-binding fragment binds 3-pyro-42 amyloid beta peptide and 11-pyro-42 amyloid beta peptide.

35. The method of claim 1, wherein the antibody or antigen-binding fragment binds amyloid beta 1-43 peptide (Aβ1-43).

36. The method of claim 1, wherein the disease or disorder is selected from the group consisting of: Alzheimer's disease, Down Syndrome, and/or macular degeneration.

37. The method of claim 36, wherein the disease or disorder is Alzheimer's Disease.

38-39. (canceled)

40. The method of claim 1, wherein the BACE inhibitor and antibody or antigen-binding fragment are administered to the subject simultaneously.

41. The method of claim 1, wherein the BACE inhibitor and antibody or antigen-binding fragment are administered separately.

42. The method of claim 1, wherein the BACE inhibitor and antibody or antigen-binding fragment are in the same composition.

43. The method of claim 1, wherein the BACE inhibitor is administered orally.

44. The method of claim 1, wherein the antibody or antigen-binding fragment is administered intravenously.

45-48. (canceled)

49. A composition comprising a BACE inhibitor for use in combination with an antibody or antigen-binding fragment for treating a disease or disorder associated with AP accumulation, wherein the BACE inhibitor is: 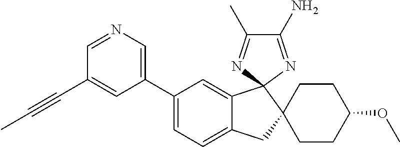 [SMILES: CC#Cc1cncc(-c2ccc3c(c2)[C@@]2(N=C(C)C(N)=N2)[C@]2(CC[C@H](OC)CC2)C3)c1]
or a pharmaceutically acceptable salt thereof; and wherein the antibody or antigen-binding fragment comprises at least 1, 2, 3, 4, 5 or 6 CDRs from any one of Abet0380, Abet0342, Abet0369, Abet 0377 or Abet0382, or a germlined variant thereof.

50-56. (canceled)

57. A kit comprising a BACE inhibitor and an antibody or antigen-binding fragment, wherein the BACE inhibitor is: 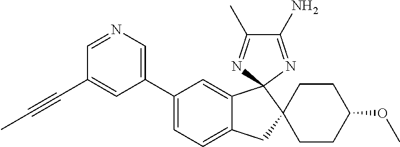 [SMILES: CC#Cc1cncc(-c2ccc3c(c2)[C@@]2(N=C(C)C(N)=N2)[C@]2(CC[C@H](OC)CC2)C3)c1]
or a pharmaceutically acceptable salt thereof; and wherein the antibody or antigen-binding fragment comprises at least 1, 2, 3, 4, 5 or 6 CDRs from any one of Abet0380, Abet0342, Abet0369, Abet 0377 or Abet0382, or a germlined variant thereof.

58-63. (canceled)


SEQUENCE LISTING (648 sequences)

SEQ ID NO: 1 - DNA, 375 bp, Homo sapiens
  source: /note=“Abet0007”
gaggtgcagctgttggagtctgggggaggcttggtacagcctggggggtccctgagactctcctgtgcagcctctggattcacctttagcgtttatactatgtggtgggtccgccaggctccagggaaggggctggagtgggtctcagttattggttctagtggtggtacgacagtttacgcagactccgtgaagggccggttcaccatctccagagacaattccaagaacacgctgtatctgcaaatgaacagcctgagagccgaggacacggccgtgtattactgtgcgagagaagggcagcagctggtacgcccctactactactacggtatggacgtctgggggcaggggaccctggtcaccgtctcctca

SEQ ID NO: 2 - protein, 125 aa, Homo sapiens
  source: /note=“Abet0007”
EVQLLESGGGLVQPGGSLRLSCAASGFTFSVYTMWWVRQAPGKGLEWVSVIGSSGGTTVYADSVKGRFTISRDNSKNTLYLQMNSLRAEDTAVYYCAREGQQLVRPYYYYGMDVWGQGTLVTVSS

SEQ ID NO: 3 - protein, 5 aa, Homo sapiens
  source: /note=“Abet0007”
VYTMW

SEQ ID NO: 4 - protein, 17 aa, Homo sapiens
  source: /note=“Abet0007”
VIGSSGGTTVYADSVKG

SEQ ID NO: 5 - protein, 16 aa, Homo sapiens
  source: /note=“Abet0007”
EGQQLVRPYYYYGMDV

SEQ ID NO: 6 - protein, 30 aa, Homo sapiens
  source: /note=“Abet0007”
EVQLLESGGGLVQPGGSLRLSCAASGFTFS

SEQ ID NO: 7 - protein, 14 aa, Homo sapiens
  source: /note=“Abet0007”
WVRQAPGKGLEWVS

SEQ ID NO: 8 - protein, 32 aa, Homo sapiens
  source: /note=“Abet0007”
RFTISRDNSKNTLYLQMNSLRAEDTAVYYCAR

SEQ ID NO: 9 - protein, 11 aa, Homo sapiens
  source: /note=“Abet0007”
WGQGTLVTVSS

SEQ ID NO: 10 - DNA, 318 bp, Homo sapiens
  source: /note=“Abet0007”
cagagcgtcttgactcagccaccctcagtgtccgtgtccccaggacagacggccagcatcacctgctctggacataacttggaagataaatttgcttcctggtatcaacagaagtcaggccagtcccctgtcctggtcatctatcgagatgacaagcggccctcagggatccctgagcgattctctgcctccaactctgggcacactgccactctgaccatcagcgggacccaggctacggatgaggctgactattactgtcaggcgcaggacagtaccactcgagtgttcggcggagggaccaagctgaccgtccta

SEQ ID NO: 11 - protein, 106 aa, Homo sapiens
  source: /note=“Abet0007”
QSVLTQPPSVSVSPGQTASITCSGHNLEDKFASWYQQKSGQSPVLVIYRDDKRPSGIPERFSASNSGHTATLTISGTQATDEADYYCQAQDSTTRVFGGGTKLTVL

SEQ ID NO: 12 - protein, 11 aa, Homo sapiens
  source: /note=“Abet0007”
SGHNLEDKFAS

SEQ ID NO: 13 - protein, 7 aa, Homo sapiens
  source: /note=“Abet0007”
RDDKRPS

SEQ ID NO: 14 - protein, 9 aa, Homo sapiens
  source: /note=“Abet0007”
QAQDSTTRV

SEQ ID NO: 15 - protein, 22 aa, Homo sapiens
  source: /note=“Abet0007”
QSVLTQPPSVSVSPGQTASITC

SEQ ID NO: 16 - protein, 15 aa, Homo sapiens
  source: /note=“Abet0007”
WYQQKSGQSPVLVIY

SEQ ID NO: 17 - protein, 32 aa, Homo sapiens
  source: /note=“Abet0007”
GIPERFSASNSGHTATLTISGTQATDEADYYC

SEQ ID NO: 18 - protein, 10 aa, Homo sapiens
  source: /note=“Abet0007”
FGGGTKLTVL

SEQ ID NO: 19 - DNA, 375 bp, Homo sapiens
  source: /note=“Abet0144-GL”
gaggtgcagctgttggagtctgggggaggcttggtacagcctggggggtccctgagactctcctgtgcagcctctggattcacctttagcgtttatactatgtggtgggtccgccaggctccagggaaggggctggagtgggtctcagttattggttctagtggtggtacgacagtttacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 20 - protein, 125 aa, Homo sapiens
  source: /note=“Abet0144-GL”
EVQLLESGGGLVQPGGSLRLSCAASGFTFSVYTMWWVRQAPGKGLEWVSVIGSSGGTTVYADSVKGRFTISRDNSKNTLYLQMNSLRAEDTAVYYCAREWMDHSRPYYYYGMDVWGQGTLVTVSS

SEQ ID NO: 21 - protein, 5 aa, Homo sapiens
  source: /note=“Abet0144-GL”
VYTMW

SEQ ID NO: 22 - protein, 17 aa, Homo sapiens
  source: /note=“Abet0144-GL”
VIGSSGGTTVYADSVKG

SEQ ID NO: 23 - protein, 16 aa, Homo sapiens
  source: /note=“Abet0144-GL”
EWMDHSRPYYYYGMDV

SEQ ID NO: 24 - protein, 30 aa, Homo sapiens; identical to SEQ ID NO: 6
  source: /note=“Abet0144-GL”

SEQ ID NO: 25 - protein, 14 aa, Homo sapiens
  source: /note=“Abet0144-GL”
WVRQAPGKGLEWVS

SEQ ID NO: 26 - protein, 32 aa, Homo sapiens; identical to SEQ ID NO: 8
  source: /note=“Abet0144-GL”

SEQ ID NO: 27 - protein, 11 aa, Homo sapiens
  source: /note=“Abet0144-GL”
WGQGTLVTVSS

SEQ ID NO: 28 - DNA, 318 bp, Homo sapiens
  source: /note=“Abet0144-GL”
tcgtacgagttgactcagccaccctcagtgtccgtgtccccaggacagacggccagcatcacctgctctggacataacttggaagataaatttgcttcctggtatcaacagaagccaggccagtcccctgtcctggtcatctatcgagatgacaagcggccctcagggatccctgagcgattctctgcctccaactctgggcacactgccactctgaccatcagcgggacccaggctatggatgaggctgactattactgtcaggcgcaggacagtaccactcgagtgttcggcggagggaccaagctgaccgtccta

SEQ ID NO: 29 - protein, 106 aa, Homo sapiens
  source: /note=“Abet0144-GL”
SYELTQPPSVSVSPGQTASITCSGHNLEDKFASWYQQKPGQSPVLVIYRDDKRPSGIPERFSASNSGHTATLTISGTQAMDEADYYCQAQDSTTRVFGGGTKLTVL

SEQ ID NO: 30 - protein, 11 aa, Homo sapiens
  source: /note=“Abet0144-GL”
SGHNLEDKFAS

SEQ ID NO: 31 - protein, 7 aa, Homo sapiens
  source: /note=“Abet0144-GL”
RDDKRPS

SEQ ID NO: 32 - protein, 9 aa, Homo sapiens
  source: /note=“Abet0144-GL”
QAQDSTTRV

SEQ ID NO: 33 - protein, 22 aa, Homo sapiens
  source: /note=“Abet0144-GL”
SYELTQPPSVSVSPGQTASITC

SEQ ID NO: 34 - protein, 15 aa, Homo sapiens
  source: /note=“Abet0144-GL”
WYQQKPGQSPVLVIY

SEQ ID NO: 35 - protein, 32 aa, Homo sapiens
  source: /note=“Abet0144-GL”
GIPERFSASNSGHTATLTISGTQAMDEADYYC

SEQ ID NO: 36 - protein, 10 aa, Homo sapiens
  source: /note=“Abet0144-GL”
FGGGTKLTVL

SEQ ID NO: 37 - DNA, 375 bp, Homo sapiens
  source: /note=“Abet0319”
gaggtgcagctgttggagtctgggggaggcttggtacagcctggggggtccctgagactctcctgtgcggcctctgtctccgtgtacaacaaggacactatgtggtgggtccgccaggctccagggaaggggctggagtgggtctcagttattggttctagtggtggcacgacagtctacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 38 - protein, 125 aa, Homo sapiens
  source: /note=“Abet0319”
EVQLLESGGGLVQPGGSLRLSCAASVSVYNKDTMWWVRQAPGKGLEWVSVIGSSGGTTVYADSVKGRFTISRDNSKNTLYLQMNSLRAEDTAVYYCAREWMDHSRPYYYYGMDVWGQGTLVTVSS

SEQ ID NO: 39 - protein, 5 aa, Homo sapiens
  source: /note=“Abet0319”
KDTMW

SEQ ID NO: 40 - protein, 17 aa, Homo sapiens
  source: /note=“Abet0319”
VIGSSGGTTVYADSVKG

SEQ ID NO: 41 - protein, 16 aa, Homo sapiens
  source: /note=“Abet0319”
EWMDHSRPYYYYGMDV

SEQ ID NO: 42 - protein, 30 aa, Homo sapiens
  source: /note=“Abet0319”
EVQLLESGGGLVQPGGSLRLSCAASVSVYN

SEQ ID NO: 43 - protein, 14 aa, Homo sapiens
  source: /note=“Abet0319”
WVRQAPGKGLEWVS

SEQ ID NO: 44 - protein, 32 aa, Homo sapiens; identical to SEQ ID NO: 8
  source: /note=“Abet0319”

SEQ ID NO: 45 - protein, 11 aa, Homo sapiens
  source: /note=“Abet0319”
WGQGTLVTVSS

SEQ ID NO: 46 - DNA, 318 bp, Homo sapiens
  source: /note=“Abet0319”
tcgtacgagttgactcagccaccctcagtgtccgtgtccccaggacagacggccagcatcacctgctctggacataacatcatggacaagtgggtctcttggtatcaacagaagccaggccggtcccctgccctggtaatctatcgagatgacaagcggccctcagggatccctgagcgattctctgcctccaactctgggcacactgccactctgaccatcagcgggacccaggctacggatgaggctgactattactgttcgtcccaggacacggtgactcgagtgttcggcggagggaccaagctgaccgtccta

SEQ ID NO: 47 - protein, 106 aa, Homo sapiens
  source: /note=“Abet0319”
SYELTQPPSVSVSPGQTASITCSGHNIMDKWVSWYQQKPGRSPALVIYRDDKRPSGIPERFSASNSGHTATLTISGTQATDEADYYCSSQDTVTRVFGGGTKLTVL

SEQ ID NO: 48 - protein, 11 aa, Homo sapiens
  source: /note=“Abet0319”
SGHNIMDKWVS

SEQ ID NO: 49 - protein, 7 aa, Homo sapiens
  source: /note=“Abet0319”
RDDKRPS

SEQ ID NO: 50 - protein, 9 aa, Homo sapiens
  source: /note=“Abet0319”
SSQDTVTRV

SEQ ID NO: 51 - protein, 22 aa, Homo sapiens
  source: /note=“Abet0319”
SYELTQPPSVSVSPGQTASITC

SEQ ID NO: 52 - protein, 15 aa, Homo sapiens
  source: /note=“Abet0319”
WYQQKPGRSPALVIY

SEQ ID NO: 53 - protein, 32 aa, Homo sapiens; identical to SEQ ID NO: 17
  source: /note=“Abet0319”

SEQ ID NO: 54 - protein, 10 aa, Homo sapiens
  source: /note=“Abet0319”
FGGGTKLTVL

SEQ ID NO: 55 - DNA, 375 bp, Homo sapiens
  source: /note=“Abet0321b”
gaggtgcagctgttggagtctgggggaggcttggtacagcctggggggtccctgagactctcctgtgcagcctctgcgtaccactcgaaccacgaccctatgtggtgggtccgccaggctccagggaaggggctggagtgggtctcagttattggttctagtggtggtacgacagcttacgcagactccgtgaagggccggttcaccatctccagagataattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 56 - protein, 125 aa, Homo sapiens
  source: /note=“Abet0321b”
EVQLLESGGGLVQPGGSLRLSCAASAYHSNHDPMWWVRQAPGKGLEWVSVIGSSGGTTAYADSVKGRFTISRDNSKNTLYLQMNSLRAEDTAVYYCAREWMDHSRPYYYYGMDVWGQGTLVTVSS

SEQ ID NO: 57 - protein, 5 aa, Homo sapiens
  source: /note=“Abet0321b”
HDPMW

SEQ ID NO: 58 - protein, 17 aa, Homo sapiens
  source: /note=“Abet0321b”
VIGSSGGTTAYADSVKG

SEQ ID NO: 59 - protein, 16 aa, Homo sapiens
  source: /note=“Abet0321b”
EWMDHSRPYYYYGMDV

SEQ ID NO: 60 - protein, 30 aa, Homo sapiens
  source: /note=“Abet0321b”
EVQLLESGGGLVQPGGSLRLSCAASAYHSN

SEQ ID NO: 61 - protein, 14 aa, Homo sapiens
  source: /note=“Abet0321b”
WVRQAPGKGLEWVS

SEQ ID NO: 62 - protein, 32 aa, Homo sapiens; identical to SEQ ID NO: 8
  source: /note=“Abet0321b”

SEQ ID NO: 63 - protein, 11 aa, Homo sapiens
  source: /note=“Abet0321b”
WGQGTLVTVSS

SEQ ID NO: 64 - DNA, 318 bp, Homo sapiens
  source: /note=“Abet0321b”
tcgtacgagttgactcagccaccctcagtgtccgtgtccccaggacagacggccagcatcacctgctctggacataacttggaagataaatttgcttcctggtatcaacagaagccaggccagtcccctgtcctgatcatctatcgagatgacaagcggccctcagggatccctgagcgattctctgcctccaactctgggcacactgccactctgaccatcagcgggacccaggctacggatgaggctgactattactgttcgtcccaggacacggtgactcgagtgttcggcggagggaccaagctgaccgtccta

SEQ ID NO: 65 - protein, 106 aa, Homo sapiens
  source: /note=“Abet0321b”
SYELTQPPSVSVSPGQTASITCSGHNLEDKFASWYQQKPGQSPVLIIYRDDKRPSGIPERFSASNSGHTATLTISGTQATDEADYYCSSQDTVTRVFGGGTKLTVL

SEQ ID NO: 66 - protein, 11 aa, Homo sapiens
  source: /note=“Abet0321b”
SGHNLEDKFAS

SEQ ID NO: 67 - protein, 7 aa, Homo sapiens
  source: /note=“Abet0321b”
RDDKRPS

SEQ ID NO: 68 - protein, 9 aa, Homo sapiens
  source: /note=“Abet0321b”
SSQDTVTRV

SEQ ID NO: 69 - protein, 22 aa, Homo sapiens
  source: /note=“Abet0321b”
SYELTQPPSVSVSPGQTASITC

SEQ ID NO: 70 - protein, 15 aa, Homo sapiens
  source: /note=“Abet0321b”
WYQQKPGQSPVLIIY

SEQ ID NO: 71 - protein, 32 aa, Homo sapiens; identical to SEQ ID NO: 17
  source: /note=“Abet0321b”

SEQ ID NO: 72 - protein, 10 aa, Homo sapiens
  source: /note=“Abet0321b”
FGGGTKLTVL

SEQ ID NO: 73 - DNA, 375 bp, Homo sapiens
  source: /note=“Abet0322b”
gaggtgcagctgttggagtctggaggaggcctggtacagcctggggggtccctgagactctcctgtgcagcctctaacgaagagttccagtacaaccctatgtggtgggtccgccaggctccagggaaggggctggagtgggtctcagttattggttctagtggtggtgcgacagtttacgcagacgccgtgaagggccggttcaccatctccagagacaattccgagaacacgctgtatctgcaaatgaacagcctaagagccgaggacacggccgtgtattactgtgcgagagagtggatggaccactcccgcccctactactactacggtatggacgtctgggggcaggggaccctggtcaccgtctcctca

SEQ ID NO: 74 - protein, 125 aa, Homo sapiens
  source: /note=“Abet0322b”
EVQLLESGGGLVQPGGSLRLSCAASNEEFQYNPMWWVRQAPGKGLEWVSVIGSSGGATVYADAVKGRFTISRDNSENTLYLQMNSLRAEDTAVYYCAREWMDHSRPYYYYGMDVWGQGTLVTVSS

SEQ ID NO: 75 - protein, 5 aa, Homo sapiens
  source: /note=“Abet0322b”
YNPMW

SEQ ID NO: 76 - protein, 17 aa, Homo sapiens
  source: /note=“Abet0322b”
VIGSSGGATVYADAVKG

SEQ ID NO: 77 - protein, 16 aa, Homo sapiens
  source: /note=“Abet0322b”
EWMDHSRPYYYYGMDV

SEQ ID NO: 78 - protein, 30 aa, Homo sapiens
  source: /note=“Abet0322b”
EVQLLESGGGLVQPGGSLRLSCAASNEEFQ

SEQ ID NO: 79 - protein, 14 aa, Homo sapiens
  source: /note=“Abet0322b”
WVRQAPGKGLEWVS

SEQ ID NO: 80 - protein, 32 aa, Homo sapiens
  source: /note=“Abet0322b”
RFTISRDNSENTLYLQMNSLRAEDTAVYYCAR

SEQ ID NO: 81 - protein, 11 aa, Homo sapiens
  source: /note=“Abet0322b”
WGQGTLVTVSS

SEQ ID NO: 82 - DNA, 318 bp, Homo sapiens
  source: /note=“Abet0322b”
tcgtacgagttgactcagccaccctcagtgtccgtgtccccaggacagacggccagcatcacctgctctggacataacttgggagataaatttgcttcctggtatcaacagaagccaggccagtcccctgtcctggtcatctatcgagatgacaagcggccctcagagatccctgagcgattctctgcctccaactctgggcacactgccactctgaccatcagcgggacccaggctacggatgaggctgactattactgttcgtcccaggacacggtgactcgagtgttcggcggagggaccaagctgaccgtcctg

SEQ ID NO: 83 - protein, 106 aa, Homo sapiens
  source: /note=“Abet0322b”
SYELTQPPSVSVSPGQTASITCSGHNLGDKFASWYQQKPGQSPVLVIYRDDKRPSEIPERFSASNSGHTATLTISGTQATDEADYYCSSQDTVTRVFGGGTKLTVL

SEQ ID NO: 84 - protein, 11 aa, Homo sapiens
  source: /note=“Abet0322b”
SGHNLGDKFAS

SEQ ID NO: 85 - protein, 7 aa, Homo sapiens
  source: /note=“Abet0322b”
RDDKRPS

SEQ ID NO: 86 - protein, 9 aa, Homo sapiens
  source: /note=“Abet0322b”
SSQDTVTRV

SEQ ID NO: 87 - protein, 22 aa, Homo sapiens
  source: /note=“Abet0322b”
SYELTQPPSVSVSPGQTASITC

SEQ ID NO: 88 - protein, 15 aa, Homo sapiens
  source: /note=“Abet0322b”
WYQQKPGQSPVLVIY

SEQ ID NO: 89 - protein, 32 aa, Homo sapiens
  source: /note=“Abet0322b”
EIPERFSASNSGHTATLTISGTQATDEADYYC

SEQ ID NO: 90 - protein, 10 aa, Homo sapiens
  source: /note=“Abet0322b”
FGGGTKLTVL

SEQ ID NO: 91 - DNA, 375 bp, Homo sapiens
  source: /note=“Abet0323b”
gaggtgcagctgttggagtctgggggaggcttggtacagcctggggggtccctgagactctcctgtgcagcctctaccagcacgttccaggaagacactatgtggtgggtccgccaggctccagggaaggggctggagtgggtctcagttattggtcccaacccgaagaacaacgcctacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 92 - protein, 125 aa, Homo sapiens
  source: /note=“Abet0323b”
EVQLLESGGGLVQPGGSLRLSCAASTSTFQEDTMWWVRQAPGKGLEWVSVIGPNPKNNAYADSVKGRFTISRDNSKNTLYLQMNSLRAEDTAVYYCAREWMDHSRPYYYYGMDVWGQGTLVTVSS

SEQ ID NO: 93 - protein, 5 aa, Homo sapiens
  source: /note=“Abet0323b”
EDTMW

SEQ ID NO: 94 - protein, 17 aa, Homo sapiens
  source: /note=“Abet0323b”
VIGPNPKNNAYADSVKG

SEQ ID NO: 95 - protein, 16 aa, Homo sapiens
  source: /note=“Abet0323b”
EWMDHSRPYYYYGMDV

SEQ ID NO: 96 - protein, 30 aa, Homo sapiens
  source: /note=“Abet0323b”
EVQLLESGGGLVQPGGSLRLSCAASTSTFQ

SEQ ID NO: 97 - protein, 14 aa, Homo sapiens
  source: /note=“Abet0323b”
WVRQAPGKGLEWVS

SEQ ID NO: 98 - protein, 32 aa, Homo sapiens; identical to SEQ ID NO: 8
  source: /note=“Abet0323b”

SEQ ID NO: 99 - protein, 11 aa, Homo sapiens
  source: /note=“Abet0323b”
WGQGTLVTVSS

SEQ ID NO: 100 - DNA, 318 bp, Homo sapiens
  source: /note=“Abet0323b”
tcgtacgagttgactcagccaccctcagtatccgtgtccccaggacagacggccagcatcacctgctctggacataacttggaagataaatttgcttcctggtatcaacagaagccaggccagtcccctgtcctggtcatctatcgagatgacaagcggccctctggggtccctgagcgattctctgcctccaactctgggcacactgccactctgaccatcagcgggacccaggctacggatgaggctgactattactgttcgtcccaggacacggtgactcgagtgttcggcggagggaccaagctgatcgtccta

SEQ ID NO: 101 - protein, 106 aa, Homo sapiens
  source: /note=“Abet0323b”
SYELTQPPSVSVSPGQTASITCSGHNLEDKFASWYQQKPGQSPVLVIYRDDKRPSGVPERFSASNSGHTATLTISGTQATDEADYYCSSQDTVTRVFGGGTKLIVL

SEQ ID NO: 102 - protein, 11 aa, Homo sapiens
  source: /note=“Abet0323b”
SGHNLEDKFAS

SEQ ID NO: 103 - protein, 7 aa, Homo sapiens
  source: /note=“Abet0323b”
RDDKRPS

SEQ ID NO: 104 - protein, 9 aa, Homo sapiens
  source: /note=“Abet0323b”
SSQDTVTRV

SEQ ID NO: 105 - protein, 22 aa, Homo sapiens
  source: /note=“Abet0323b”
SYELTQPPSVSVSPGQTASITC

SEQ ID NO: 106 - protein, 15 aa, Homo sapiens
  source: /note=“Abet0323b”
WYQQKPGQSPVLVIY

SEQ ID NO: 107 - protein, 32 aa, Homo sapiens
  source: /note=“Abet0323b”
GVPERFSASNSGHTATLTISGTQATDEADYYC

SEQ ID NO: 108 - protein, 10 aa, Homo sapiens
  source: /note=“Abet0323b”
FGGGTKLIVL

SEQ ID NO: 109 - DNA, 375 bp, Homo sapiens
  source: /note=“Abet0328”
gaggtgcagctgttggagtctgggggaggcttggtacagcctggggggtccctgagactctcctgtgcagcctccagagaccccttcaaggcggacactatgtggtgggtccgccaggctccaaggaagaggctggagtgggtctcagttattggtgcccacaccaccaacagcgcgtacgcagactccgtgaagggccggttcaccatctccagagacaattccaagaacacgctgtatctgcaaatgaacagcctgagagccgaggacacggccgtgtattactgtgcgagagagtggatggaccgctcccgcccctactactactacggtatggacgtctgggggcaggggaccctggtcaccgtctcctca

SEQ ID NO: 110 - protein, 125 aa, Homo sapiens
  source: /note=“Abet0328”
EVQLLESGGGLVQPGGSLRLSCAASRDPFKADTMWWVRQAPRKRLEWVSVIGAHTTNSAYADSVKGRFTISRDNSKNTLYLQMNSLRAEDTAVYYCAREWMDRSRPYYYYGMDVWGQGTLVTVSS

SEQ ID NO: 111 - protein, 5 aa, Homo sapiens
  source: /note=“Abet0328”
ADTMW

SEQ ID NO: 112 - protein, 17 aa, Homo sapiens
  source: /note=“Abet0328”
VIGAHTTNSAYADSVKG

SEQ ID NO: 113 - protein, 16 aa, Homo sapiens
  source: /note=“Abet0328”
EWMDRSRPYYYYGMDV

SEQ ID NO: 114 - protein, 30 aa, Homo sapiens
  source: /note=“Abet0328”
EVQLLESGGGLVQPGGSLRLSCAASRDPFK

SEQ ID NO: 115 - protein, 14 aa, Homo sapiens
  source: /note=“Abet0328”
WVRQAPRKRLEWVS

SEQ ID NO: 116 - protein, 32 aa, Homo sapiens; identical to SEQ ID NO: 8
  source: /note=“Abet0328”

SEQ ID NO: 117 - protein, 11 aa, Homo sapiens
  source: /note=“Abet0328”
WGQGTLVTVSS

SEQ ID NO: 118 - DNA, 318 bp, Homo sapiens
  source: /note=“Abet0328”
tcgtacgagttgactcagccaccctcagtgtccgtgtccccaggacagacggtcagcatcacctgctctggacgtaacttggaagataaatttgcttcctggtatcaacagaagccaggccagtcccctgtcctggtcatctatcgagatgacaagcggccctcaggggtccctgagcgattctctgcctccaactccgggcacactgccactctgaccatcagcgggacccaggctacggatgaggctgactattactgttcgtcccaggacacggtgactcgagtgttcggcggagggaccaagctgaccgtccta

SEQ ID NO: 119 - protein, 106 aa, Homo sapiens
  source: /note=“Abet0328”
SYELTQPPSVSVSPGQTVSITCSGRNLEDKFASWYQQKPGQSPVLVIYRDDKRPSGVPERFSASNSGHTATLTISGTQATDEADYYCSSQDTVTRVFGGGTKLTVL

SEQ ID NO: 120 - protein, 11 aa, Homo sapiens
  source: /note=“Abet0328”
SGRNLEDKFAS

SEQ ID NO: 121 - protein, 7 aa, Homo sapiens
  source: /note=“Abet0328”
RDDKRPS

SEQ ID NO: 122 - protein, 9 aa, Homo sapiens
  source: /note=“Abet0328”
SSQDTVTRV

SEQ ID NO: 123 - protein, 22 aa, Homo sapiens
  source: /note=“Abet0328”
SYELTQPPSVSVSPGQTVSITC

SEQ ID NO: 124 - protein, 15 aa, Homo sapiens
  source: /note=“Abet0328”
WYQQKPGQSPVLVIY

SEQ ID NO: 125 - protein, 32 aa, Homo sapiens; identical to SEQ ID NO: 107
  source: /note=“Abet0328”

SEQ ID NO: 126 - protein, 10 aa, Homo sapiens
  source: /note=“Abet0328”
FGGGTKLTVL

SEQ ID NO: 127 - DNA, 375 bp, Homo sapiens
  source: /note=“Abet0329”
gaggtgcagctgttggagtctgggggaggcttggtacagcctggggggtccctgagactctcctgtgcagcctctacgtttaacctcaagcgcgagactatgtggtgggtccgccaggctccagggaaggggctggagtgggtctccgttattggttcccaccaggagcgcacgagctacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 128 - protein, 125 aa, Homo sapiens
  source: /note=“Abet0329”
EVQLLESGGGLVQPGGSLRLSCAASTFNLKRETMWWVRQAPGKGLEWVSVIGSHQERTSYADSVKGRFTISRDNSKNTLYLQMNSLRAEDTAVYYCAREWMDHSRPYYYYGMDVWGQGTLVTVSS

SEQ ID NO: 129 - protein, 5 aa, Homo sapiens
  source: /note=“Abet0329”
RETMW

SEQ ID NO: 130 - protein, 17 aa, Homo sapiens
  source: /note=“Abet0329”
VIGSHQERTSYADSVKG

SEQ ID NO: 131 - protein, 16 aa, Homo sapiens
  source: /note=“Abet0329”
EWMDHSRPYYYYGMDV

SEQ ID NO: 132 - protein, 30 aa, Homo sapiens
  source: /note=“Abet0329”
EVQLLESGGGLVQPGGSLRLSCAASTFNLK

SEQ ID NO: 133 - protein, 14 aa, Homo sapiens
  source: /note=“Abet0329”
WVRQAPGKGLEWVS

SEQ ID NO: 134 - protein, 32 aa, Homo sapiens; identical to SEQ ID NO: 8
  source: /note=“Abet0329”

SEQ ID NO: 135 - protein, 11 aa, Homo sapiens
  source: /note=“Abet0329”
WGQGTLVTVSS

SEQ ID NO: 136 - DNA, 318 bp, Homo sapiens
  source: /note=“Abet0329”
tcgtacgagttgactcagccaccctcagtgtccgtgtccccaggacagacggccagcatcacctgctctggacataacgtgagcgacaagtggatgacgtggtatcagcagaagccaggccagtcccctgtcctggtcatctatcgagatgacaagcggccctcagggatccctgagcgattctctgcctccaactctgggcacactgccactctgaccatcagcgggacccaagctacggatgaggctgactattactgttcgtcccaggacacggtgactcgagtgttcggcggagggaccaagctgaccgtccta

SEQ ID NO: 137 - protein, 106 aa, Homo sapiens
  source: /note=“Abet0329”
SYELTQPPSVSVSPGQTASITCSGHNVSDKWMTWYQQKPGQSPVLVIYRDDKRPSGIPERFSASNSGHTATLTISGTQATDEADYYCSSQDTVTRVFGGGTKLTVL

SEQ ID NO: 138 - protein, 11 aa, Homo sapiens
  source: /note=“Abet0329”
SGHNVSDKWMT

SEQ ID NO: 139 - protein, 7 aa, Homo sapiens
  source: /note=“Abet0329”
RDDKRPS

SEQ ID NO: 140 - protein, 9 aa, Homo sapiens
  source: /note=“Abet0329”
SSQDTVTRV

SEQ ID NO: 141 - protein, 22 aa, Homo sapiens
  source: /note=“Abet0329”
SYELTQPPSVSVSPGQTASITC

SEQ ID NO: 142 - protein, 15 aa, Homo sapiens
  source: /note=“Abet0329”
WYQQKPGQSPVLVIY

SEQ ID NO: 143 - protein, 32 aa, Homo sapiens; identical to SEQ ID NO: 17
  source: /note=“Abet0329”

SEQ ID NO: 144 - protein, 10 aa, Homo sapiens
  source: /note=“Abet0329”
FGGGTKLTVL

SEQ ID NO: 145 - DNA, 375 bp, Homo sapiens
  source: /note=“Abet0332”
gaggtgcagctgttggagtctgggggaggcttggtacagcctggggagtccctgagactctcctgtgcagcctcttccgactcctggcacaccgacattatgtggtgggtccgccaggctccagggaagaggctggagtgggtctcagttattggtaactcgaacaagaagatcgcctacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcatca

SEQ ID NO: 146 - protein, 125 aa, Homo sapiens
  source: /note=“Abet0332”
EVQLLESGGGLVQPGESLRLSCAASSDSWHTDIMWWVRQAPGKRLEWVSVIGNSNKKIAYADSVKGRFTISRDNSKNTLYLQMNSLRAEDTAVYYCAREWMDHSRPYYYYGMDVWGQGTLVTVSS

SEQ ID NO: 147 - protein, 5 aa, Homo sapiens
  source: /note=“Abet0332”
TDIMW

SEQ ID NO: 148 - protein, 17 aa, Homo sapiens
  source: /note=“Abet0332”
VIGNSNKKIAYADSVKG

SEQ ID NO: 149 - protein, 16 aa, Homo sapiens
  source: /note=“Abet0332”
EWMDHSRPYYYYGMDV

SEQ ID NO: 150 - protein, 30 aa, Homo sapiens
  source: /note=“Abet0332”
EVQLLESGGGLVQPGESLRLSCAASSDSWH

SEQ ID NO: 151 - protein, 14 aa, Homo sapiens
  source: /note=“Abet0332”
WVRQAPGKRLEWVS

SEQ ID NO: 152 - protein, 32 aa, Homo sapiens; identical to SEQ ID NO: 8
  source: /note=“Abet0332”

SEQ ID NO: 153 - protein, 11 aa, Homo sapiens
  source: /note=“Abet0332”
WGQGTLVTVSS

SEQ ID NO: 154 - DNA, 318 bp, Homo sapiens
  source: /note=“Abet0332”
tcgtacgagttgactcagccaccctcagtgtccgtgtccccagggcagacggccagcatcacctgctctggacataacatcggcgcgaagtgggtgagctggtatcaacagaagccaggccagtcacctatcctggtcatctatcgagatgacaagcggccctcagggatccctgagcgattctctgcctccaactctgggcacactgccactctgaccatcagcgggacccaggctacggatgaggctgactattactgtcaggcgcagggccaggtgaccaggtcgttcggcggagggaccaagctgaccgtccta

SEQ ID NO: 155 - protein, 106 aa, Homo sapiens
  source: /note=“Abet0332”
SYELTQPPSVSVSPGQTASITCSGHNIGAKWVSWYQQKPGQSPILVIYRDDKRPSGIPERFSASNSGHTATLTISGTQATDEADYYCQAQGQVTRSFGGGTKLTVL

SEQ ID NO: 156 - protein, 11 aa, Homo sapiens
  source: /note=“Abet0332”
SGHNIGAKWVS

SEQ ID NO: 157 - protein, 7 aa, Homo sapiens
  source: /note=“Abet0332”
RDDKRPS

SEQ ID NO: 158 - protein, 9 aa, Homo sapiens
  source: /note=“Abet0332”
QAQGQVTRS

SEQ ID NO: 159 - protein, 22 aa, Homo sapiens
  source: /note=“Abet0332”
SYELTQPPSVSVSPGQTASITC

SEQ ID NO: 160 - protein, 15 aa, Homo sapiens
  source: /note=“Abet0332”
WYQQKPGQSPILVIY

SEQ ID NO: 161 - protein, 32 aa, Homo sapiens; identical to SEQ ID NO: 17
  source: /note=“Abet0332”

SEQ ID NO: 162 - protein, 10 aa, Homo sapiens
  source: /note=“Abet0332”
FGGGTKLTVL

SEQ ID NO: 163 - DNA, 375 bp, Homo sapiens
  source: /note=“Abet0342”
gaggtgcagctgttggagtctgggggaggcttggtacagcctggggggtccctgagactctcctgtgcagcctctggattcgactttcgcaggtccgtcatgtggtgggtccgccaggctccagggaaggggctggagtgggtctcagttattggtgcccagacccagaacaaggcgtacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 164 - protein, 125 aa, Homo sapiens
  source: /note=“Abet0342”
EVQLLESGGGLVQPGGSLRLSCAASGFDFRRSVMWWVRQAPGKGLEWVSVIGAQTQNKAYADSVKGRFTISRDNSKNTLYLQMNSLRAEDTAVYYCAREWMDHSRPYYYYGMDVWGQGTLVTVSS

SEQ ID NO: 165 - protein, 5 aa, Homo sapiens
  source: /note=“Abet0342”
RSVMW

SEQ ID NO: 166 - protein, 17 aa, Homo sapiens
  source: /note=“Abet0342”
VIGAQTQNKAYADSVKG

SEQ ID NO: 167 - protein, 16 aa, Homo sapiens
  source: /note=“Abet0342”
EWMDHSRPYYYYGMDV

SEQ ID NO: 168 - protein, 30 aa, Homo sapiens
  source: /note=“Abet0342”
EVQLLESGGGLVQPGGSLRLSCAASGFDFR

SEQ ID NO: 169 - protein, 14 aa, Homo sapiens
  source: /note=“Abet0342”
WVRQAPGKGLEWVS

SEQ ID NO: 170 - protein, 32 aa, Homo sapiens; identical to SEQ ID NO: 8
  source: /note=“Abet0342”

SEQ ID NO: 171 - protein, 11 aa, Homo sapiens
  source: /note=“Abet0342”
WGQGTLVTVSS

SEQ ID NO: 172 - DNA, 318 bp, Homo sapiens
  source: /note=“Abet0342”
tcgtacgagttgactcagccaccctcagtgtccgtgtccccaggacagacggccagcatcacctgctctggacataacttggaagataaatttgcttcctggtatcaacagaagccaggccagtcccccgtcctggtcatctatcgggatgacaagcggccctcagggatccctgagcgattctctgcctccaactctggggacactgccactctgaccatcagcgggacccaggctatggatgaggctgactattactgtcaggcgcaggacagtaccactcgagtgttcggcggagggactaagctgaccgtccta

SEQ ID NO: 173 - protein, 106 aa, Homo sapiens
  source: /note=“Abet0342”
SYELTQPPSVSVSPGQTASITCSGHNLEDKFASWYQQKPGQSPVLVIYRDDKRPSGIPERFSASNSGDTATLTISGTQAMDEADYYCQAQDSTTRVFGGGTKLTVL

SEQ ID NO: 174 - protein, 11 aa, Homo sapiens
  source: /note=“Abet0342”
SGHNLEDKFAS

SEQ ID NO: 175 - protein, 7 aa, Homo sapiens
  source: /note=“Abet0342”
RDDKRPS

SEQ ID NO: 176 - protein, 9 aa, Homo sapiens
  source: /note=“Abet0342”
QAQDSTTRV

SEQ ID NO: 177 - protein, 22 aa, Homo sapiens
  source: /note=“Abet0342”
SYELTQPPSVSVSPGQTASITC

SEQ ID NO: 178 - protein, 15 aa, Homo sapiens
  source: /note=“Abet0342”
WYQQKPGQSPVLVIY

SEQ ID NO: 179 - protein, 32 aa, Homo sapiens
  source: /note=“Abet0342”
GIPERFSASNSGDTATLTISGTQAMDEADYYC

SEQ ID NO: 180 - protein, 10 aa, Homo sapiens
  source: /note=“Abet0342”
FGGGTKLTVL

SEQ ID NO: 181 - DNA, 375 bp, Homo sapiens
  source: /note=“Abet0343”
gaggtgcagctgttggagtctgggggaggcttggtacagcctggggggtccctgagactctcctgtgcagcctctggattcaactttaaccaccaggtgatgtggtgggtccgccaggctccagggaaggggctggagtgggtctcagttattggtaagaccaacgagaacatcgcctacgcagactccgtgaagggccggttcaccatctccagagacaattccaagaacacgctgtatctgcaaatgaacagcctgagagccgaggacacggccgtgtattactgtgcgagagagtggatggaccactctcgcccctactactactacggtatggacgtctgggggcaggggaccctggtcaccgtctcctca

SEQ ID NO: 182 - protein, 125 aa, Homo sapiens
  source: /note=“Abet0343”
EVQLLESGGGLVQPGGSLRLSCAASGFNFNHQVMWWVRQAPGKGLEWVSVIGKTNENIAYADSVKGRFTISRDNSKNTLYLQMNSLRAEDTAVYYCAREWMDHSRPYYYYGMDVWGQGTLVTVSS

SEQ ID NO: 183 - protein, 5 aa, Homo sapiens
  source: /note=“Abet0343”
HQVMW

SEQ ID NO: 184 - protein, 17 aa, Homo sapiens
  source: /note=“Abet0343”
VIGKTNENIAYADSVKG

SEQ ID NO: 185 - protein, 16 aa, Homo sapiens
  source: /note=“Abet0343”
EWMDHSRPYYYYGMDV

SEQ ID NO: 186 - protein, 30 aa, Homo sapiens
  source: /note=“Abet0343”
EVQLLESGGGLVQPGGSLRLSCAASGFNFN

SEQ ID NO: 187 - protein, 14 aa, Homo sapiens
  source: /note=“Abet0343”
WVRQAPGKGLEWVS

SEQ ID NO: 188 - protein, 32 aa, Homo sapiens; identical to SEQ ID NO: 8
  source: /note=“Abet0343”

SEQ ID NO: 189 - protein, 11 aa, Homo sapiens
  source: /note=“Abet0343”
WGQGTLVTVSS

SEQ ID NO: 190 - DNA, 318 bp, Homo sapiens
  source: /note=“Abet0343”
cagagcgtcttgactcagccaccctcagtgtccgtgtccccaggacagacggccagcatcacctgctctggacataacttggaagataaatttgcttcctggtatcaacagaagtcaggccagtcccctgtcctggtcatctatcgagatgacaagcggccctcagggatccctgagcgattctctgcctccaactctgggcacactgccactctgaccatcagcgggacccaggctacggatgaggctgactattactgttcgtcccaggacacggtgactcgagtgttcggcggagggaccaagctgaccgtccta

SEQ ID NO: 191 - protein, 106 aa, Homo sapiens
  source: /note=“Abet0343”
QSVLTQPPSVSVSPGQTASITCSGHNLEDKFASWYQQKSGQSPVLVIYRDDKRPSGIPERFSASNSGHTATLTISGTQATDEADYYCSSQDTVTRVFGGGTKLTVL

SEQ ID NO: 192 - protein, 11 aa, Homo sapiens
  source: /note=“Abet0343”
SGHNLEDKFAS

SEQ ID NO: 193 - protein, 7 aa, Homo sapiens
  source: /note=“Abet0343”
RDDKRPS

SEQ ID NO: 194 - protein, 9 aa, Homo sapiens
  source: /note=“Abet0343”
SSQDTVTRV

SEQ ID NO: 195 - protein, 22 aa, Homo sapiens
  source: /note=“Abet0343”
QSVLTQPPSVSVSPGQTASITC

SEQ ID NO: 196 - protein, 15 aa, Homo sapiens
  source: /note=“Abet0343”
WYQQKSGQSPVLVIY

SEQ ID NO: 197 - protein, 32 aa, Homo sapiens; identical to SEQ ID NO: 17
  source: /note=“Abet0343”

SEQ ID NO: 198 - protein, 10 aa, Homo sapiens
  source: /note=“Abet0343”
FGGGTKLTVL

SEQ ID NO: 199 - DNA, 375 bp, Homo sapiens
  source: /note=“Abet0344”
gaggtgcagctattggagtctgggggaggcttggtacagcctggggggtccctgagtctctcctgtgcagcctctggattcacctttagcgtttatactatgtggtgggtccgccaggctccagggaaggggctggagtgggtctcagttattggtgggaacgagacccggaaggcctacgcagactccgtgaagggccggttcaccatctccagggacaattccaagaacaggctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 200 - protein, 125 aa, Homo sapiens
  source: /note=“Abet0344”
EVQLLESGGGLVQPGGSLSLSCAASGFTFSVYTMWWVRQAPGKGLEWVSVIGGNETRKAYADSVKGRFTISRDNSKNRLYLQMNSLRAEDTAVYYCAREWMDHSRPYYYYGMDVWGQGTLVTVSS

SEQ ID NO: 201 - protein, 5 aa, Homo sapiens
  source: /note=“Abet0344”
VYTMW

SEQ ID NO: 202 - protein, 17 aa, Homo sapiens
  source: /note=“Abet0344”
VIGGNETRKAYADSVKG

SEQ ID NO: 203 - protein, 16 aa, Homo sapiens
  source: /note=“Abet0344”
EWMDHSRPYYYYGMDV

SEQ ID NO: 204 - protein, 30 aa, Homo sapiens
  source: /note=“Abet0344”
EVQLLESGGGLVQPGGSLSLSCAASGFTFS

SEQ ID NO: 205 - protein, 14 aa, Homo sapiens
  source: /note=“Abet0344”
WVRQAPGKGLEWVS

SEQ ID NO: 206 - protein, 32 aa, Homo sapiens
  source: /note=“Abet0344”
RFTISRDNSKNRLYLQMNSLRAEDTAVYYCAR

SEQ ID NO: 207 - protein, 11 aa, Homo sapiens
  source: /note=“Abet0344”
WGQGTLVTVSS

SEQ ID NO: 208 - DNA, 318 bp, Homo sapiens
  source: /note=“Abet0344”
cagagcgtcttgactcagccaccctcagtgtccgtgtccccaggacagacggccagcatcacctgctctggacataacttggaagataaatttgcttcctggtatcaacagaagtcaggccagtcccctgtcctggtcatctatcgagatgacaagcggccctcagggatccctgagcgattctctgcctccaactctgggcacactgccactctgaccatcagcgggacccaggctacggatgaggctgactattactgtgcgacccaggacaacttcactcgagtgttcggcggaggcaccaagctgaccgtccta

SEQ ID NO: 209 - protein, 106 aa, Homo sapiens
  source: /note=“Abet0344”
QSVLTQPPSVSVSPGQTASITCSGHNLEDKFASWYQQKSGQSPVLVIYRDDKRPSGIPERFSASNSGHTATLTISGTQATDEADYYCATQDNFTRVFGGGTKLTVL

SEQ ID NO: 210 - protein, 11 aa, Homo sapiens
  source: /note=“Abet0344”
SGHNLEDKFAS

SEQ ID NO: 211 - protein, 7 aa, Homo sapiens
  source: /note=“Abet0344”
RDDKRPS

SEQ ID NO: 212 - protein, 9 aa, Homo sapiens
  source: /note=“Abet0344”
ATQDNFTRV

SEQ ID NO: 213 - protein, 22 aa, Homo sapiens
  source: /note=“Abet0344”
QSVLTQPPSVSVSPGQTASITC

SEQ ID NO: 214 - protein, 15 aa, Homo sapiens
  source: /note=“Abet0344”
WYQQKSGQSPVLVIY

SEQ ID NO: 215 - protein, 32 aa, Homo sapiens; identical to SEQ ID NO: 17
  source: /note=“Abet0344”

SEQ ID NO: 216 - protein, 10 aa, Homo sapiens
  source: /note=“Abet0344”
FGGGTKLTVL

SEQ ID NO: 217 - DNA, 375 bp, Homo sapiens
  source: /note=“Abet0368”
gaggtgcagctgttggagtctgggggaggcttagtacagccgggggggtccctgagactctcctgtgcagcctctggattcgactttgggccgagccctatgtggtgggtccgccaggctccagggaaggggctggagtgggtctcagttattggtaaggacacccagaacagcacgtacgcagactccgtgaagggccggttcaccatctccagagacaattccaaggacacgctgtatctgcaaatgaacagcctgaaagccgaggacacggccgtgtattactgtgcgagagagtggatggaccactcccgcccctactactactacggtatggacgtctgggggcaggggaccctggtcaccgtctcctca

SEQ ID NO: 218 - protein, 125 aa, Homo sapiens
  source: /note=“Abet0368”
EVQLLESGGGLVQPGGSLRLSCAASGFDFGPSPMWWVRQAPGKGLEWVSVIGKDTQNSTYADSVKGRFTISRDNSKDTLYLQMNSLKAEDTAVYYCAREWMDHSRPYYYYGMDVWGQGTLVTVSS

SEQ ID NO: 219 - protein, 5 aa, Homo sapiens
  source: /note=“Abet0368”
PSPMW

SEQ ID NO: 220 - protein, 17 aa, Homo sapiens
  source: /note=“Abet0368”
VIGKDTQNSTYADSVKG

SEQ ID NO: 221 - protein, 16 aa, Homo sapiens
  source: /note=“Abet0368”
EWMDHSRPYYYYGMDV

SEQ ID NO: 222 - protein, 30 aa, Homo sapiens
  source: /note=“Abet0368”
EVQLLESGGGLVQPGGSLRLSCAASGFDFG

SEQ ID NO: 223 - protein, 14 aa, Homo sapiens
  source: /note=“Abet0368”
WVRQAPGKGLEWVS

SEQ ID NO: 224 - protein, 32 aa, Homo sapiens
  source: /note=“Abet0368”
RFTISRDNSKDTLYLQMNSLKAEDTAVYYCAR

SEQ ID NO: 225 - protein, 11 aa, Homo sapiens
  source: /note=“Abet0368”
WGQGTLVTVSS

SEQ ID NO: 226 - DNA, 318 bp, Homo sapiens
  source: /note=“Abet0368”
tcgtacgagttgactcagccaccctcagtgtccgtgtccccaggacagacggccagcatcacctgctctggacataacttggaagataaatttacttcctggtatcaacagaagtcaggccagtcccctgtcctggtcatctatcgagatgacaagcggccctcagggatccctgagcgattctctgcctccaactctgggcacactgccactctgaccatcagcggggcccaggctacggatgaggctgactattactgttcgtcccaggacacggtgactcgagtgttcggcggagggaccaagctgaccgtccta

SEQ ID NO: 227 - protein, 106 aa, Homo sapiens
  source: /note=“Abet0368”
SYELTQPPSVSVSPGQTASITCSGHNLEDKFTSWYQQKSGQSPVLVIYRDDKRPSGIPERFSASNSGHTATLTISGAQATDEADYYCSSQDTVTRVFGGGTKLTVL

SEQ ID NO: 228 - protein, 11 aa, Homo sapiens
  source: /note=“Abet0368”
SGHNLEDKFTS

SEQ ID NO: 229 - protein, 7 aa, Homo sapiens
  source: /note=“Abet0368”
RDDKRPS

SEQ ID NO: 230 - protein, 9 aa, Homo sapiens
  source: /note=“Abet0368”
SSQDTVTRV

SEQ ID NO: 231 - protein, 22 aa, Homo sapiens
  source: /note=“Abet0368”
SYELTQPPSVSVSPGQTASITC

SEQ ID NO: 232 - protein, 15 aa, Homo sapiens
  source: /note=“Abet0368”
WYQQKSGQSPVLVIY

SEQ ID NO: 233 - protein, 32 aa, Homo sapiens
  source: /note=“Abet0368”
GIPERFSASNSGHTATLTISGAQATDEADYYC

SEQ ID NO: 234 - protein, 10 aa, Homo sapiens
  source: /note=“Abet0368”
FGGGTKLTVL

SEQ ID NO: 235 - DNA, 375 bp, Homo sapiens
  source: /note=“Abet0369”
gaggtgcagctgttggagtctgggggaggcctggtacagcctggggggtccctgagactctcctgtgcagcctcttcgttccagatctcgaagaacactatgtggtgggtccgccgggctccagggaaggggctggagtgggtctcagttattggtaaggacgagacccgcttcaactacgcagactccgtgaagggccggttcaccatctccagagacaattccaagaacaccctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 236 - protein, 125 aa, Homo sapiens
  source: /note=“Abet0369”
EVQLLESGGGLVQPGGSLRLSCAASSFQISKNTMWWVRRAPGKGLEWVSVIGKDETRFNYADSVKGRFTISRDNSKNTLYLQMNSLRAEDTAVYYCAREWMDHSRPYYYYGMDVWGQGTLVTVSS

SEQ ID NO: 237 - protein, 5 aa, Homo sapiens
  source: /note=“Abet0369”
KNTMW

SEQ ID NO: 238 - protein, 17 aa, Homo sapiens
  source: /note=“Abet0369”
VIGKDETRFNYADSVKG

SEQ ID NO: 239 - protein, 16 aa, Homo sapiens
  source: /note=“Abet0369”
EWMDHSRPYYYYGMDV

SEQ ID NO: 240 - protein, 30 aa, Homo sapiens
  source: /note=“Abet0369”
EVQLLESGGGLVQPGGSLRLSCAASSFQIS

SEQ ID NO: 241 - protein, 14 aa, Homo sapiens
  source: /note=“Abet0369”
WVRRAPGKGLEWVS

SEQ ID NO: 242 - protein, 32 aa, Homo sapiens; identical to SEQ ID NO: 8
  source: /note=“Abet0369”

SEQ ID NO: 243 - protein, 11 aa, Homo sapiens
  source: /note=“Abet0369”
WGQGTLVTVSS

SEQ ID NO: 244 - DNA, 318 bp, Homo sapiens
  source: /note=“Abet0369”
tcgtacgggttgactcagccaccctcagtgtccgtgtccccaggacagacggccagcatcacctgctctggacgtaacatcggggacagctgggtcgcgtggtatcaacagaagccaggccagtcccctgtcctggtcatctatcgagatgacaagcggccctcagggatccctgagcgattctctgcctccaactctgggcacactgccactctgaccatcagcgggacccaggctacggatgaggctgactattactgttcgtcccaggacacggtgactcgagtgttcggcggagggaccaagctgaccgtccta

SEQ ID NO: 245 - protein, 106 aa, Homo sapiens
  source: /note=“Abet0369”
SYGLTQPPSVSVSPGQTASITCSGRNIGDSWVAWYQQKPGQSPVLVIYRDDKRPSGIPERFSASNSGHTATLTISGTQATDEADYYCSSQDTVTRVFGGGTKLTVL

SEQ ID NO: 246 - protein, 11 aa, Homo sapiens
  source: /note=“Abet0369”
SGRNIGDSWVA

SEQ ID NO: 247 - protein, 7 aa, Homo sapiens
  source: /note=“Abet0369”
RDDKRPS

SEQ ID NO: 248 - protein, 9 aa, Homo sapiens
  source: /note=“Abet0369”
SSQDTVTRV

SEQ ID NO: 249 - protein, 22 aa, Homo sapiens
  source: /note=“Abet0369”
SYGLTQPPSVSVSPGQTASITC

SEQ ID NO: 250 - protein, 15 aa, Homo sapiens
  source: /note=“Abet0369”
WYQQKPGQSPVLVIY

SEQ ID NO: 251 - protein, 32 aa, Homo sapiens; identical to SEQ ID NO: 17
  source: /note=“Abet0369”

SEQ ID NO: 252 - protein, 10 aa, Homo sapiens
  source: /note=“Abet0369”
FGGGTKLTVL

SEQ ID NO: 253 - DNA, 375 bp, Homo sapiens
  source: /note=“Abet0370”
gaggtgcagctgttggagtctgggggaggcttggtacagcctggggggtccctgagactctcctgtgcagcctctggattccactttcccatgagcgccatgtggtgggtccgccaggctccagggaaggggctggagtgggtctcagtcattggtgagaccccggagaggcaggcctacgcagactccgtgaagggccggttcaccatctccagagacaattccaagagcacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 254 - protein, 125 aa, Homo sapiens
  source: /note=“Abet0370”
EVQLLESGGGLVQPGGSLRLSCAASGFHFPMSAMWWVRQAPGKGLEWVSVIGETPERQAYADSVKGRFTISRDNSKSTLYLQMNSLRAEDTAVYYCAREWMDHSRPYYYYGMDVWGQGTLVTVSS

SEQ ID NO: 255 - protein, 5 aa, Homo sapiens
  source: /note=“Abet0370”
MSAMW

SEQ ID NO: 256 - protein, 17 aa, Homo sapiens
  source: /note=“Abet0370”
VIGETPERQAYADSVKG

SEQ ID NO: 257 - protein, 16 aa, Homo sapiens
  source: /note=“Abet0370”
EWMDHSRPYYYYGMDV

SEQ ID NO: 258 - protein, 30 aa, Homo sapiens
  source: /note=“Abet0370”
EVQLLESGGGLVQPGGSLRLSCAASGFHFP

SEQ ID NO: 259 - protein, 14 aa, Homo sapiens
  source: /note=“Abet0370”
WVRQAPGKGLEWVS

SEQ ID NO: 260 - protein, 32 aa, Homo sapiens
  source: /note=“Abet0370”
RFTISRDNSKSTLYLQMNSLRAEDTAVYYCAR

SEQ ID NO: 261 - protein, 11 aa, Homo sapiens
  source: /note=“Abet0370”
WGQGTLVTVSS

SEQ ID NO: 262 - DNA, 318 bp, Homo sapiens
  source: /note=“Abet0370”
tcgtacgagttgactcagccaccctcagtgtccgtgtccccaggacagacggccagcatcacctgcacgaccccgcacttcaacagcaaatttgcttcctggtatcaacagaagccgggccagtcccctgtcctggtcatctatcgagatgacaagcggccctcagggatccctgagcgattctctgcctccaactctgggcacactgccactctgaccatcagcgggacccaggctatggatgaggctgactattactgtcaggcgcaggatagtaccactcgagtgttcggcggagggaccaggctgaccgtccta

SEQ ID NO: 263 - protein, 106 aa, Homo sapiens
  source: /note=“Abet0370”
SYELTQPPSVSVSPGQTASITCTTPHFNSKFASWYQQKPGQSPVLVIYRDDKRPSGIPERFSASNSGHTATLTISGTQAMDEADYYCQAQDSTTRVFGGGTRLTVL

SEQ ID NO: 264 - protein, 11 aa, Homo sapiens
  source: /note=“Abet0370”
TTPHFNSKFAS

SEQ ID NO: 265 - protein, 7 aa, Homo sapiens
  source: /note=“Abet0370”
RDDKRPS

SEQ ID NO: 266 - protein, 9 aa, Homo sapiens
  source: /note=“Abet0370”
QAQDSTTRV

SEQ ID NO: 267 - protein, 22 aa, Homo sapiens
  source: /note=“Abet0370”
SYELTQPPSVSVSPGQTASITC

SEQ ID NO: 268 - protein, 15 aa, Homo sapiens
  source: /note=“Abet0370”
WYQQKPGQSPVLVIY

SEQ ID NO: 269 - protein, 32 aa, Homo sapiens; identical to SEQ ID NO: 35
  source: /note=“Abet0370”

SEQ ID NO: 270 - protein, 10 aa, Homo sapiens
  source: /note=“Abet0370”
FGGGTRLTVL

SEQ ID NO: 271 - DNA, 375 bp, Homo sapiens
  source: /note=“Abet0371”
gaggtgcagctgtcggagtctgggggaggcttggtacagcctggggggtccctgagactctcctgtgcagcctctcacgacgccttccccttcgacactatgtggtgggtccgccaggctccagggaaggggctggagtgggtctcagttattggttctagtggtggtacgacagtttacgcagactccgtgaagggccggttcaccgtttccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 272 - protein, 125 aa, Homo sapiens
  source: /note=“Abet0371”
EVQLSESGGGLVQPGGSLRLSCAASHDAFPFDTMWWVRQAPGKGLEWVSVIGSSGGTTVYADSVKGRFTVSRDNSKNTLYLQMNSLRAEDTAVYYCAREWMDHSRPYYYYGMDVWGQGTLVTVSS

SEQ ID NO: 273 - protein, 5 aa, Homo sapiens
  source: /note=“Abet0371”
FDTMW

SEQ ID NO: 274 - protein, 17 aa, Homo sapiens
  source: /note=“Abet0371”
VIGSSGGTTVYADSVKG

SEQ ID NO: 275 - protein, 16 aa, Homo sapiens
  source: /note=“Abet0371”
EWMDHSRPYYYYGMDV

SEQ ID NO: 276 - protein, 30 aa, Homo sapiens
  source: /note=“Abet0371”
EVQLSESGGGLVQPGGSLRLSCAASHDAFP

SEQ ID NO: 277 - protein, 14 aa, Homo sapiens
  source: /note=“Abet0371”
WVRQAPGKGLEWVS

SEQ ID NO: 278 - protein, 32 aa, Homo sapiens
  source: /note=“Abet0371”
RFTVSRDNSKNTLYLQMNSLRAEDTAVYYCAR

SEQ ID NO: 279 - protein, 11 aa, Homo sapiens
  source: /note=“Abet0371”
WGQGTLVTVSS

SEQ ID NO: 280 - DNA, 318 bp, Homo sapiens
  source: /note=“Abet0371”
tcgtacgagttgactcagccaccctcagtgtccgtgtccccaggacagacggccagcatcacctgctccggacataacatctcgtcgagctgggtctcctggtatcaacagaagccaggccagtcccctgtcctggtcatctatcgagatgacaagcggccctcagggatccctgagcgattctctgcctccaactctgggcacactgccactctgaccatcagcgggacccaggctacggatgaggctgactattactgttcgtcccaggacacggtgactcgagtcttcggcggagggaccaagctgaccgtccta

SEQ ID NO: 281 - protein, 106 aa, Homo sapiens
  source: /note=“Abet0371”
SYELTQPPSVSVSPGQTASITCSGHNISSSWVSWYQQKPGQSPVLVIYRDDKRPSGIPERFSASNSGHTATLTISGTQATDEADYYCSSQDTVTRVFGGGTKLTVL

SEQ ID NO: 282 - protein, 11 aa, Homo sapiens
  source: /note=“Abet0371”
SGHNISSSWVS

SEQ ID NO: 283 - protein, 7 aa, Homo sapiens
  source: /note=“Abet0371”
RDDKRPS

SEQ ID NO: 284 - protein, 9 aa, Homo sapiens
  source: /note=“Abet0371”
SSQDTVTRV

SEQ ID NO: 285 - protein, 22 aa, Homo sapiens
  source: /note=“Abet0371”
SYELTQPPSVSVSPGQTASITC

SEQ ID NO: 286 - protein, 15 aa, Homo sapiens
  source: /note=“Abet0371”
WYQQKPGQSPVLVIY

SEQ ID NO: 287 - protein, 32 aa, Homo sapiens; identical to SEQ ID NO: 17
  source: /note=“Abet0371”

SEQ ID NO: 288 - protein, 10 aa, Homo sapiens
  source: /note=“Abet0371”
FGGGTKLTVL

SEQ ID NO: 289 - DNA, 375 bp, Homo sapiens
  source: /note=“Abet0372”
gaggtgcagctgttggagtctggggggggcttggtacagcctggggggtccctgagactctcctgtgcagcctctagcgacatgttcaacatcgagaccatgtggtgggtccgccaggctccagggaaggggctggagtgggtctcagttattggtaaggggatgaacaacgtctcgtacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 290 - protein, 125 aa, Homo sapiens
  source: /note=“Abet0372”
EVQLLESGGGLVQPGGSLRLSCAASSDMFNIETMWWVRQAPGKGLEWVSVIGKGMNNVSYADSVKGRFTISRDNSKNTLYLQMNSLRAEDTAVYYCAREWMDHSRPYYYYGMDVWGQGTLVTVSS

SEQ ID NO: 291 - protein, 5 aa, Homo sapiens
  source: /note=“Abet0372”
IETMW

SEQ ID NO: 292 - protein, 17 aa, Homo sapiens
  source: /note=“Abet0372”
VIGKGMNNVSYADSVKG

SEQ ID NO: 293 - protein, 16 aa, Homo sapiens
  source: /note=“Abet0372”
EWMDHSRPYYYYGMDV

SEQ ID NO: 294 - protein, 30 aa, Homo sapiens
  source: /note=“Abet0372”
EVQLLESGGGLVQPGGSLRLSCAASSDMFN

SEQ ID NO: 295 - protein, 14 aa, Homo sapiens
  source: /note=“Abet0372”
WVRQAPGKGLEWVS

SEQ ID NO: 296 - protein, 32 aa, Homo sapiens; identical to SEQ ID NO: 8
  source: /note=“Abet0372”

SEQ ID NO: 297 - protein, 11 aa, Homo sapiens
  source: /note=“Abet0372”
WGQGTLVTVSS

SEQ ID NO: 298 - DNA, 318 bp, Homo sapiens
  source: /note=“Abet0372”
tcgtacgagttgactcagccaccctcagtgtccgtgtccccaggacagacggccagcatcacctgctctggacataacttggaagataaatttgcttcctggtatcaacagaagccaggccagtcccctgtcctggtcatctatcgagatgacaagcggccctcagggatccctgagcgattctctgcctccaactctgggcacactgccactctgaccattagcgggacccaggctacggatgaggctgattattactgttcgtcccaggacacggtgactcgagtgttcggcggagggaccaagctgaccgtccta

SEQ ID NO: 299 - protein, 106 aa, Homo sapiens
  source: /note=“Abet0372”
SYELTQPPSVSVSPGQTASITCSGHNLEDKFASWYQQKPGQSPVLVIYRDDKRPSGIPERFSASNSGHTATLTISGTQATDEADYYCSSQDTVTRVFGGGTKLTVL

SEQ ID NO: 300 - protein, 11 aa, Homo sapiens
  source: /note=“Abet0372”
SGHNLEDKFAS

SEQ ID NO: 301 - protein, 7 aa, Homo sapiens
  source: /note=“Abet0372”
RDDKRPS

SEQ ID NO: 302 - protein, 9 aa, Homo sapiens
  source: /note=“Abet0372”
SSQDTVTRV

SEQ ID NO: 303 - protein, 22 aa, Homo sapiens
  source: /note=“Abet0372”
SYELTQPPSVSVSPGQTASITC

SEQ ID NO: 304 - protein, 15 aa, Homo sapiens
  source: /note=“Abet0372”
WYQQKPGQSPVLVIY

SEQ ID NO: 305 - protein, 32 aa, Homo sapiens; identical to SEQ ID NO: 17
  source: /note=“Abet0372”

SEQ ID NO: 306 - protein, 10 aa, Homo sapiens
  source: /note=“Abet0372”
FGGGTKLTVL

SEQ ID NO: 307 - DNA, 375 bp, Homo sapiens
  source: /note=“Abet0373”
gaggtgcagctgttggagtctgggggaggcttggtacagcctggggggtccctgagactctcctgtgtagcctccggattcgactttgagcggtccgtcatgtggtgggtccgccaggctccagggaagaggctggagtgggtctcagttattggtagcgggaagaccaacatcacctacgcagactccgtgaagggccggtttaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 308 - protein, 125 aa, Homo sapiens
  source: /note=“Abet0373”
EVQLLESGGGLVQPGGSLRLSCVASGFDFERSVMWWVRQAPGKRLEWVSVIGSGKTNITYADSVKGRFTISRDNSKNTLYLQMNSLRAEDTAVYYCAREWMDHSRPYYYYGMDVWGQGTLVTVSS

SEQ ID NO: 309 - protein, 5 aa, Homo sapiens
  source: /note=“Abet0373”
RSVMW

SEQ ID NO: 310 - protein, 17 aa, Homo sapiens
  source: /note=“Abet0373”
VIGSGKTNITYADSVKG

SEQ ID NO: 311 - protein, 16 aa, Homo sapiens
  source: /note=“Abet0373”
EWMDHSRPYYYYGMDV

SEQ ID NO: 312 - protein, 30 aa, Homo sapiens
  source: /note=“Abet0373”
EVQLLESGGGLVQPGGSLRLSCVASGFDFE

SEQ ID NO: 313 - protein, 14 aa, Homo sapiens
  source: /note=“Abet0373”
WVRQAPGKRLEWVS

SEQ ID NO: 314 - protein, 32 aa, Homo sapiens; identical to SEQ ID NO: 8
  source: /note=“Abet0373”

SEQ ID NO: 315 - protein, 11 aa, Homo sapiens
  source: /note=“Abet0373”
WGQGTLVTVSS

SEQ ID NO: 316 - DNA, 318 bp, Homo sapiens
  source: /note=“Abet0373”
tcgtacgagttgactcagccaccctcagtgtccgtgtccccagggcagacggccagcatcacctgctctggtcataacttggaggataaatttgcttcctggtatcaacagaagccaggccagtcccccgtcctggtcatctatcgagatgacaagcggccctcagagatccctgagcgattctctgcctccaactctgggcacaccgccactctgaccatcagcgggacccaggctacggatgaggctgactattactgttcgtcccaggacacggtgactcgagtgttcggcggagggaccaagctgaccgtccta

SEQ ID NO: 317 - protein, 106 aa, Homo sapiens
  source: /note=“Abet0373”
SYELTQPPSVSVSPGQTASITCSGHNLEDKFASWYQQKPGQSPVLVIYRDDKRPSEIPERFSASNSGHTATLTISGTQATDEADYYCSSQDTVTRVFGGGTKLTVL

SEQ ID NO: 318 - protein, 11 aa, Homo sapiens
  source: /note=“Abet0373”
SGHNLEDKFAS

SEQ ID NO: 319 - protein, 7 aa, Homo sapiens
  source: /note=“Abet0373”
RDDKRPS

SEQ ID NO: 320 - protein, 9 aa, Homo sapiens
  source: /note=“Abet0373”
SSQDTVTRV

SEQ ID NO: 321 - protein, 22 aa, Homo sapiens
  source: /note=“Abet0373”
SYELTQPPSVSVSPGQTASITC

SEQ ID NO: 322 - protein, 15 aa, Homo sapiens
  source: /note=“Abet0373”
WYQQKPGQSPVLVIY

SEQ ID NO: 323 - protein, 32 aa, Homo sapiens; identical to SEQ ID NO: 89
  source: /note=“Abet0373”

SEQ ID NO: 324 - protein, 10 aa, Homo sapiens
  source: /note=“Abet0373”
FGGGTKLTVL

SEQ ID NO: 325 - DNA, 375 bp, Homo sapiens
  source: /note=“Abet0374”
gaggtgcagctgttggagtctgggggaggcttggtacagcctggggggtccctgagactctcctgtgcagcctctggattccagtttaaggacacgcccatgtggtgggtccgccaggctccagggaaggggctagagtgggtctcagttattggtgaccagaaccacaagaaggcctacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgcctgggggcaggggaccctggtcaccgtctcctca

SEQ ID NO: 326 - protein, 125 aa, Homo sapiens
  source: /note=“Abet0374”
EVQLLESGGGLVQPGGSLRLSCAASGFQFKDTPMWWVRQAPGKGLEWVSVIGDQNHKKAYADSVKGRFTISRDNSKNTLYLQMNSLRAEDTAVYYCAREWMDHSRPYYYYGMDAWGQGTLVTVSS

SEQ ID NO: 327 - protein, 5 aa, Homo sapiens
  source: /note=“Abet0374”
DTPMW

SEQ ID NO: 328 - protein, 17 aa, Homo sapiens
  source: /note=“Abet0374”
VIGDQNHKKAYADSVKG

SEQ ID NO: 329 - protein, 16 aa, Homo sapiens
  source: /note=“Abet0374”
EWMDHSRPYYYYGMDA

SEQ ID NO: 330 - protein, 30 aa, Homo sapiens
  source: /note=“Abet0374”
EVQLLESGGGLVQPGGSLRLSCAASGFQFK

SEQ ID NO: 331 - protein, 14 aa, Homo sapiens
  source: /note=“Abet0374”
WVRQAPGKGLEWVS

SEQ ID NO: 332 - protein, 32 aa, Homo sapiens; identical to SEQ ID NO: 8
  source: /note=“Abet0374”

SEQ ID NO: 333 - protein, 11 aa, Homo sapiens
  source: /note=“Abet0374”
WGQGTLVTVSS

SEQ ID NO: 334 - DNA, 318 bp, Homo sapiens
  source: /note=“Abet0374”
tcgtacgagttgactcagccaccctcagtgtccgtgaccccaggacagacggccagcatcacctgctctggacataacttgggaggtaaatttgcttcctggtatcaacagaagccaggccagtcccctgtcctggtcatctatcgagatgacaagcggccctcagggatccctgagcgattctctgcctccaactttgggcacactgccactctgaccatcagcgggacccaggctacggatgaggctgactattactgttcgtcccaggacacggtgactcgagtgttcggcggagggaccaagctgaccgtccta

SEQ ID NO: 335 - protein, 106 aa, Homo sapiens
  source: /note=“Abet0374”
SYELTQPPSVSVTPGQTASITCSGHNLGGKFASWYQQKPGQSPVLVIYRDDKRPSGIPERFSASNFGHTATLTISGTQATDEADYYCSSQDTVTRVFGGGTKLTVL

SEQ ID NO: 336 - protein, 11 aa, Homo sapiens
  source: /note=“Abet0374”
SGHNLGGKFAS

SEQ ID NO: 337 - protein, 7 aa, Homo sapiens
  source: /note=“Abet0374”
RDDKRPS

SEQ ID NO: 338 - protein, 9 aa, Homo sapiens
  source: /note=“Abet0374”
SSQDTVTRV

SEQ ID NO: 339 - protein, 22 aa, Homo sapiens
  source: /note=“Abet0374”
SYELTQPPSVSVTPGQTASITC

SEQ ID NO: 340 - protein, 15 aa, Homo sapiens
  source: /note=“Abet0374”
WYQQKPGQSPVLVIY

SEQ ID NO: 341 - protein, 32 aa, Homo sapiens
  source: /note=“Abet0374”
GIPERFSASNFGHTATLTISGTQATDEADYYC

SEQ ID NO: 342 - protein, 10 aa, Homo sapiens
  source: /note=“Abet0374”
FGGGTKLTVL

SEQ ID NO: 343 - DNA, 375 bp, Homo sapiens
  source: /note=“Abet0377”
gaggtgcagctgttggagtctgggggaggcttggtacagcctggggggtccctgagactctcctgtgcagcctctggattcaactttaacgagcagaccctctggtgggtccgccaagccccagggaaagggctggagtgggtctcagttattggtgtggggaccaagaacatcgcctacgcagacaccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctggggacaggggaccctggtcaccgtctcctca

SEQ ID NO: 344 - protein, 125 aa, Homo sapiens
  source: /note=“Abet0377”
EVQLLESGGGLVQPGGSLRLSCAASGFNFNEQTLWWVRQAPGKGLEWVSVIGVGTKNIAYADTVKGRFTISRDNSKNTLYLQMNSLRAEDTAVYYCAREWMDHSRPYYYYGMDVWGQGTLVTVSS

SEQ ID NO: 345 - protein, 5 aa, Homo sapiens
  source: /note=“Abet0377”
EQTLW

SEQ ID NO: 346 - protein, 17 aa, Homo sapiens
  source: /note=“Abet0377”
VIGVGTKNIAYADTVKG

SEQ ID NO: 347 - protein, 16 aa, Homo sapiens
  source: /note=“Abet0377”
EWMDHSRPYYYYGMDV

SEQ ID NO: 348 - protein, 30 aa, Homo sapiens; identical to SEQ ID NO: 186
  source: /note=“Abet0377”

SEQ ID NO: 349 - protein, 14 aa, Homo sapiens
  source: /note=“Abet0377”
WVRQAPGKGLEWVS

SEQ ID NO: 350 - protein, 32 aa, Homo sapiens; identical to SEQ ID NO: 8
  source: /note=“Abet0377”

SEQ ID NO: 351 - protein, 11 aa, Homo sapiens
  source: /note=“Abet0377”
WGQGTLVTVSS

SEQ ID NO: 352 - DNA, 318 bp, Homo sapiens
  source: /note=“Abet0377”
tcgtacgagttgactcagccaccctcagtgtccgtgtccccaggacagacggccagcatcacctgctctggacataacaccgagcacaagtggatctcgtggtatcaacagaagccaggccagtcccctgtcctggtcatctatcgagatgacaagcggccctcagggatccctgagcgattctctgccaccaactctgggcacactgccactctgaccatcagcgggacccaggctacggatgaggctgactattactgttcgtcccaggacacggtgactcgagtgttcggcggagggaccaagctgaccgtccta

SEQ ID NO: 353 - protein, 106 aa, Homo sapiens
  source: /note=“Abet0377”
SYELTQPPSVSVSPGQTASITCSGHNTEHKWISWYQQKPGQSPVLVIYRDDKRPSGIPERFSATNSGHTATLTISGTQATDEADYYCSSQDTVTRVFGGGTKLTVL

SEQ ID NO: 354 - protein, 11 aa, Homo sapiens
  source: /note=“Abet0377”
SGHNTEHKWIS

SEQ ID NO: 355 - protein, 7 aa, Homo sapiens
  source: /note=“Abet0377”
RDDKRPS

SEQ ID NO: 356 - protein, 9 aa, Homo sapiens
  source: /note=“Abet0377”
SSQDTVTRV

SEQ ID NO: 357 - protein, 22 aa, Homo sapiens
  source: /note=“Abet0377”
SYELTQPPSVSVSPGQTASITC

SEQ ID NO: 358 - protein, 15 aa, Homo sapiens
  source: /note=“Abet0377”
WYQQKPGQSPVLVIY

SEQ ID NO: 359 - protein, 32 aa, Homo sapiens
  source: /note=“Abet0377”
GIPERFSATNSGHTATLTISGTQATDEADYYC

SEQ ID NO: 360 - protein, 10 aa, Homo sapiens
  source: /note=“Abet0377”
FGGGTKLTVL

SEQ ID NO: 361 - DNA, 375 bp, Homo sapiens
  source: /note=“Abet0378”
gaggtgcagctgttggagtctgggggaggcttggtacagcctggggggtccctgagactctcctgtgcagcctctggattcccctttgagaccgacatcatgtggtgggtccgccaggctccagggaaggggctggagtgggtctcagttattggtaccaacaccgacaacgtcgcctacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 362 - protein, 125 aa, Homo sapiens
  source: /note=“Abet0378”
EVQLLESGGGLVQPGGSLRLSCAASGFPFETDIMWWVRQAPGKGLEWVSVIGTNTDNVAYADSVKGRFTISRDNSKNTLYLQMNSLRAEDTAVYYCAREWMDHSRPYYYYGMDVWGQGTLVTVSS

SEQ ID NO: 363 - protein, 5 aa, Homo sapiens
  source: /note=“Abet0378”
TDIMW

SEQ ID NO: 364 - protein, 17 aa, Homo sapiens
  source: /note=“Abet0378”
VIGTNTDNVAYADSVKG

SEQ ID NO: 365 - protein, 16 aa, Homo sapiens
  source: /note=“Abet0378”
EWMDHSRPYYYYGMDV

SEQ ID NO: 366 - protein, 30 aa, Homo sapiens
  source: /note=“Abet0378”
EVQLLESGGGLVQPGGSLRLSCAASGFPFE

SEQ ID NO: 367 - protein, 14 aa, Homo sapiens
  source: /note=“Abet0378”
WVRQAPGKGLEWVS

SEQ ID NO: 368 - protein, 32 aa, Homo sapiens; identical to SEQ ID NO: 8
  source: /note=“Abet0378”

SEQ ID NO: 369 - protein, 11 aa, Homo sapiens
  source: /note=“Abet0378”
WGQGTLVTVSS

SEQ ID NO: 370 - DNA, 318 bp, Homo sapiens
  source: /note=“Abet0378”
tcgtacgagttgacccagccaccctcagtgtccgtgtccccaggacagacggccagcatcacctgctctggacataacttggaagataaatttgcttcctggtatcaacagaagccaggccagtcccctgtcctggtcatctatcgagatgacaagcggccctcagggatccctgagcgattctctgcctccaactctgggcacactgccactctgaccatcagcgggacccaggctacggatgaggctgactattactgctcgtcctaggacacggtgactcgggtgttcggcggagggaccaagctgaccgtccta

SEQ ID NO: 371 - protein, 108 aa, Homo sapiens
  source: /note=“Abet0378”
SYELTQPPSVSVSPGQTASITCSGHNLEDKFASWYQQKPGQSPVLVIYRDDKRPSGIPERFSASNSGHTATLTISGTQATDEADYYCSSTERDTVTRVFGGGTKLTVL

SEQ ID NO: 372 - protein, 11 aa, Homo sapiens
  source: /note=“Abet0378”
SGHNLEDKFAS

SEQ ID NO: 373 - protein, 7 aa, Homo sapiens
  source: /note=“Abet0378”
RDDKRPS

SEQ ID NO: 374 - protein, 11 aa, Homo sapiens
  source: /note=“Abet0378”
SSTERDTVTRV

SEQ ID NO: 375 - protein, 22 aa, Homo sapiens
  source: /note=“Abet0378”
SYELTQPPSVSVSPGQTASITC

SEQ ID NO: 376 - protein, 15 aa, Homo sapiens
  source: /note=“Abet0378”
WYQQKPGQSPVLVIY

SEQ ID NO: 377 - protein, 32 aa, Homo sapiens; identical to SEQ ID NO: 17
  source: /note=“Abet0378”

SEQ ID NO: 378 - protein, 10 aa, Homo sapiens
  source: /note=“Abet0378”
FGGGTKLTVL

SEQ ID NO: 379 - DNA, 375 bp, Homo sapiens
  source: /note=“Abet0379”
gaggtgcagctgttggagtctgggggaggcttggtacagcctggggggtccctgagactctcctgtgcagcctctggattcgactttgccgagacgcctttgtggtgggtccgccaggctccaggggagaggctggagtgggtctcagttattggtagcaaccagaacaagaccgcctacgcagactccgtgaagggccggttcaccatctccagagacaattccaaggacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 380 - protein, 125 aa, Homo sapiens
  source: /note=“Abet0379”
EVQLLESGGGLVQPGGSLRLSCAASGFDFAETPLWWVRQAPGERLEWVSVIGSNQNKTAYADSVKGRFTISRDNSKDTLYLQMNSLRAEDTAVYYCAREWMDHSRPYYYYGMDVWGQGTLVTVSS

SEQ ID NO: 381 - protein, 5 aa, Homo sapiens
  source: /note=“Abet0379”
ETPLW

SEQ ID NO: 382 - protein, 17 aa, Homo sapiens
  source: /note=“Abet0379”
VIGSNQNKTAYADSVKG

SEQ ID NO: 383 - protein, 16 aa, Homo sapiens
  source: /note=“Abet0379”
EWMDHSRPYYYYGMDV

SEQ ID NO: 384 - protein, 30 aa, Homo sapiens
  source: /note=“Abet0379”
EVQLLESGGGLVQPGGSLRLSCAASGFDFA

SEQ ID NO: 385 - protein, 14 aa, Homo sapiens
  source: /note=“Abet0379”
WVRQAPGERLEWVS

SEQ ID NO: 386 - protein, 32 aa, Homo sapiens
  source: /note=“Abet0379”
RFTISRDNSKDTLYLQMNSLRAEDTAVYYCAR

SEQ ID NO: 387 - protein, 11 aa, Homo sapiens
  source: /note=“Abet0379”
WGQGTLVTVSS

SEQ ID NO: 388 - DNA, 318 bp, Homo sapiens
  source: /note=“Abet0379”
cagagcgtcttgactcagccaccctcagtgtccgtgtccccaggacagacggccagcatcacctgctctggacataacttggaagataaatttgcttcctggtatcaacagaagtcaggccagtcccctgtcctggtcatctatcgagatgacaagcggccctcagggatccctgagcgattctctgcctccaactccgggcacactgccactctgaccatcagcgggacccaggctacggatggggctgactattactgtgcgacccaggacaacttcactcgagtgttcggcggagggaccaagctgaccgtccta

SEQ ID NO: 389 - protein, 106 aa, Homo sapiens
  source: /note=“Abet0379”
QSVLTQPPSVSVSPGQTASITCSGHNLEDKFASWYQQKSGQSPVLVIYRDDKRPSGIPERFSASNSGHTATLTISGTQATDGADYYCATQDNFTRVFGGGTKLTVL

SEQ ID NO: 390 - protein, 11 aa, Homo sapiens
  source: /note=“Abet0379”
SGHNLEDKFAS

SEQ ID NO: 391 - protein, 7 aa, Homo sapiens
  source: /note=“Abet0379”
RDDKRPS

SEQ ID NO: 392 - protein, 9 aa, Homo sapiens
  source: /note=“Abet0379”
ATQDNFTRV

SEQ ID NO: 393 - protein, 22 aa, Homo sapiens
  source: /note=“Abet0379”
QSVLTQPPSVSVSPGQTASITC

SEQ ID NO: 394 - protein, 15 aa, Homo sapiens
  source: /note=“Abet0379”
WYQQKSGQSPVLVIY

SEQ ID NO: 395 - protein, 32 aa, Homo sapiens
  source: /note=“Abet0379”
GIPERFSASNSGHTATLTISGTQATDGADYYC

SEQ ID NO: 396 - protein, 10 aa, Homo sapiens
  source: /note=“Abet0379”
FGGGTKLTVL

SEQ ID NO: 397 - DNA, 375 bp, Homo sapiens
  source: /note=“Abet0380”
gaggtgcagctgttggagtctgggggaggcttggtacagcctggggggtccctgagactctcctgtgcagcctctatgggcaacttcaactaccagactatgtggtgggtccgccaggctccagggagggggctggagtgggtctcagttattggtaagaccaacgagaacatcgcctacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 398 - protein, 125 aa, Homo sapiens
  source: /note=“Abet0380”
EVQLLESGGGLVQPGGSLRLSCAASMGNFNYQTMWWVRQAPGRGLEWVSVIGKTNENIAYADSVKGRFTISRDNSKNTLYLQMNSLRAEDTAVYYCAREWMDHSRPYYYYGMDVWGQGTLVTVSS

SEQ ID NO: 399 - protein, 5 aa, Homo sapiens
  source: /note=“Abet0380”
YQTMW

SEQ ID NO: 400 - protein, 17 aa, Homo sapiens
  source: /note=“Abet0380”
VIGKTNENIAYADSVKG

SEQ ID NO: 401 - protein, 16 aa, Homo sapiens
  source: /note=“Abet0380”
EWMDHSRPYYYYGMDV

SEQ ID NO: 402 - protein, 30 aa, Homo sapiens
  source: /note=“Abet0380”
EVQLLESGGGLVQPGGSLRLSCAASMGNFN

SEQ ID NO: 403 - protein, 14 aa, Homo sapiens
  source: /note=“Abet0380”
WVRQAPGRGLEWVS

SEQ ID NO: 404 - protein, 32 aa, Homo sapiens; identical to SEQ ID NO: 8
  source: /note=“Abet0380”

SEQ ID NO: 405 - protein, 11 aa, Homo sapiens
  source: /note=“Abet0380”
WGQGTLVTVSS

SEQ ID NO: 406 - DNA, 318 bp, Homo sapiens
  source: /note=“Abet0380”
tcgtacgagttgactcagccaccctcagtgtccgtgtccccaggacagacggccagcatcacctgctctggacataacttggaagataaatttgcttcctggtatcaacagaagccaggccagtcccctgtcctggtcatctatcgagatgacaagcggccctcagggatccctgagcgattctctgcctccaactctgggcacactgccactctgaccatcagcgggacccaggctacggatgaggctgactattactgttcgtcccaggacacggtgactcgagtgttcggcggagggaccaagctgaccgtccta

SEQ ID NO: 407 - protein, 106 aa, Homo sapiens; identical to SEQ ID NO: 299
  source: /note=“Abet0380”

SEQ ID NO: 408 - protein, 11 aa, Homo sapiens
  source: /note=“Abet0380”
SGHNLEDKFAS

SEQ ID NO: 409 - protein, 7 aa, Homo sapiens
  source: /note=“Abet0380”
RDDKRPS

SEQ ID NO: 410 - protein, 9 aa, Homo sapiens
  source: /note=“Abet0380”
SSQDTVTRV

SEQ ID NO: 411 - protein, 22 aa, Homo sapiens
  source: /note=“Abet0380”
SYELTQPPSVSVSPGQTASITC

SEQ ID NO: 412 - protein, 15 aa, Homo sapiens
  source: /note=“Abet0380”
WYQQKPGQSPVLVIY

SEQ ID NO: 413 - protein, 32 aa, Homo sapiens; identical to SEQ ID NO: 17
  source: /note=“Abet0380”

SEQ ID NO: 414 - protein, 10 aa, Homo sapiens
  source: /note=“Abet0380”
FGGGTKLTVL

SEQ ID NO: 415 - DNA, 375 bp, Homo sapiens
  source: /note=“Abet0381”
gaggtgcagctgttggagtctgggggaggcttggtacagcctggggggtccttgagactctcctgtgcagcctcttccccgtcgttcccgcgggagaccatgtggtgggtccgccaggctccagggaaggggcttgagtgggtctcagttattggtacccagccgaaccgcttgacgtacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatagacgtctgggggcaggggaccctggtcaccgtctcccca

SEQ ID NO: 416 - protein, 125 aa, Homo sapiens
  source: /note=“Abet0381”
EVQLLESGGGLVQPGGSLRLSCAASSPSFPRETMWWVRQAPGKGLEWVSVIGTQPNRLTYADSVKGRFTISRDNSKNTLYLQMNSLRAEDTAVYYCAREWMDHSRPYYYYGIDVWGQGTLVTVSP

SEQ ID NO: 417 - protein, 5 aa, Homo sapiens
  source: /note=“Abet0381”
RETMW

SEQ ID NO: 418 - protein, 17 aa, Homo sapiens
  source: /note=“Abet0381”
VIGTQPNRLTYADSVKG

SEQ ID NO: 419 - protein, 16 aa, Homo sapiens
  source: /note=“Abet0381”
EWMDHSRPYYYYGIDV

SEQ ID NO: 420 - protein, 30 aa, Homo sapiens
  source: /note=“Abet0381”
EVQLLESGGGLVQPGGSLRLSCAASSPSFP

SEQ ID NO: 421 - protein, 14 aa, Homo sapiens
  source: /note=“Abet0381”
WVRQAPGKGLEWVS

SEQ ID NO: 422 - protein, 32 aa, Homo sapiens; identical to SEQ ID NO: 8
  source: /note=“Abet0381”

SEQ ID NO: 423 - protein, 11 aa, Homo sapiens
  source: /note=“Abet0381”
WGQGTLVTVSP

SEQ ID NO: 424 - DNA, 318 bp, Homo sapiens
  source: /note=“Abet0381”
tcgtacgagttgactcagccaccctcagtgtccgcgtccccaggacagacggccagcatcacctgctctggacataacttggaagataaatttgtttcctggtatcaacagaagccaggccagtcccctgtcctggtcatctatcgagatgacaagcgaccctcagggatccctgagcgattctctgcctccaactctgggcacactgccactctgaccatcagcgggacccaggctacggatgaggctaactattactgttcgtcccaggacacggtgactcgagcgttcggcggagggaccaagctgaccgtccta

SEQ ID NO: 425 - protein, 106 aa, Homo sapiens
  source: /note=“Abet0381”
SYELTQPPSVSASPGQTASITCSGHNLEDKFVSWYQQKPGQSPVLVIYRDDKRPSGIPERFSASNSGHTATLTISGTQATDEANYYCSSQDTVTRAFGGGTKLTVL

SEQ ID NO: 426 - protein, 11 aa, Homo sapiens
  source: /note=“Abet0381”
SGHNLEDKFVS

SEQ ID NO: 427 - protein, 7 aa, Homo sapiens
  source: /note=“Abet0381”
RDDKRPS

SEQ ID NO: 428 - protein, 9 aa, Homo sapiens
  source: /note=“Abet0381”
SSQDTVTRA

SEQ ID NO: 429 - protein, 22 aa, Homo sapiens
  source: /note=“Abet0381”
SYELTQPPSVSASPGQTASITC

SEQ ID NO: 430 - protein, 15 aa, Homo sapiens
  source: /note=“Abet0381”
WYQQKPGQSPVLVIY

SEQ ID NO: 431 - protein, 32 aa, Homo sapiens
  source: /note=“Abet0381”
GIPERFSASNSGHTATLTISGTQATDEANYYC

SEQ ID NO: 432 - protein, 10 aa, Homo sapiens
  source: /note=“Abet0381”
FGGGTKLTVL

SEQ ID NO: 433 - DNA, 375 bp, Homo sapiens
  source: /note=“Abet0382”
gaggtgcagctgttggagtctgggggaggcttggtacagcctggggggtccctgaggctctcctgtgcagcctctggattccactttaccaactccatcatgtggtgggtccgccaggctccagggaaggggctggagtgggtctcagttattggtagcgaggcgcaccgcgtcacgtacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 434 - protein, 125 aa, Homo sapiens
  source: /note=“Abet0382”
EVQLLESGGGLVQPGGSLRLSCAASGFHFTNSIMWWVRQAPGKGLEWVSVIGSEAHRVTYADSVKGRFTISRDNSKNTLYLQMNSLRAEDTAVYYCAREWMDHSRPYYYYGMDVWGQGTLVTVSS

SEQ ID NO: 435 - protein, 5 aa, Homo sapiens
  source: /note=“Abet0382”
NSIMW

SEQ ID NO: 436 - protein, 17 aa, Homo sapiens
  source: /note=“Abet0382”
VIGSEAHRVTYADSVKG

SEQ ID NO: 437 - protein, 16 aa, Homo sapiens
  source: /note=“Abet0382”
EWMDHSRPYYYYGMDV

SEQ ID NO: 438 - protein, 30 aa, Homo sapiens
  source: /note=“Abet0382”
EVQLLESGGGLVQPGGSLRLSCAASGFHFT

SEQ ID NO: 439 - protein, 14 aa, Homo sapiens
  source: /note=“Abet0382”
WVRQAPGKGLEWVS

SEQ ID NO: 440 - protein, 32 aa, Homo sapiens; identical to SEQ ID NO: 8
  source: /note=“Abet0382”

SEQ ID NO: 441 - protein, 11 aa, Homo sapiens
  source: /note=“Abet0382”
WGQGTLVTVSS

SEQ ID NO: 442 - DNA, 318 bp, Homo sapiens
  source: /note=“Abet0382”
tcgtacgagttgattcagccaccctcagtgtccgtgtccccaggacagacagccagcatcacctgctctggacataacttggaagataaatttgcttcctggtatcaacagaagccaggccagtcccctgtcctggtcatctatcgagatgacaagcggccctcagggatccctgagcgattctctgccaccaactctgggcacactgccactctgaccatcagcgggacccaggctacggatgaggctgactattactgttcgtcccaggactcggtgactcgagtgttcggcggagggaccaagctgaccgtccta

SEQ ID NO: 443 - protein, 106 aa, Homo sapiens
  source: /note=“Abet0382”
SYELIQPPSVSVSPGQTASITCSGHNLEDKFASWYQQKPGQSPVLVIYRDDKRPSGIPERFSATNSGHTATLTISGTQATDEADYYCSSQDSVTRVFGGGTKLTVL

SEQ ID NO: 444 - protein, 11 aa, Homo sapiens
  source: /note=“Abet0382”
SGHNLEDKFAS

SEQ ID NO: 445 - protein, 7 aa, Homo sapiens
  source: /note=“Abet0382”
RDDKRPS

SEQ ID NO: 446 - protein, 9 aa, Homo sapiens
  source: /note=“Abet0382”
SSQDSVTRV

SEQ ID NO: 447 - protein, 22 aa, Homo sapiens
  source: /note=“Abet0382”
SYELIQPPSVSVSPGQTASITC

SEQ ID NO: 448 - protein, 15 aa, Homo sapiens
  source: /note=“Abet0382”
WYQQKPGQSPVLVIY

SEQ ID NO: 449 - protein, 32 aa, Homo sapiens; identical to SEQ ID NO: 359
  source: /note=“Abet0382”

SEQ ID NO: 450 - protein, 10 aa, Homo sapiens
  source: /note=“Abet0382”
FGGGTKLTVL

SEQ ID NO: 451 - DNA, 375 bp, Homo sapiens
  source: /note=“Abet0383”
gaggtgcagctgttggagtccgggggaggcttggtacagcctggggggtccctgaaactctcctgtgcagcctctggattcacgtttgactggtacccgatgtggtgggtccgccaggctccagggaagaggctggagtggatctcagttattggtgcggacaacgccaagatcgcctacgcagactccgtgaagggccggtttaccatctccagagacaattccaagaacacgctgtatctgcaaatgaacagcctgagagccgaggacacggccgtgtattactgtgcgagagagtggatgggccactcccgcccctactactactacggtatggacgtctgggggcaggggaccccggtcaccgtctcctca

SEQ ID NO: 452 - protein, 125 aa, Homo sapiens
  source: /note=“Abet0383”
EVQLLESGGGLVQPGGSLKLSCAASGFTFDWYPMWWVRQAPGKRLEWISVIGADNAKIAYADSVKGRFTISRDNSKNTLYLQMNSLRAEDTAVYYCAREWMGHSRPYYYYGMDVWGQGTPVTVSS

SEQ ID NO: 453 - protein, 5 aa, Homo sapiens
  source: /note=“Abet0383”
WYPMW

SEQ ID NO: 454 - protein, 17 aa, Homo sapiens
  source: /note=“Abet0383”
VIGADNAKIAYADSVKG

SEQ ID NO: 455 - protein, 16 aa, Homo sapiens
  source: /note=“Abet0383”
EWMGHSRPYYYYGMDV

SEQ ID NO: 456 - protein, 30 aa, Homo sapiens
  source: /note=“Abet0383”
EVQLLESGGGLVQPGGSLKLSCAASGFTFD

SEQ ID NO: 457 - protein, 14 aa, Homo sapiens
  source: /note=“Abet0383”
WVRQAPGKRLEWIS

SEQ ID NO: 458 - protein, 32 aa, Homo sapiens; identical to SEQ ID NO: 8
  source: /note=“Abet0383”

SEQ ID NO: 459 - protein, 11 aa, Homo sapiens
  source: /note=“Abet0383”
WGQGTPVTVSS

SEQ ID NO: 460 - DNA, 318 bp, Homo sapiens
  source: /note=“Abet0383”
tcgtacgagttgactcagccaccctcagtatccgtgtccccaggacagacggccagcatcacctgctctggacataacttgggagataaatttgcttcctggtatcaacagaagccaggccagtcccctgtcctggtcatctatcgagatgacaagcggccctcagggatccctgagcgattctctgcctccaactctgggcacactgccactctgaccattagcgggacccaggctacggatgaggctgactattactgttcgtcccaggacacggtgactcgagtgttcggcggagggaccaagctgaccgtcctg

SEQ ID NO: 461 - protein, 106 aa, Homo sapiens
  source: /note=“Abet0383”
SYELTQPPSVSVSPGQTASITCSGHNLGDKFASWYQQKPGQSPVLVIYRDDKRPSGIPERFSASNSGHTATLTISGTQATDEADYYCSSQDTVTRVFGGGTKLTVL

SEQ ID NO: 462 - protein, 11 aa, Homo sapiens
  source: /note=“Abet0383”
SGHNLGDKFAS

SEQ ID NO: 463 - protein, 7 aa, Homo sapiens
  source: /note=“Abet0383”
RDDKRPS

SEQ ID NO: 464 - protein, 9 aa, Homo sapiens
  source: /note=“Abet0383”
SSQDTVTRV

SEQ ID NO: 465 - protein, 22 aa, Homo sapiens
  source: /note=“Abet0383”
SYELTQPPSVSVSPGQTASITC

SEQ ID NO: 466 - protein, 15 aa, Homo sapiens
  source: /note=“Abet0383”
WYQQKPGQSPVLVIY

SEQ ID NO: 467 - protein, 32 aa, Homo sapiens; identical to SEQ ID NO: 17
  source: /note=“Abet0383”

SEQ ID NO: 468 - protein, 10 aa, Homo sapiens
  source: /note=“Abet0383”
FGGGTKLTVL

SEQ ID NO: 469 - DNA, 375 bp, Homo sapiens; identical to SEQ ID NO: 181
  source: /note=“Abet0343-GL”

SEQ ID NO: 470 - protein, 125 aa, Homo sapiens; identical to SEQ ID NO: 182
  source: /note=“Abet0343-GL”

SEQ ID NO: 471 - protein, 5 aa, Homo sapiens
  source: /note=“Abet0343-GL”
HQVMW

SEQ ID NO: 472 - protein, 17 aa, Homo sapiens
  source: /note=“Abet0343-GL”
VIGKTNENIAYADSVKG

SEQ ID NO: 473 - protein, 16 aa, Homo sapiens
  source: /note=“Abet0343-GL”
EWMDHSRPYYYYGMDV

SEQ ID NO: 474 - protein, 30 aa, Homo sapiens; identical to SEQ ID NO: 186
  source: /note=“Abet0343-GL”

SEQ ID NO: 475 - protein, 14 aa, Homo sapiens
  source: /note=“Abet0343-GL”
WVRQAPGKGLEWVS

SEQ ID NO: 476 - protein, 32 aa, Homo sapiens; identical to SEQ ID NO: 8
  source: /note=“Abet0343-GL”

SEQ ID NO: 477 - protein, 11 aa, Homo sapiens
  source: /note=“Abet0343-GL”
WGQGTLVTVSS

SEQ ID NO: 478 - DNA, 318 bp, Homo sapiens
  source: /note=“Abet0343-GL”
tcgtacgagttgactcagccaccctcagtgtccgtgtccccaggacagacggccagcatcacctgctctggacataacttggaagataaatttgcttcctggtatcaacagaagccaggccagtcccctgtcctggtcatctatcgagatgacaagcggccctcagggatccctgagcgattctctgcctccaactctgggcacactgccactctgaccatcagcgggacccaggctatggatgaggctgactattactgttcgtcccaggacacggtgactcgagtgttcggcggagggaccaagctgaccgtccta

SEQ ID NO: 479 - protein, 106 aa, Homo sapiens
  source: /note=“Abet0343-GL”
SYELTQPPSVSVSPGQTASITCSGHNLEDKFASWYQQKPGQSPVLVIYRDDKRPSGIPERFSASNSGHTATLTISGTQAMDEADYYCSSQDTVTRVFGGGTKLTVL

SEQ ID NO: 480 - protein, 11 aa, Homo sapiens
  source: /note=“Abet0343-GL”
SGHNLEDKFAS

SEQ ID NO: 481 - protein, 7 aa, Homo sapiens
  source: /note=“Abet0343-GL”
RDDKRPS

SEQ ID NO: 482 - protein, 9 aa, Homo sapiens
  source: /note=“Abet0343-GL”
SSQDTVTRV

SEQ ID NO: 483 - protein, 22 aa, Homo sapiens
  source: /note=“Abet0343-GL”
SYELTQPPSVSVSPGQTASITC

SEQ ID NO: 484 - protein, 15 aa, Homo sapiens
  source: /note=“Abet0343-GL”
WYQQKPGQSPVLVIY

SEQ ID NO: 485 - protein, 32 aa, Homo sapiens; identical to SEQ ID NO: 35
  source: /note=“Abet0343-GL”

SEQ ID NO: 486 - protein, 10 aa, Homo sapiens
  source: /note=“Abet0343-GL”
FGGGTKLTVL

SEQ ID NO: 487 - DNA, 375 bp, Homo sapiens
  source: /note=“Abet0369-GL”
gaggtgcagctgttggagtctgggggaggcttggtacagcctggggggtccctgagactctcctgtgcagcctcttcgttccagatctcgaagaacactatgtggtgggtccgccaggctccagggaaggggctggagtgggtctcagttattggtaaggacgagacccgcttcaactacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 488 - protein, 125 aa, Homo sapiens
  source: /note=“Abet0369-GL”
EVQLLESGGGLVQPGGSLRLSCAASSFQISKNTMWWVRQAPGKGLEWVSVIGKDETRFNYADSVKGRFTISRDNSKNTLYLQMNSLRAEDTAVYYCAREWMDHSRPYYYYGMDVWGQGTLVTVSS

SEQ ID NO: 489 - protein, 5 aa, Homo sapiens
  source: /note=“Abet0369-GL”
KNTMW

SEQ ID NO: 490 - protein, 17 aa, Homo sapiens
  source: /note=“Abet0369-GL”
VIGKDETRFNYADSVKG

SEQ ID NO: 491 - protein, 16 aa, Homo sapiens
  source: /note=“Abet0369-GL”
EWMDHSRPYYYYGMDV

SEQ ID NO: 492 - protein, 30 aa, Homo sapiens; identical to SEQ ID NO: 240
  source: /note=“Abet0369-GL”

SEQ ID NO: 493 - protein, 14 aa, Homo sapiens
  source: /note=“Abet0369-GL”
WVRQAPGKGLEWVS

SEQ ID NO: 494 - protein, 32 aa, Homo sapiens; identical to SEQ ID NO: 8
  source: /note=“Abet0369-GL”

SEQ ID NO: 495 - protein, 11 aa, Homo sapiens
  source: /note=“Abet0369-GL”
WGQGTLVTVSS

SEQ ID NO: 496 - DNA, 318 bp, Homo sapiens
  source: /note=“Abet0369-GL”
tcgtacgagttgactcagccaccctcagtgtccgtgtccccaggacagacggccagcatcacctgctctggacgtaacatcggggacagctgggtcgcgtggtatcaacagaagccaggccagtcccctgtcctggtcatctatcgagatgacaagcggccctcagggatccctgagcgattctctgcctccaactctgggcacactgccactctgaccatcagcgggacccaggctatggatgaggctgactattactgttcgtcccaggacacggtgactcgagtgttcggcggagggaccaagctgaccgtccta

SEQ ID NO: 497 - protein, 106 aa, Homo sapiens
  source: /note=“Abet0369-GL”
SYELTQPPSVSVSPGQTASITCSGRNIGDSWVAWYQQKPGQSPVLVIYRDDKRPSGIPERFSASNSGHTATLTISGTQAMDEADYYCSSQDTVTRVFGGGTKLTVL

SEQ ID NO: 498 - protein, 11 aa, Homo sapiens
  source: /note=“Abet0369-GL”
SGRNIGDSWVA

SEQ ID NO: 499 - protein, 7 aa, Homo sapiens
  source: /note=“Abet0369-GL”
RDDKRPS

SEQ ID NO: 500 - protein, 9 aa, Homo sapiens
  source: /note=“Abet0369-GL”
SSQDTVTRV

SEQ ID NO: 501 - protein, 22 aa, Homo sapiens
  source: /note=“Abet0369-GL”
SYELTQPPSVSVSPGQTASITC

SEQ ID NO: 502 - protein, 15 aa, Homo sapiens
  source: /note=“Abet0369-GL”
WYQQKPGQSPVLVIY

SEQ ID NO: 503 - protein, 32 aa, Homo sapiens; identical to SEQ ID NO: 35
  source: /note=“Abet0369-GL”

SEQ ID NO: 504 - protein, 10 aa, Homo sapiens
  source: /note=“Abet0369-GL”
FGGGTKLTVL

SEQ ID NO: 505 - DNA, 375 bp, Homo sapiens; identical to SEQ ID NO: 343
  source: /note=“Abet0377-GL”

SEQ ID NO: 506 - protein, 125 aa, Homo sapiens; identical to SEQ ID NO: 344
  source: /note=“Abet0377-GL”

SEQ ID NO: 507 - protein, 5 aa, Homo sapiens
  source: /note=“Abet0377-GL”
EQTLW

SEQ ID NO: 508 - protein, 17 aa, Homo sapiens
  source: /note=“Abet0377-GL”
VIGVGTKNIAYADTVKG

SEQ ID NO: 509 - protein, 16 aa, Homo sapiens
  source: /note=“Abet0377-GL”
EWMDHSRPYYYYGMDV

SEQ ID NO: 510 - protein, 30 aa, Homo sapiens; identical to SEQ ID NO: 186
  source: /note=“Abet0377-GL”

SEQ ID NO: 511 - protein, 14 aa, Homo sapiens
  source: /note=“Abet0377-GL”
WVRQAPGKGLEWVS

SEQ ID NO: 512 - protein, 32 aa, Homo sapiens; identical to SEQ ID NO: 8
  source: /note=“Abet0377-GL”

SEQ ID NO: 513 - protein, 11 aa, Homo sapiens
  source: /note=“Abet0377-GL”
WGQGTLVTVSS

SEQ ID NO: 514 - DNA, 318 bp, Homo sapiens
  source: /note=“Abet0377-GL”
tcgtacgagttgactcagccaccctcagtgtccgtgtccccaggacagacggccagcatcacctgctctggacataacaccgagcacaagtggatctcgtggtatcaacagaagccaggccagtcccctgtcctggtcatctatcgagatgacaagcggccctcagggatccctgagcgattctctgcctccaactctgggcacactgccactctgaccatcagcgggacccaggctatggatgaggctgactattactgttcgtcccaggacacggtgactcgagtgttcggcggagggaccaagctgaccgtccta

SEQ ID NO: 515 - protein, 106 aa, Homo sapiens
  source: /note=“Abet0377-GL”
SYELTQPPSVSVSPGQTASITCSGHNTEHKWISWYQQKPGQSPVLVIYRDDKRPSGIPERFSASNSGHTATLTISGTQAMDEADYYCSSQDTVTRVFGGGTKLTVL

SEQ ID NO: 516 - protein, 11 aa, Homo sapiens
  source: /note=“Abet0377-GL”
SGHNTEHKWIS

SEQ ID NO: 517 - protein, 7 aa, Homo sapiens
  source: /note=“Abet0377-GL”
RDDKRPS

SEQ ID NO: 518 - protein, 9 aa, Homo sapiens
  source: /note=“Abet0377-GL”
SSQDTVTRV

SEQ ID NO: 519 - protein, 22 aa, Homo sapiens
  source: /note=“Abet0377-GL”
SYELTQPPSVSVSPGQTASITC

SEQ ID NO: 520 - protein, 15 aa, Homo sapiens
  source: /note=“Abet0377-GL”
WYQQKPGQSPVLVIY

SEQ ID NO: 521 - protein, 32 aa, Homo sapiens; identical to SEQ ID NO: 35
  source: /note=“Abet0377-GL”

SEQ ID NO: 522 - protein, 10 aa, Homo sapiens
  source: /note=“Abet0377-GL”
FGGGTKLTVL

SEQ ID NO: 523 - DNA, 375 bp, Homo sapiens
  source: /note=“Abet0380-GL”
gaggtgcagctgttggagtctgggggaggcttggtacagcctggggggtccctgagactctcctgtgcagcctctatgggcaacttcaactaccagactatgtggtgggtccgccaggctccagggaaggggctggagtgggtctcagttattggtaagaccaacgagaacatcgcctacgcagactccgtgaagggccggttcaccatctccagagacaattccaagaacacgctgtatctgcaaatgaacagcctgagagccgaggacacggccgtgtattactgtgcgagagagtggatggaccactcccgcccctactactactacggtatggacgtctgggggcaggggaccctggtcaccgtctcctca

SEQ ID NO: 524 - protein, 125 aa, Homo sapiens
  source: /note=“Abet0380-GL”
EVQLLESGGGLVQPGGSLRLSCAASMGNFNYQTMWWVRQAPGKGLEWVSVIGKTNENIAYADSVKGRFTISRDNSKNTLYLQMNSLRAEDTAVYYCAREWMDHSRPYYYYGMDVWGQGTLVTVSS

SEQ ID NO: 525 - protein, 5 aa, Homo sapiens
  source: /note=“Abet0380-GL”
YQTMW

SEQ ID NO: 526 - protein, 17 aa, Homo sapiens
  source: /note=“Abet0380-GL”
VIGKTNENIAYADSVKG

SEQ ID NO: 527 - protein, 16 aa, Homo sapiens
  source: /note=“Abet0380-GL”
EWMDHSRPYYYYGMDV

SEQ ID NO: 528 - protein, 30 aa, Homo sapiens; identical to SEQ ID NO: 402
  source: /note=“Abet0380-GL”

SEQ ID NO: 529 - protein, 14 aa, Homo sapiens
  source: /note=“Abet0380-GL”
WVRQAPGKGLEWVS

SEQ ID NO: 530 - protein, 32 aa, Homo sapiens; identical to SEQ ID NO: 8
  source: /note=“Abet0380-GL”

SEQ ID NO: 531 - protein, 11 aa, Homo sapiens
  source: /note=“Abet0380-GL”
WGQGTLVTVSS

SEQ ID NO: 532 - DNA, 318 bp, Homo sapiens; identical to SEQ ID NO: 478
  source: /note=“Abet0380-GL”

SEQ ID NO: 533 - protein, 106 aa, Homo sapiens; identical to SEQ ID NO: 479
  source: /note=“Abet0380-GL”

SEQ ID NO: 534 - protein, 11 aa, Homo sapiens
  source: /note=“Abet0380-GL”
SGHNLEDKFAS

SEQ ID NO: 535 - protein, 7 aa, Homo sapiens
  source: /note=“Abet0380-GL”
RDDKRPS

SEQ ID NO: 536 - protein, 9 aa, Homo sapiens
  source: /note=“Abet0380-GL”
SSQDTVTRV

SEQ ID NO: 537 - protein, 22 aa, Homo sapiens
  source: /note=“Abet0380-GL”
SYELTQPPSVSVSPGQTASITC

SEQ ID NO: 538 - protein, 15 aa, Homo sapiens
  source: /note=“Abet0380-GL”
WYQQKPGQSPVLVIY

SEQ ID NO: 539 - protein, 32 aa, Homo sapiens; identical to SEQ ID NO: 35
  source: /note=“Abet0380-GL”

SEQ ID NO: 540 - protein, 10 aa, Homo sapiens
  source: /note=“Abet0380-GL”
FGGGTKLTVL

SEQ ID NO: 541 - DNA, 375 bp, Homo sapiens; identical to SEQ ID NO: 433
  source: /note=“Abet0382-GL”

SEQ ID NO: 542 - protein, 125 aa, Homo sapiens; identical to SEQ ID NO: 434
  source: /note=“Abet0382-GL”

SEQ ID NO: 543 - protein, 5 aa, Homo sapiens
  source: /note=“Abet0382-GL”
NSIMW

SEQ ID NO: 544 - protein, 17 aa, Homo sapiens
  source: /note=“Abet0382-GL”
VIGSEAHRVTYADSVKG

SEQ ID NO: 545 - protein, 16 aa, Homo sapiens
  source: /note=“Abet0382-GL”
EWMDHSRPYYYYGMDV

SEQ ID NO: 546 - protein, 30 aa, Homo sapiens; identical to SEQ ID NO: 438
  source: /note=“Abet0382-GL”

SEQ ID NO: 547 - protein, 14 aa, Homo sapiens
  source: /note=“Abet0382-GL”
WVRQAPGKGLEWVS

SEQ ID NO: 548 - protein, 32 aa, Homo sapiens; identical to SEQ ID NO: 8
  source: /note=“Abet0382-GL”

SEQ ID NO: 549 - protein, 11 aa, Homo sapiens
  source: /note=“Abet0382-GL”
WGQGTLVTVSS

SEQ ID NO: 550 - DNA, 318 bp, Homo sapiens; identical to SEQ ID NO: 478
  source: /note=“Abet0382-GL”

SEQ ID NO: 551 - protein, 106 aa, Homo sapiens; identical to SEQ ID NO: 479
  source: /note=“Abet0382-GL”

SEQ ID NO: 552 - protein, 11 aa, Homo sapiens
  source: /note=“Abet0382-GL”
SGHNLEDKFAS

SEQ ID NO: 553 - protein, 7 aa, Homo sapiens
  source: /note=“Abet0382-GL”
RDDKRPS

SEQ ID NO: 554 - protein, 9 aa, Homo sapiens
  source: /note=“Abet0382-GL”
SSQDTVTRV

SEQ ID NO: 555 - protein, 22 aa, Homo sapiens
  source: /note=“Abet0382-GL”
SYELTQPPSVSVSPGQTASITC

SEQ ID NO: 556 - protein, 15 aa, Homo sapiens
  source: /note=“Abet0382-GL”
WYQQKPGQSPVLVIY

SEQ ID NO: 557 - protein, 32 aa, Homo sapiens; identical to SEQ ID NO: 35
  source: /note=“Abet0382-GL”

SEQ ID NO: 558 - protein, 10 aa, Homo sapiens
  source: /note=“Abet0382-GL”
FGGGTKLTVL

SEQ ID NO: 559 - protein, 42 aa, Homo sapiens [sequence omitted]

SEQ ID NO: 560 - protein, 42 aa, Homo sapiens [sequence omitted]

SEQ ID NO: 561 - protein, 40 aa, Homo sapiens
DAEFRHDSGYEVHHQKLVFFAEDVGSNKGAIIGLMVGGVV

SEQ ID NO: 562 - protein, 40 aa, Homo sapiens; identical to SEQ ID NO: 561

SEQ ID NO: 563 - protein, 42 aa, Mus sp.
DAEFGHDSGFEVRHQKLVFFAEDVGSNKGAIIGLMVGGVVIA

SEQ ID NO: 564 - protein, 42 aa, Mus sp.; identical to SEQ ID NO: 563

SEQ ID NO: 565 - protein, 42 aa, Mus sp.; identical to SEQ ID NO: 563

SEQ ID NO: 566 - protein, 40 aa, Mus sp.
DAEFGHDSGFEVRHQKLVFFAEDVGSNKGAIIGLMVGGVV

SEQ ID NO: 567 - protein, 40 aa, Mus sp.; identical to SEQ ID NO: 566

SEQ ID NO: 568 - protein, 42 aa, Unknown
  source: /note=“Description of Unknown biotinylated scrambled amyloid beta sequence”
AIAEGDSHVLKEGAYMEIFDVQGHVFGGLIFRVVDLGSHNVA

SEQ ID NO: 569 - protein, 43 aa, Homo sapiens
DAEFRHDSGYEVHHQKLVFFAEDVGSNKGAIIGLMVGGVVIAT

SEQ ID NO: 570 - protein, 17 aa, Homo sapiens
KKKGAIIGLMVGGVVIA

SEQ ID NO: 571 - protein, 15 aa, Homo sapiens
KKKGAIIGLMVGGVV

SEQ ID NO: 572 - protein, 16 aa, Homo sapiens
DAEFRHDSGYEVHHQK

SEQ ID NO: 573 - protein, 12 aa, Homo sapiens
EVRHQKLVFFAE

SEQ ID NO: 574 - protein, 17 aa, Homo sapiens
VRHQKLVFFAEDVGSNK

SEQ ID NO: 575 - protein, 26 aa, Homo sapiens
LVFFAEDVGSNKGAIIGLMVGGVVIA

SEQ ID NO: 576 - protein, 32 aa, Homo sapiens
EVRHQKLVFFAEDVGSNKGAIIGLMVGGVVIA

SEQ ID NO: 577 - protein, 40 aa, Homo sapiens
EFRHDSGYEVHHQKLVFFAEDVGSNKGAIIGLMVGGVVIA

SEQ ID NO: 578 - protein, 125 aa, Homo sapiens; identical to SEQ ID NO: 20

SEQ ID NO: 579 - protein, 125 aa, Homo sapiens; identical to SEQ ID NO: 38

SEQ ID NO: 580 - protein, 125 aa, Homo sapiens; identical to SEQ ID NO: 56

SEQ ID NO: 581 - protein, 125 aa, Homo sapiens; identical to SEQ ID NO: 74

SEQ ID NO: 582 - protein, 125 aa, Homo sapiens; identical to SEQ ID NO: 92

SEQ ID NO: 583 - protein, 125 aa, Homo sapiens
EVQLLESGGGLVQPGGSLRLSCAASSDAFKTDTMWWVRQAPRKGLEWVSVIGAHTNNSAYADSVKGRFTISRDNSKNTLYLQMNSLRAEDTAVYYCAREWMDRSRPYYYYGMDVWGQGTLVTVSS

SEQ ID NO: 584 - protein, 125 aa, Homo sapiens; identical to SEQ ID NO: 128

SEQ ID NO: 585 - protein, 125 aa, Homo sapiens; identical to SEQ ID NO: 146

SEQ ID NO: 586 - protein, 125 aa, Homo sapiens; identical to SEQ ID NO: 164

SEQ ID NO: 587 - protein, 125 aa, Homo sapiens; identical to SEQ ID NO: 182

SEQ ID NO: 588 - protein, 125 aa, Homo sapiens; identical to SEQ ID NO: 200

SEQ ID NO: 589 - protein, 125 aa, Homo sapiens; identical to SEQ ID NO: 218

SEQ ID NO: 590 - protein, 125 aa, Homo sapiens; identical to SEQ ID NO: 236

SEQ ID NO: 591 - protein, 125 aa, Homo sapiens; identical to SEQ ID NO: 254

SEQ ID NO: 592 - protein, 125 aa, Homo sapiens; identical to SEQ ID NO: 272

SEQ ID NO: 593 - protein, 125 aa, Homo sapiens; identical to SEQ ID NO: 290

SEQ ID NO: 594 - protein, 125 aa, Homo sapiens; identical to SEQ ID NO: 308

SEQ ID NO: 595 - protein, 125 aa, Homo sapiens; identical to SEQ ID NO: 326

SEQ ID NO: 596 - protein, 125 aa, Homo sapiens; identical to SEQ ID NO: 344

SEQ ID NO: 597 - protein, 125 aa, Homo sapiens; identical to SEQ ID NO: 362

SEQ ID NO: 598 - protein, 125 aa, Homo sapiens; identical to SEQ ID NO: 380

SEQ ID NO: 599 - protein, 125 aa, Homo sapiens; identical to SEQ ID NO: 398

SEQ ID NO: 600 - protein, 125 aa, Homo sapiens; identical to SEQ ID NO: 416

SEQ ID NO: 601 - protein, 125 aa, Homo sapiens; identical to SEQ ID NO: 434

SEQ ID NO: 602 - protein, 125 aa, Homo sapiens; identical to SEQ ID NO: 452

SEQ ID NO: 603 - protein, 106 aa, Homo sapiens; identical to SEQ ID NO: 29

SEQ ID NO: 604 - protein, 106 aa, Homo sapiens; identical to SEQ ID NO: 47

SEQ ID NO: 605 - protein, 106 aa, Homo sapiens; identical to SEQ ID NO: 65

SEQ ID NO: 606 - protein, 106 aa, Homo sapiens; identical to SEQ ID NO: 83

SEQ ID NO: 607 - protein, 106 aa, Homo sapiens; identical to SEQ ID NO: 101

SEQ ID NO: 608 - protein, 106 aa, Homo sapiens; identical to SEQ ID NO: 119

SEQ ID NO: 609 - protein, 106 aa, Homo sapiens; identical to SEQ ID NO: 137

SEQ ID NO: 610 - protein, 106 aa, Homo sapiens; identical to SEQ ID NO: 155

SEQ ID NO: 611 - protein, 106 aa, Homo sapiens; identical to SEQ ID NO: 173

SEQ ID NO: 612 - protein, 106 aa, Homo sapiens; identical to SEQ ID NO: 191

SEQ ID NO: 613 - protein, 106 aa, Homo sapiens; identical to SEQ ID NO: 209

SEQ ID NO: 614 - protein, 106 aa, Homo sapiens; identical to SEQ ID NO: 227

SEQ ID NO: 615 - protein, 106 aa, Homo sapiens; identical to SEQ ID NO: 245

SEQ ID NO: 616 - protein, 106 aa, Homo sapiens; identical to SEQ ID NO: 263

SEQ ID NO: 617 - protein, 106 aa, Homo sapiens; identical to SEQ ID NO: 281

SEQ ID NO: 618 - protein, 106 aa, Homo sapiens; identical to SEQ ID NO: 299

SEQ ID NO: 619 - protein, 106 aa, Homo sapiens; identical to SEQ ID NO: 317

SEQ ID NO: 620 - protein, 106 aa, Homo sapiens; identical to SEQ ID NO: 335

SEQ ID NO: 621 - protein, 106 aa, Homo sapiens; identical to SEQ ID NO: 353

SEQ ID NO: 622 - protein, 106 aa, Homo sapiens; identical to SEQ ID NO: 299

SEQ ID NO: 623 - protein, 106 aa, Homo sapiens; identical to SEQ ID NO: 389

SEQ ID NO: 624 - protein, 106 aa, Homo sapiens; identical to SEQ ID NO: 299

SEQ ID NO: 625 - protein, 106 aa, Homo sapiens; identical to SEQ ID NO: 425

SEQ ID NO: 626 - protein, 106 aa, Homo sapiens
SYELIQPPSVSVSPGQTASITCSGHNLEDKFASWYQQKPGQSPVLVIYRDDKRPSGIPERFSATNSGHTATLTISGTQATDEADYYCSSQDKVTRVFGGGTKLTVL

SEQ ID NO: 627 - protein, 106 aa, Homo sapiens; identical to SEQ ID NO: 461

SEQ ID NO: 628 - protein, 125 aa, Homo sapiens; identical to SEQ ID NO: 20

SEQ ID NO: 629 - protein, 125 aa, Homo sapiens; identical to SEQ ID NO: 182

SEQ ID NO: 630 - protein, 125 aa, Homo sapiens; identical to SEQ ID NO: 236

SEQ ID NO: 631 - protein, 125 aa, Homo sapiens; identical to SEQ ID NO: 344

SEQ ID NO: 632 - protein, 125 aa, Homo sapiens; identical to SEQ ID NO: 398

SEQ ID NO: 633 - protein, 125 aa, Homo sapiens; identical to SEQ ID NO: 434

SEQ ID NO: 634 - protein, 106 aa, Homo sapiens; identical to SEQ ID NO: 29

SEQ ID NO: 635 - protein, 106 aa, Homo sapiens; identical to SEQ ID NO: 191

SEQ ID NO: 636 - protein, 106 aa, Homo sapiens; identical to SEQ ID NO: 245

SEQ ID NO: 637 - protein, 106 aa, Homo sapiens; identical to SEQ ID NO: 353

SEQ ID NO: 638 - protein, 106 aa, Homo sapiens; identical to SEQ ID NO: 299

SEQ ID NO: 639 - protein, 106 aa, Homo sapiens; identical to SEQ ID NO: 626

SEQ ID NO: 640 - protein, 17 aa, Homo sapiens
  VARIANT (4)..(4): /replace=“Ser” or “Ala”
  VARIANT (5)..(5): /replace=“Ser” or “Asn” or “Asp” or “Gly” or “Gln”
  VARIANT (6)..(6): /replace=“Gly” or “Thr” or “Pro”
  VARIANT (7)..(7): /replace=“Gly” or “Asn” or “Lys” or “Thr”
  VARIANT (8)..(8): /replace=“Thr” or “Arg” or “Lys”
  VARIANT (9)..(9): /replace=“Thr” or “Lys” or “Val”
  VARIANT (10)..(10): /replace=“Val” or “Thr”
  MISC_FEATURE (1)..(17): /note=“Variant residues given in the sequence have no preference with respect to those in the annotations for variant positions”
VIGKTNENIAYADSVKG

SEQ ID NO: 641 - protein, 16 aa, Homo sapiens
EWMDHSRPYYYYGMDV

SEQ ID NO: 642 - protein, 11 aa, Homo sapiens
  VARIANT (5)..(5): /replace=“Ile”
  VARIANT (6)..(6): /replace=“Gly”
  VARIANT (9)..(9): /replace=“Trp”
  VARIANT (10)..(10): /replace=“Val”
  MISC_FEATURE (1)..(11): /note=“Variant residues given in the sequence have no preference with respect to those in the annotations for variant positions”
SGHNLEDKFAS

SEQ ID NO: 643 - protein, 7 aa, Homo sapiens
RDDKRPS

SEQ ID NO: 644 - protein, 9 aa, Homo sapiens
  VARIANT (1)..(1): /replace=“Gln”
  VARIANT (2)..(2): /replace=“Ala”
  VARIANT (5)..(5): /replace=“Ser”
  VARIANT (6)..(6): /replace=“Thr”
  MISC_FEATURE (1)..(9): /note=“Variant residues given in the sequence have no preference with respect to those in the annotations for variant positions”
SSQDTVTRV

SEQ ID NO: 645 - protein, 17 aa, Artificial Sequence
  source: /note=“Description of Artificial Sequence Synthetic peptide”
  VARIANT (4)..(4): /replace=“Ser” or “Pro” or “Ala” or “Asn” or “Gly” or “Glu” or “Asp” or “Val” or “Thr”
  VARIANT (5)..(5): /replace=“Ser” or “Asn” or “His” or “Gln” or “Asp” or “Gly” or “Glu”
  VARIANT (6)..(6): /replace=“Gly” or “Pro” or “Thr” or “Gln” or “Glu” or “Met” or “Lys” or “Ala”
  VARIANT (7)..(7): /replace=“Gly” or “Lys” or “Asn” or “Gln” or “Thr” or “His” or “Asp” or “Ala”
  VARIANT (8)..(8): /replace=“Thr” or “Ala” or “Arg” or “Lys”
  VARIANT (9)..(9): /replace=“Thr” or “Asn” or “Ser” or “Lys” or “Phe” or “Gln” or “Val” or “Leu”
  VARIANT (10)..(10): /replace=“Val” or “Ser” or “Thr” or “Asn”
  VARIANT (14)..(14): /replace=“Ala” or “Thr”
  MISC_FEATURE (1)..(17): /note=“Variant residues given in the sequence have no preference with respect to those in the annotations for variant positions”
VIGKTNENIAYADSVKG

SEQ ID NO: 646 - protein, 16 aa, Artificial Sequence
  source: /note=“Description of Artificial Sequence Synthetic peptide”
  VARIANT (4)..(4): /replace=“Gly”
  VARIANT (5)..(5): /replace=“Arg”
  VARIANT (14)..(14): /replace=“Ile”
  VARIANT (16)..(16): /replace=“Ala”
  MISC_FEATURE (1)..(16): /note=“Variant residues given in the sequence have no preference with respect to those in the annotations for variant positions”
EWMDHSRPYYYYGMDV

SEQ ID NO: 647 - protein, 7 aa, Artificial Sequence
  source: /note=“Description of Artificial Sequence Synthetic peptide”
RDDKRPS

SEQ ID NO: 648 - protein, 10 aa, Artificial Sequence
  source: /note=“Description of Artificial Sequence Synthetic peptide”
CEVNLDAEFK